(19) United States
(12) Patent Application Publication
(10) Pub. No.: US 2021/0267957 A1
(43) Pub. Date: Sep. 2, 2021

(54) METHYLPHENIDATE COMPOSITIONS FOR TREATMENT OF ATTENTION DEFICIT HYPERACTIVITY DISORDER

(71) Applicant: Ironshore Pharmaceuticals & Development, Inc., Camana Bay (KY)

(72) Inventors: David Lickrish, Camana Bay (KY); Beverly J. Incledon, George Town (KY); Roberto Gomeni, Camana Bay (KY); Joga Gobburu, Herndon, VA (US)

(21) Appl. No.: 17/316,252

(22) Filed: May 10, 2021

Related U.S. Application Data
(63) Continuation of application No. 17/315,133, filed on May 7, 2021.
Application No. 17/315,133 is a continuation of application No. PCT/US2020/014926, filed on Jan. 24, 2020.
(60) Provisional application No. 62/962,355, filed on Jan. 17, 2020.
(60) Provisional application No. 62/796,918, filed on Jan. 25, 2019.

(51) Int. Cl. A61K 31/4458; A61K 9/00; A61K 9/48; A61K 9/20; A61K 9/28

(52) U.S. Cl. CPC A61K 31/4458; A61K 9/0053; A61K 9/4866; A61K 9/4891; A61K 9/2009; A61K 9/2013; A61K 9/284; A61K 9/2054; A61K 9/4858

(57) ABSTRACT

A solid, oral pharmaceutical composition is described. The solid, oral pharmaceutical composition includes methylphenidate or a pharmaceutical salt thereof, wherein an in vivo absorption model of the solid, oral pharmaceutical composition has a function selected from the group consisting of: a single Weibull function, a double Weibull function, and a sigmoid eMax function. A correlation of a plurality of fractions of an in vitro dissolution of the solid, oral pharmaceutical composition with a same plurality of fractions of an in vivo absorption of the solid, oral pharmaceutical composition is non-linear. A method of treating a condition in a subject having a disorder or condition responsive to the administration of methylphenidate is also described. The method includes orally administering to the subject an effective amount of the solid, oral pharmaceutical composition.

### RELATED APPLICATIONS

This application claims priority to U.S. Provisional Application No. 62/796,918 filed Jan. 25, 2019 and U.S. Provisional Application No. 62/962,355 filed Jan. 17, 2020, the disclosures of which are hereby incorporated by reference in their entireties.

### TECHNICAL FIELD

The present disclosure relates to pharmaceutical compositions and methods for treating attention deficit disorder (ADD) or attention deficit hyperactivity disorder (ADHD) and related disorders.

### BACKGROUND

Methylphenidate is a central nervous system stimulant used to treat ADD and ADHD in children and adults. Despite its usefulness, methylphenidate is associated with rebound as the drug is metabolized and its effects are reduced. Rebound may cause a marked change in demeanor, excessive moodiness, irritability, anger, nervousness, sadness, crying, fatigue, and even an increase in the severity of ADD or ADHD symptoms during the rebound period.

### SUMMARY

According to a first aspect, a solid, oral pharmaceutical composition is described. The solid, oral pharmaceutical composition includes methylphenidate or a pharmaceutical salt thereof, wherein an in vivo absorption model of the solid, oral pharmaceutical composition has a function selected from the group consisting of: a single Weibull function:

$$r 1 ( t ) = {e}^{- ( {( \frac{t i m e}{t d} )}^{s s} )}$$

wherein td is a time necessary to absorb 63.2% of the methylphenidate or a pharmaceutical salt thereof released, and ss is a sigmoidicy factor; a double Weibull function:

$$r 2 ( t ) = ff \cdot {e}^{- ( {( \frac{t i m e}{t d} )}^{S S} )} + ( 1 - ff ) \cdot {e}^{- ( {( \frac{t i m e}{t d 1} )}^{S S 1} )}$$

wherein ff is a fraction of a dose released in a 1st process, td is a time necessary to absorb 63.2% of the dose released in the 1st process, td1 is a time necessary to absorb 63.2% of a dose released in the 2nd process, ss is a sigmoidicy factor for the 1st process, and ss1 is a sigmoidicity factor for the 2nd process; and a sigmoid eMax function:

$${r}_{v i t r o} ( t ) = \frac{t i m {e}^{g a}}{{EC}^{ga} + t i m {e}^{g a}}$$

wherein EC is a time to release 50% of the methylphenidate or a pharmaceutical salt thereof, and ga is a parameter characterizing the shape of an absorption curve of the methylphenidate or a pharmaceutical salt thereof. A correlation of a plurality of fractions of an in vitro dissolution of the solid, oral pharmaceutical composition with a same plurality of fractions of an in vivo absorption of the solid, oral pharmaceutical composition is non-linear.

In any of the disclosed implementations, the solid, oral pharmaceutical composition may further include the following details, which may be combined with one another in any combinations unless clearly mutually exclusive:

(i) the non-linear correlation of the plurality of fractions of the in vitro dissolution of the solid, oral pharmaceutical composition with the plurality of fractions of the in vivo absorption may best fit a fifth-degree polynomial function.

(ii) the non-linear correlation of the plurality of fractions of the in vitro dissolution of the solid, oral pharmaceutical composition with the plurality of fractions of the in vivo absorption may best fit a second-degree polynomial function, a third-degree polynomial function, a fourth-degree polynomial function, a fifth-degree polynomial function, or a sixth-degree polynomial function.

(iii) the plurality of fractions of the in vitro dissolution of the solid, oral pharmaceutical composition and the plurality of fractions of the in vivo absorption may include a plurality of values from 0 to 1.

(iv) the solid, oral pharmaceutical composition may be a multilayered solid, oral pharmaceutical composition including the methylphenidate or a pharmaceutical salt thereof, a sustained release layer; and a delayed release layer.

(v) the solid, oral pharmaceutical composition may include a core including methylphenidate or a pharmaceutical salt thereof, wherein the core, the sustained release layer and the delayed release layer each have a surface; and the sustained release layer and the delayed release layer may enclose the core.

(vi) the sustained release layer may enclose the core and the delayed release layer may enclose the sustained release layer.

(vii) the sustained release layer and the delayed release layer may incompletely enclose the surface of the core.

(viii) the delayed release layer may incompletely enclose the surface of the sustained release layer and/or the surface of the core.

(ix) the sustained release layer and the delayed release layer may enclose at least 90%, 91%, 92%, 93%, 94%, 95%, 96%, 97%, 98%, 99%, or 99.9% of the surface of the core.

(x) the delayed release layer may enclose at least 90%, 91%, 92%, 93%, 94%, 95%, 96%, 97%, 98%, 99%, or 99.9% of the surface of the core and/or the surface of the sustained release layer.

(xi) the multilayered solid, oral pharmaceutical composition may include a multilayered core, wherein the multilayered core has a surface, and the multilayered core includes a first layer including the methylphenidate or a pharmaceutical salt thereof and a second layer including a swellable layer including a superdisintegrant or an osmotic agent.

(xii) the multilayered solid, oral pharmaceutical composition may include a multilayered core, wherein the multilayered core has a surface, and the multilayered core includes a first layer including the methylphenidate or a pharmaceutical salt thereof and a second layer including the sustained release layer.

(xiii) the multilayered core may further include a third layer including the sustained release layer.

(xiv) the multilayered core may further include a third layer including a swellable layer including a superdisintegrant or an osmotic agent.

(xv) the delayed release layer may enclose the multilayered core.

(xvi) the delayed release layer may incompletely enclose the surface of the multilayered core.

(xvii) the delayed release layer may enclose at least 90%, 91%, 92%, 93%, 94%, 95%, 96%, 97%, 98%, 99%, or 99.9% of the surface of the multilayered core.

According to a second aspect, a method of treating a condition in a subject having a disorder or condition responsive to the administration of methylphenidate is described. The method includes orally administering to the subject an effective amount of a solid, oral pharmaceutical composition including methylphenidate or a pharmaceutical salt thereof, wherein an in vivo absorption model of the solid, oral pharmaceutical composition has a function selected from the group consisting of: a single Weibull function:

$$r 1 ( t ) = {e}^{- ( {( \frac{t i m e}{t d} )}^{s s} )}$$

wherein td is a time necessary to absorb 63.2% of the methylphenidate or a pharmaceutical salt thereof released, and ss is a sigmoidicy factor; a double Weibull function:

$$r 2 ( t ) = ff \cdot {e}^{- ( {( \frac{t i m e}{t d} )}^{S S} )} + ( 1 - ff ) \cdot {e}^{- ( {( \frac{t i m e}{t d 1} )}^{S S 1} )}$$

wherein ff is a fraction of a dose released in a 1st process, td is a time necessary to absorb 63.2% of the dose released in the 1st process, td1 is a time necessary to absorb 63.2% of a dose released in the 2nd process, ss is a sigmoidicy factor for the 1st process, and ss1 is a sigmoidicity factor for the 2nd process; and a sigmoid eMax function:

$${r}_{v i t r o} ( t ) = \frac{t i m {e}^{g a}}{{EC}^{ga} + t i m {e}^{g a}}$$

wherein EC is a time to release 50% of the methylphenidate or a pharmaceutical salt thereof, and ga is a parameter characterizing the shape of an absorption curve of the methylphenidate or a pharmaceutical salt thereof. A correlation of a plurality of fractions of an in vitro dissolution of the solid, oral pharmaceutical composition with a same plurality of fractions of an in vivo absorption of the solid, oral pharmaceutical composition is non-linear. The method produces an improvement in a behavior or an ability related to the disorder or condition over a period of time, wherein the administering reduces the variation in efficacy, or likelihood or severity of rebound, or both, of over the period of time.

In any of the disclosed implementations, the method may further include the following details, which may be combined with one another in any combinations unless clearly mutually exclusive:

(i) the period of time may begin at 8:00 am, 9:00 am, 10:00 am, 11:00 am, 12:00 pm, 1:00 pm, 2:00 pm, 3:00 pm, 4:00 pm, 5:00 pm, 6:00 pm, or 7:00 pm.

(ii) the period of time may begin at 8, 9, 10, 11, 12, 13, 14, 15, or 16 hours after administration of the composition.

(iii) the improvement may be measured by a validated rating scale, score or combined score.

(iv) the validated rating scale, score or combined score may be a Swanson, Kotkin, Agler, M-Flynn and Pelham (SKAMP) score, or a SKAMP-CS combined score.

(v) the variation in efficacy may be measured by a fluctuation index (FI):

$$FI = \frac{[ max ( C H P ) - min ( C H P ) ]}{average ( CHP )}$$

wherein CHP is a change from placebo of the SKAMP score during the period of time.

(vi) the fluctuation index (FI) may have an absolute value less than 1.0.

(vii) the period of time may end at 9:00 am, 10:00 am, 11:00 am, 12:00 pm, 1:00 pm, 2:00 pm, 3:00 pm, 4:00 pm, 5:00 pm, 6:00 pm, 7:00 pm, or 8:00 pm.

(viii) the period of time may end at 6, 7, 8, 9, 10, 11, 12, 13, 14, 15, or 16 hours after the start of the period of time.

(ix) the period of time may end when a plasma concentration of methylphenidate in the subject is below 5 ng/mL.

(x) the period of time may end at 3, 4, 5, 6, 7, 8, 9, 10, 11, or 12 hours after a methylphenidate Tmax in the subject.

(xi) the period of time may end when the subject falls asleep following Tmax.

(xii) during the period of time, the value of the SKAMP scores may not change by more than, or than about, 6, 7, 8, 9, or 10.

(xiii) during the period of time, a rate of change of the methylphenidate plasma concentration over time may not be greater than +2.5 ng·hr/mL following a dose of up to 100 mg methylphenidate.

(xiv) the period of time may be between Tmax and 6 hours after Tmax, and the rate of change of the methylphenidate plasma concentration is not less than −1.2 ng·hr/mL following a dose of up to 100 mg methylphenidate.

(xv) the period of time may include a period wherein a methylphenidate plasma concentration is between Cmax and at least 40% Cmax and a rate of change of the methylphenidate plasma concentration is not greater than +1.5 ng·hr/mL and not less than −1.5 ng·hr/mL.

(xvi) the methylphenidate or pharmaceutical salt thereof may be absorbed in the colon.

(xvii) at least 90% of the methylphenidate or pharmaceutical salt thereof may be absorbed in the colon.

(xviii) the subject may have attention deficit hyperactivity disorder (ADHD) or attention deficit hyperactivity disorder (ADD), and an autism spectrum disorder (ASD).

(xix) the improvement may be dose-dependent.

(xx) the dose-dependent improvement may include a dose-dependent increase in the period of time.

(xxi) the increase in the period of time may include an increase in a period of time after Tmax.

(xxii) the solid, oral pharmaceutical composition may be a multilayered solid, oral pharmaceutical composition including the methylphenidate or a pharmaceutical salt thereof, a sustained release layer; and a delayed release layer.

(xxiii) the solid, oral pharmaceutical composition may include a core including methylphenidate or a pharmaceutical salt thereof, wherein the core, the sustained release layer and the delayed release layer each have a surface; and the sustained release layer and the delayed release layer may enclose the core.

(xxiv) the sustained release layer may enclose the core and the delayed release layer may enclose the sustained release layer.

(xxv) the sustained release layer and the delayed release layer may incompletely enclose the surface of the core.

(xxvi) the delayed release layer may incompletely enclose the surface of the sustained release layer and/or the surface of the core.

(xxvii) the sustained release layer and the delayed release layer may enclose at least 90%, 91%, 92%, 93%, 94%, 95%, 96%, 97%, 98%, 99%, or 99.9% of the surface of the core.

(xxviii) the delayed release layer may enclose at least 90%, 91%, 92%, 93%, 94%, 95%, 96%, 97%, 98%, 99%, or 99.9% of the surface of the core and/or the surface of the sustained release layer.

(xxix) the multilayered solid, oral pharmaceutical composition may include a multilayered core, wherein the multilayered core has a surface, and the multilayered core includes a first layer including the methylphenidate or a pharmaceutical salt thereof and a second layer including a swellable layer including a superdisintegrant or an osmotic agent.

(xxx) the multilayered solid, oral pharmaceutical composition may include a multilayered core, wherein the multilayered core has a surface, and the multilayered core includes a first layer including the methylphenidate or a pharmaceutical salt thereof and a second layer including the sustained release layer.

(xxxi) the multilayered core may further include a third layer including the sustained release layer.

(xxxii) the multilayered core may further include a third layer including a swellable layer including a superdisintegrant or an osmotic agent.

(xxxiii) the delayed release layer may enclose the multilayered core.

(xxxiv) the delayed release layer may incompletely enclose the surface of the multilayered core.

(xxxv) the delayed release layer may enclose at least 90%, 91%, 92%, 93%, 94%, 95%, 96%, 97%, 98%, 99%, or 99.9% of the surface of the multilayered core.

### BRIEF DESCRIPTION OF THE DRAWINGS

For a more complete understanding of the present disclosure and the associated features and advantages, reference is now made to the following description, taken in conjunction with the accompanying drawings, which are not to scale, and in which:

FIG. 1 is a schematic of the pharmacokinetic (PK) model for the delayed-release/extended release methylphenidate formulation HLD200;

FIG. 2 are exemplary scatter plots of the individual subject PK profiles by dose of HLD 200 in linear scale;

FIG. 3 are exemplary scatter plots of the individual subject PK profiles by dose of HLD 200 in log-linear scale;

FIG. 4 is an exemplary scatter plot of the mean (±standard deviation (SD)) PK profiles by dose of HLD200 in linear scale in a total test adult subject population;

FIG. 5A is an exemplary scatter plot of the mean (±SD) PK profiles for a 20 mg dose of HLD200 in linear scale by subject gender;

FIG. 5B is an exemplary scatter plot of the mean (±SD) PK profiles for a 100 mg dose of HLD200 in linear scale by subject gender;

FIG. 6 is an exemplary scatter plot of the mean (±SD) PK profiles by dose of HLD200 in log-linear scale in the total test subject population;

FIG. 7A is an exemplary scatter plot of the mean (±SD) PK profiles for a 20 mg dose of HLD200 in log-linear scale by subject gender;

FIG. 7B is an exemplary scatter plot of the mean (±SD) PK profiles for a 100 mg dose of HLD200 in log-linear scale by subject gender;

FIG. 8 are exemplary goodness-of-fit plots for the base subject population PK model at the dose of 20 mg of HLD200;

FIG. 9 are exemplary goodness-of-fit plots for the base subject population PK model at the dose of 100 mg of HLD 200;

FIG. 10 is a set of exemplary exploratory covariates analysis plots of the relationship between weight and kel, V, TD, and SS in subjects administered HLD200;

FIG. 11 is a set of exemplary exploratory covariates analysis plots of the relationship between gender (0=F, 1=M) and kel, V, TD, and SS in subjects administered HLD200;

FIG. 12 is a set of exemplary goodness-of-fit plots for the final test subject population PK model at a dose of 20 mg HLD200;

FIG. 13 is a set of exemplary goodness-of-fit plots for the final test population PK model at a dose of 100 mg HLD200;

FIG. 14 is a set of exemplary graphs reporting model predicted and observed HLD200 PK concentrations vs time for individual subjects.

FIG. 15 is a set of exemplary graphs reporting model predicted and observed HLD200 PK concentrations vs. time for individual subjects;

FIG. 16 is a set of exemplary graphs reporting model predicted and observed HLD200 PK concentrations vs. time for individual subjects;

FIG. 17 is a set of exemplary graphs reporting model predicted and observed HLD200 PK concentrations vs. time for individual subjects;

FIG. 18 is a set of exemplary graphs reporting model predicted and observed HLD 200 PK concentrations vs. time for individual subjects;

FIG. 19 is a set of graphs reporting exemplary Visual Predictive Check. The solid lines represent the median predicted MPH concentrations, the circles represent the observed MPH concentrations, the thick dashed lines represent the median observed concentration and the area between the light dashed lines represents the 90% prediction interval of the simulated data. If most of the observed data fit within the confidence intervals of the model (the area between the light dashed lines) then the Visual Predictive Check is successful;

FIG. 20 is a set of graphs reporting exemplary impact of weight and gender on the expected PK profiles of HLD200 at a dose of 20 mg or 100 mg;

FIG. 21 is a graph reporting exemplary Simulated methylphenidate (MPH) exposure after repeated administrations of HLD200;

FIG. 22 is a set of graphs reporting exemplary comparison of the HLD200 exposure to the exposure of Concerta® (ALZA Corporation) in the test subject population. The time of HLD drug intake is in the evening at 8 hours (top panel) or 10 hours (bottom panel) before the start of school at 8:00 am;

FIG. 23 is a set of graphs reporting exemplary comparison of the HLD200 exposure to the exposure of Metadate® (UCB, Inc) in the test subject population. The time of HLD drug intake is in the evening at 8 hours (top panel) or 10 hours (bottom panel) before the start of school at 8:00 am;

FIG. 24 is a set of graphs reporting exemplary comparison of the HLD200 exposure to the exposure of Ritalin LA® (Novartis AG) in the test subject population. The time of HLD drug intake is in the evening at 8 hours (top panel) or 10 hours (bottom panel) before the start of school at 8:00 am;

FIG. 25 is a set of graphs reporting exemplary comparison of the HLD200 exposure to the exposure of Quillivant XR® (NextWave Pharmaceuticals, Inc.) in the test subject population. The time of HLD drug intake is in the evening at 8 hours (top panel) or 10 hours (bottom panel) before the start of school at 8:00 am;

FIG. 26 is a set of plots reporting exemplary individual patient Swanson, Kotkin, Agler, M-Flynn, and Pelham Scale (SKAMP) scores by treatment;

FIG. 27 is a graph reporting exemplary mean (±SD) SKAMP score profiles by treatment in the total test patient population;

FIG. 28 is a graph reporting exemplary mean (±SD) SKAMP score profiles by treatment and gender in the total test patient population;

FIG. 29 is a graph reporting exemplary mean (±SD) estimated MPH concentrations in the total test patient population;

FIG. 30 is a graph reporting exemplary mean (±SD) estimated MPH concentrations by gender in the total test patient population;

FIG. 31 is a set of graphs reporting exemplary goodness-of-fit plots for a comparative placebo response model;

FIG. 32 is a graph reporting exemplary Visual Predictive Check on the placebo response model. The thick dashed line represents the median predicted SKAMP scores, the circles represent the observed SKAMP scores, the solid thick line represents the median observed SKAMP scores and the area between the light dashed lines represents the 90% prediction interval of the simulated data;

FIG. 33 is a set of graphs reporting exemplary goodness-of-fit plots for a base population PK/PD model;

FIG. 34 is graphs reporting exemplary exploratory analysis on covariates: relationship between weight, age and gender on EMAX and EC50;

FIG. 35 is graphs reporting exemplary goodness-of-fit plots for the final population PK/PD model;

FIG. 36 is a graph reporting exemplary PK/pharmacodynamic (PD) model—Visual Predictive Check. The thick dashed line represents the median predicted concentrations, the circles represent the observed scores, the solid thick line represents the median scores and the area between the light dashed lines represents the 90% prediction interval of the simulated data;

FIG. 37 is a graph reporting exemplary relationship between HLD200 drug exposure and clinical response in test patients defined by the change from the placebo response with the 90% prediction interval (shaded area);

FIG. 38 is a schematic illustrating an exemplary definition of clinical benefit (CB);

FIG. 39 is a set of graphs reporting exemplary impact of time of HLD200 intake on the clinical benefit in the total test patient population;

FIG. 40 is a graph reporting exemplary simulated time course of the HLD200 plasma concentrations (heavy solid line) and of SKAMP scores after a 60 mg HLD200 dose (dashed heavy line), and after a placebo dose (light solid line);

FIG. 41 is a graph reporting exemplary simulated time course of the HLD200 plasma concentrations (heavy solid line) and of SKAMP scores after an 80 mg HLD200 dose (dashed heavy line), and after a placebo dose (light solid line);

FIG. 42 is a graph reporting exemplary simulated time course of the HLD200 plasma concentrations (heavy solid line), and of SKAMP scores after a 100 mg HLD200 dose (dashed heavy line), and after a placebo dose (light solid line);

FIG. 43 is a graph reporting an exemplary simulation of the time course of SKAMP scores after doses of 60 mg, 80 mg, and 100 mg of HLD200 and of SKAMP scores after a placebo dose;

FIG. 44 is a set of graphs reporting exemplary data from a COMACS study: Mean SKAMP total scores versus time by MCD dose level. From Sonuga-Barke E J, Swanson J M, Coghill D, DeCory H H, Hatch S J. Efficacy of two once-daily methylphenidate formulations compared across dose levels at different times of the day: preliminary indications from a secondary analysis of the COMACS study data. BMC Psychiatry. 2004 Sep. 30; 4:28, incorporated by reference herein;

FIG. 45 is a graph reporting exemplary data from a d-MPH ER study: Mean SKAMP-Combined raw scores from predose (hour 0) through hour 12. From Raul R. Silva et al. Efficacy and Duration of Effect of Extended-Release Dexmethylphenidate Versus Placebo in School children With Attention-Deficit/Hyperactivity Disorder. Journal Of Child And Adolescent Psychopharmacology. Volume 16, Number 3, 2006, incorporated by reference herein;

FIG. 46 is a graph reporting exemplary data from a NWP06 (Quillivant XR® (NextWave Pharmaceuticals, Inc.)) study: Mean SKAMP-Combined scores from predose (hour 0) through hour 12. From Sharon B. Wigal et al., NWP06, an Extended-Release Oral Suspension of Methylphenidate, Improved Attention-Deficit/Hyperactivity Disorder Symptoms Compared with Placebo in a Laboratory Classroom Study. Journal Of Child And Adolescent Psychopharmacology. Volume 23, Number 1, 2013, incorporated by reference herein;

FIG. 47 is a graph reporting an exemplary comparison of the change as compared to a placebo dose of SKAMP scores of patients receiving HLD200 with the data on Concerta® (ALZA Corporation) (at doses of 18 mg, 36 mg and 54 mg), with the data on Quillivant XR® (NextWave Pharmaceuticals, Inc.), and with the data on d-MPH;

FIG. 48 is a graph reporting an exemplary comparison of the change as compared to a placebo of SKAMP scores of patients receiving HLD200 with the data on Metadate CD® (UCB, Inc) (at the dose of 20 mg, 40 mg and 60 mg), with the data on Quillivant XR® (NextWave Pharmaceuticals, Inc.), and with the data on d-MPH;

FIG. 49 is a set of graphs reporting exemplary dissolution model predicted and observed fraction release in vitro for fast, slow and final formulations of delayed-release/extended-release MPH;

FIG. 50 is a set of graphs reporting exemplary observed (dots) and predicted (line) mean MPH concentrations after the administration of 20 mg Ritalin® (Novartis) IR;

FIG. 51 is a graph reporting exemplary fraction of methylphenidate released In Vitro vs. In Vivo of HLD200 for the fast and slow54 formulations combined;

FIG. 52 is a set of graphs reporting exemplary internal validation of the IVIVC model: observed (dots) and IVIVC predicted (line) methylphenidate concentrations for the fast (study HLD200-101, 54 mg) and slow (study HLD200-101, 54 mg) formulations;

FIG. 53 is a graph reporting exemplary External Validation of the IVIVC model: Observed (Dots) and IVIVC Predicted (Line) Methylphenidate Concentrations for the Slow100 and Final formulations;

FIG. 54A is an exemplary schematic representation of a bead pharmaceutical composition with a drug containing core surrounded by a sustained release layer and a delayed release layer.

FIG. 54B is an exemplary schematic representation of a composition as in FIG. 54A with an added swellable layer disposed between the sustained release layer and the drug containing core;

FIG. 55A is an exemplary schematic representation of a minitablet pharmaceutical composition with a drug containing core surrounded by a sustained release layer and a delayed release layer;

FIG. 55B is an exemplary schematic representation of a composition as in FIG. 55A with an added swellable layer disposed between the sustained release layer and the drug containing core;

FIG. 56 is an exemplary schematic representation of a bead pharmaceutical composition that includes a core surrounded by four layers, an inert inner core, a swelling polymer, a drug layer, a sustained release layer and an enteric layer;

FIG. 57 is a graph reporting exemplary data on the fraction of methylphenidate dissolved in vitro (FDISS) versus the fraction of methylphenidate absorbed in vivo (Fabs) from Concerta® (ALZA Corporation). From R. Gomeni, F. Bressolle, T. J. Spencer, S. V. Faraone. Meta-analytic approach to evaluate alternative models for characterizing the PK profiles of extended release formulations of MPH. ASCPT 2016 Annual Meeting, Mar. 8-12, 2016, Hilton Bayfront, San Diego, Calif., incorporated by reference herein;

FIG. 58 is a set of graphs reporting exemplary observed (dots) and predicted (line) mean methylphenidate concentrations after the administration of the indicated drugs. From R. Gomeni, F. Bressolle, T. J. Spencer, S. V. Faraone. Meta-analytic approach to evaluate alternative models for characterizing the PK profiles of extended release formulations of MPH. ASCPT 2016 Annual Meeting, Mar. 8-12, 2016, Hilton Bayfront, San Diego, Calif., incorporated by reference herein.

FIG. 59 is a graph reporting exemplary data of the cumulative fraction absorbed for Concerta® (ALZA Corporation);

FIG. 60 is a graph reporting exemplary “initial” and “optimized” fractional absorption profiles as determined by R. Gomeni et al. (ASCPT 2016 Annual Meeting, Mar. 8-12, 2016, Hilton Bayfront, San Diego, Calif., incorporated by reference herein);

FIG. 61 is a graph reporting exemplary fractional absorption profile of HLD200.

FIG. 62 is a graph reporting exemplary data on mean rate of change of methylphenidate plasma concentration over time (ng/mL/hour) for HLD20054 mg (study 200-101), HLD200100 mg (study 200-109) and CONCERTA® 54 mg;

FIG. 63 is a graph reporting exemplary fraction of amphetamine released In Vitro vs. In Vivo from an exemplary amphetamine formulation HLD100-102 having similar composition as HLD200 methylphenidate medium formulation;

FIG. 64 is a graph reporting exemplary cumulative % colon arrival time for surrogate beads radio-labelled with not more than 1 MBq ¹¹¹Indium. The plots show results from two separate experiments, indicated as “F1” and “F2”;

FIG. 65 is a graph reporting exemplary Visual predictive checks of the PK data after IR formulation. The heavy solid line represents the median prediction with the 95% prediction interval (shaded area). The dots represent the observed MPH concentrations;

FIG. 66 is a graph reporting exemplary Mean dissolution data for the Slow, Med, and Fast dissolution rate formulations (dots) with the model predicted dissolution curves (solid and dashed lines as indicated);

FIG. 67 is a schematic showing exemplary Convolution-based model used to fit the in-vivo PK of the fast, medium and slow HLD200 formulations;

FIG. 68 is graphs reporting exemplary mean PK data for the 3 formulations with the model predicted curves (top panel) and the in-vivo release rate (bottom panel);

FIG. 69 is a graph reporting exemplary mean PK observed concentrations (dots) with the predicted values by the convolution model (solid and dashed lines as indicated) for the fast, medium and slow HLD200 formulations;

FIG. 70 is a graph reporting exemplary regression analysis of the in-vivo absorption rate versus the in-vitro dissolution rate for the Slow, Med, and Fast release HLD200 formulations;

FIG. 71 is a graph reporting exemplary regression analysis of the predicted concentrations by the convolution model versus the observed concentrations (dots) for the Slow, Med, and Fast release HLD200 formulations;

FIG. 72 is graphs reporting exemplary scatter plots of the TD parameters characterizing the in-vitro dissolution and the in-vivo BI (left panel) and Kel (right panel) with the model predicted relationship (dashed line);

FIG. 73 is a schematic showing exemplary refined IVIVC model including the dependency between dissolution properties and the estimated in-vivo relative bioavailability;

FIG. 74 is a graph reporting exemplary mean PK observed concentrations (dots) with the predicted values by the refined convolution model (solid and dashed lines as indicated) for the Slow, Med, and Fast release HLD200 formulations;

FIG. 75 is a graph reporting exemplary regression analysis of the predicted concentrations by the refined convolution model versus the observed concentrations (dots) for the Slow, Med, and Fast release HLD200 formulations;

FIG. 76 is graphs reporting exemplary mean dissolution data (dots) for the formulation used in the external validation (upper panel) and the formulations with Slow, Med, and Fast dissolution rate formulations used in model development (lower panel) with the model predicted dissolution curves (solid and dashed lines as indicated);

FIG. 77 is a graph reporting exemplary comparison of the mean PK concentrations time-course for the formulation with median dissolution rate in the study HLD200-111 and the PK concentrations time-course in the study HLD200-109 (Fasted arm);

FIG. 78 is a graph reporting exemplary Visual predictive checks of the PK data of the study HLD200-109. The thick solid line represents the median prediction with the 95% prediction interval (shaded area). The dots represent the observed MPH concentrations;

FIG. 79 is a graph reporting exemplary comparison of the typical PK time course in the study HLD200-109 (solid curve) with the convolution-based estimate of the expected PK profile (dashed curve);

FIG. 80 is a graph reporting exemplary external validation: regression analysis of the predicted concentrations by the refined convolution model versus the observed concentrations (dots);

FIG. 81 is a graph reporting an exemplary in vitro dissolution plot of the HLD200 medium formulation (also referred to herein as the final formulation) together with an exemplary in vitro dissolution plot of an amphetamine formulation (referred to herein as HLD100-102);

FIG. 82 is a graph reporting exemplary regression analysis of the in-vivo absorption rate versus the in-vitro dissolution rate for the fast release HLD200 formulation;

FIG. 83 is a graph reporting exemplary regression analysis of the in-vivo absorption rate versus the in-vitro dissolution rate for the medium release HLD200 formulation; and

FIG. 84 is a graph reporting exemplary regression analysis of the in-vivo absorption rate versus the in-vitro dissolution rate for the slow release HLD200 formulation.

FIG. 85 is an exemplary schematic depicting a refined IVIVC model including the dependency between dissolution properties and the estimated in-vivo relative bioavailability.

FIG. 86A is a graph reporting exemplary values for plasma MPH concentration versus time (hours) allowing visual predictive check for the IR MPH model (step 1).

FIG. 86B is a graph reporting exemplary values for fraction of dose released in vitro (%) versus time (hours) for model-predicted in vitro DR/ER-MPH Dissolution Profiles (step 2).

FIG. 86C is a graph reporting exemplary values for Median Model-Predicted DR/ER-MPH PK Curves (step 3).

FIG. 86D is a graph reporting exemplary values for Mean Plasma MPH Concentrations Predicted by the Convolution Model (step 4).

FIG. 87 is a graph reporting exemplary regression analysis of the in-vivo absorption rate versus the in-vitro dissolution rate for the Slow, Med, and Fast release formulations.

FIG. 88 is a graph reporting an exemplary comparison of the observed dissolution data and the model predicted dissolution data using a single Weibull model (solid lines) and a double Weibull model (dotted lines).

FIG. 89 is a graph reporting an exemplary comparison of the observed dissolution data and the model predicted dissolution data using a sigmoid Emax (solid lines) and a double Weibull model (dotted lines).

FIG. 90 is an exemplary schematic depicting a convolution model used for the assessment of an IVIVC relationship.

FIG. 91 is graphs reporting exemplary values for Fast release formulation: Fraction absorbed vs. fraction dissolved (upper panel) and Levy plot (lower panel).

FIG. 92 is graphs reporting exemplary values for Med release formulation: Fraction absorbed vs. fraction dissolved (upper panel) and Levy plot (lower panel).

FIG. 93 is graphs reporting exemplary values for Slow release formulation: Fraction absorbed vs. fraction dissolved (upper panel) and Levy plot (lower panel).

### DETAILED DESCRIPTION

In the following description, details are set forth by way of example to facilitate discussion of the disclosed subject matter. It should be apparent to a person of ordinary skill in the art, however, that the disclosed implementations are exemplary and not exhaustive of all possible implementations.

The present disclosure relates to compositions and methods providing minimal variation in methylphenidate efficacy in a patient during a period of time, for reducing the likelihood or severity of rebound, or both.

The term “subject” as used herein refers to an individual human being, including without limitation in various implementations, a child, an adult, or an adolescent. In some implementations, the subject can be a “patient”, meaning a subject who may be under the care of a physician and/or who may have been diagnosed as having ADD or ADHD, and also possibly other disorders such as autism or autism spectrum disorder.

Methylphenidate is used to treat a variety of disorders, particularly ADD and ADHD. Methylphenidate is generally effective over a period of time. Often the period of time includes time during the day when a child is at school. For example, the period may typically last between 4 and 12 hours. Actual efficacy of many methylphenidate drugs during the period can exhibit significant variation. For example, efficacy of improving performance according to a validated rating scale, such as a SKAMP score, may vary. In some implementations, compositions and methods disclosed herein can reduce variation in efficacy, which may be measured, for example, by reduced variation in SKAMP scores. In some implementations, composition and methods disclosed herein can reduce adverse events reporting in the late afternoon or evening, such as one or more of increased aggression, affectability, mood instability, and irritability, among others.

At or near the end of the efficacious period of time, many patients exhibit rebound, during which other symptoms appear or ADD and ADHD symptoms reappear and may be worse than they typically are without treatment. Rebound can interfere with homework and bedtime. Compositions and methods disclosed herein can have a longer period of efficacy with a more gradual reduction in methylphenidate plasma levels at the later times during the day. The longer efficacy and the lack of a sharp drop in methylphenidate plasma levels can help reduce or avoid rebound.

Some patients are particularly likely to experience or be sensitive to the negative effects of variations in efficacy, rebound, or both. These variation-sensitive, rebound-sensitive, and variation- and rebound-sensitive patients are particularly likely to benefit from the compositions and methods disclosed herein.

Variation-sensitive, rebound-sensitive, and variation and rebound-sensitive patients often include patients with comorbidity, particularly with another behavior disorder, or mental health disorder. In particular, patients with both ADD or ADHD and an autism spectrum disorder (ASD) may be variation-sensitive, rebound-sensitive, and variation- and rebound-sensitive patients.

The present disclosure provides therapeutic compositions and methods for treatment of attention deficit disorder (ADD), attention deficit hyperactivity disorder (ADHD) or other conditions or disorders responsive to CNS stimulants by providing dosage forms that deliver a therapeutic amount of active drug in a delayed and controlled release pattern in order to maintain a therapeutic amount of drug through the active portion of the day. For pediatric patients including adolescents and also for adults, a therapeutic amount is desirable upon arising and throughout the morning, as well as through the afternoon hours in which work or homework needs to be done.

Compositions of the present disclosure include a solid, oral pharmaceutical composition that has a core that includes methylphenidate or a pharmaceutical salt thereof and at least one pharmaceutically acceptable excipient, a sustained release layer enclosing the core, and a delayed release layer enclosing the sustained release layer.

The disclosed formulations can provide a therapeutic amount of drug during extended periods of the day with a single administration. The dosage forms provide a delayed release such that the dosage form can be administered conveniently prior to the patient's sleeping. A small percentage of the drug can be released over the first 6-hours after administration such that the patient has already received a minimal therapeutic dose at the normal awakening time. The patient thus does not need to be awakened, given a pill, and then required to have breakfast and prepare for their day prior to experiencing a therapeutic effect.

Stimulant medications (e.g., methylphenidate and amphetamines and prodrugs) are often prescribed to treat individuals diagnosed with ADHD. According to the National Institute of Health, all stimulants work by increasing dopamine levels in the brain. Dopamine is a brain chemical (or neurotransmitter) associated with pleasure, movement, and attention. The therapeutic effect of stimulants is achieved by slow and steady increases of dopamine, which are similar to the natural production by the brain. The doses prescribed by physicians start low and increase gradually until a therapeutic effect is reached.

Treatment of ADHD with stimulants, often in conjunction with psychotherapy, helps to improve the symptoms of ADHD, as well as the self-esteem, cognition, and social and family interactions of the patient. The most commonly prescribed medications include amphetamines and methylphenidate. These medications have a paradoxically calming and “focusing” effect on individuals with ADHD. Researchers speculate that because methylphenidate amplifies the release of dopamine, it can improve attention and focus in individuals who have dopamine signals that are weak.

Methylphenidate can be prescribed in a racemic mix of dextro and levo conformations or as the pure dextro isomer. Methylphenidate has two chiral centers in the molecule and thus can also be further refined to enrich the d threo isomer. The use of pharmaceutically acceptable salts of methylphenidate, such as methylphenidate hydrochloride, is also contemplated by the present disclosure.

It is understood that the active pharmaceutical ingredients of the present disclosure can be present as prodrugs that are activated in the body of a user. One form of prodrug has an amino acid conjugated to the active ingredient. When the amino acid is enzymatically cleaved, the active drug is released. Prodrugs comprising a lysyl, isoleucyl or aspartyl conjugate are contemplated to be useful in the practice of the present disclosure.

The formulations of the disclosure are designed to provide novel release and plasma profiles that include a first lag phase followed by a sigmoidal release phase. By providing this profile, the dosage forms provide a timed, prolonged therapeutic effect when taken once a day. Based on the release characteristics, in which the dosage form passes through the stomach prior to release, the formulations disclosed herein provide at least the following further advantages: low variability in gastric emptying, low risk of sudden dose dumping, low incidence of gastric discomfort and low intra- and inter-individual variability.

In some embodiments, the solid, oral pharmaceutical composition of the present disclosure may be multilayered. In some embodiments, the solid, oral pharmaceutical composition of the present disclosure may include methylphenidate or a pharmaceutical salt thereof, a sustained release layer, and a delayed release layer. In some embodiments, the solid, oral pharmaceutical composition of the present disclosure may include a core that includes methylphenidate or a pharmaceutical salt thereof, and a sustained release layer and a delayed release layer may enclose the core. In some embodiments, the sustained release layer may enclose the core and the delayed release layer may enclose the sustained release layer. In some embodiments, the solid, oral pharmaceutical composition of the present disclosure has a core that includes methylphenidate or a pharmaceutical salt thereof and at least one pharmaceutically acceptable excipient, a sustained release layer enclosing the core, and a delayed release layer enclosing the sustained release layer.

In some embodiments, the sustained release layer and the delayed release layer may incompletely enclose the core. For example, in some embodiments, the sustained release layer and the delayed release layer may individually or together enclose at least 90%, 91%, 92%, 93%, 94%, 95%, 96%, 97%, 98%, 99%, or 99.9% of the surface of the core. In some embodiments, the delayed release layer may incompletely enclose the sustained release layer and/or the core. For example, in some embodiments, the delayed release layer may enclose at least 90%, 91%, 92%, 93%, 94%, 95%, 96%, 97%, 98%, 99%, or 99.9% of the surface of the core and/or the sustained release layer.

In some embodiments, the multilayered solid, oral pharmaceutical composition of the present disclosure may include a multilayered core. In some embodiments, the multilayered core may include methylphenidate or a pharmaceutical salt thereof and at least one pharmaceutically acceptable excipient.

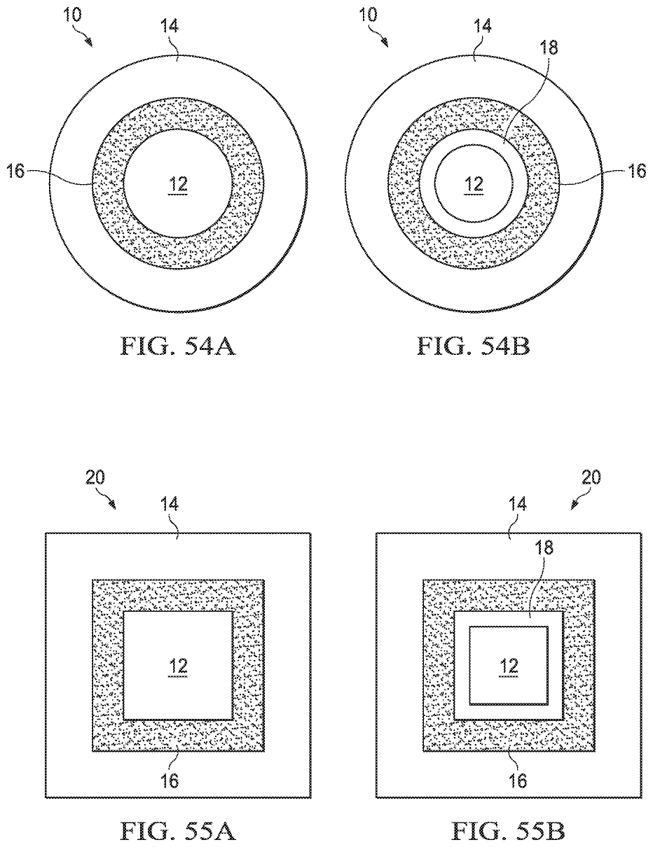

A first example of a dosage form is a single population of beads that can be administered in a capsule or a liquid or gel suspension containing the beads. An example of a bead structure 10 is shown in schematic form in FIG. 54A and FIG. 54B. In FIG. 54A, the inner circle represents a drug containing core, which includes the active ingredient or prodrug, the appropriate excipients and optionally a superdisintegrant or osmagent. A core can include, for example, an active agent, a disintegrant, osmagent, or pore-forming agent, and a binder. An exemplary core includes about 20-25% active agent, about 45-60% microcrystalline cellulose, about 10-30% potassium chloride and about 3-5% binder such as polyvinyl pyrrolidone or hydroxypropyl cellulose, for example. The drug containing core can be made by a variety of processes known in the art, including wet granulation, extrusion, and spheronization. In some embodiments, two layers enclose the core. In some embodiments, the first layer is a sustained release layer and the outer layer is a delayed release layer that is optionally pH dependent. In certain embodiments, the core as shown in FIG. 54A can be an inert non-pareil bead. The inner core may be a bead of sugar and starch or it can be composed of microcrystalline cellulose. Any spherical bead that is suitable for forming the core bead and is pharmaceutically acceptable can be used. In such embodiments, the drug and excipients of the core may be layered onto the core bead, providing a three layer formulation.

In some embodiments, the outermost layer 14 is a delayed release or an enteric coating. In certain embodiments this layer comprises a water-soluble polymer, a water-insoluble polymer, a plasticizer and a lubricant. The time of delay of drug release may be controlled by the ratio of water-soluble and insoluble polymers, the plasticizer concentration, amount of lubricant, and the coating weight gain, which can be up to 35-45%. In some embodiments, the delayed release layer may be a pH dependent polymer that dissolves at pH above 5.5.

In some embodiments, a sustained release layer 16 is designed to provide a slower initial rate of release that increases over a period of up to 8-10 hours after the layer is exposed to an aqueous environment. In some embodiments, the delayed-release layer and/or the sustained release layer forms a semi-permeable membrane. The increasing drug profile can be achieved by a membrane that becomes more permeable over time. An example of a sustained release layer includes a water-soluble polymer, a water-insoluble polymer, a plasticizer and a lubricant. The rate of drug release can be controlled or sustained by varying the ratio of water-soluble and water-insoluble polymers and by varying the coating thickness up to 15-45% weight gain.

In some embodiments, the solid, oral pharmaceutical composition of the present disclosure may include a swellable layer including a superdisintegrant or osmotic agent. In some embodiments, the solid, oral pharmaceutical composition of the present disclosure may include a delayed release layer enclosing a swellable layer, a sustained release layer, and a core comprising methylphenidate or a pharmaceutical salt thereof. In some embodiments, the sustained delayed release layer may incompletely enclose the swellable layer, sustained release layer, and/or the core comprising methylphenidate or a pharmaceutical salt thereof. For example, in some embodiments, the delayed release layer may enclose at least 90%, 91%, 92%, 93%, 94%, 95%, 96%, 97%, 98%, 99%, or 99.9% of the surface of swellable layer, sustained release layer, and/or the core comprising methylphenidate or a pharmaceutical salt thereof. In some embodiments, the solid, oral pharmaceutical composition of the present disclosure may include a multilayered core that includes a swellable layer, sustained release layer, and/or the core comprising methylphenidate or a pharmaceutical salt thereof.

In some embodiments, the solid, oral pharmaceutical composition of the present disclosure includes a multilayered core. In some embodiments, the multilayered core includes a first layer including methylphenidate or a pharmaceutical salt thereof and a second layer including a superdisintegrant or an osmotic agent.

In some embodiments the solid, oral pharmaceutical composition of the present disclosure includes a delayed release layer enclosing the multilayered core. In some embodiments, the delayed release layer may incompletely enclose the multilayered core. For example, in some embodiments, the delayed release layer may enclose at least 90%, 91%, 92%, 93%, 94%, 95%, 96%, 97%, 98%, 99%, or 99.9% of the surface of the multilayered core.

For example, in some embodiments such as is shown in FIG. 54B, a swellable layer 18, including a superdisintegrant or osmotic agent is disposed between the core and the sustained release layer.

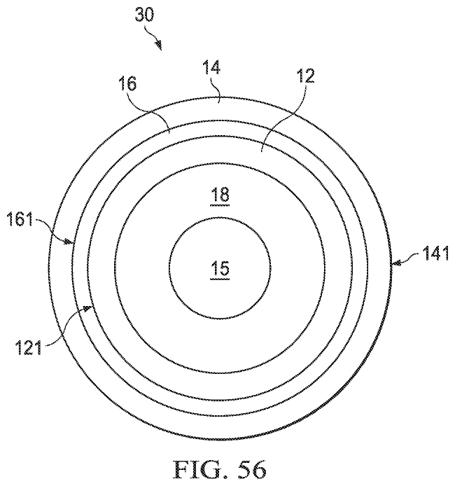

In certain embodiments, the compositions and methods of the present disclosure include a formulation of 4 layers 30 as shown in FIG. 56. This formulation can include an inner core 15 of a non-pareil bead and 4 concentric layers from inner to outer described as, a swelling polymer layer 18, drug layer 12, a sustained release layer 16 and a pH dependent delayed release layer 14, which can be a pH dependent layer. It will be understood that a layer may have a surface. FIG. 56 shows, for example, a surface 121 of the drug layer 12, a surface 161 of the sustained release layer 16, and a surface 141 of the delayed release layer 14.

In certain embodiments, the 4 layer composition can be made in a step-wise fashion. In the first step, a hydrophilic polymer suspended in ethanol with a binder is coated onto nonpareil beads to a 30-50% weight gain. In certain embodiments PolyOx Coagulant SFP (PEO) marketed by the Dow Chemical Company is the hydrophilic polymer and hydroxypropyl cellulose (HPC LF) is added as the binder. The PolyOx layer is then sealed with a hydroxypropyl cellulose such as Klucel® EF to a 10% weight gain. The active pharmaceutical ingredient (API) is then suspended in ethanol with a binder and coated onto the layered bead and the sustained release and delayed release coatings are applied as described herein.

FIG. 55A and FIG. 55B represent embodiments in which the core is a minitablet 20 rather than a bead. The core and layers in FIG. 55A and FIG. 55B are functionally the same as the like numbered layers on the beads in FIG. 54A and FIG. 54B, except there is no optional inert core.

Various water-soluble polymers can be used in the disclosed formulations. Such polymers include, but are not limited to polyethylene oxide (PEO), ethylene oxide-propylene oxide copolymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin or the like.

In certain embodiments, at least the delayed release layer includes one or more polymers such as an acrylic polymer, acrylic copolymer, methacrylic polymer or methacrylic copolymer, including but not limited to Eudragit® L100, Eudragit® L100-55, Eudragit® L 30 D-55, Eudragit® 5100, Eudragit® 4135F, Eudragit® RS, acrylic acid and methacrylic acid copolymers, methyl methacrylate, methyl methacrylate copolymers, ethoxyethyl methacrylates, cyanoethyl methacrylate, aminoalkyl methacrylate copolymer, polyacrylic acid, polymethacrylic acid, methacrylic acid alkylamine copolymer, polymethyl methacrylate, polymethacrylic acid anhydride, polymethacrylate, polyacrylamide, polymethacrylic acid anhydride and glycidyl methacrylate copolymers, an alkylcellulose such as ethylcellulose, methylcellulose, calcium carboxymethyl cellulose, certain substituted cellulose polymers such as hydroxypropyl methylcellulose phthalate, and hydroxypropyl methylcellulose acetate succinate, cellulose acetate butyrate, cellulose acetate phthalate, and cellulose acetate trimaleate, polyvinyl acetate phthalate, polyester, waxes, shellac, zein, or the like.

Eudragits are well known polymers and copolymers useful for controlled release applications. The EUDRAGIT® grades for enteric coatings are based on anionic polymers of methacrylic acid and methacrylates. They contain —COOH as a functional group. They dissolve at ranges from pH 5.5 to pH 7. EUDRAGIT® FS 30 D is the aqueous dispersion of an anionic copolymer based on methyl acrylate, methyl methacrylate and methacrylic acid. It is insoluble in acidic media, but dissolves by salt formation above pH 7.0. Eudragit L100-55 and L30-55 dissolve at pH above 5.5. Eudragit L100 and S100 dissolve at pH above 6.0.

Sustained-release EUDRAGIT® formulations are employed for many oral dosage forms to enable time-controlled release of active ingredients. Drug delivery can be controlled throughout the whole gastro-intestinal tract for increased therapeutic effect and patient compliance. Different polymer combinations of EUDRAGIT® RL (readily permeable) and RS (sparingly permeable) grades allow custom-tailored release profiles and enable a wide range of alternatives to achieve the desired drug delivery performance. The EUDRAGIT® NE polymer is a neutral ester dispersion which requires no plasticizer and is particularly suitable for granulation processes in the manufacture of matrix tablets and sustained release coatings.

Exemplary osmagents or osmotic agents include organic and inorganic compounds such as salts, acids, bases, chelating agents, sodium chloride, lithium chloride, magnesium chloride, magnesium sulfate, lithium sulfate, potassium chloride, sodium sulfite, calcium bicarbonate, sodium sulfate, calcium sulfate, calcium lactate, d-mannitol, urea, tartaric acid, raffinose, sucrose, alpha-d-lactose monohydrate, glucose, combinations thereof and other similar or equivalent materials which are widely known in the art.

As used herein, the term “disintegrant” is intended to mean a compound used in solid dosage forms to promote the disruption of a solid mass (layer) into smaller particles that are more readily dispersed or dissolved. Exemplary disintegrants include, by way of example and without limitation, starches such as corn starch, potato starch, pre-gelatinized and modified starches thereof, sweeteners, clays, bentonite, microcrystalline cellulose (e.g., Avicel™) carboxymethylcellulose calcium, croscarmellose sodium, alginic acid, sodium alginate, cellulose polyacrilin potassium (e.g., Amberlite™), alginates, sodium starch glycolate, gums, agar, guar, locust bean, karaya, pectin, tragacanth, crospovidone and other materials known to one of ordinary skill in the art. A superdisintegrant is a rapidly acting disintegrant. Exemplary superdisintegrants include crospovidone and low substituted HPC.

In preferred embodiments, a plasticizer is also included in the oral dosage form. Plasticizers suitable for use in the present invention include, but are not limited to, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, acetyl triethyl citrate, tributyl citrate and allyl glycolate.

It is an aspect of the compositions and methods of the present disclosure that the formulations or dosage forms can also incorporate one or more ingredients that discourage or prevent abuse of the active ingredients by crushing and inhaling a powdered form of the formulations. As such, a nasal irritant can be included, either as a separate layer, or incorporated into an outer layer, a sustained release layer or the core of the dosage forms. Exemplary irritants include, but are not limited to sodium lauryl sulfate, which is also called sodium dodecyl sulfate or capsaicinoids including capsaicin and synthetic capsaicins. In certain embodiments, the dosage forms include from 1% to 10% sodium lauryl sulfate.

The compositions of the present disclosure can also include one or more functional excipients such as lubricants, thermal lubricants, antioxidants, buffering agents, alkalinizing agents, binders, diluents, sweeteners, chelating agents, colorants, flavorants, surfactants, solubilizers, wetting agents, stabilizers, hydrophilic polymers, hydrophobic polymers, waxes, lipophilic materials, absorption enhancers, preservatives, absorbents, cross-linking agents, bioadhesive polymers, retardants, pore formers, and fragrance.

Lubricants or thermal lubricants useful in the present invention include, but are not limited to fatty esters, glyceryl monooleate, glyceryl monostearate, wax, camauba wax, beeswax, vitamin E succinate, and a combination thereof.

As used herein, the term “antioxidant” is intended to mean an agent that inhibits oxidation and thus is used to prevent the deterioration of preparations by oxidation due to the presence of oxygen free radicals or free metals in the composition. Such compounds include, by way of example and without limitation, ascorbic acid, ascorbyl palmitate, butylated hydroxyanisole (BHA), butylated hydroxytoluene (BHT), hypophophorous acid, monothioglycerol, sodium ascorbate, sodium formaldehyde sulfoxylate and sodium metabisulfite and others known to those of ordinary skill in the art. Other suitable antioxidants include, for example, vitamin C, sodium bisulfate, vitamin E and its derivatives, propyl gallate or a sulfite derivative.

Binders suitable for use in the present invention include beeswax, camauba wax, cetyl palmitate, glycerol behenate, glyceryl monostearate, glyceryl palmitostearate, glyceryl stearate, hydrogenated castor oil, microcrystalline wax, paraffin wax, stearic acid, stearic alcohol, stearate 6000 WL1644, gelucire 50/13, poloxamer 188, and polyethylene glycol (PEG) 2000, 3000, 6000, 8000, 10000 or 20000.

A buffering agent is used to resist change in pH upon dilution or addition of acid or alkali. Such compounds include, by way of example and without limitation, potassium metaphosphate, potassium phosphate, monobasic sodium acetate and sodium citrate anhydrous and dihydrate, salts of inorganic or organic acids, salts of inorganic or organic bases, and others known to those of ordinary skill in the art,

As used herein, the term “alkalizing agent” is intended to mean a compound used to provide alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art.

Exemplary binders include: polyethylene oxide; polypropylene oxide; polyvinylpyrrolidone; polyvinylpyrrolidone-co-vinylacetate; acrylate and methacrylate copolymers; polyethylene; polycaprolactone; polyethylene-co-polypropylene; alkylcelluloses and cellulosic derivatives such as low substituted HPC (L-HPC), methylcellulose; hydroxyalkylcelluloses such as hydroxymethylcellulose, hydroxy ethylcellulose, hydroxypropylcellulose, and hydroxybutylcellulose; hydroxyalkyl alkylcelluloses such as hydroxyethyl methylcellulose and hydroxypropyl methylcellulose; starches, pectins; PLA and PLGA, polyesters (shellac), wax such as carnauba wax, beeswax; polysaccharides such as cellulose, tragacanth, gum arabic, guar gum, and xanthan gum.

Exemplary chelating agents include EDTA and its salts, alphahydroxy acids such as citric acid, polycarboxylic acids, polyamines, derivatives thereof, and others known to those of ordinary skill in the art.

As used herein, the term “colorant” is intended to mean a compound used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red, other F.D. & C. dyes and natural coloring agents such as grape skin extract, beet red powder, beta carotene, annato, carmine, turmeric, paprika, and other materials known to one of ordinary skill in the art. The amount of coloring agent used will vary as desired.

As used herein, the term “flavorant” is intended to mean a compound used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors that have been found to be particularly useful include commercially available orange, grape, cherry and bubble gum flavors and mixtures thereof. The amount of flavoring may depend on a number of factors, including the organoleptic effect desired. Flavors will be present in any amount as desired by those of ordinary skill in the art. Particular flavors are the grape and cherry flavors and citrus flavors such as orange.

Suitable surfactants include Polysorbate 80, sorbitan monooleate, polyoxymer, sodium lauryl sulfate or others known in the art. Soaps and synthetic detergents may be employed as surfactants. Suitable soaps include fatty acid alkali metal, ammonium, and triethanolamine salts. Suitable detergents include cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl β-aminopropionates and 2-alkylimidazoline quaternary ammonium salts; and mixtures thereof.

A wetting agent is an agent that decreases the surface tension of a liquid. Wetting agents would include alcohols, glycerin, proteins, peptides water miscible solvents such as glycols, hydrophilic polymers Polysorbate 80, sorbitan monooleate, sodium lauryl sulfate, fatty acid alkali metal, ammonium, and triethanolamine salts, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl β-aminopropionates and 2-alkylimidazoline quaternary ammonium salts; and mixtures thereof.

Solubilizers include cyclodextrins, povidone, combinations thereof, and others known to those of ordinary skill in the art.

Exemplary waxes include carnauba wax, beeswax, microcrystalline wax and others known to one of ordinary skill in the art.

Exemplary absorption enhancers include dimethyl sulfoxide, Vitamin E PGS, sodium cholate and others known to one of ordinary skill in the art.

Preservatives include compounds used to prevent the groweighth of microorganisms. Suitable preservatives include, by way of example and without limitation, benzalkonium chloride, benzethonium chloride, benzyl alcohol, cetylpyridinium chloride, chlorobutanol, phenol, phenylethyl alcohol, phenylmercuric nitrate and thimerosal and others known to those of ordinary skill in the art.

Examples of absorbents include sodium starch glycolate (Explotab™, Primojel™) and croscarmellose sodium (Ac-Di-Sol™), cross-linked PVP (Polyplasdone™ XL 10), veegum, clays, alginates, PVP, alginic acid, carboxymethylcellulose calcium, microcrystalline cellulose (e.g., Avicel™), polacrillin potassium (e.g., Amberlite™), sodium alginate, corn starch, potato starch, pregelatinized starch, modified starch, cellulosic agents, montmorrilonite clays (e.g., bentonite), gums, agar, locust bean gum, gum karaya, pectin, tragacanth, and other disintegrants known in to those of ordinary skill in the art.

A cross-linking agent is defined as any compound that will form cross-links between the moieties of the polymer. A cross-linking agent can include, by way of example and without limitation, an organic acid, an alpha-hydroxy acid, and a beta-hydroxy acid. Suitable cross-linking agents include tartaric acid, citric acid, fumaric acid, succinic acid and others known to those of ordinary skill in the art.

Bioadhesive polymers include polyethylene oxide, KLUCEL (hydroxypropylcellulose), CARBOPOL, polycarbophil, GANTREZ, Poloxamer, and combinations thereof, and others known to one of ordinary skill in the art.

Retardants are agents that are insoluble or slightly soluble polymers with a glass transition temperature (Tg) above 45° C., or above 50° C. before being plasticized by other agents in the formulation including other polymers and other excipients needed for processing. The excipients include waxes, acrylics, cellulosics, lipids, proteins, glycols, and the like.

Exemplary pore formers include water-soluble polymers such as polyethylene glycol, propylene glycol, polaxamer and povidone; binders such as lactose, calcium sulfate, calcium phosphate and the like; salts such as sodium chloride, magnesium chloride and the like; combinations thereof and other similar or equivalent materials which are widely known in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

It should be understood that compounds used in the art of pharmaceutical formulation generally serve a variety of functions or purposes. Thus, if a compound named herein is mentioned only once or is used to define more than one term herein, its purpose or function should not be construed as being limited solely to that or those named purpose(s) or function(s).

In some implementations, the present disclosure relates to pharmaceutical preparations for once daily administration of a CNS stimulant for treatment of conditions that respond to such drugs, such as ADD, ADHD, bipolar depression, narcolepsy, sleeping disorders, and fatigue. The dosage is formulated to be taken prior to going bed and starts to release after a lag of several hours so the patient has absorbed a sufficient amount of drug to have a therapeutic effect while awakening and preparing to leave for work or school. It is a further aspect of the formulations that the drug is released in an ascending dose through the day to overcome any acute tolerance effect and maintain a therapeutic level of drug.

An embodiment of the compositions and methods of the present disclosure is a dosage form that includes a capsule enclosing a single population of beads or minitablets that include a core and 2 or more coatings surrounding the core. The inner core is a bead or minitablet containing an API and one or more excipients. The core is enclosed in a sustained release layer, and an outer, delayed release layer.

In certain embodiments the sustained release layer includes a combination of water-soluble polymers and water-insoluble polymers. The sustained release coating can contain a combination of polyethylene oxide and an ethylcellulose, for example, or a hydroxypropylmethyl cellulose and ethylcellulose. An ethylcellulose product that can be used in the disclosed dosage forms is Ethocel™, marketed under a trade mark of The Dow Chemical Company. The rate of dissolution of the sustained release layer can be controlled by adjusting the ratio of water-soluble polymer to water-insoluble polymer in the coating or layer. The weight ratio of water-insoluble to water-soluble polymers can be adjusted, for example and without limitation, from 90:10 to 10:90, from 80:20 to 20:80, from 75:25 to 25:75, from 70:30 to 30:70, from 67.5:33.5 to 33.5:67.5 from 60:40 to 40:60, from 56:44 to 44:56, or to 50:50.

The sustained release coating can also contain plasticizers such as triethyl citrate (TEC) at levels of from 3% to 50% of the combined weight of the polymers. Other additives to the coating can include titanium dioxide, talc, colloidal silicone dioxide or citric acid.

Some examples of sustained release layers are shown in Example 29. The various formulations include those in which the ratios of water-insoluble to water-soluble polymers are varied and one in which the ratios are reversed. In certain embodiments, the active ingredient, or API can be included in the sustained release layer. Any of the disclosed API's can be added to the sustained release layer.

While the disclosed compositions of a capsule containing a single population of beads or minitablets with a sustained release layer and an outer, delayed release layer are shown here in to be an effective delivery system with novel release characteristics and surprisingly low variability in absorption when administered to humans, it is understood that alternative compositions can be used in light of the present disclosure.

In certain embodiments a drug containing core bead or minitablet is coated with a delayed release layer that includes one or more water-insoluble polymers, one or more water-soluble polymers and a silicone oil to achieve a desired delay or lag time prior to release as in the present disclosure. Lag time and release are controlled by the proportion of the two types of polymers and the thickness of the layer. In such embodiments, the delayed release layer can include, but is not limited to cellulose acetate phthalate, cellulose acetate trimaletate, hydroxyl propyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, Eudragit L30D, Eudragit L100, Eudragit FS30D, Eudragit S100 or combinations of any thereof. The delayed release layer can also include a plasticizer, or in cer-tain embodiments the delayed release layer can include methacrylic acid copolymer Type B, mono- and diglycerides, dibutyl sebacate and polysorbate 80. The delayed release layer can also include a cellulose ether derivative, an acrylic resin, a copolymer of acrylic acid and meth-acrylic acid esters with quaternary ammonium groups, a copolymer of acrylic acid and methacryl-ic acid esters or a combination of any thereof. The layer can further include a powder component such as talc as a carrier for the silicone oil.

In certain embodiments, a CNS stimulant can be contained in a delayed and/or controlled release capsule. In such embodiments a water-insoluble capsule contains one or more compartments in which the drug or active agent is held. Additionally one or more absorbents, superabsorbents or osmagents are included in the drug containing compartments. The capsules also include one more apertures plugged with a water-soluble polymer, at least one in fluid communication with each compartment and a delayed release layer enclosing the entire capsule.

In such embodiments the length of initial delay can be controlled by the composition and thick-ness of the outer, delayed release layer. This layer can be a pH dependent layer or a pH inde-pendent layer as disclosed herein. When the capsule is administered to a human, the delayed release layer begins to lose integrity as the capsule passes through the GI tract. When the water-soluble plugs are exposed and dissolve, aqueous fluid enters the drug containing compartment(s) and is absorbed by the absorbent or osmagent, thus driving the active agent from the capsule through the aperture. The release profile can be controlled by the concentration and absorption characteristics of the absorbent or osmagent to obtain the desired profile.

Further Examples of the compositions described herein are provided in Examples 30 to 42.

In some implementations described herein, “HLD200” refers to exemplary compositions of the present disclosure, such as described in the Examples. An FDA-approved formulation of HLD200 is also known as JORNAY PM® (Ironshore Pharmaceuticals & Development, Inc.).

As described herein, the compositions of the present disclosure exhibit markedly different pharmacokinetics, including in vitro dissolution profile, in vivo absorbance profile, and in vitro-in vivo correlation (IVIVC), compared to other methylphenidate drugs, such as Concerta® (ALZA Corporation), Ritalin LA® (Novartis AG), Metadate CD® (UCB, Inc), and Quillivant XR® (NextWave Pharmaceuticals, Inc.). In contrast to the compositions of the present disclosure, these other methylphenidate drugs are formulated to have an immediate release (IR) component and an extended release (ER) component. A model that can be used to describe the pharmacokinetics of these other drugs exhibits a linear and time-invariant relationship between dissolution and absorption, wherein the multi-phase (IR and ER) release profile of in vivo absorption is modeled using a double Weibull function r2(t):

$$r 2 ( t ) = ff \cdot {e}^{- ( {( \frac{t i m e}{t d} )}^{S S} )} + ( 1 - ff ) \cdot {e}^{- ( {( \frac{t i m e}{t d 1} )}^{S S 1} )} \qquad ( Eq . 4 )$$

where: ff is the fraction of the dose released in the 1st process (IR), td is the time necessary to absorb 63.2% of the dose released in the 1st process (IR), td1 is the time necessary to absorb 63.2% of the dose released in the 2nd process (ER), ss is the sigmoidicy factor for the 1st process (IR), and ss1 is the sigmoidicity factor for the 2nd process (ER) (R. Gomeni, F. Bressolle, T. J. Spencer, S. V. Faraone. Meta-analytic approach to evaluate alternative models for characterizing the PK profiles of extended release formulations of MPH. ASCPT 2016 Annual Meeting, Mar. 8-12, 2016, Hilton Bayfront, San Diego, Calif., incorporated by reference herein). The in vivo absorption model is assumed to follow a Weibull distribution, and accordingly the absorption profile is assumed to be sigmoidal.

The pharmacokinetics of a drug can be described in terms of in vitro dissolution-in vivo absorption correlation (IVIVC). For example, IVIVC has been generally defined by the U.S. Food and Drug Administration (FDA) as a predictive mathematical model describing the relationship between an in-vitro property of a dosage form and an in-vivo response. Generally, the in-vitro property can be the rate or extent of drug dissolution or release while the in-vivo response can be the plasma drug concentration or amount of drug absorbed. The United States Pharmacopoeia (USP) also defines IVIVC as the establishment of a relationship between a biological property, or a parameter derived from a biological property produced from a dosage form, and a physicochemical property of the same dosage form. Typically, the parameter derived from the biological property may be, for example, AUC or Cmax, while the physicochemical property may be the in vitro dissolution profile.

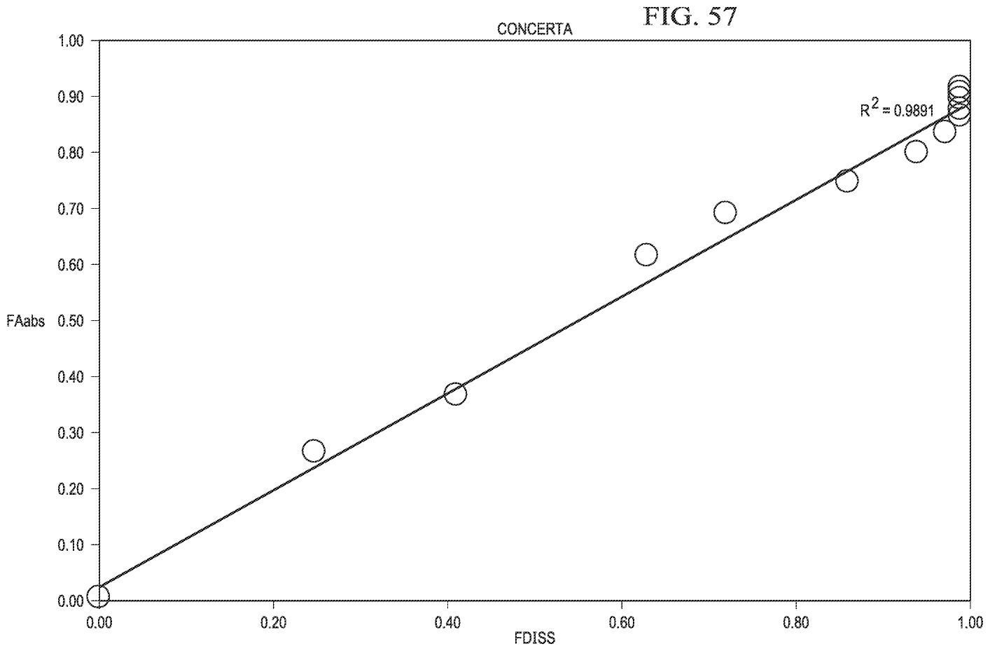

For example, with regard to drugs such as Concerta® (ALZA Corporation), Ritalin LA® (Novartis AG), Metadate CD® (UCB, Inc), and Quillivant XR® (NextWave Pharmaceuticals, Inc.), the relationship between the fraction of methylphenidate dissolved in vitro and the fraction of methylphenidate absorbed in vivo is linear, or approximately linear. For example, FIG. 57 is a graph reporting an exemplary correlation of the fraction of methylphenidate dissolved in vitro (FDISS) versus the fraction of methylphenidate absorbed in vivo (Fabs) from Concerta® (ALZA Corporation). Ibid. FIG. 57 shows that the in vitro-in vivo correlation (IVIVC) between the fraction of methylphenidate dissolved in vitro (FDISS) versus the fraction of methylphenidate absorbed in vivo (Fabs) from Concerta® (ALZA Corporation) is linear, or approximately linear.

As would be understood by skilled persons, a linear relationship can be described by a linear polynomial function, otherwise known as a first-degree polynomial. The degree of a polynomial is generally understood to mean the highest degree of its monomials (individual terms or variables, e.g. x) with non-zero coefficients. Accordingly, a first-degree polynomial function may be generally understood to mean a mathematical function, or equation, in which a variable, such as x, in the function has a non-negative integer exponent having a maximum value of 1. Similarly, for example, a fifth-degree polynomial function may be generally understood to mean a mathematical function, or equation, in which the highest degree of its monomials with non-zero coefficients has a sum of non-negative integer exponents having a maximum value of 5. For example, when referring to a “degree” of a function, the degrees of each indeterminate are added, e.g. x³+x²=5^{th} degree.

The term “approximately linear” can in some instances mean that a linear polynomial function best fits a series of data points. For example, a series of data points can be said to be approximately linear when a first-degree polynomial function best fits the series of data points better than, for example, a second-degree polynomial function, a third-degree polynomial function, a fourth-degree polynomial function, a fifth-degree polynomial function, or a sixth-degree polynomial function, and so on.

A process of curve fitting can be used construct a polynomial function that has the best fit to a series of data points. Examples of curve fitting include polynomial regression and polynomial interpolation, among others. Statistical packages such as R and numerical software such as the GNU Scientific Library, MLAB, Maple, MATLAB, Mathematica, GNU Octave, and SciPy include commands for doing curve fitting in a variety of scenarios.

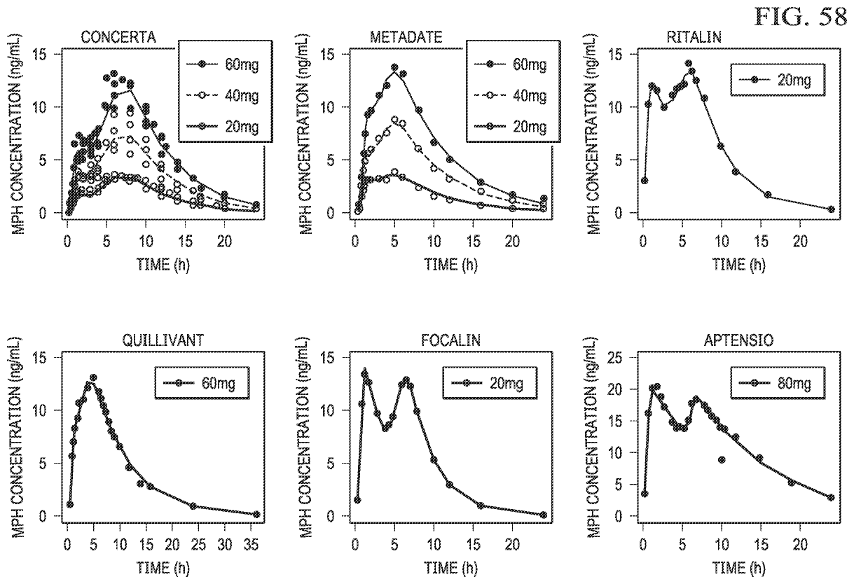

FIG. 58 is a set of graphs reporting exemplary observed (dots) and predicted (line) mean methylphenidate concentrations after the administration of the indicated drugs. Ibid. As shown in FIG. 58, the double Weibull function model can accurately predict in vivo plasma levels of Concerta® (ALZA Corporation), Ritalin LA® (Novartis AG), Metadate CD® (UCB, Inc), and Quillivant XR® (NextWave Pharmaceuticals, Inc.), and also Aptensio® (Rhodes Pharmaceuticals L.P.), confirming a linear, or approximately linear, relationship with Weibull absorption in multiple phases for each of these drugs (Ibid).

In some embodiments, pharmacokinetics of the methylphenidate compositions described herein may be modeled using a double Weibull function r2(t):

$$r 2 ( t ) = ff \cdot {e}^{- ( {( \frac{t i m e}{t d} )}^{S S} )} + ( 1 - ff ) \cdot {e}^{- ( {( \frac{t i m e}{t d 1} )}^{S S 1} )} \qquad ( Eq . 4 )$$

where: ff is the fraction of the dose released in the 1st process (IR), td is the time necessary to absorb 63.2% of the dose released in the 1st process (IR), td1 is the time necessary to absorb 63.2% of the dose released in the 2nd process (ER), ss is the sigmoidicy factor for the 1st process (IR), and ss1 is the sigmoidicity factor for the 2nd process (ER). The in vivo absorption model is assumed to follow a Weibull distribution, and accordingly the absorption profile is assumed to be sigmoidal.

In some embodiments, pharmacokinetics of the methylphenidate compositions described herein can be described by a time varying in-vivo absorption model that includes a single [r1(t)] Weibull function, as follows:

$$r 1 ( t ) = {e}^{- ( {( \frac{t i m e}{t d} )}^{s s} )} \qquad ( Eq . 3 )$$

where: td is the time necessary to absorb 63.2% of the dose released, and ss is the sigmoidicity factor, for example as described in Examples 1 to 22.

In some embodiments, pharmacokinetics of the methylphenidate compositions described herein can be described by an-vivo absorption model that includes a sigmoid eMax function, as follows:

$${r}_{v i t r o} ( t ) = \frac{t i m {e}^{g a}}{{EC}^{ga} + t i m {e}^{g a}} \qquad ( Eq . 9 )$$

where EC is the time to release 50% of the dose and ga is a parameter characterizing the shape of the absorption curve (see Examples 50-53).

The compositions described herein are formulated such that, when administered to a human subject, the methylphenidate is released and absorbed according to a mechanism that is markedly different to other methylphenidate drugs, such as Concerta® (ALZA Corporation), Ritalin LA® (Novartis AG), Metadate CD® (UCB, Inc), and Quillivant XR® (NextWave Pharmaceuticals, Inc.). In various implementations, the compositions described herein do not show a linear, or approximately linear, IVIVC. For example, see Examples 22 to 25 and Example 50 to 53. For example, as described in Example 24, in some implementations, a fifth-degree polynomial function can best fit the IVIVC for compositions of the present disclosure.

Accordingly, in some implementations, the present disclosure relates to a solid, oral pharmaceutical composition that includes methylphenidate or a pharmaceutical salt thereof, wherein an in vivo absorption model of the solid, oral pharmaceutical composition has a single Weibull function:

$$r 1 ( t ) = {e}^{- ( {( \frac{t i m e}{t d} )}^{s s} )}$$

or a double Weibull function r2(t):

$$r 2 ( t ) = ff \cdot {e}^{- ( {( \frac{t i m e}{t d} )}^{S S} )} + ( 1 - ff ) \cdot {e}^{- ( {( \frac{t i m e}{t d 1} )}^{S S 1} )}$$

or a sigmoid eMax function:

$${r}_{v i t r o} ( t ) = \frac{t i m {e}^{g a}}{{EC}^{ga} + t i m {e}^{g a}} ,$$

and wherein a correlation of a plurality of fractions of an in vitro dissolution of the solid, oral pharmaceutical composition with a plurality of fractions of an in vivo absorption of the solid, oral pharmaceutical composition is non-linear.

In some implementations, the non-linear correlation of the plurality of fractions of the in vitro dissolution of the solid, oral pharmaceutical composition with the plurality of fractions of the in vivo absorption may best fit a fifth-degree polynomial function.

In some implementations, the non-linear correlation of the plurality of fractions of the in vitro dissolution of the solid, oral pharmaceutical composition with the plurality of fractions of the in vivo absorption may best fit a second-degree polynomial function, a third-degree polynomial function, a fourth-degree polynomial function, a fifth-degree polynomial function, or a sixth-degree polynomial function.

As would be understood by persons of ordinary skill in the art upon reading the present disclosure, an IVIVC plot can be produced using data sets from individual subjects or a population of subjects following various methods. For example, in one method, PK curves of individual subjects can be predicted and then a mean value of the individual subjects' data calculated to produce an IVIVC plot for a population of subjects. Alternatively, for example, in another method, an IVIVC plot for a population of subjects can be produced by using the mean observed PK curve from a population of subjects.

In some implementations, the plurality of fractions of the in vitro dissolution of the solid, oral pharmaceutical composition and the plurality of fractions of the in vivo absorption may include a plurality of values from 0 to 1.

Methods of the present disclosure include a method of treating a condition in a subject with a disorder or condition responsive to the administration of a methylphenidate. The method involves orally administering to the subject an effective amount of a solid, oral pharmaceutical composition described herein.

In some implementations, administration of a composition described herein to a population of subjects provides a significant improvement in ADHD related behavior or ability over a period of time, such as at least 12 continuous hours, as measured by a validated rating scale, score or combined score. As used herein a validated rating scale is a scale, score or combined score that is validated or published in a peer reviewed journal, or is recognized as valid by those of skill in the art, or considered by a pharmaceutical regulatory agency such as the U.S. Food and Drug Administration or the European Medicines Agency, for example, as providing a valid measure of therapeutic efficacy.

The validated rating scale, score or combined score as used herein includes methods that are recognized by a government pharmaceutical regulatory agency, are published and/or relied upon in a peer reviewed journal, or in a clinical trial reported in a peer-reviewed journal or are endorsed by an association of physicians or clinicians. Examples of such methods or measurements include, but are not limited to the ADHD-RS-IV scale based on the Diagnostic and Statistical Manual of Mental Disorders, Fourth Edition criteria, the Before School Functioning Questionnaire (BSFQ), Clinical Global Impression (CGI), Clinical Global Impression of Improvement (CGI-I), Clinical Global Impresssion of Severity (CGI-S), Conors Global Index Parent (CGI-P), Diagnostic and Statistical Manual of Mental Disorders—Fourth Edition Text Revision (DSM-IV-TR), Parents Rating of Evening and Morning Behavior Revised (PREMB-R), Permanent Product Measure of Performance (PERMP) scores, Swanson, Kotkin, Agler, M-Flynn and Pelham (SKAMP) rating scale, SKAMP-CS combined score, Adult ADHD Medication Rebound Scale (AMRS) for AM or PM, or Adult ADHD Medication Smoothness of Effect Scale (AMSES) for AM or PM.

In some implementations, the present disclosure relates to a method wherein when the composition is administered to a population, such as a population of adolescent or child subjects, the composition provides a significant improvement in SKAMP scores during a period of about 12 continuous hours and where the composition provides a significant improvement in SKAMP scores during a period from about 11 through about 23 hours post-dose, or wherein when the composition is administered at about 8-10 PM, the composition provides a significant improvement in SKAMP scores during a 10-12 hour period from about 7 AM to about 9 PM the following day.

Methods of the present disclosure may reduce variations in efficacy, or reduce the likelihood or severity of a rebound, or both, of an improvement in an ADHD related behavior or ability over a period of time, as measured by a validated rating scale. In some implementations, administration of the compositions described herein to a population of subjects provides a clinical response, such as an improvement in SKAMP score over a period of time, wherein the improvement provides minimal variation in efficacy, reduced likelihood or severity of rebound, or both, over the period of time.

In some implementations, a model is described herein that can accurately predict a change in SKAMP score when using a methylphenidate PK profile as an input variable. See for example Examples 10 to 22. In particular, for example, Examples 15 to 18 describe sensitivity of the SKAMP response with respect to the time of day of administration and sigmoidicy factor (ss) of a composition of the present disclosure. In some implementations, the compositions and methods of the present disclosure provide minimal variation in efficacy, reduced likelihood or severity of rebound, or both as a function of the in-vivo release rate of compositions of methylphenidate described herein and/or as a function of the time of administration on the evening before the morning classroom. In some exemplary implementations (e.g. see Examples 15 to 18), the efficacy can be defined as the area under change from a population of placebo-treated subjects of the SKAMP score (AUEC) during a period of time, e.g. from 8:00 am to 8:00 pm. In other implementations, the efficacy can be defined as the area under change from a population of untreated subjects of the SKAMP score during a period of time, e.g. from 8:00 am to 8:00 pm. An exemplary graphical representation of evaluation of efficacy, also referred to herein in certain implementations as “clinical benefit” or “CB” is shown in FIG. 38, wherein the light dashed curve represents the SKAMP score for placebo, the thick dashed curve represents the SKAMP score for HLD200, and the shaded region the AUEC.

In some implementations, the present disclosure relates to compositions and methods providing minimal variation in efficacy, reduced likelihood or severity of rebound, or both, in improving an ADHD related behavior or ability, such as a SKAMP score, over a period of time.

In some implementations, the period of time may begin at, or begin at about, 8:00 am, 9:00 am, 10:00 am, 11:00 am, 12:00 pm, 1:00 pm, 2:00 pm, 3:00 pm, 4:00 pm, 5:00 pm, 6:00 pm, or 7:00 pm.

In some implementations, the period of time may begin at, or begin at about, 8, 9, 10, 11, 12, 13, 14, 15, or 16 hours after administration of the composition. For example, in some implementations, the composition may be administered at, or at about, 4:00 pm, 5:00 pm, 6:00 pm, 7:00 pm, 8:00 pm, 9:00 pm, 10:00 pm, 11:00 pm on the day before the period of time, or 12:00 am on the day of the period of time. For example, as described in Examples 16 and 17, the composition may be administered 12 hours before the morning classroom start at 8:00 am.

In some implementations, wherein a time varying in vivo absorption model of the solid, oral pharmaceutical composition described herein that has a core that includes methylphenidate or a pharmaceutical salt thereof has a single Weibull function:

$$r 1 ( t ) = {e}^{- ( {( \frac{t i m e}{t d} )}^{ss} )}$$

wherein td is the time necessary to absorb 63.2% of the methylphenidate or a pharmaceutical salt thereof released, and ss is the sigmoidicy factor, the sigmoidicity factor (ss) may have a value of, or of about, 4.5 to 8.5, such as, or such as about, 4.5, 5.5, 6.5, 7.5, or 8.5. For example, in some implementations, as described in Example 17, the sigmoidicity factor (ss) may have a value of, or of about, 4.5 to 8.5, such as, or such as about, 4.5, 5.5, 6.5, 7.5, or 8.5 and the period of time may begin at, or begin at about, 10-12 hours after administration of the composition (e.g., see Table 21).

In some implementations, the variation of efficacy can be described by a fluctuation index (FI). For example, Examples 20 to 22 describe comparison of the efficacy of an exemplary composition of the present disclosure (HLD200) with Concerta® (ALZA Corporation), Metadate CD® (UCB, Inc), NWP06 (Quillivant XR® (NextWave Pharmaceuticals, Inc.)), and d-MPH ER (Focalin XR® (Novartis AG)). As described in the Examples 20 to 22, in some implementations, the efficacy can be calculated as mean simulated values of the change from placebo of the SKAMP score (CHP) computed from 8:00 am to 8:00 μm. The response and the variability in the response of the different drugs can be evaluated using the average value of the change from placebo of the SKAMP scores and using a fluctuation index (FI). The fluctuation index can be defined as:

$$FI = \frac{[ max ( C H P ) - min ( C H P ) ]}{average ( CHP )}$$

In some implementations, the compositions and methods described herein show improvement in ADHD related behavior or ability, such as reduction in SKAMP score, with reduced variation of efficacy during the day and starting at an earlier time in the day, for example in comparison with other drugs such as Concerta® (ALZA Corporation), Metadate CD® (UCB, Inc), NWP06 (Quillivant XR® (NextWave Pharmaceuticals, Inc.)), and d-MPH ER (Focalin XR® (Novartis AG). As described in Examples 20 to 22, the earlier onset in the improvement of SKAMP score is associated with less variation of efficacy in improving the SKAMP score during the day. In an exemplary implementation described in Example 22, administration of an exemplary composition of the present disclosure, HLD200, provided a fluctuation index of −0.87. The fluctuating index of HLD200 was more that 50% lower that the fluctuation index of the other drugs evaluated.

Accordingly, in some implementations, the present disclosure relates to compositions and methods providing a minimal variation in improvement in an ADHD related behavior or ability, such as a SKAMP score, or for reducing the likelihood or severity of rebound, or both, over a period of time. In some implementations, the variation may be measured by a fluctuation index (FI):

$$FI = \frac{[ max ( C H P ) - min ( C H P ) ]}{average ( CHP )}$$

wherein CHP is a change from placebo of the SKAMP score during the period of time. In some implementations, FI may have an absolute value less than 1.0.

In some implementations, the period of time may begin upon a measurable increase in plasma MPH concentration. For example, in various implementations the measurable increase in plasma MPH concentration may begin at, or at about, 4, 5, 6, 7, 8, 9, 10, 11, or 12 hours after administration of a composition described herein.

In some implementations, the period of time may end at the time at which Cmax occurs (Tmax).

In some implementations, the period of time may begin at the time at which Cmax occurs (Tmax).

In some implementations, the period of time may end at, or end at about, 9:00 am, 10:00 am, 11:00 am, 12:00 pm, 1:00 pm, 2:00 pm, 3:00 pm, 4:00 pm, 5:00 pm, 6:00 pm, 7:00 pm, or 8:00 pm.

In some implementations, the period of time may last until, or until about, 6, 7, 8, 9, 10, 11, 12, 13, 14, 15, or 16 hours after the start of the period of time.

In some implementations, the period of time may last until, or until about, the plasma concentration of MPH is below, or below about, 5 ng/mL. In some implementations, the period of time may last until, or until about, the plasma concentration of MPH is below, or below about, from 3 to 5 ng/mL.

In some implementations, the period of time may last until, or until about, 3, 4, 5, 6, 7, 8, 9, 10, 11, or 12 hours after methylphenidate Tmax in the subject.

In some implementations, the period of time may last until, the subject falls asleep after a Tmax.

In some implementations, during the entire period of time, the value of the SKAMP scores may not change by more than, or than about, 6, 7, 8, 9, or 10.

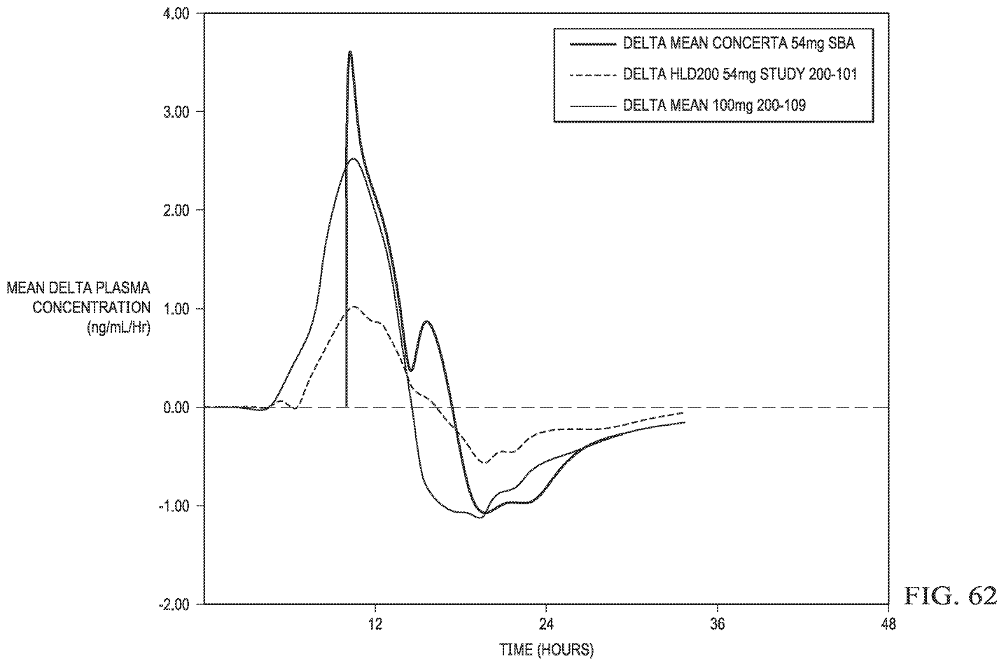

In some implementations of the compositions and methods described herein, during the entire period of time the rate of change of a MPH plasma concentration over time may not be greater than +2.5 ng·hr/mL following a dose of up to 100 mg MPH. For example, as described in Example 46, FIG. 62 shows the rate of change of exemplary HLD200 formulations compared to CONCERTA® 54 mg dose. For example, HLD20054 mg has a maximum rate of change in increasing methylphenidate plasma concentration of about +1.0 ng/mL/hour and a maximum rate of change in decreasing methylphenidate plasma concentration of about −0.5 ng/mL/hour. FIG. 62 also shows HLD200100 mg has a maximum rate of change in increasing methylphenidate plasma concentration of about +2.5 ng/mL/hour and a maximum rate of change in decreasing methylphenidate plasma concentration of about −1.2 ng/mL/hour. In contrast, CONCERTA® 54 mg has a maximum rate of change in increasing methylphenidate plasma concentration of about +3.6 ng/mL/hour and a maximum rate of change in decreasing methylphenidate plasma concentration of about −1.0 ng/mL/hour. Normalization of the CONCERTA® data to a dose of 100 mg CONCERTA® by multiplying by 100/54=1.85 would be expected to give a maximum rate of change in increasing methylphenidate plasma concentration of about +3.6×1.85=+6.7 ng/mL/hour and a maximum rate of change in decreasing methylphenidate plasma concentration of about −1.0×1.85=−1.85 ng/mL/hour for CONCERTA® 100 mg.

In some implementations of the compositions and methods described herein, during the entire period of time the rate of change of a MPH plasma concentration over time may not be greater than +2.5 ng·hr/mL following a dose of 20 mg to 100 mg MPH. In some implementations, the rate of change of a MPH plasma concentration may not have a value less than −1.2 ng/hr/mL between Tmax and 6 hours after Tmax, following a dose of 20 mg to 100 mg MPH.

In some implementations, the slope of the MPH plasma concentration curve can be calculated to take into account body weight, dosage amount, and so on, and so can be determined taking into account mg/kg dose in a subject.

Accordingly, in various implementations, when the rate of change of the MPH plasma concentration over time has a value not less than −1.2 ng/hr/mL following a dose of up to 100 mg MPH between time points Tmax and 6 hours after Tmax, the SKAMP scores may not have a fluctuation index greater than 1, and the subject may experience less variation and/or less rebound compared to other MPH drugs described herein.

In some implementations, the period of time may include a period wherein the MPH plasma concentration is between Cmax and at least 40% Cmax. During the period of time, the SKAMP scores may not have a fluctuation index greater than 1 in the period, and/or the rate of change of the MPH plasma concentration over time may not have a value greater than +1.5 ng·hr/mL or less than −1.5 ng·hr/mL. In other words, in some implementations, such as during a period wherein the MPH plasma concentration is between Cmax and at least 40% Cmax, the rate of change of the MPH plasma concentration over time may not have an absolute value greater than 1.5. Accordingly, in such implementations, the subject may experience minimal variation in efficacy or reduced likelihood and/or severity of rebound in efficacy during the period.

In some implementations, the present disclosure relates to compositions and methods providing minimal variation in methylphenidate efficacy during a window of time, for reducing the likelihood or severity of rebound, or both, wherein the methylphenidate or pharmaceutical salt thereof is absorbed in the colon. In some implementations, at least 90% is absorbed in the colon.

Typical gastrointestinal transit times are: gastric emptying 90 minutes, small bowel transit time 4-6 hours, colon arrival times approximately 8 hours.

For example, as described in Example 45, using a PK/PD model described herein, exemplary maximal therapeutic efficacy over a 24-hour period was estimated to be associated with absorption of methylphenidate beginning after approximately 8 hours, associated with arrival time in the colon. In some implementations, the compositions and methods described herein provide absorption of methylphenidate beginning after approximately 8 hours, associated with arrival time in the colon. In contrast, for example, the exemplary modeling results show that for other methylphenidate drugs, such as Concerta® (ALZA Corporation), 50% of methylphenidate is absorbed in 4-6 hours, associated with absorption in the small bowel and wherein all the methylphenidate is absorbed by approximately 10-12 hours, which is much faster than an exemplary optimal rate described in Example 45.

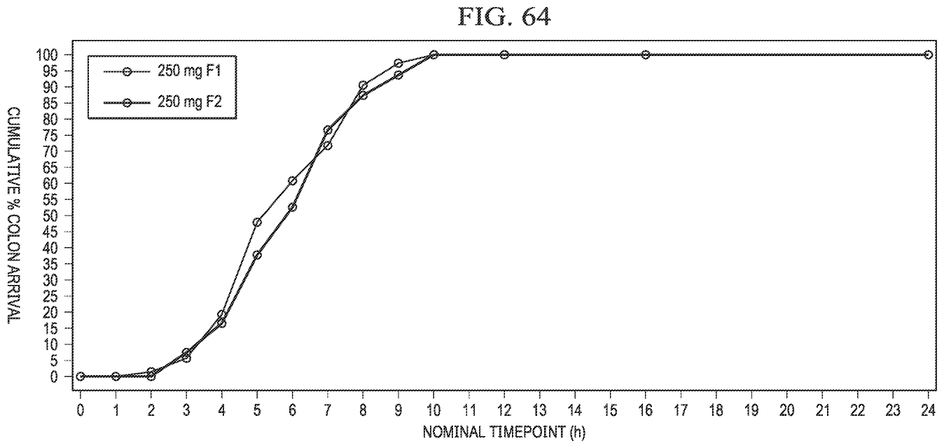

FIG. 64 is a graph reporting exemplary cumulative % colon arrival time for surrogate beads radio-labelled with not more than 1 MBq ¹¹¹Indium. The surrogate beads have the same size shape and density as beads of the methylphenidate formulations, but lack the methylphenidate active ingredient and are not coated. The plots show results from two separate experiments, indicated as “F1” and “F2”. FIG. 64 shows that all radio-labelled beads have arrived in the colon by 10 hours after administration.

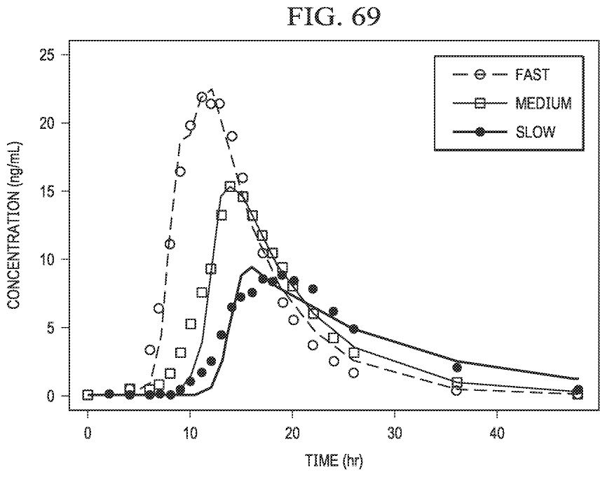

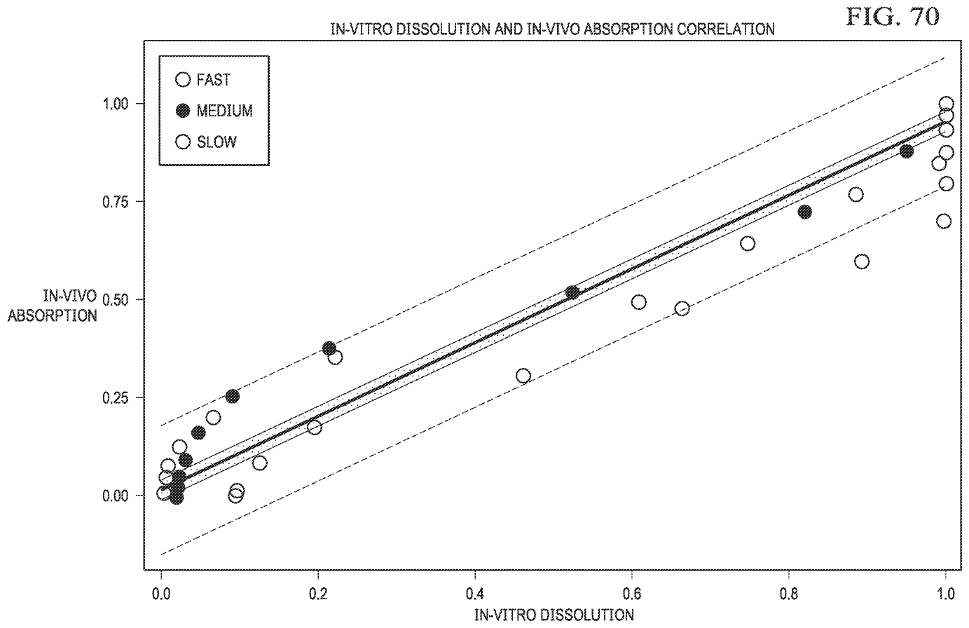

Examples 48 and 49 of the present disclosure describes differences in IVIVC of exemplary fast, medium, and slow formulations of HLD200. For example, FIG. 69 shows that the medium formulation (also referred to herein as the final formulation, corresponding to Jornay PM®) has increasing methylphenidate plasma concentration from about 8 hours post-administration, whereas the fast formulation has an earlier increase of methylphenidate plasma concentration from about 6 hours post-administration, corresponding to more upper bowel absorption than the medium formulation. In addition, the slow formulation shows increasing methylphenidate plasma concentration from about 10 hours post-administration, corresponding to release further into the colon than the medium formulation. FIG. 70 and FIGS. 82-84 show that the patterns of the data points (dots) for the slow, medium and fast formulations have different curvature. The data points (dots) of the slow release formulation best fit a fifth-degree polynomial. In contrast, the plot of the dots for the IVIVC of the fast formulation is more linear, associated with the most upper bowel absorption. The IVIVC plot for the medium formulation best fits a third-degree polynomial.

In some implementations, the present disclosure relates to methods of treating subjects who are sensitive to the negative effects of variations in efficacy, rebound, or both. In some implementations, the present disclosure relates to composition and methods for treating subjects with comorbidity, particularly with another behavior disorder, or mental health disorder, such as patients with both ADD or ADHD and an autism spectrum disorder (ASD).

For example, in children having both ADHD and ASD, a methylphenidate dose effect on rebound hyperactivity and aggression has been observed (Soo-Jeong Kim et al., 2017) Autism Dev Disord 47:2307-2313, incorporated herein by reference).

Accordingly, in some implementations, the present disclosure relates to compositions and methods of treating a subject having ADD or ADHD and an autism spectrum disorder (ASD). The method includes orally administering to the subject an effective amount of a solid, oral pharmaceutical composition described herein, wherein the administering provides minimal variation in methylphenidate efficacy during a window of time, for reducing the likelihood or severity of rebound, or both.

### EXAMPLES

The methods and compositions herein disclosed are further illustrated in the following examples, which are provided by way of illustration and are not intended to be limiting.

Examples 1 to 22 relate to PK/PD modeling and simulation with respect to formulations of methylphenidate described herein. The following methods were used:

PK Data. HLD200 (20 mg and 100 mg) was administered to 20 adult volunteers at 21:00 h using a Latin square two sequence, two period crossover design study (Study HLD200-104). The next morning the subjects received a medium fat breakfast. Gender and weight where collected for each individual and were explored as potential covariate in the PK model.

PK model development. The population PK analysis was performed in the following sequence of steps: 1. Exploratory data analysis, 2. Base structural model development, 3. Covariate analysis, 4. Model refinement, 5. Model evaluation, as follows:

1. Exploratory data analysis: Exploratory data analyses was performed to understand the informational content of the analysis dataset with respect to the anticipated models, to search for extreme values and/or potential outliers, to examine the correlation between covariates, and to assess possible trends in the data. Linear and log-linear scatterplots of concentrations vs time were generated using the individual PK values and the mean values at the different sampling times.

2. Base structural model development: An initial evaluation of the PK results indicated that HLD200 concentration-time profiles exhibit a disposition/elimination shape consistent with a one-compartment PK model. Hence, PK models based on a one-compartment was evaluated.

An initial evaluation of the PK results indicated that HLD200 concentration-time profiles exhibit a disposition/elimination shape consistent with a one-compartment PK model. Hence, PK models based on a one-compartment was evaluated.

$$\begin{matrix}\frac{d {c}_{p}}{d c} = f ( t ) - kel \cdot {C}_{p} & ( Eq . 1 ) \\ f ( t ) = \frac{dr}{dt} & ( Eq . 2 )\end{matrix}$$

where kel is the elimination rate constant, f(t) is the time varying in-vivo release rate, r(t) is the input function, and Cp is the MPH concentration.

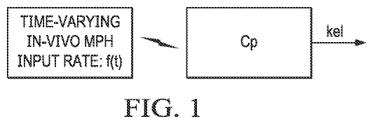

A convolution-based modeling approach was applied using a prescribed input function (R. Gomeni, F. Bressolle, T. J. Spencer, S. V. Faraone. Meta-analytic approach to evaluate alternative models for characterizing the PK profiles of extended release formulations of MPH. ASCPT 2016 Annual Meeting, Mar. 8-12, 2016, Hilton Bayfront, San Diego, Calif., incorporated by reference herein). FIG. 1 is a schematic of the HLD200 PK model.

Two time varying in-vivo absorption models were evaluated: single [r1(t)] and dual [r2(t)] Weibull functions.

$$\begin{matrix}r 1 ( t ) = {e}^{- ( {( \frac{t i m e}{t d} )}^{s s} )} & ( Eq . 3 ) \\ r 2 ( t ) = ff \cdot {e}^{- ( {( \frac{t i m e}{t d} )}^{ss} )} + ( 1 - ff ) \cdot {e}^{- ( {( \frac{t i m e}{t d 1} )}^{ss 1} )} & ( Eq . 4 )\end{matrix}$$

where: ff is fraction of the dose released in the 1st process, td is the time necessary to absorb 63.2% of the dose released in the 1st process, td1 is the time necessary to absorb 63.2% of the dose released in the 2nd process, ss is the sigmoidicy factor for the 1st process, and ss1 is the sigmoidicity factor for the 2nd process.

The estimated model parameters were: the mean structural model parameters, the magnitude of inter-individual variability (IIV), the magnitude of intra-occasion variability (IOV), and the magnitude of residual variability.

The following variance component model were evaluated:

Inter-individual Variability: The inter-individual variability (IIV) model describes the unexplained random variability in individual values of structural model parameters (Fixed effect). It was assumed that the IIV of the model parameters was log-normally distributed. The relationship between a model's parameter (P) and its variance was therefore expressed as:

Pⱼ=P_{TV}e^{ηp}

where Pⱼ is the value of parameter for the jₜₕ individual, P_{TV} is the typical value of P for the population, and ηp denotes the difference between Pⱼ and P_{TV}, normally distributed with a mean zero and variance ωp². If the goodness-of-fit plots reveal potential biases in the error model, alternative error models may be considered.

Inter-Occasion Variability: As all patients had PK samples on two occasions, inter-occasion variability was evaluated for the population PK parameters using an exponential error model.

Residual Variability: The residual variability, which comprised of, but not limited to, intra-individual variability, experimental errors, process noise and/or model misspecifications, was modeled using additive, proportional and combined error structures as described below:

Additive error: yij=ytij+ε1ij

Proportional error: yij=ytij(1+ε1ij)

Combined additive and proportional error: yij=ytij(1+ε1ij)+ε2ij

where yij is the jth observation in the ith individual, ytij is the corresponding model prediction, and ε1ij (or ε2ij) is a normally distributed random error with a mean of zero and a variance of σ2. If the goodness-of-fit plots revealed potential biases in the residual variability model, other residual error models.

The parameters estimation procedure was conducted using a non-linear mixed effect modeling approach as implemented in the NONMEM software (version 7.3). The following parameters were estimated:

Fixed effect:
kel: first order elimination rate constant
V: volume of distribution of the central compartment (V=V/F)
ss, td: parameters of the Weibull time varying input function

Random effect
IIV on kel, V, ss, td
IOV on V, ss, and td (no IOV was considered on kel assuming that this parameters did not change from one occasion to another)

Residual error:
Additive (Add_error) and proportional (Prop_erro) model components

Comparison of alternative models: The comparison of alternative models was performed using the log-likelihood ratio test. An alternative model was considered as a significantly better descriptor of data when the reduction in the objective function value (OFV) associated with this model was ≥3.84, χ2<0.05 for 1 degree of freedom (df).

Model diagnostics: Goodness of fit plot were generated for evaluating the results of model fitting. These plots included the plots of: a) the observed data versus individual and model predicted concentrations, b) the absolute weighted residuals versus individual predictions, and c) the conditional weighted residual versus time.

3. Covariate analysis: Graphical and statistical approaches along with consideration of the underlying scientific rationale was used to identify which covariates had to be examined on the population pharmacokinetic parameters and to assess the mathematical form of their relationships. Covariate effects were explored for all the model parameters. The prospectively identified covariates are: weight and gender.

Covariate model building was a step-wise process consisting on a forward and a backward selection procedure. The likelihood ratio test was used to evaluate the significance of incorporating or removing fixed effects into the population model based on alpha levels that are set a priori. For forward selections, a significance level of 0.05 for FOCE-I was used.

Step-wise forward addition procedure: Initially, each covariate individually was included in the base model to identify significant covariates where significance is a reduction in the objective function value (OFV) of >3.84, χ2<0.05 for 1 degree of freedom (df) using FOCE-I. Next, the significant covariates were added to the base model one covariate into one parameter at a time. The most significant covariate was included into the model first. This new model served as a new starting model for the next iteration. The test of significance and adding-on steps was repeated until all significant covariates are included and the final model is defined.

Backward elimination procedure: After the full model has been defined, the significance of each covariate was tested individually by removal one at a time from the full model. The following evaluation criteria was used to determine the significance of the covariates tested:

A covariate was retained in the model if, upon removal, the OFV increases by more than 6.63 points (χ2<0.01 for 1 df) using FOCE-I.

In addition to the OFV, the same criteria described above was used to assess the significance of a covariate in the procedure. The least non-significant covariate was excluded from the model. The elimination steps were repeated until all non-significant covariates are excluded and the final model is defined.

4. Model refinement: Simulations were performed to illustrate the effect of the retained covariates and their combinations on MPH exposure.

PD data. The clinical data used in this analysis were the SKAMP scores collected in a Phase 3 (HLD200-107 study), multicenter, open-label treatment-optimized, double-blind, randomized, placebo-controlled, forced-withdrawal, parallel group study to evaluate the safety and efficacy of evening dosed HLD200, a novel delayed and extended release formulation (DELEXIS) of methylphenidate hydrochloride, in children aged 6-12 with attention deficit hyperactivity disorder (ADHD) in a laboratory classroom setting.

PK/PD model development. In this study, no MPH samples were collected. Therefore the individual exposure, used in the PK/PD analysis were estimated based on the outcomes of population PK model and on the individual demographic (weight and gender) covariate values.

The model included a parametric description of the trajectories of SKAMP scores for placebo and HLD200 treatments.

An indirect response model [R(0] was used to describe the trajectories of SKAMP scores following placebo administration. The HLD200 effect was described by a drug-related change from placebo using an Emax model:

$$SKAMP ( t ) = R ( t ) \cdot ( 1 - \frac{E max \cdot {C}_{p}^{g}}{{EC}_{50}^{g} + {C}_{p}^{g}} ) \qquad ( Eq . 5 )$$

R(t) is the placebo response defined by:

$$\begin{matrix}\frac{d R}{d t} = {k}_{i n} \cdot ( 1 + p ( t ) ) - {k}_{o u t} R & ( Eq . 6 ) \\ p ( t ) = AA \cdot {e}^{- t \cdot P 1} & ( Eq . 7 )\end{matrix}$$

where kin represents the zero-order rate constant for production of response (R), kₒᵤₜ is the first-order rate constant for the loss of response, AA is the amplitude of the placebo effect, Pi is the rate of change of the placebo effect, Emax is the maximal achievable HLD200-related effect, EC₅₀ is the MPH concentration associated with 50% of the maximal response, and g is the shape of the exposure-response relationship. As the system is assumed to be stationary, the response variable (R) begins at a predetermined baseline value (Bas), changes with time and returns back to Bas. Thus:

$$R ( t = 0 ) = Bas = \frac{{k}_{i n}}{{k}_{o u t}} \qquad ( Eq . 8 )$$

Model Performance. Models performance/validation and stability was assessed using visual predictive checks. Visual predictive check (VPC) method was utilized to evaluate the adequacy of final model, including the effects of statistically significant covariates. This assumes that parameter uncertainty is negligible, relative to inter-individual and residual variance. The basic premise is that a model and parameters derived from an observed data set should produce simulated data that are similar to the original observed data. Five hundred replicates of the original dataset were simulated, based on the final model, and the 90% prediction interval was computed based on the simulated datasets. The observed concentration versus time data were plotted on the prediction interval to visually assess the concordance between the simulated and observed data. The quantiles (5th, median, 95th) of simulated data were compared graphically to the observed data quantiles.

Software. All data preparation, summary statistics (mean, median, standard deviation, and other measures, as appropriate), report and graphical display presentation were performed using SAS (version 9.3) and R (version 3.2.5). Any SAS and R scripts used for data preparation and final analyses were achieved. The population PK and PK/PD analyses were conducted using the NONMEM software, Version 7.3 (ICON Development Solutions). The R-based package Xpose (version 4.3) was used as a model building aid for population analysis using NONMEM. The SAS statistical package (version 9.3) was used to perform the statistical evaluation of the outcomes of the clinical trial simulations.

A total of 20 subjects were included in population PK analysis dataset with a total of 960 PK measurements.

The following abbreviations are used herein: ADHD (Attention deficit hyperactivity disorder), AUEC (Area under the effect curve), CB (Clinical benefit), CI (Confidence interval), df (Degree of freedom), ER (Extended release), F (Bioavailability), FI (Fluctuation index), FOCE-I (First order conditional method with interactions), IIV (Intra-subject variability), MAX (Maximum value), MIN (Minimum value), MPH (Methylphenidate), OFV (Objective function value), PK (Pharmacokinetics), RSE (Relative square error), SD (Standard deviation), SE (Standard error), VPC (Visual predictive checks).

Examples 1 to 9 relate to PK analysis results.

### Example 1. PK Population: Demographic Data

Table 1 shows the distribution of the subjects included in the study by gender. Table 2 shows the descriptive statistics on weigh and Table 3 shows the descriptive statistics on weight by gender.

TABLE 1
Distribution of subjects by gender:

| | | | Cumulative | Cumulative |
|---|---|---|---|---|
| gender | Frequency | Percent | Frequency | Percent |
| Female | 14 | 70.00 | 14 | 70.00 |
| Male | 6 | 30.00 | 20 | 100.00 |

TABLE 2
Descriptive statistics on weight:
Analysis Variable: weight

| N | Mean | Median | Minimum | Maximum |
|---|---|---|---|---|
| 20 | 67.59 | 65.65 | 51.80 | 90.10 |

TABLE 3
Descriptive statistics on weight by gender:
Analysis Variable: weight

| gender | N Obs | Mean | Median | Minimum | Maximum |
|---|---|---|---|---|---|
| Female | 14 | 63.36 | 60.90 | 51.80 | 79.20 |
| Male | 6 | 77.45 | 77.05 | 64.80 | 90.10 |

### Example 2. PK Population: Descriptive Analysis

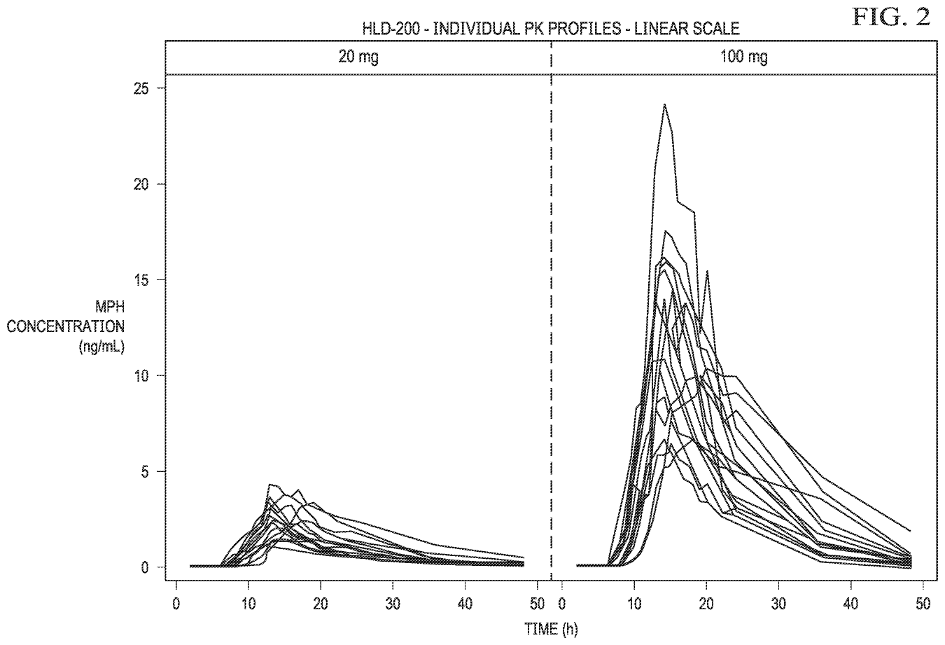

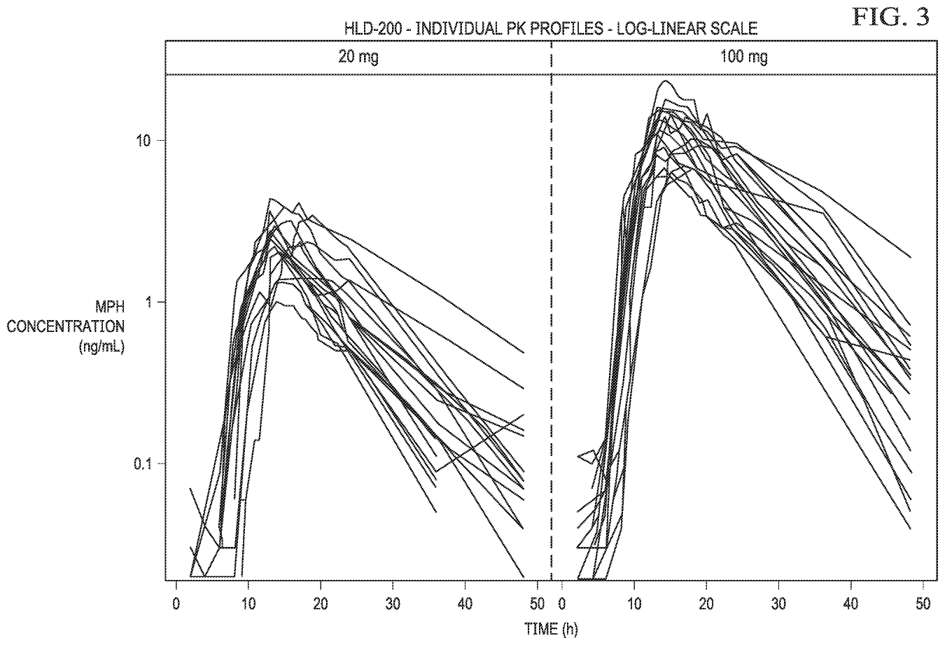

FIG. 2 and FIG. 3 show exemplary scatter plots of the individual PK profiles by dose in linear and log-linear scale respectively.

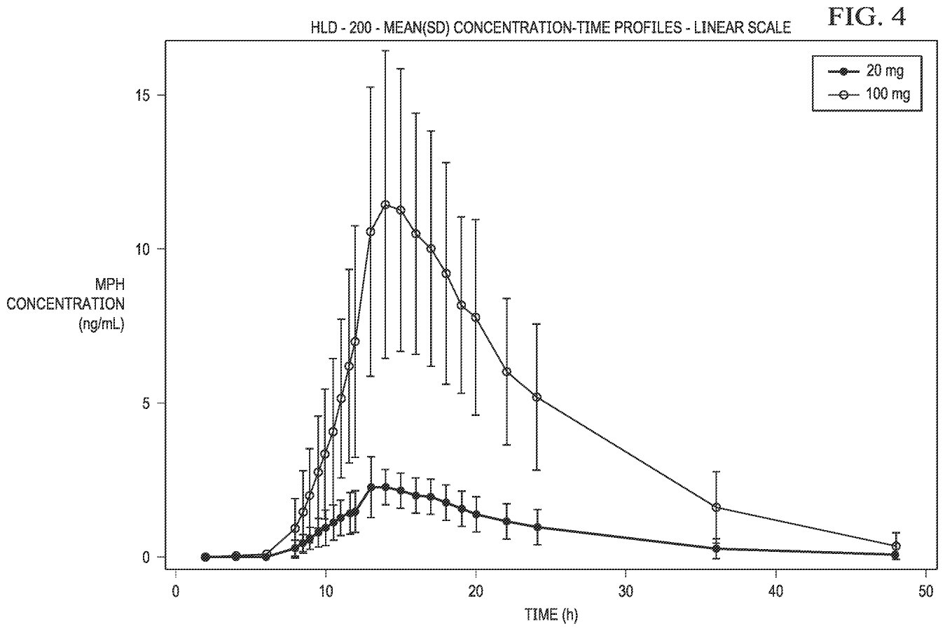

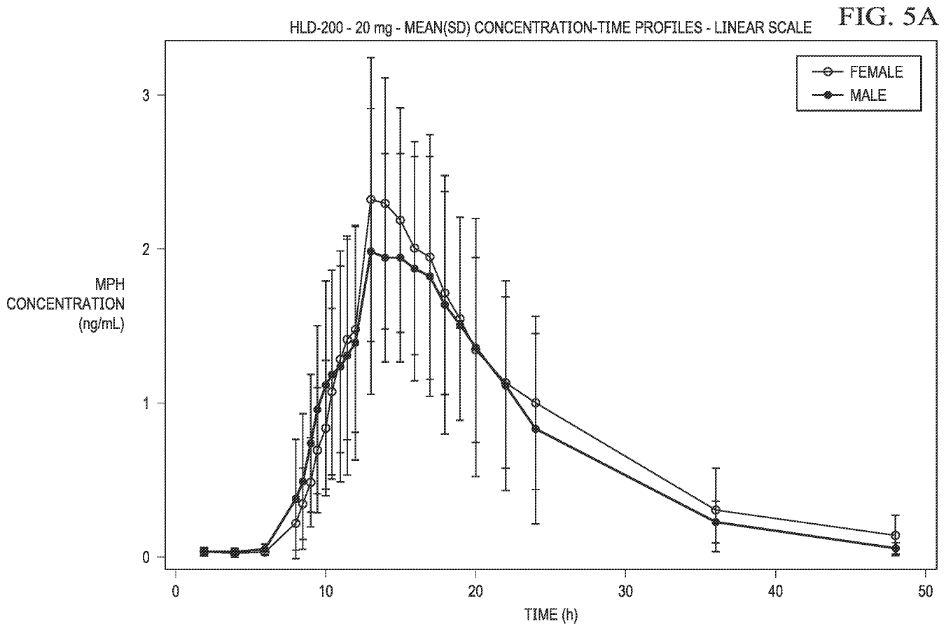

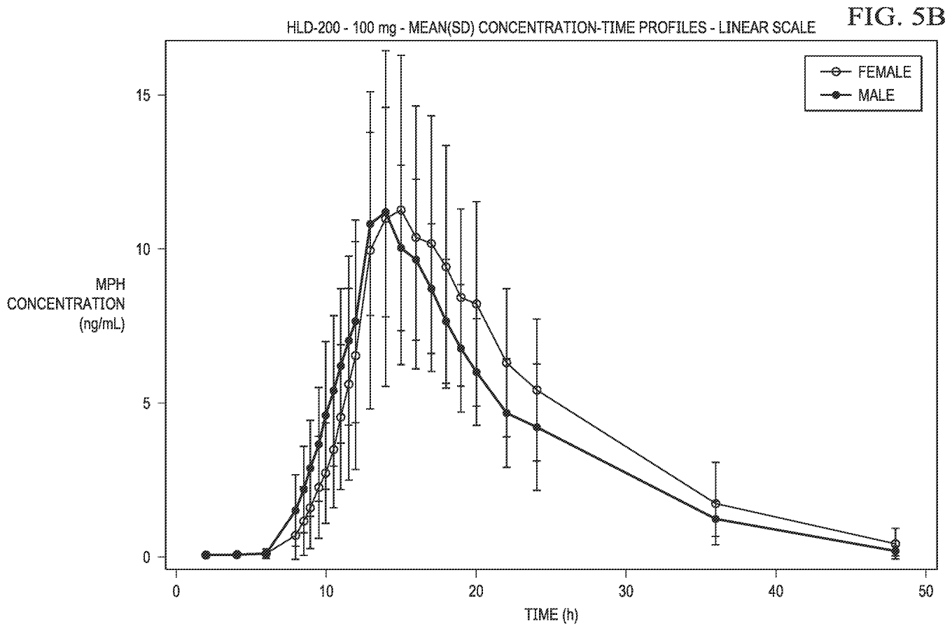

FIG. 4 and FIG. 5A and FIG. 5B show exemplary scatter plots of the mean (±SD) PK profiles by dose in linear scale in the total population and by gender.

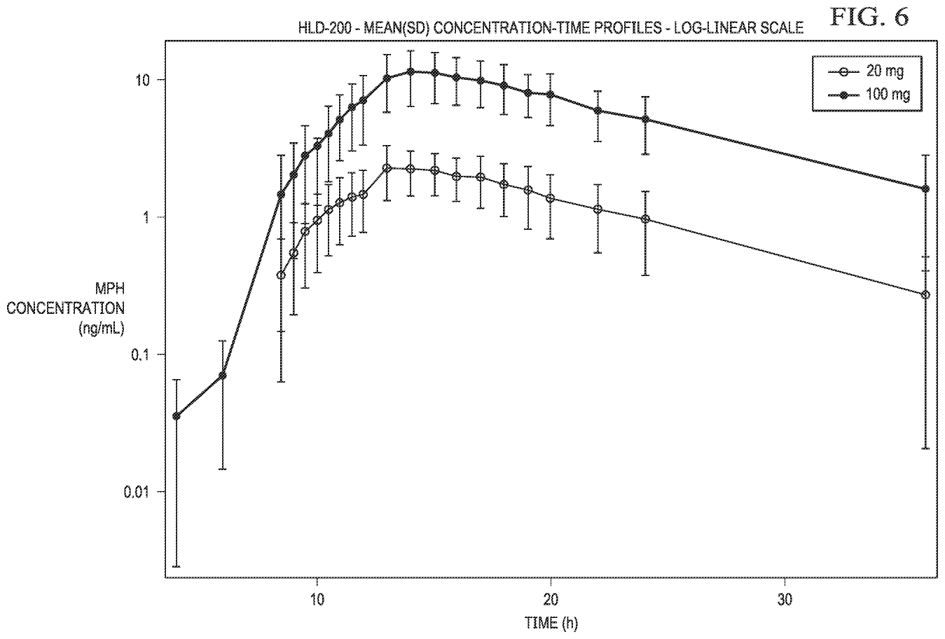

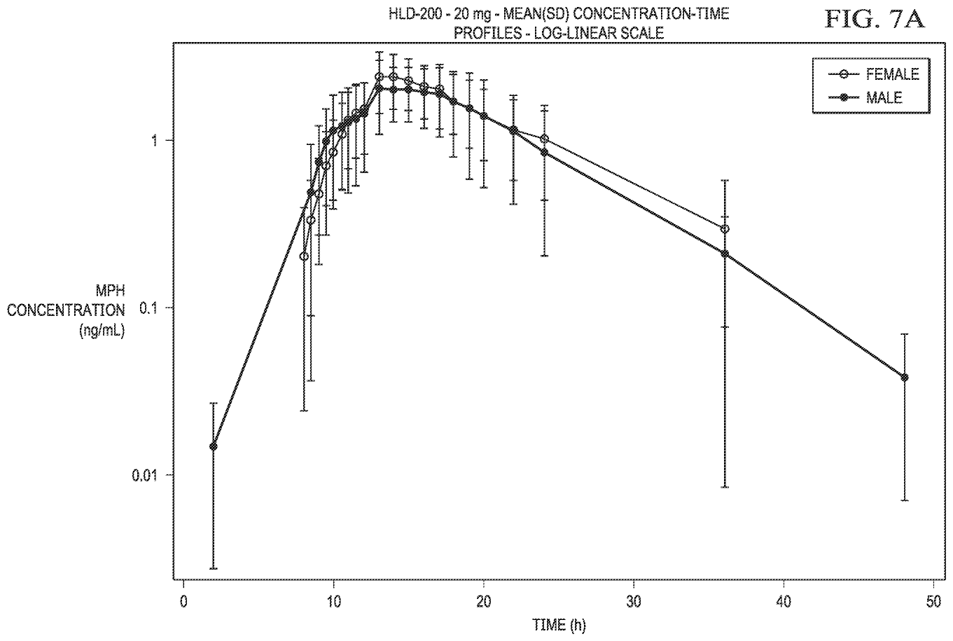

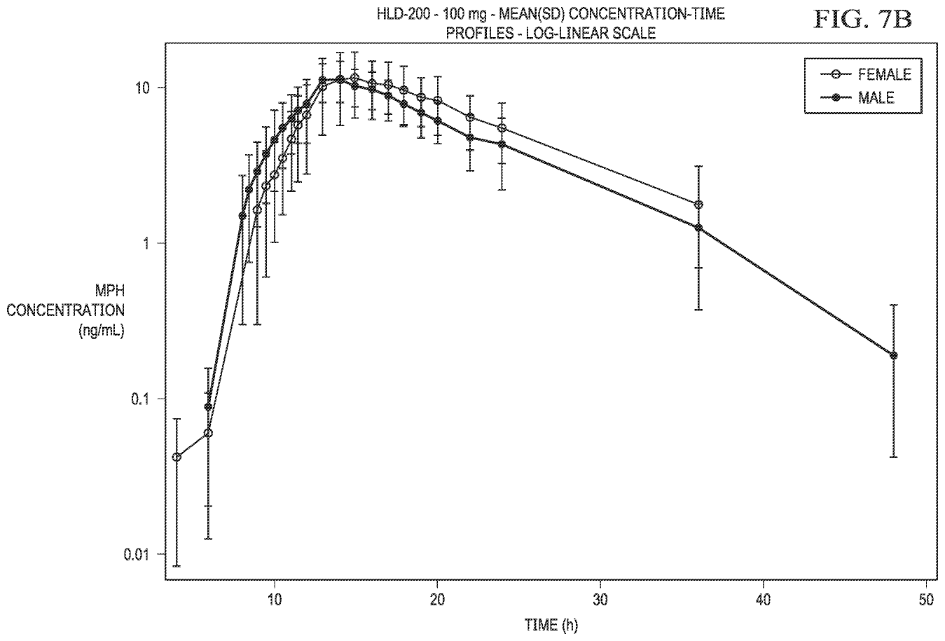

FIG. 6 and FIG. 7A and FIG. 7B show exemplary scatter plots of the mean (±SD) PK profiles by dose in log-linear scale in the total population and by gender.

### Example 3. PK Model Development: Comparison Between One and Two Weibull Input Functions

The comparison between one and two Weibull input functions is presented in Table 4 in the chronological order in which they were evaluated. The first column of the table lists each model tested. The second column lists the reference run to which the test run was compared. The third and fourth columns list the OFV for each test run and the change in OFV from the reference run (test-reference), respectively. The fifth column describes briefly the hypothesis or objective that was tested by the model. The test outcome in the last column describes the conclusion, which was either not statistically significant or statistically significant, that can be drawn from the comparison with the reference run based on chi-squared statistics.

TABLE 40
Comparison between one and two Weibull input functions:

| | | | | | Description | |
|---|---|---|---|---|---|---|
| | Reference | | Change | | of the | p |
| Run | Run | OFV | in OFV | df | Model Tested | value |
| Run 1 | | −410.352 | | | One Weibull | |
| | | | | | function | |
| Run 2 | Run 1 | −365.879 | 44.473 | 1 | Two Weibull | 1 |
| | | | | | function | |
| Preferred | Run 1 | | | | | |
| model | | | | | | |

OFV = maximum likelihood objective function value
(*) = Statistically significant

The results of the comparison indicated that the single Weibull input function model is an appropriate model. Therefore, this model was considered as the reference base model.

### Example 4. PK Model Development: Evaluation of the Inter-Occasion Variability

Considering that two treatments (20 mg or 100 mg) were randomly administered to each subject in a separate treatment period (occasion), the inter-occasion variability (IOV) in addition to the intra-individual variability (IIV) in the PK response was evaluated. The comparison of the performances of the base model with and without the assessment of the inter-occasion variability is presented in Table 5.

TABLE 5
Evaluation of the inter-occasion variability:

| | | | | | Description | |
|---|---|---|---|---|---|---|
| | Reference | | Change | | of the | p |
| Run | Run | OFV | in OFV | df | Model Tested | value |
| Run 1 | | −410.352 | | | No IOV | |
| Run 3 | Run 1 | −1163.63 | −753.278 | 3 | With IOV | <0.0001* |
| Preferred | Run 3 | | | | | |
| model | | | | | | |

OFV = maximum likelihood objective function value
*= Statistically significant

The results of the comparison indicated that the presence of IOV parameters in the significantly (p<0.0001) improved the performances of the model. Therefore, the model ‘Run 3’ was considered as the new reference base model.

### Example 5. PK Model Development: Base Model Parameter Estimates

The estimated fixed effect and random effect parameters are shown in Table 6 and 7 respectively. In these tables, SE represents the standard error of the parameter estimates, and RSE represents the root mean square error and CV % represents the coefficient of variation of the IIV and IOV variability.

TABLE 6
Base population PK model: fixed effect parameter estimates:

| Fixed | | | | |
|---|---|---|---|---|
| Effect | Parameter | Value | SE | RSE |
| | TD(hr) | 12.50 | 0.38 | 3.10% |
| | VL(L) | 3830.00 | 312.00 | 8.10% |
| | SS | 7.53 | 0.38 | 5% |
| | KEL(hr-1) | 0.11 | 0.01 | 6% |
| | Add_error | 0.04 | 0.01 | 21.90% |
| | Prop_error | 0.17 | 0.02 | 10.10% |

TABLE 7
Base population PK model: random effect parameter estimates:

| | Parameter | Variability | Value | CV % | SE | RSE |
|---|---|---|---|---|---|---|
| Random | TD | IIV | 0.013 | 11.30% | 0.0041 | 32.30% |
| Effect | | IOV | 0.009 | 9.60% | 0.0032 | 34.70% |
| | V | IIV | 0.136 | 36.90% | 0.0482 | 35.40% |
| | | IOV | 0.008 | 9.20% | 0.0039 | 46.80% |
| | SS | IIV | 0.038 | 19.40% | 0.0234 | 61.90% |
| | | IOV | 0.015 | 12.30% | 0.0082 | 54.10% |
| | KEL | IIV | 0.069 | 26.30% | 0.0196 | 28.30% |

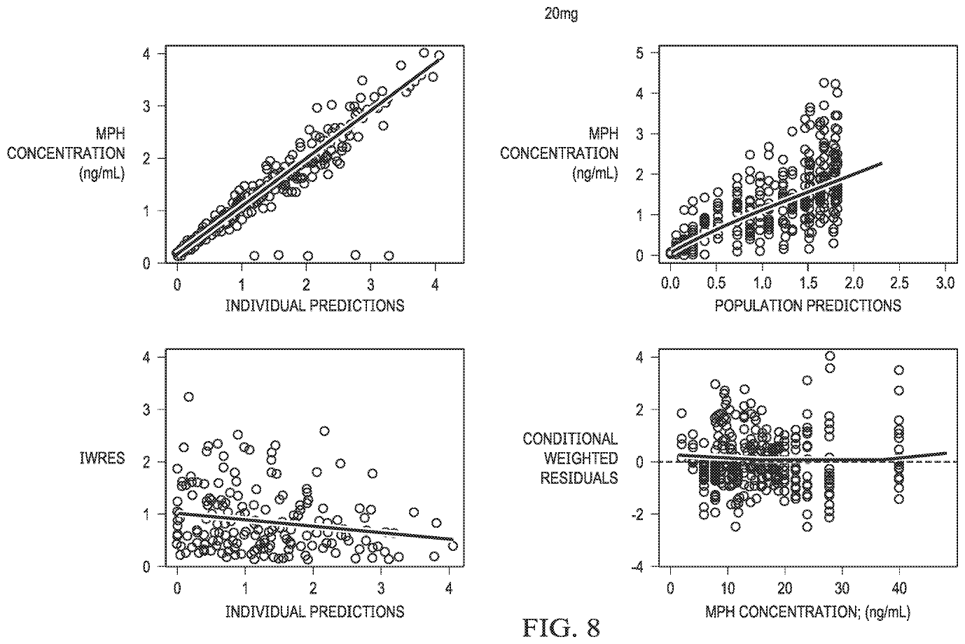

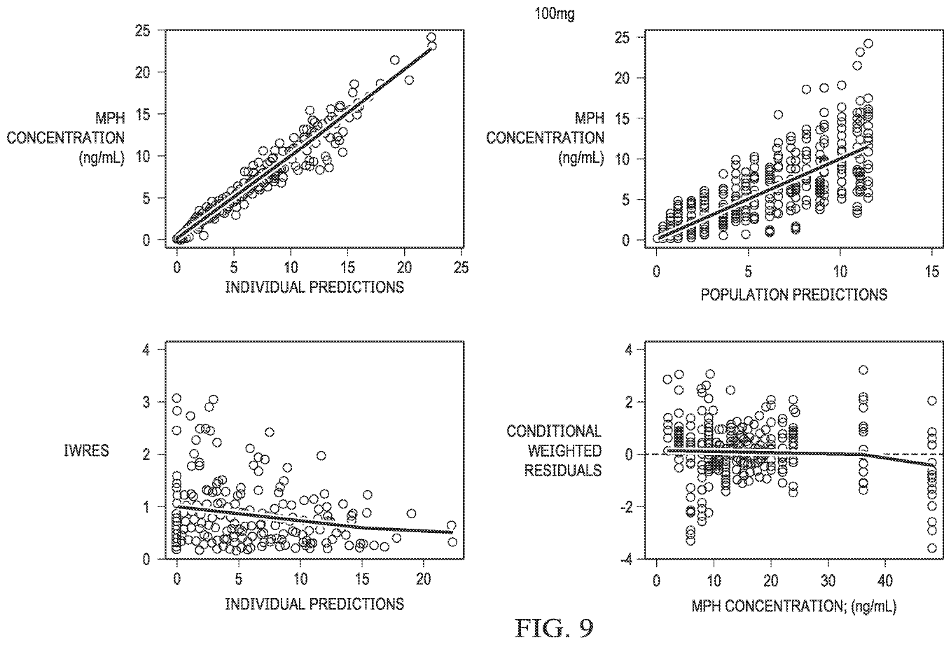

The goodness-of-fit (GOF) diagnostic plots for the base population PK model are shown in FIG. 8 and FIG. 9 by dose. Lines of identity, zero lines, and trend lines are overlaid when appropriate.

Overall, there was no apparent bias in these diagnostic plots, suggesting that the base population PK model was adequate in describing the HLD200 PK.

### Example 6. PK Model Development: Covariate Analysis

Graphical and statistical approaches along with consideration of the underlying scientific rationale were used to identify the covariates to be included in the base population PK model and to assess the mathematical form of the relationships between parameters and covariates. The prospectively identified covariates were: gender, and weight.

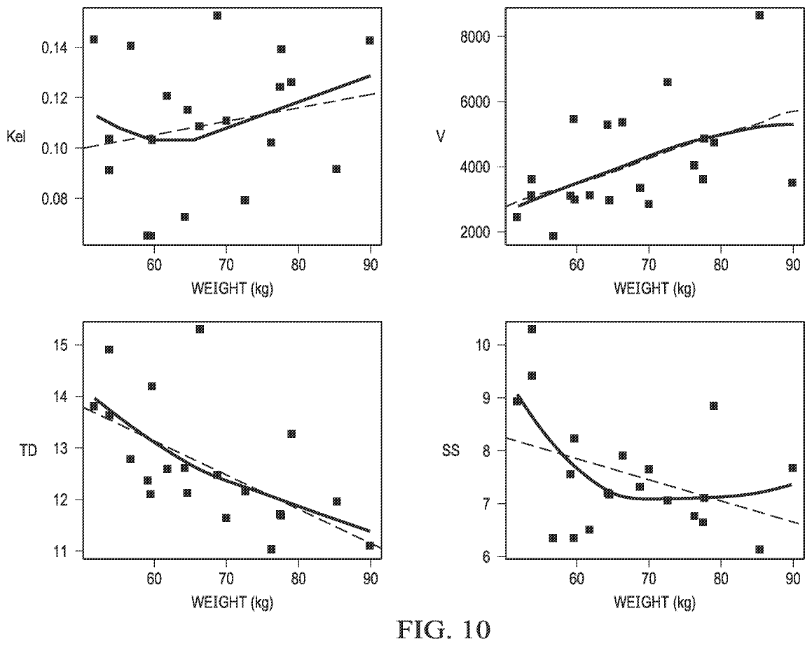

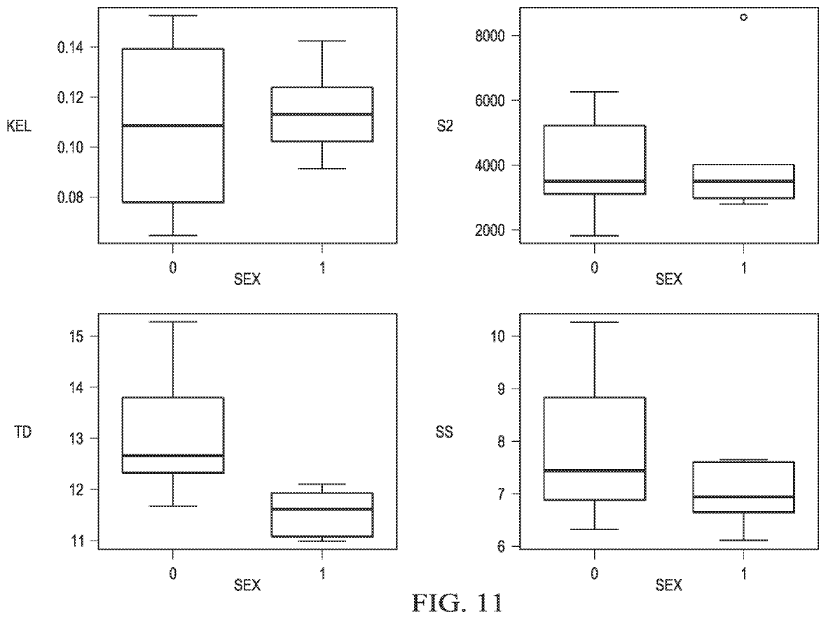

Empirical Bayesian estimates of individual parameters were obtained from the base model (Run 3) in the NONMEM analysis. A graphical exploration of the potential impact of covariates on the PK parameters variability was conducted by analyzing the scatter plot of the individual PK parameter versus the selected covariates (FIG. 10 and FIG. 11).

The analysis of the distribution of the individual parameter estimates versus the selected covariates indicated a potential dependency of V, TD, and SS to the weight and a dependency of TD to the gender.

The characteristics of the reference base model (in absence of covariate) and the different models explored to account for the covariate effect are summarized in Table 8.

TABLE 8
List of all intermediate population PK models evaluated:

| | Reference | | Change | | Description of the | |
|---|---|---|---|---|---|---|
| Run | Run | OFV | in OFV | df | Model Tested | p value |
| Run 3 | | −1163.63 | | | Reference base model | |
| Run 4 | Run 3 | −1170.081 | −6.451 | 0 | Weight on V | <0.0001(*) |

Forward addition procedure

| | | | | | | |
|---|---|---|---|---|---|---|
| Run 5 | Run 4 | −1180.08 | −9.999 | 1 | Weight on V + Weight | <0.0016(*) |
| | | | | | on TD | |
| Run 6 | Run 4 | −1181.652 | −11.571 | 1 | Weight on V + Gender | <0.001(*) |
| | | | | | on TD | |
| Run 7 | Run 6 | −1181.443 | 0.209 | 1 | Weight on V + Weight | 0.65 |
| | | | | | on TD and on SS | |
| Run 8 | Run 6 | −1184.905 | −3.253 | 1 | Weight on V + Weight | 0.071 |
| | | | | | on TD and Gender on | |
| | | | | | TD | |
| Best Performing | Run 6 | −1181.652 | | | | |
| model | | | | | | |

Backward elimination procedure

| | | | | | |
|---|---|---|---|---|---|
| Run 9 | Run 6 | −1175.043 | 6.609 | remove Weight on V | 1 |
| Final model | Run 6 | | | | |

OFV = maximum likelihood objective function value
(*)= Statistically significant

The covariate analysis indicated that the best performing model was the one with an effect of weight on volume of distribution and of gender on TD: the volume increase with weight and the time for release 63.8% of the dose (TD) is longer (˜20%) in female with respect to male.

The details of the models accounting for the covariate effect is presented below:

Run 3 (Reference base model):

TD=θ₁

kel=θ₂

V=θ₃

SS=θ₄

Run 4 (Weight on V using an allometric scaling model):

$$V = {θ}_{3} \cdot ( \frac{W T}{70} )$$

Run 5 (Weight on V and Weight on TD):

$$V = {θ}_{3} \cdot ( \frac{W T}{70} )$$

$$T D = {θ}_{1} \cdot {e}^{( - {θ}_{5} \cdot WT )}$$

Run 6 (Weight on V and Gender on TD):

$$V = {θ}_{3} \cdot ( \frac{W T}{70} )$$

where If gender=M then TD=θ₁ else TD=θ_{S}

Run 7 (Weight on V and on SS):

$$V = {θ}_{3} \cdot ( \frac{W T}{70} )$$

$$S S = {θ}_{4} \cdot {e}^{( - {θ}_{5} \cdot WT )}$$

Run 8 (Weight on V, Weight on TD and Gender on TD):

$$V = {θ}_{3} \cdot ( \frac{W T}{70} )$$

where if gender=M then TD=θ₁ else TD=θ_{S}

TD=θ₁·e^{(-θ}^{₆}^{·WT)}

Run 9 (Gender on TD): where, if gender=M then TD=θᵢ else TD=θ_{S}

The estimated fixed effect and the random effect parameters of the final model are presented in Table 9 and 10 respectively. In these tables, SE represents the standard error of the parameter estimates, and RSE represents the root mean square error and CV % represents the coefficient of variation of the IIV and IOV variability.

TABLE 9
Final population PK model: fixed effect parameter estimates:

| Fixed | | | | |
|---|---|---|---|---|
| Effect | Parameter | Value | SE | RSE |
| | TD(hr) Male | 10.90 | 0.34 | 3.10% |
| | VL(L) | 4000.00 | 282.00 | 7% |
| | SS | 7.52 | 0.39 | 5.20% |
| | KEL(hr-1) | 0.11 | 0.01 | 6.10% |
| | TD (hr) Female | 13.20 | 0.39 | 3% |
| | Add_error | 0.04 | 0.01 | 22.50% |
| | Prop_error | 0.17 | 0.02 | 10% |

TABLE 10
Final population PK model: random effect parameter estimates:

| | Parameter | Variability | Value | CV % | SE | RSE |
|---|---|---|---|---|---|---|
| Random | TD | IIV | 0.005 | 6.86% | 0.0026 | 55.50% |
| Effect | | IOV | 0.009 | 9.59% | 0.0032 | 35% |
| | V | IIV | 0.094 | 30.66% | 0.0281 | 29.90% |
| | | IOV | 0.008 | 9.17% | 0.0039 | 46.50% |
| | SS | IIV | 0.04 | 19.95% | 0.0240 | 60.30% |
| | | IOV | 0.016 | 12.77% | 0.0085 | 51.90% |
| | KEL | IIV | 0.067 | 25.83% | 0.0185 | 27.70% |

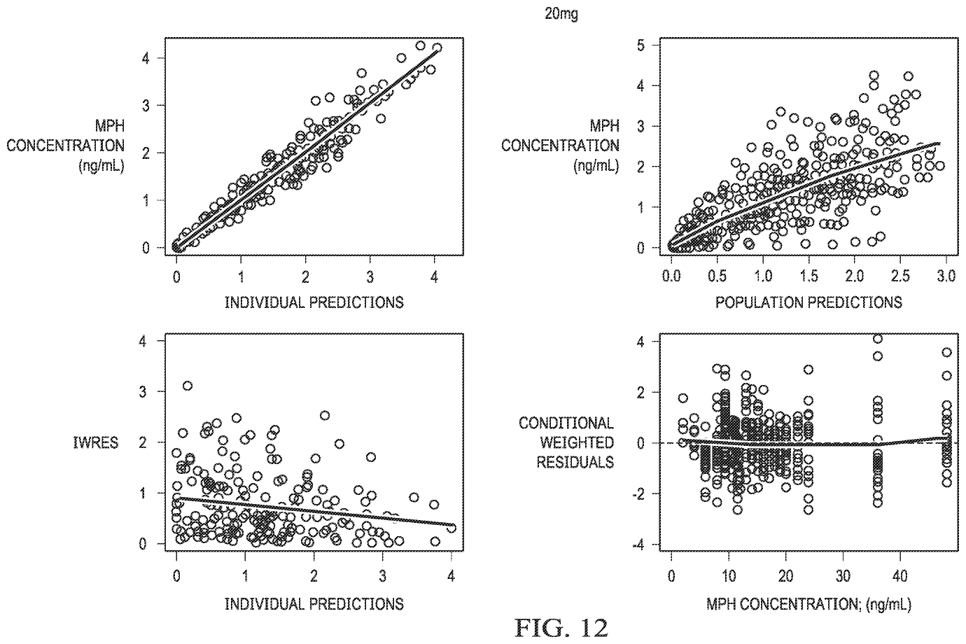

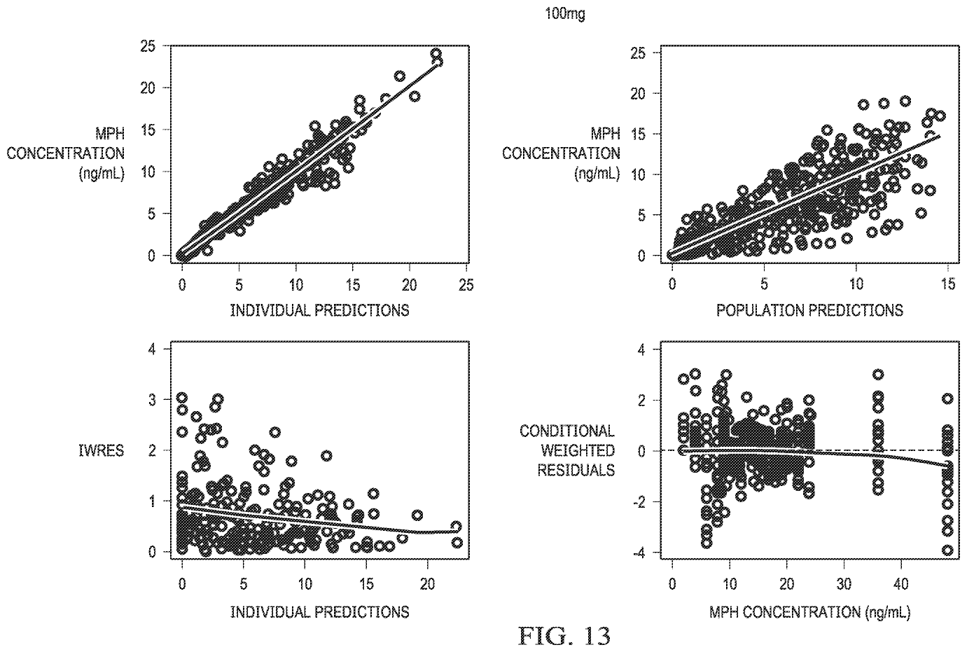

The goodness-of-fit (GOF) diagnostic plots for the base population PK model are shown in FIG. 12 and FIG. 13 by dose. Lines of identity, zero lines, and trend lines are overlaid when appropriate.

Overall, there was no apparent bias in these diagnostic plots, suggesting that the base population PK model was adequate in describing the HLD200 PK.

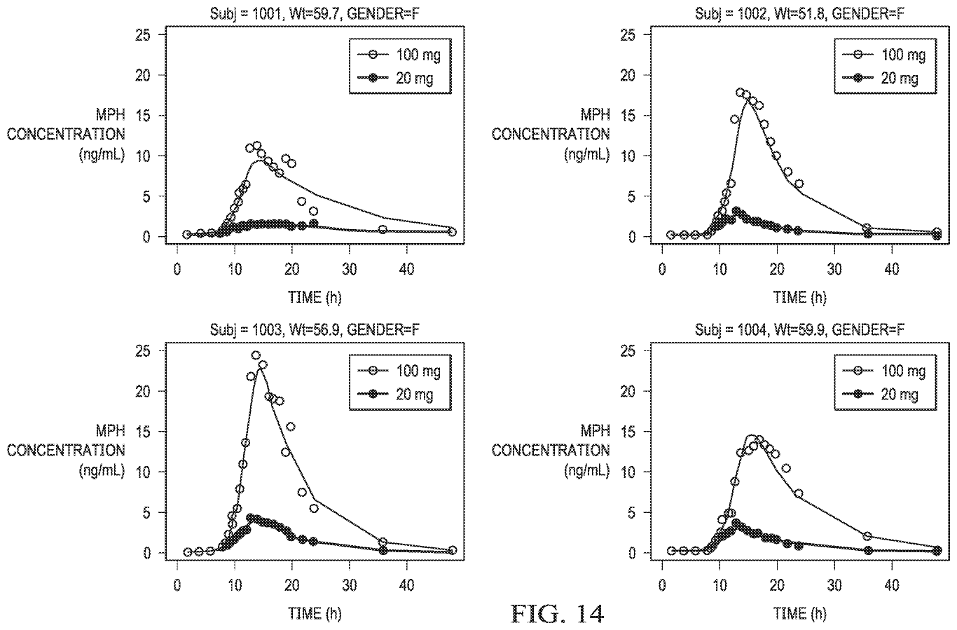

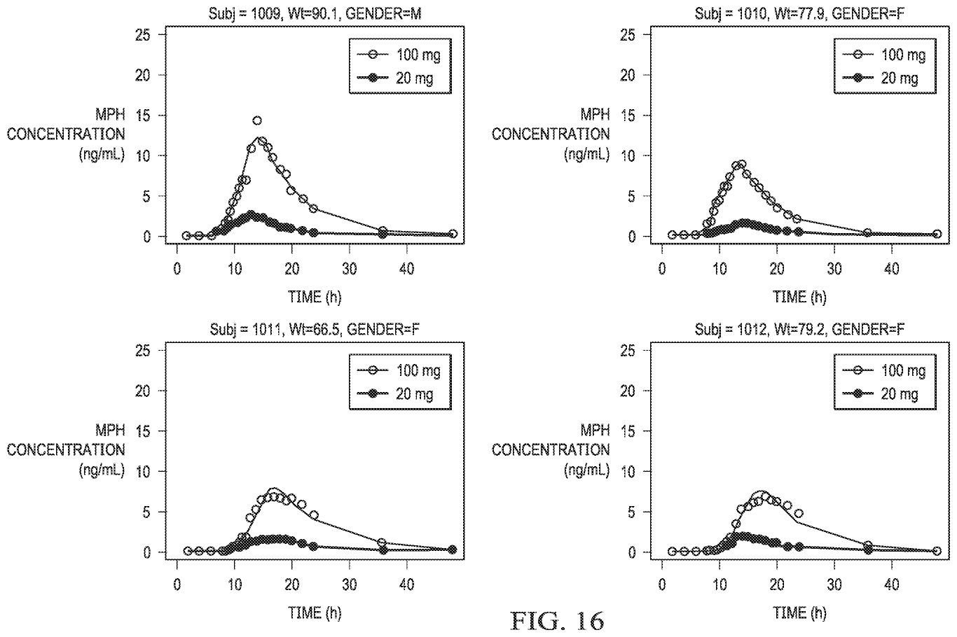

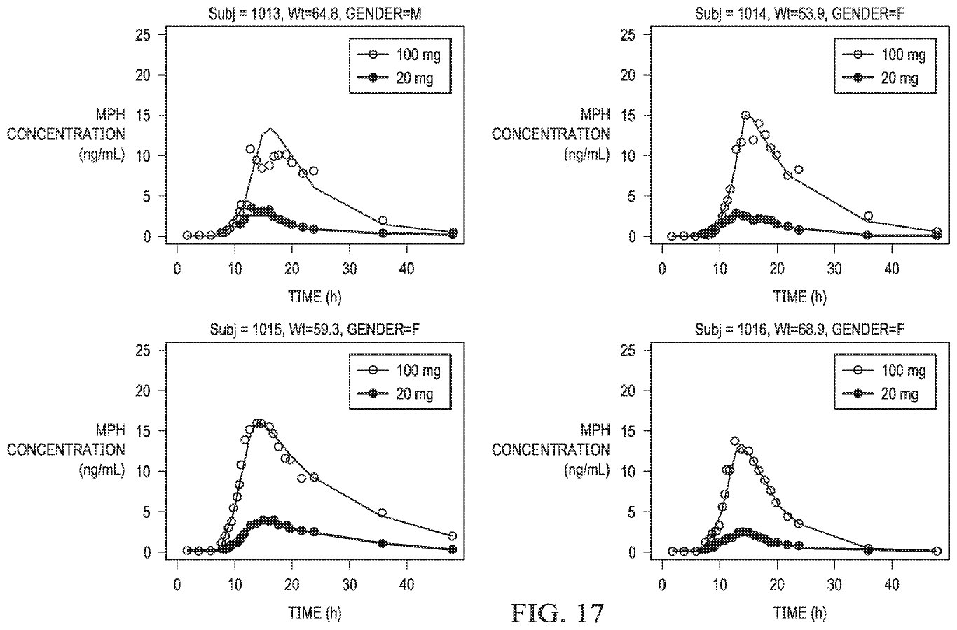

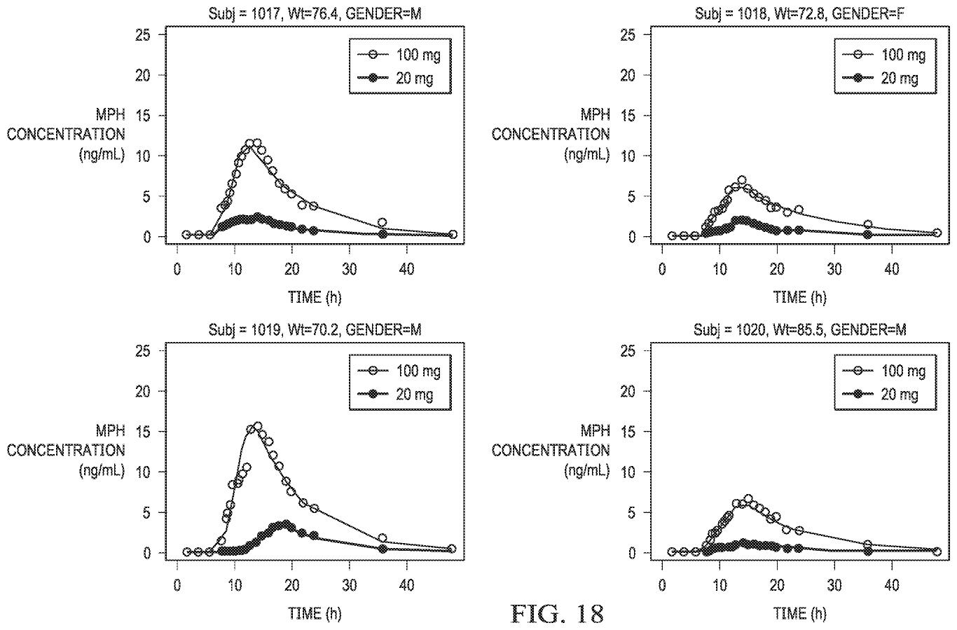

The individual model predicted and observed PK concentrations versus time are shown in FIGS. 14 to 18.

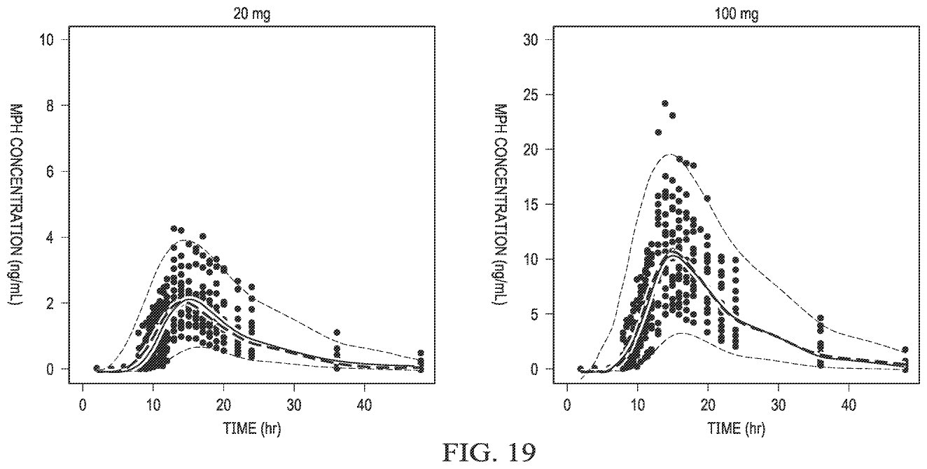

The VPCs for HLD200 at the dose of 20 mg and 100 mg are shown in FIG. 19.

The VPCs showed that the model performs well at the different doses: The median PK values as well as the dispersion of the data around the median were well described by the 5th percentile, the 50th (median) and the 95th percentile indicating that the population model properly described the observed data.

### Example 7. PK Model Development: Impact of Covariates on the HLD200 Exposure after Single HLD200 Dose

Simulations were performed to evaluate the effect of the retained covariates (weight and gender) and their combinations on MPH exposure.

To illustrate the expected impact of the covariates on the MPH exposure the covariate values were categorized as follow: Gender: F and M, Weight: 55 kg, 65 kg, and 75 kg, Dose: 20 mg and 100 mg.

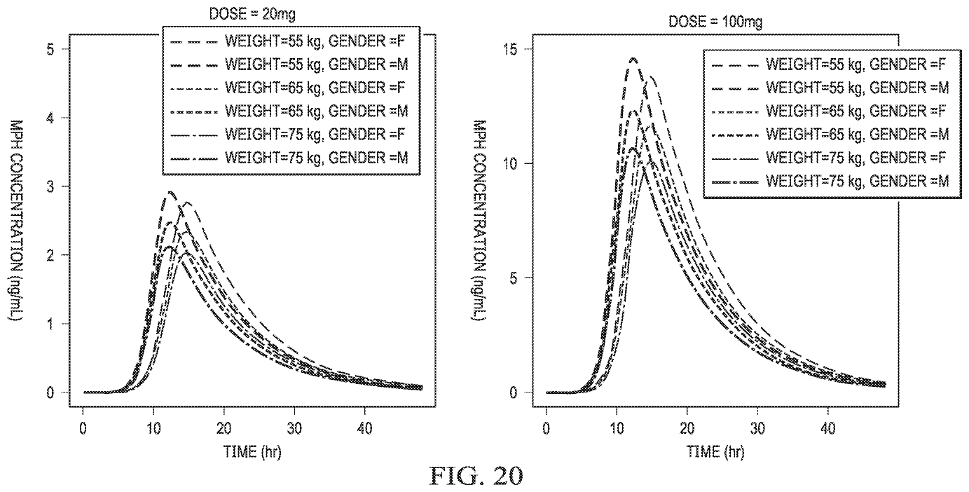

The median MPH concentrations were computed for each category and graphically presented in the FIG. 20.

The results of this evaluation indicated that the peak value increase with the decrease of the weight while the time to peak increase in the female with respect to male subjects.

### Example 8. PK Model Development: Impact of Covariates on the HLD200 Exposure after Repeated HLD200 Doses

Simulations were performed to evaluate the expected MPH exposure after repeated administrations of HLD200 as a function of the selected covariates.

The following simulation scenarios were considered: Gender: F and M, Weight: 63 kg for F and 77 kg for M (these values corresponds to the mean weight in the study population), Dose: 20 mg and 100 mg administered once a day during 6 days.

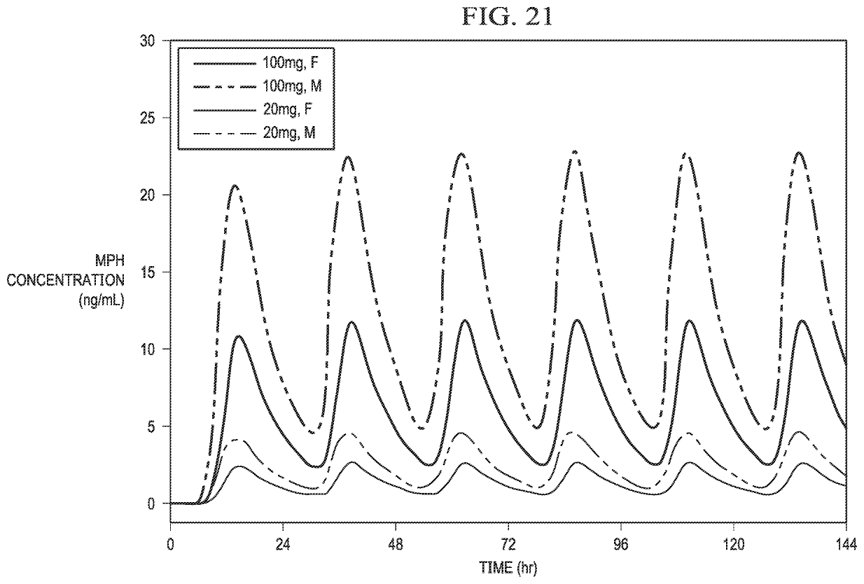

The median MPH concentrations were computed for each category and graphically presented in the FIG. 21. The Estimated Ctrough and Cmax values after repeated administrations of HLD200 are reported in Table 11.

TABLE 11
Estimated Ctrough and Cmax values after
repeated administrations of HLD200:

| | | | | Time post- | | Time post- |
|---|---|---|---|---|---|---|
| | | | | dose of | | dose of |
| | | Weight | Ctrough | Ctrough | Cmax | Cmax |
| Dose | Gender | (kg) | (ng/mL) | (hr) | (ng/mL) | (hr) |
| 20 mg | F | 63 | 0.6 | 7 | 2.6 | 14.5 |
| | M | 77 | 1 | 6 | 4.6 | 14 |
| 100 mg | F | 63 | 2.5 | 7 | 11.8 | 14.5 |
| | M | 77 | 4.9 | 6 | 22.7 | 13.5 |

### Example 9. PK Model Development: Comparison of the Exposure of HLD200 with the Exposure of Other ER MPH Formulations

A comparison of the MPH exposure of HLD200 at the dose of 20 mg and 100 mg has been done with respect to the exposure observed after administration of Concerta® (ALZA Corporation) (18 mg, 36 mg, 54 mg), Ritalin LA® (Novartis AG) (40 mg), Metadate CD® (UCB, Inc) (20 mg, 40 mg, and 60 mg), and Quillivant XR® (NextWave Pharmaceuticals, Inc.) (60 mg).

The comparison has been done assuming that HLD200 was administered the evening before bed time at 8 or 10 hours before school starting at 8:00 am the day after. The other ER MPH formulation was expected to be administered in the morning at 8:00 am. The simulated MPH concentrations after the administration of HLD200 have been generated using the population PK parameter values previously estimated.

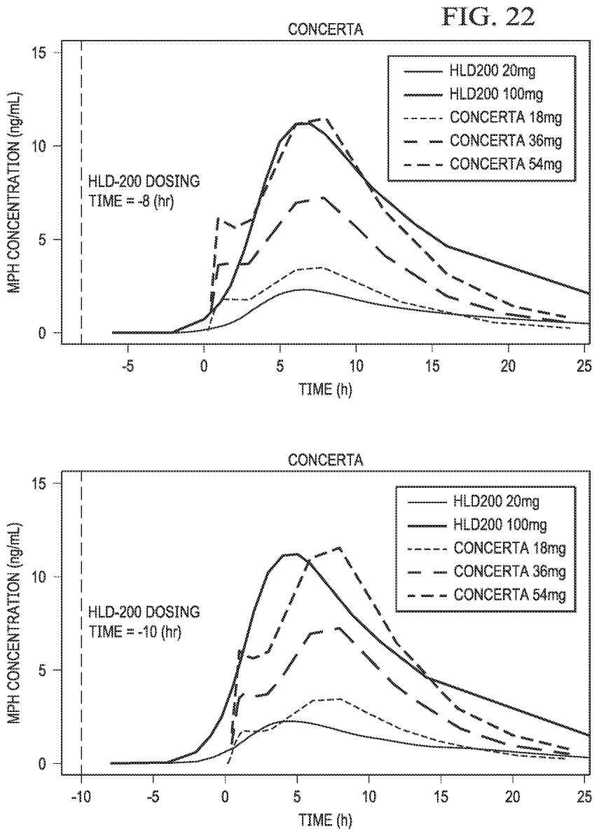

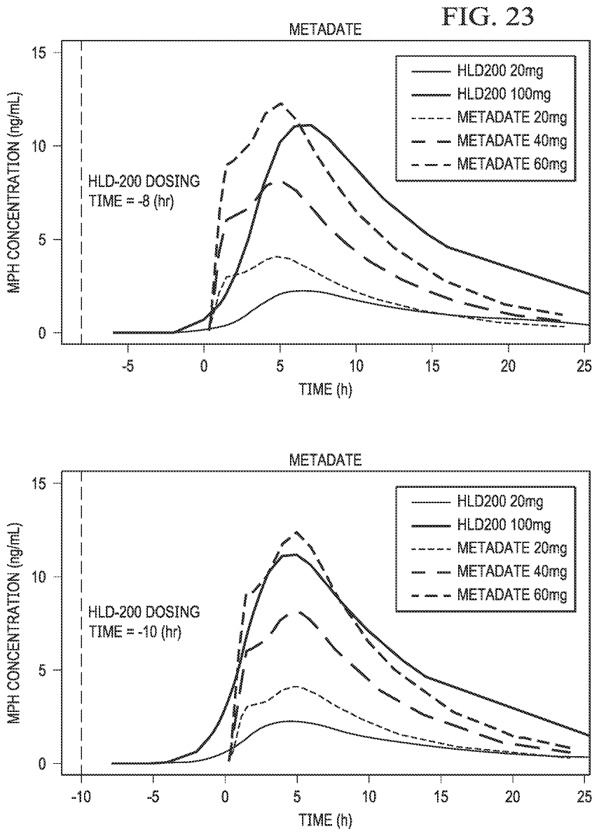

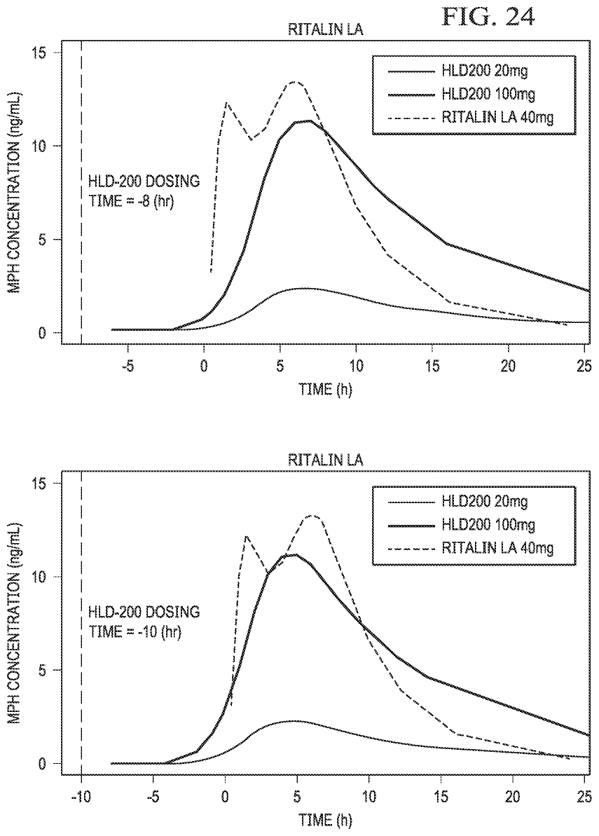

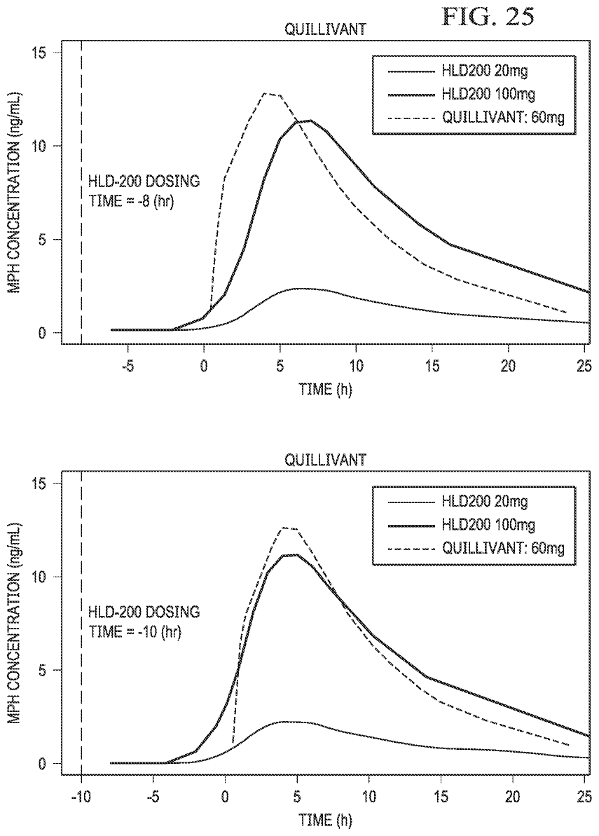

The results of the comparisons are presented in FIGS. 22 to 25.

Examples 10 to 15 relate to PK/PD analysis results. In the study HLD200-107 a total of 117 subjects were included: 53 were treated with placebo and 64 were treated with HLD200. A sequential modeling approach was applied. In the first step, the individual MPH concentrations were derived using the population PK model previously developed jointly with the individual demographic data and the individual HLD200 dosing history, then the placebo data were modelled, and finally the SKAMP scores were analyzed by fixing the placebo and the PK exposure values previously estimated.

### Example 10. PK/PD Analysis Results: PK/PD Population: Descriptive Analysis

Table 12 shows the distribution of the subjects included in the study by gender and treatment. Table 13 shows the descriptive statistics on age, weight, and height by gender and treatment.

TABLE 12
Distribution of the subjects included in the study by gender
and treatment:

| Frequency | | | | |
|---|---|---|---|---|
| Percent | Table of Gender by Treatment | | | |
| Row % | | Treatment | | |
| Col % | Gender | Placebo | HLD200 | Total |
| | Female | 15 | 22 | 37 |
| | | 12.82 | 18.80 | 31.62 |
| | | 40.54 | 59.46 | |
| | | 28.30 | 34.38 | |
| | Male | 38 | 42 | 80 |
| | | 32.48 | 35.90 | 68.38 |
| | | 47.50 | 52.50 | |
| | | 71.70 | 65.63 | |
| | Total | 53 | 64 | 117 |
| | | 45.30 | 54.70 | 100.00 |

TABLE 13
Descriptive statistics on the demographic data:

| Treatment | N Obs | Variable | N | Mean | SD | Median | Minimum | Maximum |
|---|---|---|---|---|---|---|---|---|
| Placebo | 53 | Age | 53 | 9.28 | 1.68 | 9.00 | 6.00 | 12.00 |
| | | Wt | 53 | 32.76 | 8.16 | 31.50 | 20.90 | 50.10 |
| | | Ht | 53 | 137.03 | 12.47 | 137.00 | 114.30 | 163.80 |
| HLD200 | 64 | Age | 64 | 9.58 | 1.58 | 10.00 | 6.00 | 12.00 |
| | | Wt | 64 | 32.68 | 8.88 | 30.85 | 19.80 | 56.10 |
| | | Ht | 64 | 136.49 | 10.73 | 136.10 | 114.60 | 169.00 |

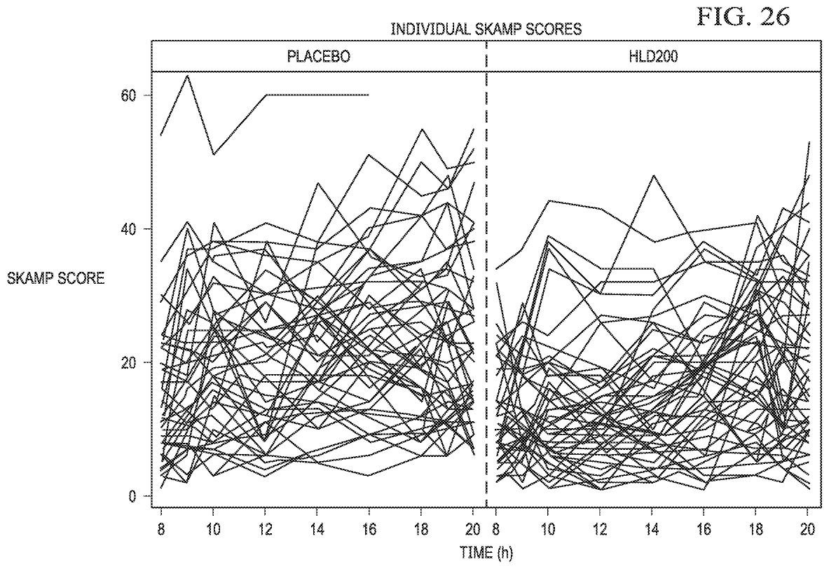

FIG. 26 show the scatter plots of the individual SKAMP score profiles by treatment.

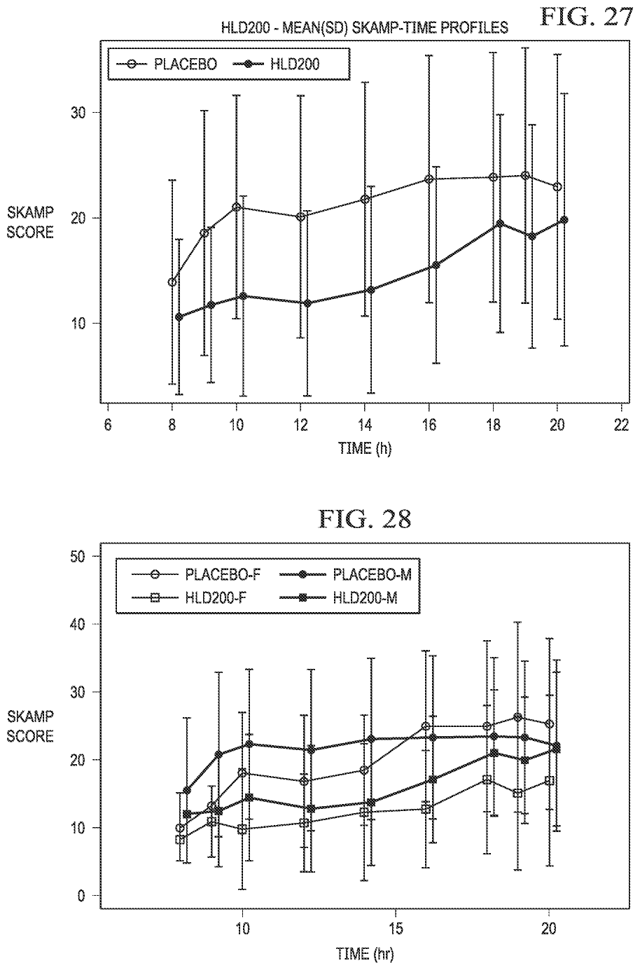

FIG. 27 and FIG. 28 show the scatter plots of the mean (±SD) SKAMP scores profiles by treatment in the total population and by gender.

This descriptive analysis seems to indicate that the a more robust response (change from placebo) is observed in the female with respect to male.

### Example 11. PK/PD Analysis Results: Estimated PK Exposure

The individual MPH concentrations were derived for each subject treated with HLD200 using the previously developed population PK model jointly with the individual demographic data (weight and gender) and the individual HLD200 dosing history. The MPH concentrations were estimate in order to match with the time of measurement of the SKAMP scores.

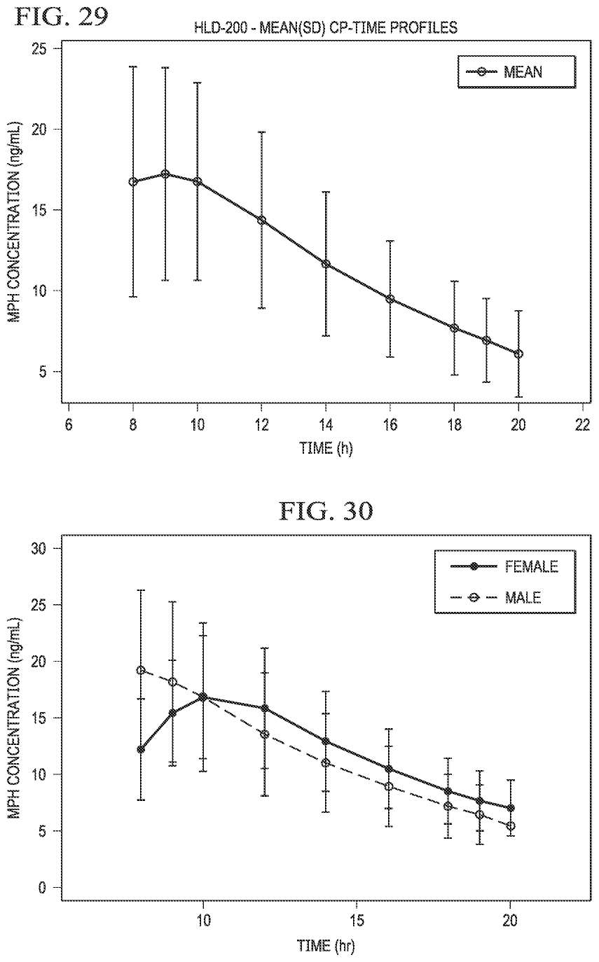

The mean (±SD) MPH concentration values are shown in FIG. 29 and the mean MPH concentration by gender are shown in FIG. 30.

### Example 12. PK/PD Analysis Results: Placebo Model Development

The analysis data set included data collected on 53 subjects with a total of 470 SKAMP measurements.

The estimated fixed effect and the random effect parameters are shown in Table 14 and 15 respectively. In these tables, SE represents the standard error of the parameter estimates, and RSE represents the root mean square error and CV % represents the coefficient of variation of the IIV variability.

TABLE 14
Placebo response model: fixed effect parameter estimates:

| Fixed | | | | |
|---|---|---|---|---|
| Effect | Parameter | Value | SE | RSE |
| | BAS | 13.100 | 1.060 | 8.10% |
| | Kout | 0.037 | 0.006 | 16.90% |
| | A1 | 0.265 | 0.060 | 22.70% |
| | P1 | 5.690 | 0.765 | 13.40% |
| | Add_error | 0.277 | 0.016 | 5.80% |
| | Prop_error | 1* | | |
| | *fixed value | | | |

TABLE 15
Placebo response model: random effect parameter estimates:

| | Parameter | Variability | Value | CV % | SE | RSE |
|---|---|---|---|---|---|---|
| Random | Bas | IIV | 0.235 | 48.48% | 0.051 | 21.60% |
| Effect | A1 | IIV | 1.230 | 110.91% | 0.504 | 41.00% |

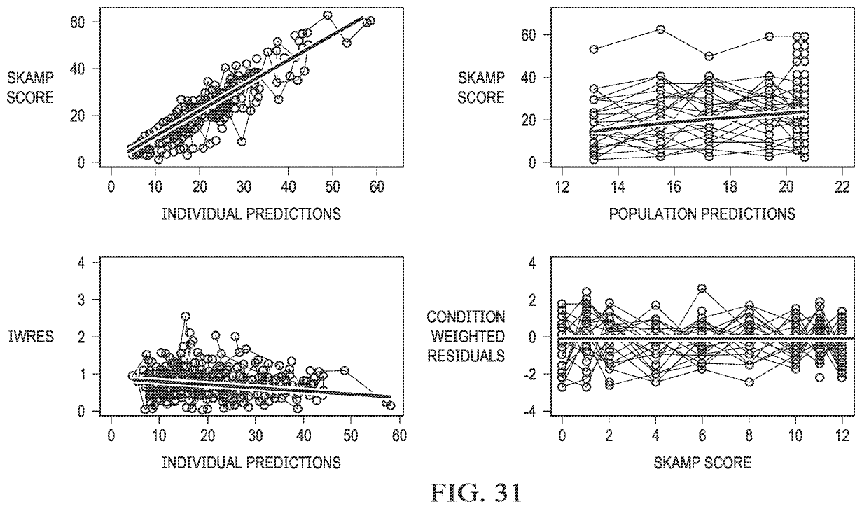

The goodness-of-fit (GOF) diagnostic plots for the placebo response model are shown in FIG. 31. Lines of identity, zero lines, and trend lines are overlaid when appropriate.

Overall, there was no apparent bias in these diagnostic plots, suggesting that the placebo response model was adequate in describing the SKAMP scores in the placebo treated subjects.

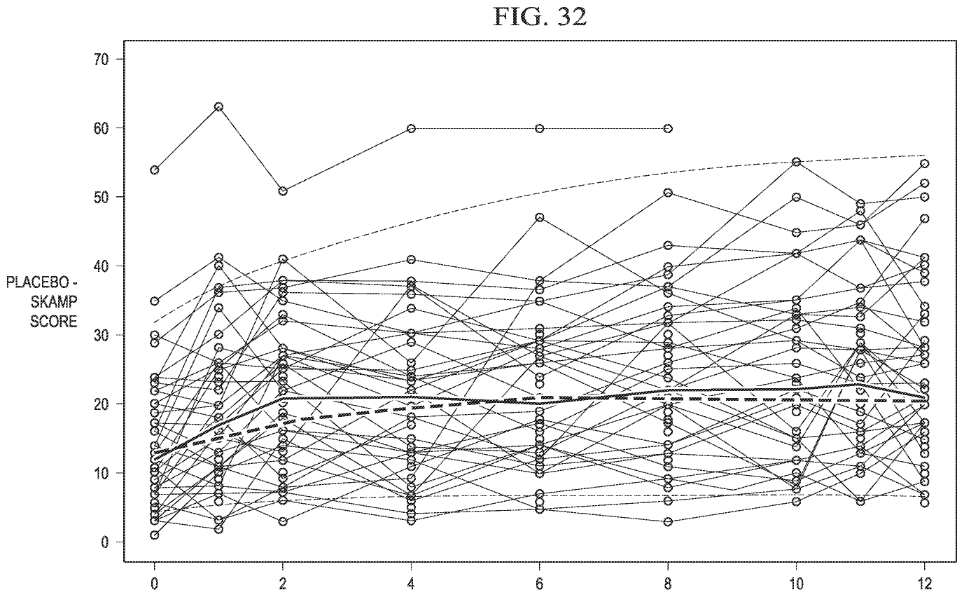

The VPC for the placebo response model are shown in FIG. 32. The VPCs showed that the model performs well. The median SKAMP scores as well as the dispersion of the data around the median were well described by the 5th percentile, the 50th (median) and the 95th percentile indicating that the population model properly described the observed data.

### Example 13. PK/PD Analysis Results: PK/PD Model Development

The analysis data set includes data collected in 64 subjects for a total of 557 SKAMP score measurements.

Base model development. The base model was developed by using the following information: (1) The individual MPH concentration estimated by the population PK model previously developed, and (2) The placebo response defined by the fixed and random effect parameters estimated in the placebo response model previously developed.

The parameters estimated in this analysis (Run 1) were: EMAX, EC50 and g.

The estimated fixed effect and the random effect parameters are shown in Table 16 and 17 respectively. In these tables, SE represents the standard error of the parameter estimates, and RSE represents the root mean square error and CV % represents the coefficient of variation of the IIV variability.

TABLE 16
Base population PK/PD model: fixed effect parameter estimates:

| Fixed | | | | |
|---|---|---|---|---|
| Effect | Parameter | Value | SE | RSE |
| | EMAX | 0.415 | 0.037 | 8.90% |
| | EC50(ng/mL) | 5.860 | 0.664 | 11.30% |
| | g | 6.760 | 2.710 | 40.10% |
| | Add_error | 2.290 | 0.360 | 15.70% |
| | Prop_error | 0.302 | 0.030 | 10% |

TABLE 17
Base population PK/PD model: random effect parameter estimates:

| | Parameter | Variability | Value | CV % | SE | RSE |
|---|---|---|---|---|---|---|
| Random | EMAX | IIV | 0.016 | 12.77% | 0.038 | 233.10% |
| Effect | EC50 | IIV | 0.269 | 51.87% | 0.098 | 36.40% |

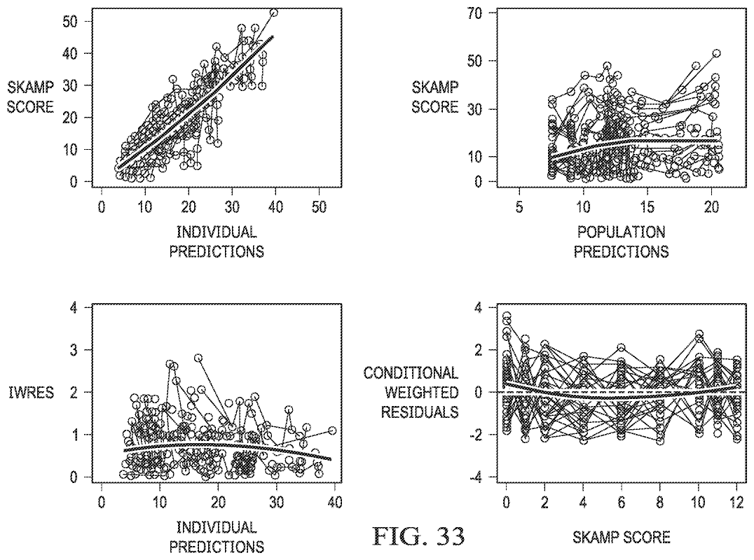

In this analysis it was not possible to estimate the random effect for the ‘g’ parameters. The goodness-of-fit (GOF) diagnostic plots for the base population PK/PD model are shown in FIG. 33. Lines of identity, zero lines, and trend lines are overlaid when appropriate.

Overall, there was no apparent bias in these diagnostic plots, suggesting that the base population PK/PD model was adequate in describing the SKAMP scores associated with the HLD200 treatment.

### Example 14. PK/PD Analysis Results: PK/PD Model Development: Covariate Analysis

Graphical and statistical approaches along with consideration of the underlying scientific rationale were used to identify the covariates to be included in the base population PK/PD model and to assess the mathematical form of the relationships between parameters and covariates. The prospectively identified covariates were: gender, and weight.

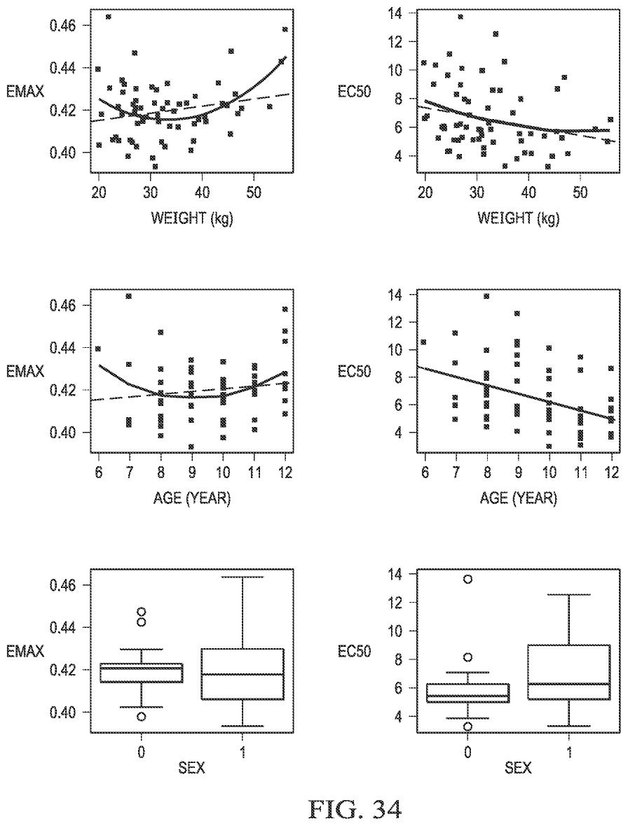

Empirical Bayesian estimates of individual parameters were obtained from the base model (Run 1) in the NONMEM analysis. A graphical exploration of the potential impact of covariates on the PK/PD parameters variability was conducted by analyzing the scatter plot of the individual PK/PD parameter versus the selected covariates (FIG. 34).

The analysis of the distribution of the individual parameter estimates versus the selected covariates indicated a potential dependency of EC50 to the weight, age and gender.

The characteristics of the reference base model (in absence of covariate) and the different models explored to account for the covariate effect are summarized in Table 18.

TABLE 18
List of all intermediate population PK/PD models evaluated:

| | Reference | | Change | | Description of the | |
|---|---|---|---|---|---|---|
| Run | Run | OFV | in OFV | df | Model Tested | p value |
| Run 1 | | 2546.223 | | | Reference base | |
| | | | | | model | |

Forward addition procedure

| | | | | | | |
|---|---|---|---|---|---|---|
| Run 2 | Run 1 | 2542.852 | −3.371 | 1 | Weight on EC50 | 0.066 |
| Run 3 | Run 1 | 2537.38 | −8.813 | 1 | Age on EC50 | 0.003* |
| Run 4 | Run 1 | 2534.163 | −12.06 | 1 | Gender on EC 50 | 0.0005* |
| Run 5 | Run 4 | 2532.689 | −1.474 | 1 | Gender on EC 50 and | 0.224 |
| | | | | | Age on EC50 | |
| Best performing | Run 4 | 2534.163 | | | | |
| model | | | | | | |
| Final model | Run 4 | | | | | |

OFV = maximum likelihood objective function value
*= Statistically significant

The covariate analysis indicated that the best performing model was the one with an effect of gender on the EC50 parameters (Run 4): the EC50 seems to be twice higher in male than in female.

The details of the models accounting for the covariate effect is presented below:

Run 1 (Reference base model):

EMAX=θ₁

EC50=θ₂

Run 2:

$$E C 50 = {θ}_{2} \cdot {( \frac{W T}{30.86} )}^{{θ}_{3}}$$

where 30.86 is the median value of weight in the analysis dataset.

Run 3:

$$E C 50 = {θ}_{2} \cdot {( \frac{AGE}{10} )}^{{θ}_{3}}$$

where 10 is the median value of age in the analysis dataset.

Run 4:

EC50=θ₂ if gender=F

EC50=θ₃ if gender=M

Run 5:

$$E C = {θ}_{2} if gender = F$$

$$EC = {θ}_{3} if gender = M$$

$$EC 50 = EC \cdot {( \frac{AGE}{10} )}^{{θ}_{4}}$$

where 10 is the median value or age in me analysis dataset.

The estimated fixed effect and the random effect parameters are shown in Table 19 and 20 respectively. In these tables, SE represents the standard error of the parameter estimates, and RSE represents the root mean square error and CV % represents the coefficient of variation of the IIV variability.

TABLE 19
Final population PK/PD model: fixed effect parameter estimates:

| Fixed | | | | |
|---|---|---|---|---|
| Effect | Parameter | Value | SE | RSE |
| | EMAX | 0.402 | 0.035 | 8.70% |
| | EC50(ng/mL)-F | 3.720 | 0.536 | 14.40% |
| | g | 12.600 | 6.260 | 49.70% |
| | EC50(ng/mL)-M | 8.400 | 0.572 | 6.80% |
| | Add_error | 2.270 | 0.350 | 15.40% |
| | Prop_error | 0.298 | 0.031 | 10.30% |

TABLE 20
Final population PK/PD model: random effect parameter estimates:

| | Parameter | Variability | Value | CV % | SE | RSE |
|---|---|---|---|---|---|---|
| Random | EMAX | IIV | 0.0437 | 20.90% | 0.0188 | 43% |
| Effect | EC50 | IIV | 0.0712 | 26.68% | 0.0218 | 30.60% |

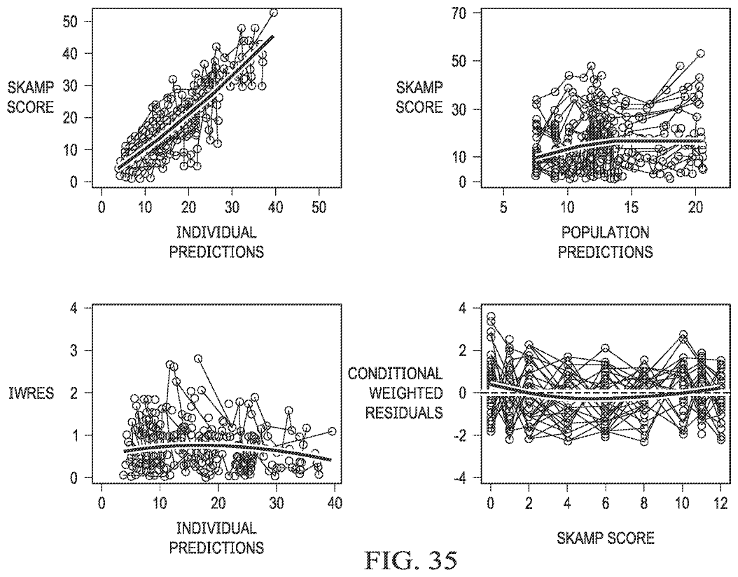

The goodness-of-fit (GOF) diagnostic plots for the base population PK/PD model are shown in FIG. 35. Lines of identity, zero lines, and trend lines are overlaid when appropriate.

Overall, there was no apparent bias in these diagnostic plots, suggesting that the base population PK/PD model was adequate in describing the SKAMP scores in the subject treated with HLD200.

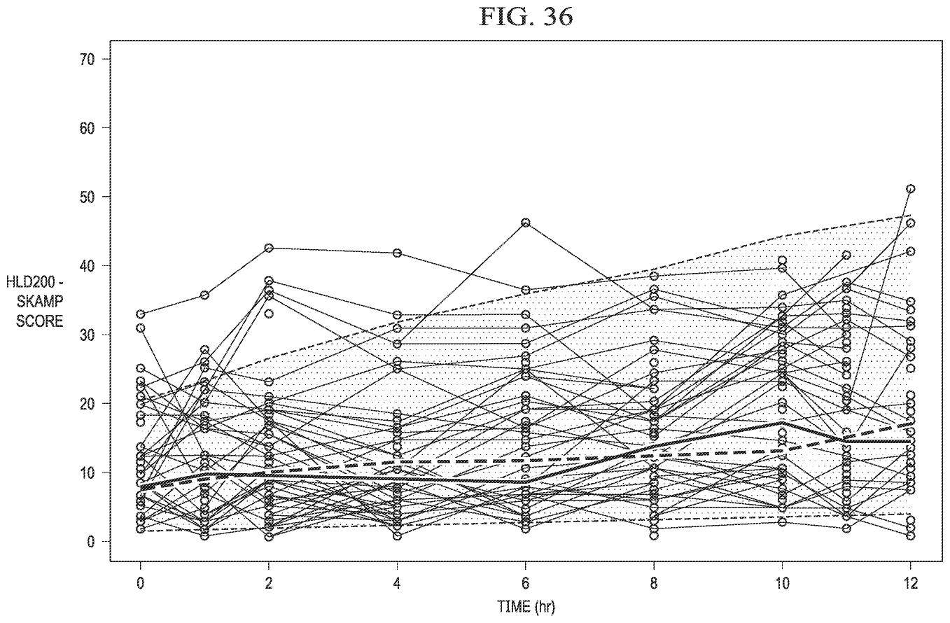

The VPCs for the SKAMP scores associated with HLD200 treatment are shown in FIG. 36.

The VPCs showed that the model performs well at the different doses: The median SKAMP score values as well as the dispersion of the data around the median were well described by the 5th percentile, the 50th (median) and the 95th percentile indicating that the population model properly described the observed data.

### Example 15. PK/PD Analysis Results: PK/PD Model Development: Exposure-Response Relationship

The PK/PD model identified the relationship between MPH exposure and the clinical response evaluated by the ability of the drug to reverse the placebo response.

In absence of drug (Cp=0) the clinical response was assumed to coincide with the response observed following the administration of placebo.

In presence of drug, the SKAMP clinical scores are improved with respect to the placebo response by a factor proportional to the drug exposure.

The PK/PD model was defined by the following equation:

$$SKAMP ( t ) = R ( t ) \cdot ( 1 - \frac{E max \cdot {C}_{p}^{g}}{{EC}_{50}^{g} + {C}_{p}^{g}} )$$

where R(t) is the placebo response and the % change from placebo is defined by:

$$Change from placebo ( \% ) = \frac{E max \cdot {C}_{p}^{g}}{{EC}_{50}^{g} + {C}_{p}^{g}}$$

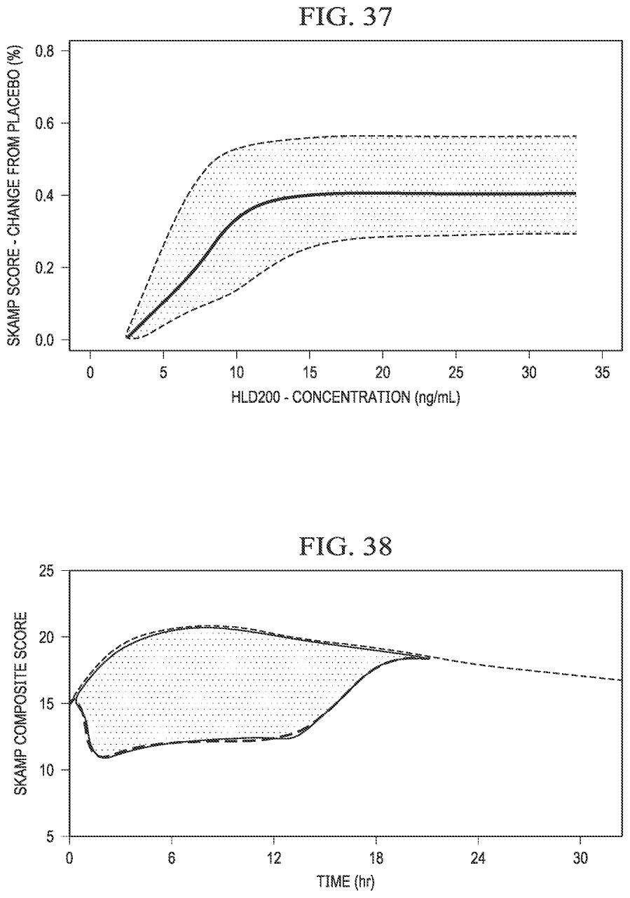

FIG. 37 shows the relationship between the % change from placebo and the MPH concentration. This analysis indicated that a drug concentration of ˜15 ng/ml is required to induce a change from placebo of ˜40%.

Examples 16 to 19 relate to sensitivity of the clinical response with respect to the in-vivo release and the time of HLD200 intake. The objective of this analysis was to evaluate the expected clinical benefit (CB) as a function of the in-vivo release rate of HLD200 and as a function of the HLD200 drug intake time on the evening before the morning classroom.

The impact of different in-vivo release rates and of different times of HLD200 intake on the expected clinical benefit were evaluated by using trial simulations.

### Example 16. Sensitivity of the Clinical Response with Respect to the In-Vivo Release and the Time of HLD200 Intake: Clinical Benefit Definition

CB was defined as the area under change from placebo of the SKAMP score (AUEC) estimated from 8:00 am to 20:00 μm. The evaluation of CB is pictorially presented in FIG. 38.

### Example 17. Sensitivity of the Clinical Response with Respect to the In-Vivo Release and the Time of HLD200 Intake: Impact of the In-Vivo Release Rate on CB

The rate of absorption was characterized by two parameters: td and ss in the PK model developed for HLD200. Therefore, CB was estimated assuming that the td parameter varied from 8 hours to 16 hours and the ss parameter varied from 4.5 to 8.5. The typical values of these parameters estimated in the population PK analysis were: td=12 hr and ss=7.5. The results of the simulation are shown in Table 21.

TABLE 21
Impact of in-vivo release rate on the CB. The shaded cells identify the
values of the in-vivo release for the current formulation of HLD200
and the associated CB value:

| td | ss | AUEC |
|---|---|---|
| 8 | 4.5 | 166.31 |
| 8 | 5.5 | 167.78 |
| 8 | 6.5 | 168.96 |
| 8 | 7.5 | 169.93 |
| 8 | 8.5 | 170.72 |
| 10 | 4.5 | 184.71 |
| 10 | 5.5 | 185.68 |
| 10 | 6.5 | 186.47 |
| 10 | 7.5 | 187.11 |
| 10 | 8.5 | 187.62 |
| 12 | 4.5 | 193.63 |
| 12 | 5.5 | 193.92 |
| 12 | 6.5 | 193.91 |
| 12 | 7.5 | 193.47 |
| 12 | 8.5 | 192.47 |
| 14 | 4.5 | 191.57 |
| 14 | 5.5 | 186.85 |
| 14 | 6.5 | 182.08 |
| 14 | 7.5 | 178.01 |
| 14 | 8.5 | 174.60 |
| 16 | 4.5 | 174.77 |
| 16 | 5.5 | 166.52 |
| 16 | 6.5 | 159.90 |
| 16 | 7.5 | 154.56 |
| 16 | 8.5 | 150.17 |

The results of the simulations indicated that only marginal improvement in the clinical benefit are expected with altered in-vivo release rates are used.

### Example 18. Sensitivity of the Clinical Response with Respect to the In-Vivo Release and the Time of HLD200 Intake: Impact of the HLD200 Drug Intake Time on CB

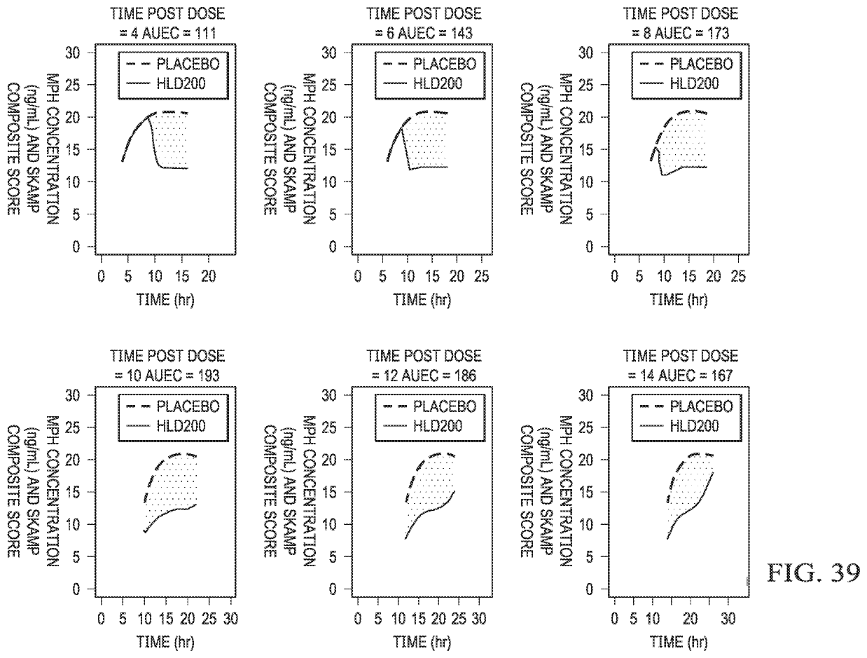

CB was estimated assuming that the time of dose intake varied from 4 to 14 hours before the start of the morning classroom session at 8:00 am. The results of the simulation are shown in Table 22 and in FIG. 39.

TABLE 22
Clinical benefit as a function of HLD200 drug intake time:

| Time of dose intake | |
|---|---|
| (hr) before the | |
| morning classroom | AUEC |
| 4 | 111 |
| 6 | 143 |
| 8 | 173 |
| 10 | 193 |
| 12 | 186 |
| 14 | 167 |

The results of the simulation indicated that the clinical benefit is strongly dependent from the time of HLD200 drug intake before the morning classroom start. The optimal time of drug intake was estimated at 10-12 hours before the morning classroom start.

### Example 19. Evaluation of the Shape of the Clinical Response of HLD200 at Different Doses

The objective of this analysis was to evaluate the shape of the clinical response defined by the trajectories of the SKAMP composite scores at the doses of 60 mg, 80 mg and 100 mg using the PK/PD model previously developed.

The following assumptions for the simulations were retained: HLD200 was administered on the evening, 10 hours before the start of the classroom session. The HLD200 concentrations were simulated in a subject of 34 kg. The td parameter value (that was found to be different in male and female) was fixed to the average value of male and female=12.05 hr. The maximal change from placebo expected for a HLD200 dose up to 100 mg was of ˜40% as shown by the previously developed PK/PD model. The simulated response was evaluated from 8:00 am to 30 hours post dose.

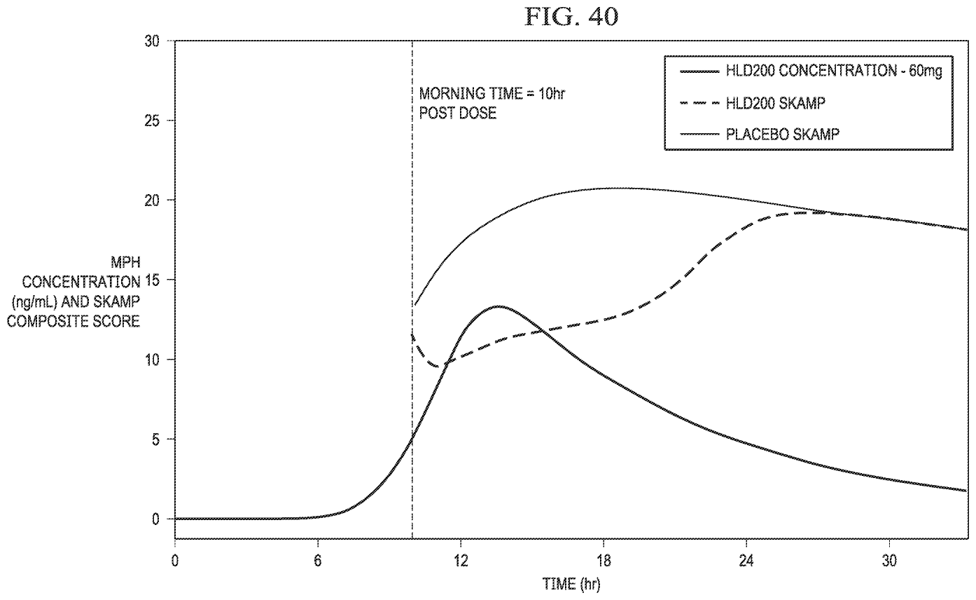

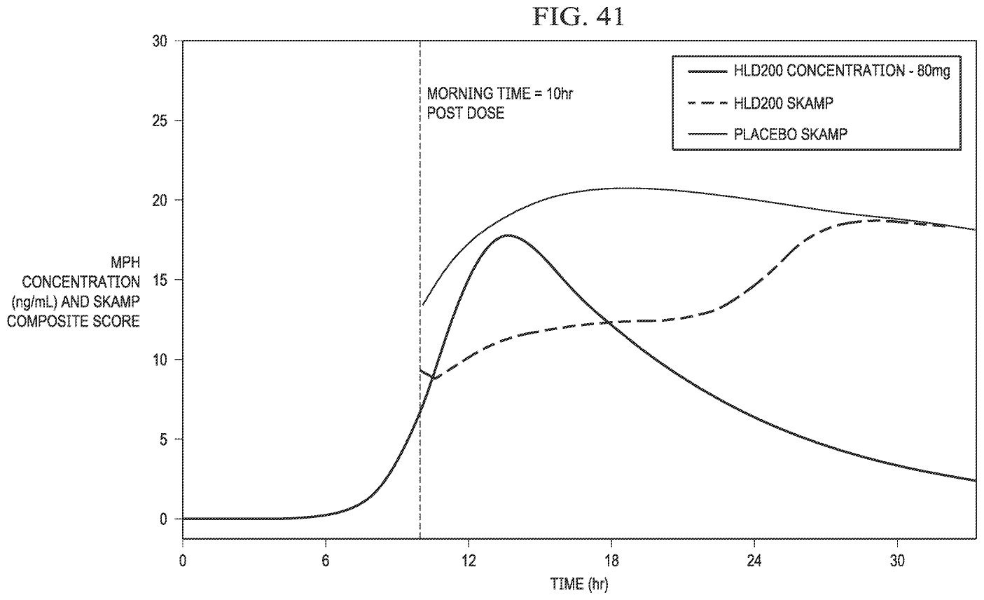

The simulated trajectories of the SKAMP scores are shown in FIGS. 40, 41, and 42 for a HLD200 dose of 60 mg, 80 mg, and 100 mg, respectively.

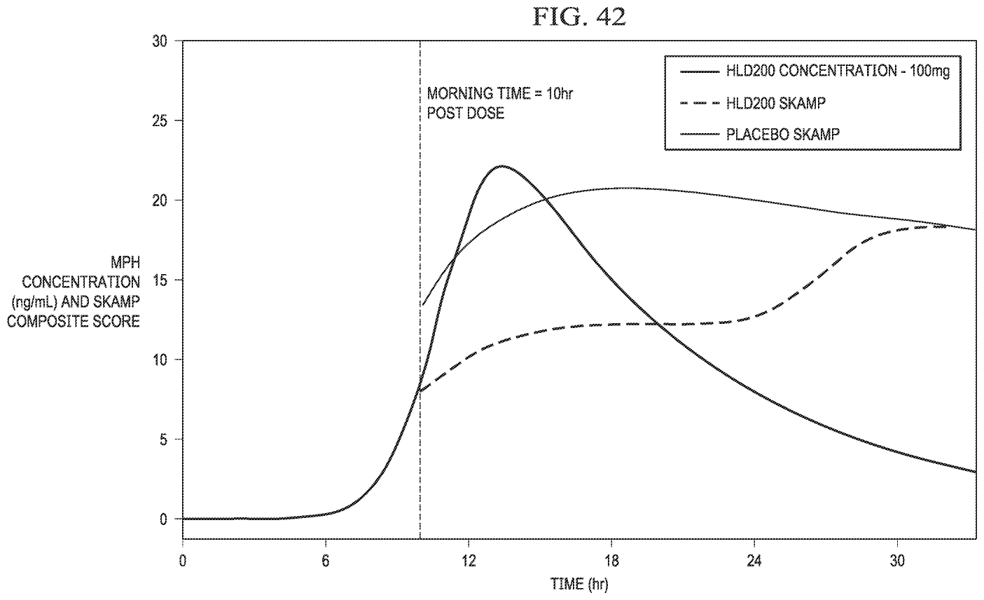

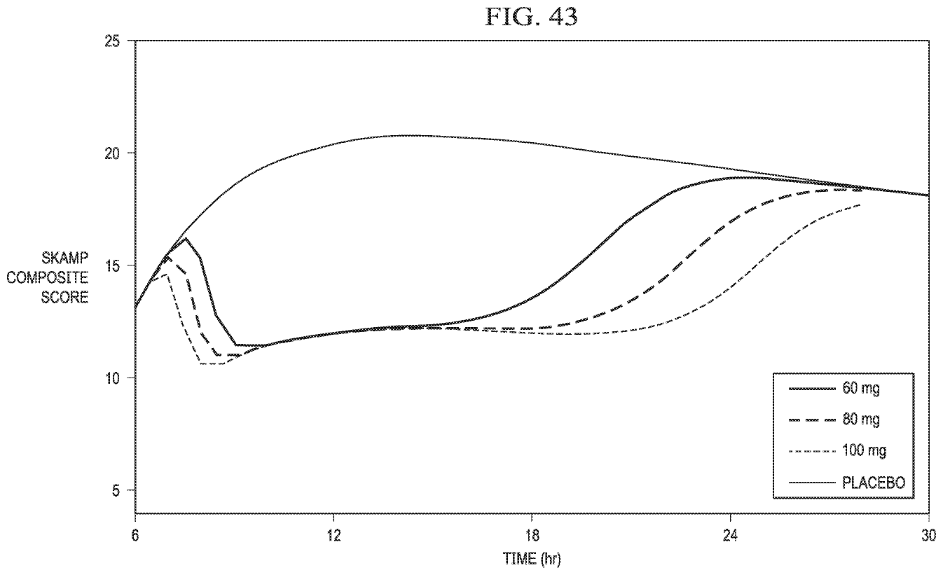

FIG. 43 shows the comparative profiles of the SKAMP scores after the three doses of HLD200.

Accordingly, this Example shows a dose-dependent improvement in clinical benefit. In particular, this Example shows a dose-dependent increase in the period of time showing clinical benefit. Notably, the period of time after Tmax shows a dose-dependent increase in the period of time showing clinical benefit.

### Example 20. Comparison of the Clinical Performance of HLD200 with Concerta® (ALZA Corporation), Metadate CD® (UCB, Inc), NWP06 (Quillivant XR® (NextWave Pharmaceuticals, Inc.)), and d-MPH ER (Focalin XR® (Novartis AG))

Examples 20 to 22 relate to comparison of the clinical performance of HLD200 with Concerta® (ALZA Corporation), Metadate CD® (UCB, Inc), NWP06 (Quillivant XR® (NextWave Pharmaceuticals, Inc.)), and d-MPH ER (Focalin XR® (Novartis AG)). The objective of the analysis was to compare the clinical performance of different MPH products including HLD200, Concerta® (ALZA Corporation), Metadate CD® (UCB, Inc), Quillivant XR® (NextWave Pharmaceuticals, Inc.) and Focalin XR® (Novartis AG) using the composite SKAMP clinical scores. At variance than HLD200, the PK time courses of Concerta® (ALZA Corporation), Metadate CD® (UCB, Inc), Quillivant XR® (NextWave Pharmaceuticals, Inc.) and Focalin XR® (Novartis AG) were described by a convolution based model using a double Weibull function to characterize the in-vivo MPH delivery (Eq. 4) (R. Gomeni, F. Bressolle, T. J. Spencer, S. V. Faraone. Meta-analytic approach to evaluate alternative models for characterizing the PK profiles of extended release formulations of MPH. ASCPT 2016 Annual Meeting, Mar. 8-12, 2016, Hilton Bayfront, San Diego, Calif., incorporated by reference herein). The estimated PK parameters are shown in Table 23.

TABLE 23
PK parameters of Concerta ® (ALZA Corporation),
Metadate CD ® (UCB. Inc), Quillivant XR ® (NextWave
Pharmaceuticals, Inc.) and Focalin XR ® (Novartis AG):

| | td | | td1 | | ff | V/F | Kel |
|---|---|---|---|---|---|---|---|
| | (hs) | ss | (hs) | ss1 | (%) | (L) | (hr−1) |
| Metadate | 1.04 | 2.87 | 4.21 | 3.68 | 39% | 1920 | 0.15 |
| CD ® | | | | | | | |
| Quillivant | 1.03 | 2.99 | 3.64 | 2.98 | 32% | 1960 | 0.14 |
| XR ® | | | | | | | |
| Focalin | 1.02 | 3.41 | 5.96 | 6.56 | 40% | 380 | 0.29 |
| XR ® | | | | | | | |
| Concerta ® | 0.76 | 3.18 | 6.33 | 3.4 | 19% | 1520 | 0.18 |

The clinical performances of the different products were compared to the performance of HLD200 using the mean simulated values of the change from placebo of the SKAMP score (CHP) computed from 8:00 am to 8:00 μm. The response and the variability in the response of the different products was evaluated using the average value of the change from placebo of the SKAMP scores and using the fluctuation index (FI).

The fluctuation index was defined as:

$$FI = \frac{[ max ( C H P ) - min ( C H P ) ]}{average ( CHP )}$$

### Example 21. Comparison of the Clinical Performance of HLD200 with Concerta® (ALZA Corporation), Metadate CD® (UCB, Inc), NWP06 (Quillivant XR® (NextWave Pharmaceuticals, Inc.)), and d-MPH ER (Focalin XR® (Novartis AG)): The Reference Clinical Trials

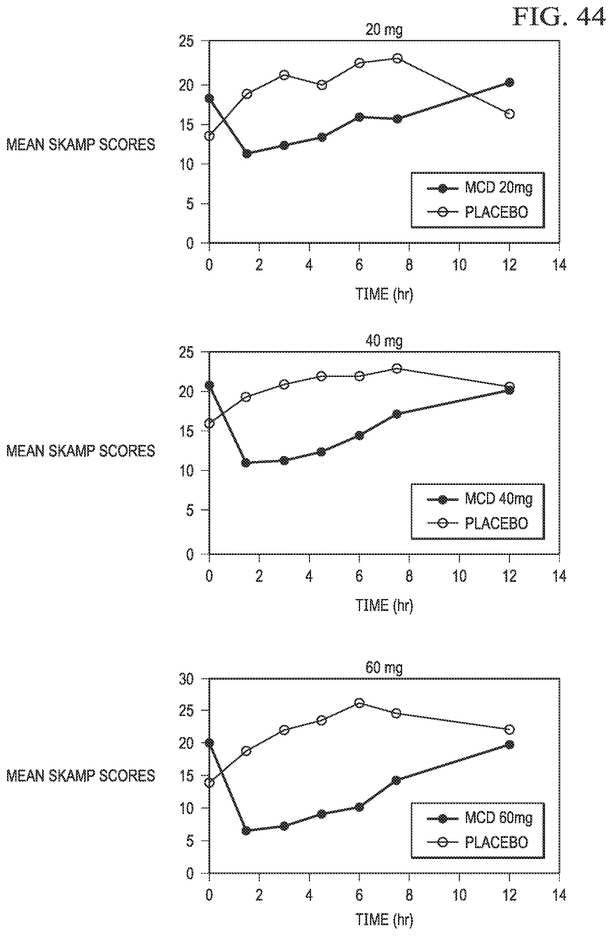

The COMACS study. This was a multi-center, double-blind crossover study of Metadate CD® (UCB, Inc), Concerta® (ALZA Corporation) and placebo with each treatment administered for 1 week (Sonuga-Barke E J, Swanson J M, Coghill D, DeCory H H, Hatch S J. Efficacy of two once-daily methylphenidate formulations compared across dose levels at different times of the day: preliminary indications from a secondary analysis of the COMACS study data. BMC Psychiatry. 2004 Sep. 30; 4:28, incorporated by reference herein). Children were assigned on the basis of their pre-trial dosage to either high (Metadate CD® (UCB, Inc) 60 mg; Concerta® (ALZA Corporation) 54 mg), medium (Metadate CD® (UCB, Inc) 40 mg; Concerta® (ALZA Corporation) 36 mg) or low doses (Metadate CD® (UCB, Inc) 20 mg; Concerta® (ALZA Corporation) 18 mg) of MPH, and attended a laboratory school on the 7th day for assessment at 7 sessions across the day. The values of the SKAMP scores are shown in Table 24 and in FIG. 44.

TABLE 24
COMACS study - Mean (±SD) SKAMP Total scores at each observation session for each treatment at each dose level:

| Observation | | MCD | | | CON | | | PLA | |
|---|---|---|---|---|---|---|---|---|---|
| Session (hrs) | Low | Med | High | Low | Med | High | Low | Med | High |
| 0 | 18.48(11.82) | 20.88(12.95) | 19.91(13.15) | 18.04(10.13) | 19.14(12.14) | 21.47(13.06) | 13.58(9.72) | 16.02(11.84) | 13.96(11.14) |
| 1.5 | 11.44(7.99) | 10.98(8.62) | 6.55(5.85) | 14.04(9.85) | 14.86(12.01) | 11.34(9.71) | 19.10(12.83) | 19.47(12.56) | 18.88(13.48) |
| 3.0 | 12.57(9.92) | 11.03(9.66) | 7.31(6.10) | 16.44(12.43) | 15.29(12.72) | 12.62(11.00) | 21.47(14.61) | 20.98(14.11) | 22.11(14.10) |
| 4.5 | 13.46(11.53) | 12.39(10.32) | 9.15(8.62) | 17.55(13.3T) | 15.09(12.60) | 13.55(11.91) | 20.23(11.92) | 22.09(15.46) | 23.44(12.55) |
| 6.0 | 16.08(13.2T) | 14.47(11.53) | 10.30(9.71) | 17.00(12.12) | 14.28(11.73) | 12.04(11.62) | 22.98(12.79) | 22.15(13.91) | 26.02(14.56) |
| 7.5 | 15.85(11.21) | 17.26(13.63) | 14.29(12.55) | 18.62(12.66) | 15.19(13.4T) | 13.47(12.9T) | 23.54(12.96) | 23.13(14.72) | 24.48(14.68) |
| 12.0 | 20.44(13.75) | 20.28(15.02) | 19.85(14.41) | 16.90(13.36) | 17.81(13.84) | 16.74(14.98) | 19.45(13.46) | 20.73(13.46) | 22.02(15.1T) |

where lower SKAMP scores indicate greater efficacy. CON=Concerta® (ALZA Corporation), MCD=Metadate CD® (UCB, Inc), PLA=placebo, Low=low dose (CON 18 mg; MCD 20 mg), Med=Medium dose (CON 36 mg; MCD 40 mg), Hi=High dose (CON 54 mg; MCD 60 mg).

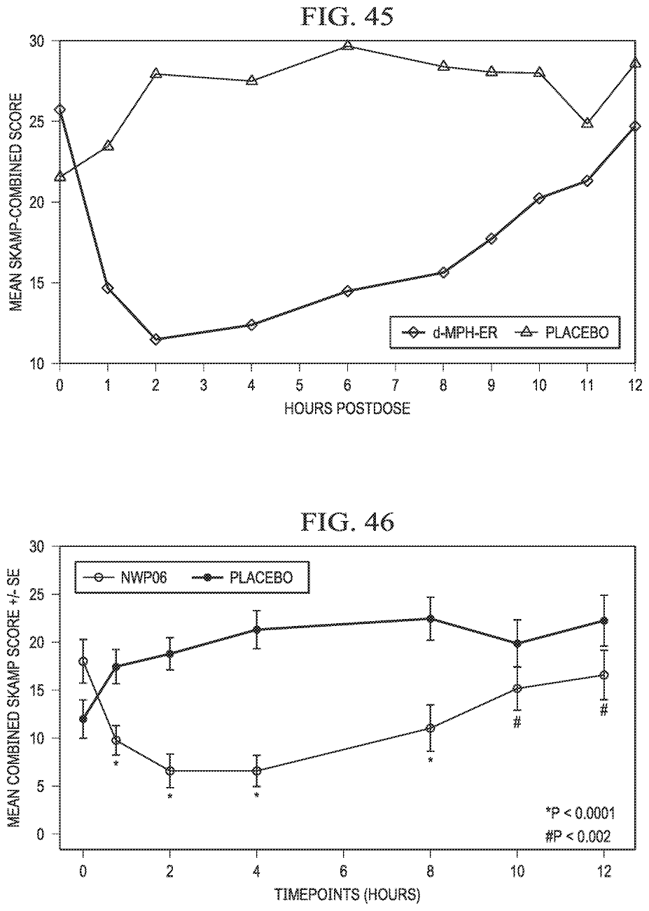

The d-MPH-ER (Focalin XR® (Novartis AG)) Study. This was a double-blind, placebo-controlled, crossover study including 54 children 6-12 years of age, stabilized on MPH 20-40 mg/day (Raul R. Silva et al. Efficacy and Duration of Effect of Extended-Release Dexmethylphenidate Versus Placebo in Schoolchildren With Attention-Deficit/Hyperactivity Disorder. Journal Of Child And Adolescent Psychopharmacology. Volume 16, Number 3, 2006, incorporated by reference herein). Patients participated in a practice day, then received 5 days of treatment with d-MPH-ER 20 mg/day or placebo. After a 1-day wash-out, they returned to the classroom and received 1 dose of their assigned treatment. Evaluations occurred predose and at postdose hours 1, 2, 4, 6, 8, 9, 10, 11, and 12. Children were then crossed over to the alternate treatment, using identical protocol. Primary efficacy variable was the SKAMP-Combined scores. FIG. 45 is a graph reporting exemplary data from the d-MPH ER study: Mean SKAMP-Combined raw scores from predose (hour 0) through hour 12.

The NWP06 (Quillivant XR® (NextWave Pharmaceuticals, Inc.)) study. This was a double-blind, placebo-controlled, crossover design, laboratory classroom study designed to evaluate the efficacy and safety of NWP06 in pediatric patients from 6 to 12 years of age with ADHD (Sharon B. Wigal et al., NWP06, an Extended-Release Oral Suspension of Methylphenidate, Improved Attention-Deficit/Hyperactivity Disorder Symptoms Compared with Placebo in a Laboratory Classroom Study. Journal Of Child And Adolescent Psychopharmacology. Volume 23, Number 1, 2013, incorporated by reference herein). A total of 45 subjects ages 6-12 years were enrolled in this dose-optimized study. Following open-label dose optimization, subjects received 2 weeks of double-blind treatment (1 week of NWP06 and 1 week of placebo). Efficacy measures were based on the SKAMP Rating Scale-Combined measured evaluated at pre-dose and at 0.75, 2, 4, 8, 10, and 12 hours post-dose on each laboratory classroom day. FIG. 46 is a graph reporting exemplary data from the NWP06 (Quillivant XR® (NextWave Pharmaceuticals, Inc.)) study: Mean SKAMP-Combined scores from predose (hour 0) through hour 12.

The mean composite SKAMP score for the placebo arms and the arms treated with d-MPH-ER and NWP06 were digitalized from the figures reported in the reference publications using the Plot Digitizer software (University of South Alabama, Version 2.0).

### Example 22. Comparison of the Clinical Performance of HLD200 with Concerta® (ALZA Corporation), Metadate CD® (UCB, Inc), NWP06 (Quillivant XR® (NextWave Pharmaceuticals, Inc.)), and d-MPH ER (Focalin XR® (Novartis AG)): Clinical Performances

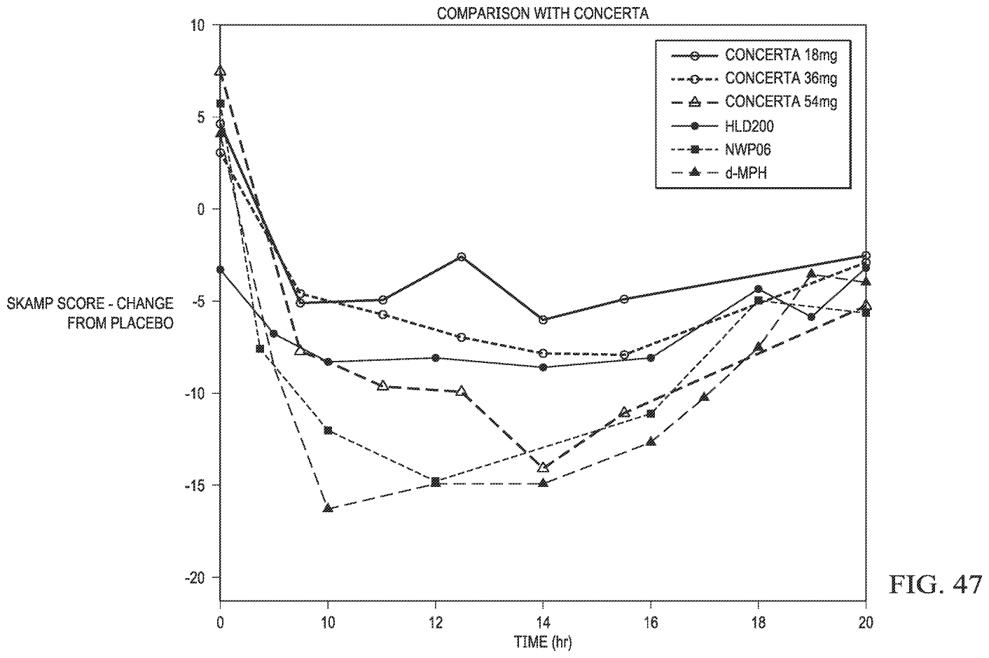

The results of the comparison of the change from placebo of the SKAMP scores of HLD200 (observed in the 107 study) with the data on Concerta® (ALZA Corporation) (at the dose of 18 mg, 36 mg and 54 mg), with the data on Quillivant XR® (NextWave Pharmaceuticals, Inc.), and with the data on d-MPH are shown in FIG. 47.

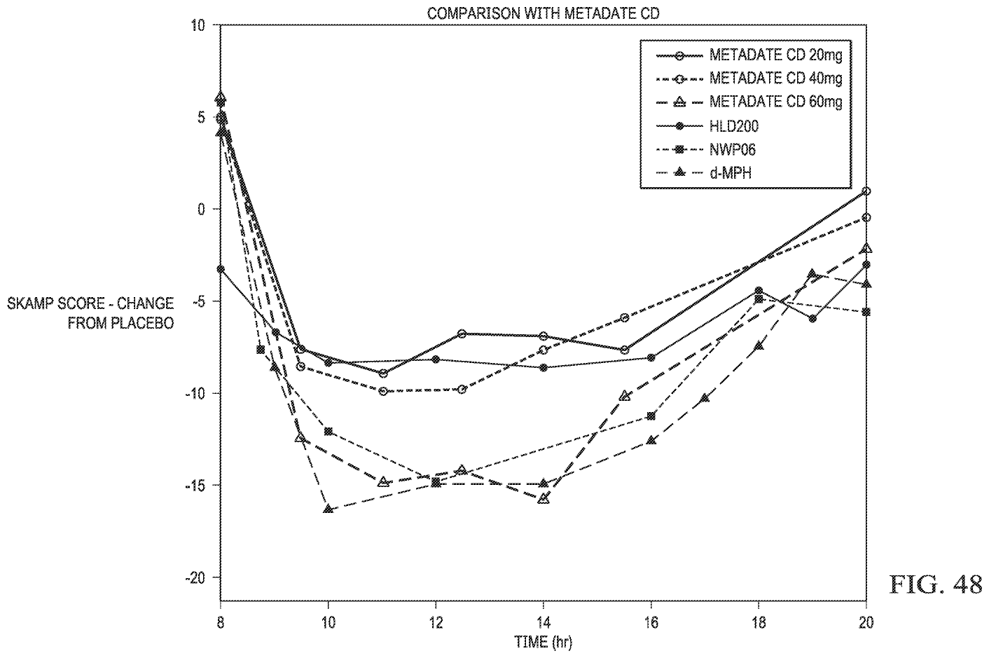

The results of the comparison of the change from placebo of the SKAMP scores of HLD200 (observed in the 107 study) with the data on Metadate CD® (UCB, Inc) (at the dose of 20 mg, 40 mg and 60 mg), with the data on Quillivant XR® (NextWave Pharmaceuticals, Inc.), and with the data on d-MPH are shown in FIG. 48.

Table 25 shows the estimated average value of the change from placebo of the SKAMP scores and the fluctuation index for the different treatments evaluated.

TABLE 25
Estimated average value of the change from placebo of the SKAMP
scores and fluctuation index for the different treatment evaluated:

| | | | | Fluctuation |
|---|---|---|---|---|
| | Average | Min | Max | index |
| Concerta 18 mg | −3.11 | −5.89 | 4.46 | −3.33 |
| Concerta 36 mg | −4.70 | −7.94 | 3.12 | −2.35 |
| Concerta 54 mg | −7.10 | −13.98 | 7.51 | −3.03 |
| NWP06 | −7.17 | −14.85 | 5.79 | −2.88 |
| d-MPH | −8.86 | −16.27 | 4.05 | −2.29 |
| Metadate CD | −4.58 | −8.90 | 4.90 | −3.02 |
| 20 mg | | | | |
| Metadate CD | −5.33 | −9.95 | 4.86 | −2.78 |
| 40 mg | | | | |
| Metadate CD | −9.08 | −15.72 | 5.95 | −2.39 |
| 60 mg | | | | |
| HLD200 | −6.30 | −8.59 | −3.13 | −0.87 |

HLD200 shows the lowest FI indicating a less fluctuating response during a day, as indicated in the shaded cell of Table 25.

In conclusion, Examples 1 to 22 describe the following:

A population PK model was developed to characterize the MPH concentrations after administration of HLD200. An appropriate model was a one-compartment model with a time varying absorption rate well described by a single Weibull in-vivo release function.

The covariate analysis identified an effect of weight on volume of distribution and of gender on the time to release MPH from the HLD200 formulation: the volume increase with weight and the time for release 63.8% of the dose (td) was longer (˜20%) in female with respect to male.

On overall, the population PK model can be considered as adequate to characterize the MPH PK after administration of HLD200 given the goodness-of-fit plots, the good adequacy of the individual model predictions with respect to the observations, and the visual predictive check analysis.

The clinical study (HLD200-107) did not plan to collect PK measurements and, given the parallel study design, no placebo measurements of SKAMP score were available for the subject treated with HLD200.

The development of a PK/PD model required an estimate of the individual PK exposure and an estimate of the individual placebo response. Therefore, the PK/PD analysis was conducted according to a sequential modeling approach. In the first step, the individual MPH concentrations were derived using the population PK model previously developed jointly with the individual demographic data and the HLD200 dosing history, then the placebo response were estimated using the data collected in the placebo arm of the study. Finally the SKAMP scores of the subjects treated with HLD200 were modelled as a function of HLD200 exposure and the placebo response model.

The placebo response model properly described the shape of the SKAMP score trajectories given the goodness-of-fit plots, and the visual predictive check analysis.

The population PK/PD model provided a reasonable estimate of the HLD200 effect. The covariate analysis indicated that the best performing model was the one with an effect of gender on the EC50 parameters: the EC50 appeared to be twice higher in male than in female indicating a possible higher sensitivity in the response in the female population.

Finally, it was possible to establish an exposure-response relationship. This analysis indicated that a drug concentration of ˜15 ng/ml was requested to induce an improvement in the clinical response of ˜40%.

The PK/PD model was developed using the data collected in the HLD200-107 study. The objective of this study was to select the individual dose of HLD200 appropriate for maximizing the clinical response and not to establish a dose (or exposure response) relationship. Investigators were permitted to titrate the dose in 20 mg to 40 mg increments (up or down) until either achieving the “optimal” daily dose or reaching a maximum daily dose of 100 mg/day and/or a maximum dose not to exceed 3.7 mg/kg, whichever is achieved first. The optimal treatment dosage was defined as one that produces maximal symptom control during the morning and throughout the day while remaining safe and well tolerated with a minimum improvement from at least one-third (33%) from the baseline (Visit 2) total score to the total score at randomization (Visit 8) for each of the 3 scales.

The comparison of the expected clinical benefit (CB) of a treatment with HLD200 as a function of the in-vivo release rate indicated that the current formulation provides an optimal response and that only a marginal improvement in the clinical benefit is expected with any modified in-vivo release rate.

The analysis of the change in clinical benefit as a function of the drug intake time on the evening before the morning classroom indicated that the clinical benefit is strongly dependent from the time of HLD200 drug intake. The optimal time of drug intake was estimated at 12 hours before the morning classroom start.

Simulations were conducted using the PK/PD model outcomes to evaluate the shape of the clinical response (trajectories of the SKAMP composite scores) at the doses of 60 mg, 80 mg and 100 mg. The results of the simulations indicated that the increase of the HLD200 dosing provides an extended duration of the clinical response from the morning into the evening.

A final analysis was conducted to compare the clinical performance of different MPH products including HLD200, Concerta® (ALZA Corporation), Metadate CD® (UCB, Inc), Quillivant XR® (NextWave Pharmaceuticals, Inc.) and d-MPH ER using the composite SKAMP clinical scores.

The comparison indicated that HLD200 provided a clinical benefit comparable with the medium-high doses of Concerta® (ALZA Corporation) and Metadate CD® (UCB, Inc). However, the clinical performances of HLD200 showed a more stable clinical response during the day with a clinical benefit starting at earlier time in the day at variance of the other compounds. The earlier onset in the response was also associated at a more stable control of the SKAMP response during the day. This feature was evaluated using the fluctuating index (variability in the response during a day): the fluctuating index of HLD200 that was more that 50% lower that the fluctuating index of the other products evaluated.

### Example 23. In Vitro—In Vivo Correlation (IVIVC) Analysis

Examples 23 to 28 relate to in vitro-in vivo correlation (IVIVC) with respect to formulations of methylphenidate described herein. The analysis was performed to develop and validate an in vitro—in vivo correlation (IVIVC) for HLD200. Three formulations of HLD200, fast, slow and final were included in the current IVIVC analysis.

The 2 formulations used in clinical study HLD200-101 were identified as B-HLD200 (batch N450137) which had a “slow” dissolution profile, and C-HLD200 (batch N451299) which had a “fast” dissolution profile for the clinical study HLD200-101. The difference in dissolution profile was achieved by adjusting the amount of DR coating applied using the same DR coating formulation (30% WG vs 15% WG respectively). Both batches were coated to 20% WG of ER coating. For the clinical study HLD200-104, the final formulation (batch 3125683) was used and was identified as HLD200. This batch was coated to 21% WG of ER coating and 30% WG of DR coating. Refer to Table 26 for description of batches.

TABLE 26
Description of Batches Used:

| Finished Product | | | | In Vivo Study |
|---|---|---|---|---|
| (Material | | Batch | | Used for |
| Number) | Release Profile | Number | Strength | IVVIC |
| B-HLD200 | Slow (20% ER + | N450137 | 54 mg | HLD200-101, |
| (MPH00400) | 30% DR | | | HLD200-103* |
| C-HLD200 | Fast (20% ER + | N451299 | 54 mg | HLD200-101 |
| (MPH00500) | 15% DR) | | | |
| HLD200 | Slow (21% ER + | 3125683 | 100 mg | HLD200-104 |
| | 30% DR) | | | |

wherein * denotes 100 mg prepared from 54 mg capsules.

The following methods were used:

Data. In vitro dissolution and in vivo pharmacokinetic data were available for the formulations considered for the IVIVC modeling and validation.

In vitro Dissolution. The details of the formulations are provided in Table 26. All the formulations were subjected to identical dissolution experimentation. The dissolution testing took place for 2 hours (T=0-2 hour) in 0.1N HCl, then in pH 6.0 phosphate buffer for 4 hours (T=2-6 hour), and finally in pH 7.2 phosphate buffer for the remaining time.

Refer to Table 27, Table 28 and Table 29 for details of the dissolution method.

TABLE 27
Dissolution Apparatus and Conditions:

| | |
|---|---|
| Apparatus | USP <711> Apparatus 1, Baskets (40-mesh) |
| Vessel Size/Type | 1000 mL, round bottom, clear glass |
| Rotation Speed | 75 rpm |
| Test Temperature | 37° C. ± 0.5° C. |
| Pull Volume | 5 mL |
| Replacement | No |
| Sinker | No |
| Cannula | Stainless Steel |
| Filter Type/Size | 10 μm, Polyethylene full flow filter |
| Volume Discard | None |
| Sampling Time Points | Stage 1: 2 hours |
| | Stage 2: 4 and 6 hours |
| | Stage 3: 8, 10, 12, 14, 16, 20, 22 and 24 hours |
| Prime | 60 seconds |
| Purge | 60 seconds |
| Repeat Prime/Purge | 2 times |

where with respect to “Sampling Time Points”, sampling up to 6 hours may be performed manually or with an autosampler. Samples after 6 hours will be collected with an autosampler.

TABLE 28
HPLC Conditions for Dissolution:

| | |
|---|---|
| Column | Zorbax Eclipse ® |
| Dimension | 150 mmx 4.6 mm (i.d) |
| Packing | XDB-CN, 5 μm |
| Pump | Isocratic |
| Detection | UV @ 205 nm |
| Injection Volume | 5 μL |
| Flow Rate | 2.0 mL/min |
| Column Temperature | 30° C. |
| Autosampler | Ambient |
| Temperature | |
| Run Time | 7 minutes |

TABLE 29
Media, Mobile Phase and Diluent for Dissolution:

| | |
|---|---|
| Dissolution | Stage 1 (0-2 Hours) - 700 mL of 0.1N Hydrochloric Acid |
| Media | Stage 2 (2-6 Hours) - 700 mL of 0.1N Hydrochloric Acid |
| | and 200 mL of 0.2 M Sodium Phosphate Tribasic Buffer, |
| | pH 6.0 ± 0.05 |
| | Stage 3 (6-24 hours) - 700 mL of 0.1N Hydrochloric Acid |
| | and 200 mL of 0.2M Sodium Phosphate Tribasic, and |
| | 10 mL of 2N Sodium Hydroxide, pH 7.2 ± 0.05 |
| Mobile | Sodium Octanesulfonate Buffer pH 3.0/Acetonitrile |
| Phase | (80:20 v/v) |
| Diluent | 0.1% Phosphoric Acid |

The amount of methylphenidate released in vitro by dissolution at the 6 hour time point for both the fast and slow formulations was reported as 0.0%. The amount of methylphenidate released at the 4 hour and 7 hour time points was reported as 1.0% Dissolution results for levels of drug release below 5% will be variable since the demonstrated range of the dissolution method is 5% to 130% drug release as determined during the dissolution method validation. Values below 5% would be below the limit of quantitation of the method which would lead to variability in the results at this low level. Therefore, a value of 1.0% was assumed at 6 hours for the purpose of this model.

Study HLD200-101. The study and procedures are described in the Study HLD200-101 clinical protocol. The single dose 3 way cross over Latin Square design with three sequences involved the comparison of 2 Methylphenidate HCl MR Capsule formulations, 54 mg MPH00400 (Test 1, referred to herein as Slow54, Treatment B HLD200) and 54 mg MPH00500 (Test 2, referred to herein as Fast, Treatment C-HLD200) with the reference formulation, Ritalin® (Novartis) 20 mg immediate release (IR) tablet (Treatment A). The subjects were healthy volunteers (6 males and 6 females) and were organized as 3 cohorts comprising 4 subjects each. The MR formulations were administered to the 12 subjects at ˜9:00 pm, whereas the IR comparator (Ritalin® (Novartis)) was dosed at ˜8:00 am in the morning, both under fasted conditions. The PK data for the IR formulation was used to derive the unit impulse function of methylphenidate and the PK data for the slow (B-HLD200) and fast (C-HLD200) formulation was used for the IVIVC analysis.

Study HLD200-104. The study and procedures are described in the Study HLD200-104 clinical protocol. The dose proportionality of the pharmacokinetic parameters of the lowest dose to the highest dose of final formulation of HLD200, 20 mg and 100 mg respectively administered in the evening under fasting conditions was investigated in 20 normal volunteers in Part 1 of this study. The breakfast provided the next morning for both treatments was a medium fat composition (medium calorific value). The PK data from the 100 mg dose, which represents the final formulation, was used for the IVIVC analysis.

Study HLD200-103. The study and procedures are described in the Study HLD200-103 clinical protocol. Eighteen subjects were given a single dose of 100 mg B-HLD200 administered p.o. in the late evening (approximately 9:00 pm) with 240 ml of ambient temperature water. For the purpose of this study, formulation B-HLD-200 (54 mg) was modified to prepare doses equivalent to 100 mg strength in a single capsule. The three treatments compared were given with a high fat meal (A), sprinkled on food (applesauce) (B) and fasted (C) randomly crossed-over to different treatment sequences in 6 cohorts with 7 days of washout between the three treatment periods. The PK data from the 100 mg dose fasted arm (C), which represents the slow formulation (referred to herein as Slow 100), was used for the IVIVC analysis.

Dissolution model. The dissolution model was developed using the in vitro dissolution data for slow, fast and final formulation. An Emax-type model with a sigmoidicity factor (gamma) was employed for this purpose.

Unit impulse response. The mean PK data for the Ritalin® (Novartis) IR formulation were utilized to derive the unit impulse function. A one-compartment model with an absorption lag was adequate to describe the PK for each subject.

Deconvolution and convolution. The individual PK data for the fast and slow formulations from Study HLD200-101 were deconvolved using the unit impulse function for each subject to estimate the cumulative in vivo release rates. The mean cumulative in vivo release rates for the fast and slow formulations were then calculated. These mean in vivo release rates were then correlated with the in vitro release rates using a polynomial function (IVIVC model). The internal validation was conducted by convolving the in vitro release data and the IVIVIC model predicted cumulative in vivo release rates. The predicted and observed PK profiles were compared using non-compartmental parameters (AUC (zero to infinity) and Cmax), according to the recommendations of the IVIVC Guidance (Guidance for Industry: Extended Release Oral Dosage Forms: Development, Evaluation, and Application of In Vitro/In Vivo Correlations, U.S. Department of Health and Human Services Food and Drug Administration Center for Drug Evaluation and Research (CDER) September 1997 BP 2, incorporated by reference herein).

For external validation, the IVIVC model was then used to predict the in vivo PK of the final formulation. The predicted PK was compared to the observed PK (AUC, Cmax) from Study HLD200-103 and Study HLD200-104.

Software and estimation. All estimations were conducted using Phoenix® 6.4 (Certara, Cary, N.C.) and Microsoft Excel® (Redmont, Wash.).

### Example 24. In Vitro-In Vivo Correlation (IVIVC) Analysis: Dissolution Model

The average fraction in vitro release data for the 3 formulations are shown in Table 30 and Table 31.

TABLE 30
Average Fraction In Vitro Release for Fast and Slow Formulations:

Fraction Released

| Time, hr | Fast | Slow |
|---|---|---|
| 2 | 0 | 0 |
| 4 | 0.01 | 0.01 |
| 6 | 0.01 | 0.01 |
| 7 | 0.01 | 0.01 |
| 8 | 0.02 | 0.01 |
| 9 | 0.06 | 0.02 |
| 10 | 0.16 | 0.03 |
| 12 | 0.46 | 0.17 |
| 14 | 0.68 | 0.45 |
| 16 | 0.81 | 0.69 |
| 20 | 0.94 | 0.91 |

TABLE 31
Average Fraction In Vitro Release for the Final Formulation:

| Time, hr | Fraction Released (Final) |
|---|---|
| 0.167 | 0.001 |
| 0.333 | 0.001 |
| 0.5 | 0.001 |
| 1 | 0.001 |
| 2 | 0.002 |
| 4 | 0.002 |
| 6 | 0.003 |
| 7 | 0.003 |
| 8 | 0.005 |
| 9 | 0.011 |
| 10 | 0.029 |
| 11 | 0.079 |
| 12 | 0.18 |
| 13 | 0.305 |
| 14 | 0.442 |
| 15 | 0.581 |
| 16 | 0.689 |
| 17 | 0.773 |
| 18 | 0.833 |
| 19 | 0.875 |
| 20 | 0.907 |
| 22 | 0.946 |
| 24 | 0.964 |
| 28 | 0.981 |

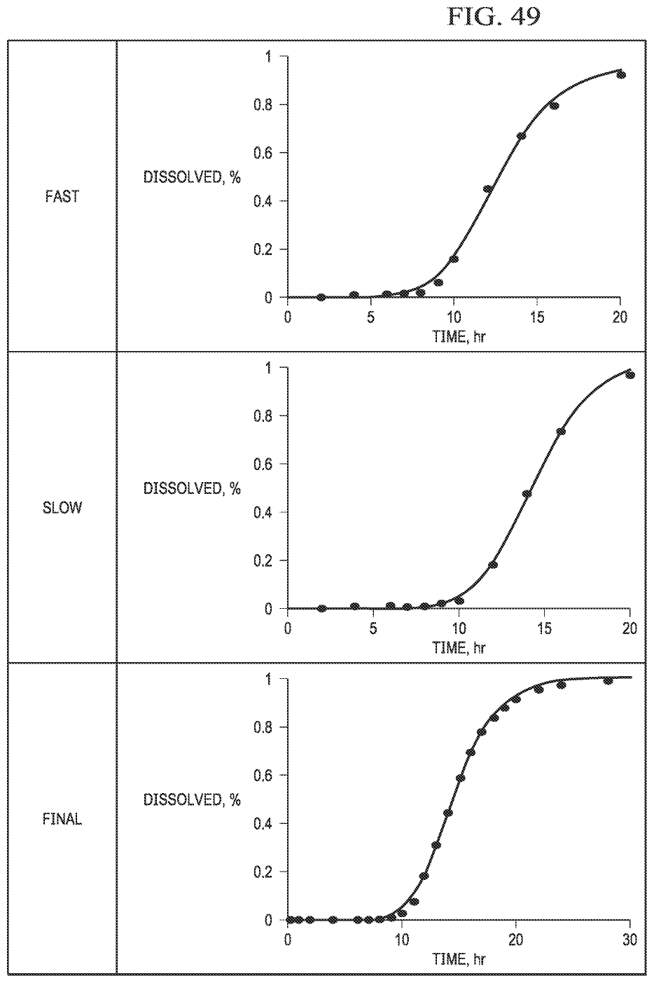

Data at more time points were collected for the final formulation. An Emax-type model adequately described the observed dissolution data, as shown in FIG. 49.

The maximum possible dissolution fraction was fixed at 1 (100%). The time to half-maximal dissolution was estimated to be 14.48 hr, 12.57 hr and 14.49 hr for the slow, fast and final formulations, respectively. The gamma for the slow, fast and final formulations are 8.05, 6.87 and 7.98, respectively as shown Table 32.

TABLE 32
Dissolution Model Parameters (Slow, Fast and Final Formulations):

| Parameter | Slow | Fast | Final |
|---|---|---|---|
| Dmax | 100 | 100 | 100 |
| DT50 | 14.48 | 12.57 | 14.49 |
| gamma | 8.05 | 6.87 | 7.98 |

where Dmax=Maximum fraction dissolved; DT50=time to half-maximal dissolution; and

gamma is the sigmoidicity factor.

### Example 25. In Vitro-In Vivo Correlation (IVIVC) Analysis: Unit Impulse Response

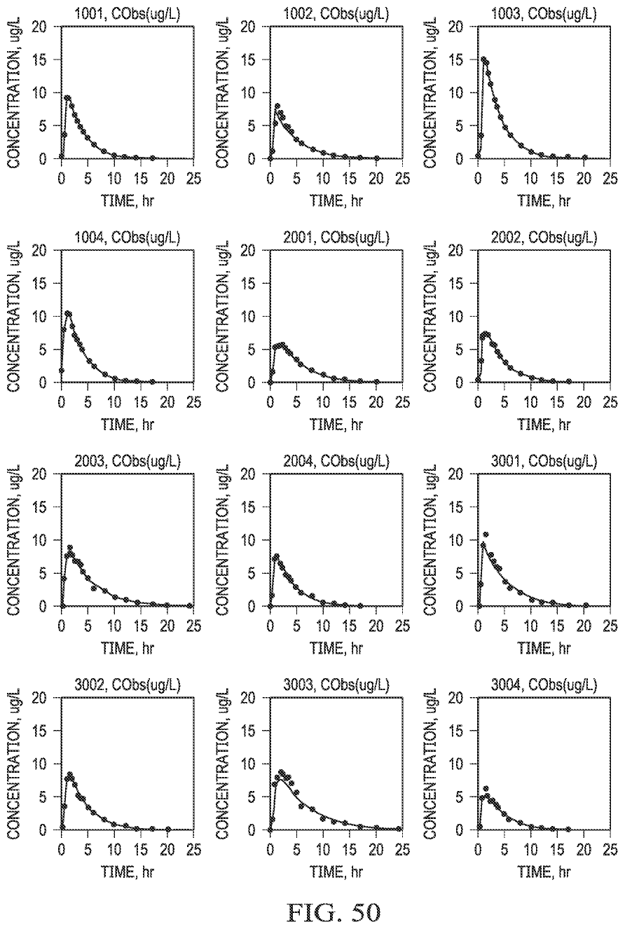

Ritalin® (Novartis) IR data was used to derive the unit impulse function parameters. The methylphenidate concentrations rapidly increased to a maximum concentration in about 1.5 hr and declined rapidly thereafter. A one-compartment model with a time lagged absorption was used for this purpose. For each of the 12 subjects, the model parameters were estimated and the predictions are shown in FIG. 50. FIG. 50 is graphs reporting exemplary observed (dots) and predicted (line) mean methylphenidate concentrations after the administration of 20 mg Ritalin® (Novartis) IR. The model parameters were Absorption rate constant, 1/hr, Volume, L, Elimination rate constant, 1/hr, Absorption lag time, hr, Proportional Error, %, and Additive Error, ug/L. The model describes the observed data very well. The reciprocal of the volume of distribution provides a measure of the magnitude and the elimination rate constant dictates the speed at which concentrations decline. Because these properties are inherent to methylphenidate, they can be employed to estimate the in vivo release rate by deconvolution.

### Example 26. In Vitro-In Vivo Correlation (IVIVC) Analysis: IVIVC Model

The unit impulse function and the observed individual methylphenidate concentrations for the fast and slow54 formulations were used to estimate the cumulative in vivo drug release fraction. The unit impulse function for each subject was used to derive the in vivo drug release in that subject. The individual in vivo cumulative drug release fractions were used to calculate the mean cumulative drug release fraction. Table 33 depicts the mean cumulative fraction drug release in vitro and in vivo rates.

TABLE 33
Fraction Released In Vitro and In Vivo for the Fast
and Slow54 Formulations used in Study HLD200-101.
The in vivo fraction released is calculated as
a mean of the individual release profiles:

Fraction Released

| Formulation | Time, hr | In vitro | In vivo |
|---|---|---|---|
| Fast | 2 | 0 | 0 |
| Fast | 4 | 0.01 | 6.39E−05 |
| Fast | 6 | 0.01 | 0.002297 |
| Fast | 7 | 0.01 | 0.01162 |
| Fast | 8 | 0.02 | 0.041642 |
| Fast | 9 | 0.06 | 0.093782 |
| Fast | 10 | 0.16 | 0.157841 |
| Fast | 12 | 0.46 | 0.373183 |
| Fast | 14 | 0.68 | 0.475953 |
| Fast | 16 | 0.81 | 0.544533 |
| Fast | 20 | 0.94 | 0.625675 |
| Fast | 36 | 1.00 | 0.725893 |
| Slow54 | 2 | 0 | 0 |
| Slow54 | 4 | 0.01 | 0 |
| Slow54 | 6 | 0.01 | 0.000933 |
| Slow54 | 7 | 0.01 | 0.004718 |
| Slow54 | 8 | 0.01 | 1.17E−02 |
| Slow54 | 9 | 0.02 | 0.027169 |
| Slow54 | 10 | 0.03 | 0.058905 |
| Slow54 | 12 | 0.17 | 0.219238 |
| Slow54 | 14 | 0.45 | 0.329213 |
| Slow54 | 16 | 0.69 | 0.439027 |
| Slow54 | 20 | 0.91 | 0.571979 |
| Slow54 | 36 | 1.00 | 0.722196 |

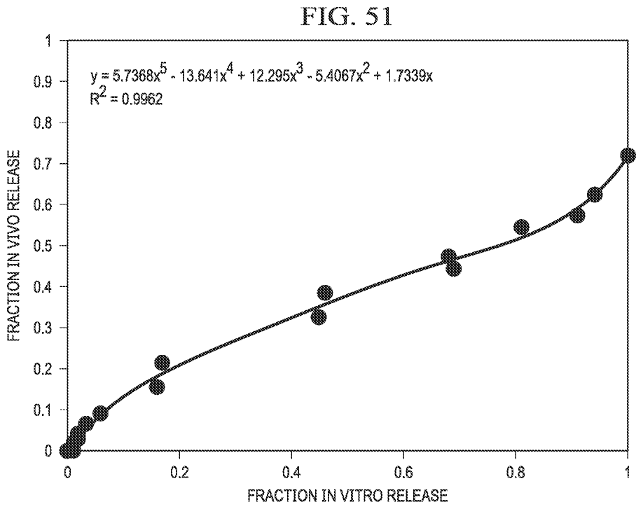

The in vitro and in vivo release rates then were correlated to derive the IVIVC model, as shown in FIG. 51, which shows the IVIVC for both the fast and slow54 formulations combined. A 5th-degree polynomial function successfully described the relationship between the in vitro and in vivo release. As suggested by FIG. 51, the IVIVC relationship is not linear. Lower order polynomial functions were also attempted but the 5th-degree polynomial provided the best fit and prediction quality. The IVIVC model parameters are presented in Table 34.

TABLE 34
IVIVC Model Parameters Relating Fraction Dissolved
In Vitro (Fdiss) and Fraction Absorbed In Vivo (Fabs)
Derived Using Fast and Slow54 Formulations:

| Parameter | Estimate |
|---|---|
| (Fdiss)⁵ | 5.7368 |
| (Fdiss)⁴ | −13.641 |
| (Fdiss)³ | 12.295 |
| (Fdiss)² | −5.4067 |
| (Fdiss)¹ | 1.7339 |

where a 5th degree polynomial best described the relationship: Fabs=(Fdiss)⁵+(Fdiss)⁴+(Fdiss)³+(Fdiss)²+(Fdiss).

### Example 27. In Vitro-In Vivo Correlation (IVIVC) Analysis: Internal Validation

The in vitro dissolution data and the IVIVC model were employed to predict the in vivo PK profiles of the fast and slow54 formulations. The cumulative input predictions for the fast and slow54 formulations are shown in Table 35 and Table 36, respectively.

TABLE 35
Cumulative Fraction Absorbed Predictions for
the Fast Formulation (54 mg) Based on the In
Vitro Dissolution Data and the IVIVC Model:

| Time, | Fdiss | | Cumulative |
|---|---|---|---|
| hr | (Fast) | Fabs | Input |
| 2 | 0 | 0 | 0 |
| 4 | 0.01 | 0.01681 | 907.7664 |
| 6 | 0.01 | 0.01681 | 907.7664 |
| 7 | 0.01 | 0.01681 | 907.7664 |
| 8 | 0.02 | 0.032612 | 1761.022 |
| 9 | 0.06 | 0.087053 | 4700.877 |
| 10 | 0.16 | 0.181035 | 9775.867 |
| 12 | 0.46 | 0.357669 | 19314.15 |
| 14 | 0.68 | 0.462394 | 24969.3 |
| 16 | 0.81 | 0.519485 | 28052.22 |
| 20 | 0.94 | 0.6246 | 33728.41 |
| 36 | 1.00 | 0.718 | 38772 |

where Fdiss=fraction dissolved In Vitro and Fabs=fraction absorbed In Vitro.

TABLE 36
Cumulative Fraction Absorbed Predictions for the
Slow Formulation (54 mg and 100 mg) Based on the
In Vitro Dissolution Data and the IVIVC Model:

| | | Slow (54 mg) | | Slow (100 mg) | |
|---|---|---|---|---|---|
| | | | Cumulative | | Cumulative |
| Time, hr | FdISS | Fabs | Input | Fabs | Input |
| 2 | 0 | 0 | 0 | 0 | 0 |
| 4 | 0.01 | 0.01681 | 907.7664 | 0.01681 | 1681.049 |
| 6 | 0.01 | 0.01681 | 907.7664 | 0.01681 | 1681.049 |
| 7 | 0.01 | 0.01681 | 907.7664 | 0.01681 | 1681.049 |
| 8 | 0.01 | 0.01681 | 907.7664 | 0.01681 | 1681.049 |
| 9 | 0.02 | 0.032612 | 1761.022 | 0.032612 | 3261.152 |
| 10 | 0.03 | 0.047472 | 2563.489 | 0.047472 | 4747.203 |
| 12 | 0.17 | 0.188336 | 10170.15 | 0.188336 | 18833.61 |
| 14 | 0.45 | 0.352274 | 19022.79 | 0.352274 | 35227.4 |
| 16 | 0.69 | 0.466511 | 25191.58 | 0.466511 | 46651.08 |
| 20 | 0.91 | 0.591353 | 31933.08 | 0.591353 | 59135.34 |
| 24 | 0.98 | 0.68766 | 37133.65 | 0.68766 | 68766.02 |
| 36 | 1.00 | 0.71675 | 38704.52 | 0.71675 | 71675.04 |

where Fdiss=fraction dissolved In Vitro and Fabs=fraction absorbed In Vitro.

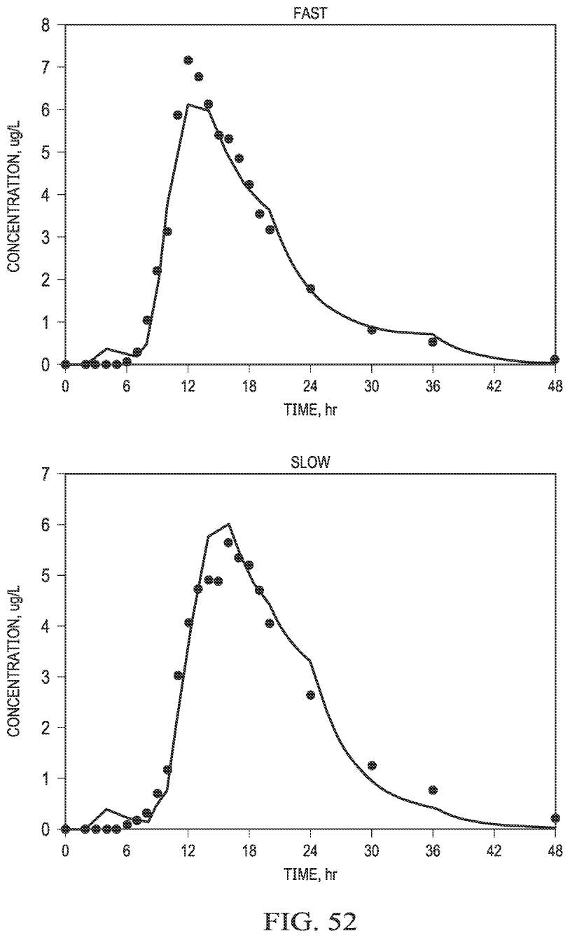

The in vivo predictions and the mean observed methylphenidate concentrations for the fast and slow54 formulations are shown in FIG. 52, and Table 37.

TABLE 37
Predicted In Vivo PK Profiles for the Fast (Internal Validation).
Final (Internal Validation) and Slow (Internal and External
Validation) Formulations using the IVIVC Model:

| | Concentrations, ug/L | | | |
|---|---|---|---|---|
| Time, hr | Fast | Slow 54 mg | Slow 100 mg | Final |
| 0 | 0 | 0 | 0 | 0 |
| 1 | 0 | 0 | 0 | 0 |
| 2 | 0 | 0 | 0 | 0.07892 |
| 3 | 0.20853 | 0.20853 | 0.38617 | 0.06128 |
| 4 | 0.37045 | 0.37045 | 0.68601 | 0.04758 |
| 5 | 0.28764 | 0.28764 | 0.53266 | 0.07616 |
| 6 | 0.22334 | 0.22334 | 0.4136 | 0.09835 |
| 7 | 0.17342 | 0.17342 | 0.32114 | 0.07637 |
| 8 | 0.52667 | 0.13465 | 0.24936 | 0.2147 |
| 9 | 1.75961 | 0.49657 | 0.91957 | 0.6215 |
| 10 | 3.6979 | 0.75425 | 1.39676 | 1.75022 |
| 11 | 5.06241 | 2.33303 | 4.32042 | 4.24225 |
| 12 | 6.1219 | 3.5589 | 6.59056 | 7.27314 |
| 13 | 6.05254 | 4.79697 | 8.88328 | 9.15864 |
| 14 | 5.99869 | 5.7583 | 10.66351 | 10.60838 |
| 15 | 5.36599 | 5.88821 | 10.90409 | 11.5055 |
| 16 | 4.87471 | 5.98908 | 11.09088 | 11.09412 |
| 17 | 4.43702 | 5.42464 | 10.04564 | 10.24065 |
| 18 | 4.09716 | 4.98638 | 9.23404 | 9.36679 |
| 19 | 3.83327 | 4.64608 | 8.60385 | 8.57755 |
| 20 | 3.62837 | 4.38185 | 8.11454 | 7.93351 |
| 21 | 2.96214 | 3.9997 | 7.40684 | 7.16557 |
| 22 | 2.44483 | 3.70296 | 6.85734 | 6.56928 |
| 23 | 2.04316 | 3.47256 | 6.43067 | 5.67263 |
| 24 | 1.73127 | 3.29366 | 6.09937 | 4.97641 |
| 25 | 1.4891 | 2.61756 | 4.84734 | 4.17145 |
| 26 | 1.30106 | 2.0926 | 3.87518 | 3.54643 |
| 27 | 1.15506 | 1.68498 | 3.12033 | 3.06112 |
| 28 | 1.04169 | 1.36847 | 2.53421 | 2.68429 |
| 29 | 0.95366 | 1.12272 | 2.07911 | 2.27095 |
| 30 | 0.88531 | 0.9319 | 1.72574 | 1.95 |
| 31 | 0.83224 | 0.78373 | 1.45135 | 1.7008 |
| 32 | 0.79103 | 0.66868 | 1.2383 | 1.5073 |
| 33 | 0.75904 | 0.57935 | 1.07288 | 1.35705 |
| 34 | 0.73419 | 0.50999 | 0.94443 | 1.24039 |
| 35 | 0.7149 | 0.45614 | 0.8447 | 1.14981 |
| 36 | 0.69992 | 0.41432 | 0.76725 | 1.07947 |
| 37 | 0.54347 | 0.3217 | 0.59575 | 0.83817 |
| 38 | 0.42199 | 0.24979 | 0.46258 | 0.65081 |
| 39 | 0.32766 | 0.19396 | 0.35918 | 0.50534 |
| 40 | 0.25442 | 0.1506 | 0.27889 | 0.39238 |
| 41 | 0.19755 | 0.11694 | 0.21655 | 0.30467 |
| 42 | 0.15339 | 0.0908 | 0.16814 | 0.23657 |
| 43 | 0.1191 | 0.0705 | 0.13056 | 0.18369 |
| 44 | 0.09248 | 0.05474 | 0.10137 | 0.14263 |
| 45 | 0.07181 | 0.04251 | 0.07871 | 0.11074 |
| 46 | 0.05576 | 0.033 | 0.06112 | 0.08599 |
| 47 | 0.03863 | 0.02563 | 0.04746 | 0.06677 |
| 48 | 0.02999 | 0.0199 | 0.03685 | 0.05184 |

The IVIVC model predictions are in close agreement with the observed concentrations. As shown in Table 38, the absolute prediction errors (APE) for the fast formulation are 4.13% and 14.64% for AUC and Cmax, respectively. The absolute prediction errors (APE) for the slow54 formulation are 5.20% and 6.21% for AUC and Cmax, respectively. The average APEs across the two formulations are 4.67% and 10.43% for AUC and Cmax, respectively.

TABLE 38
Internal and External Validation Results:

| | | | |
|---|---|---|---|
| | | | Absolute |
| | Observed | Predicted | Prediction |
| Parameter | Value | Value | Error |

INTERNAL VALIDATION
Fast (Study HLD200-101,54 mg)

| | | | | |
|---|---|---|---|---|
| Formulation | AUC, ug · hr/L | 83.04 | 79.61 | 4.13 |
| | Cmax, ug/L | 7.17 | 6.12 | 14.64 |

Slow54 (Study HLD200-101, 54 mg)

| | | | |
|---|---|---|---|
| AUC, ug · hr/L | 83.83 | 79.47 | 5.20 |
| Cmax, ug/L | 5.64 | 5.99 | 6.21 |

AVERAGE APE

| | |
|---|---|
| AUC, ug · hr/L | 4.67% |
| Cmax, ug/L | 10.43% |

EXTERNAL VALIDATION
Slow100 (Study HLD200-103, 100 mg)

| | | | |
|---|---|---|---|
| AUC, ug · hr/L | 162.08 | 147.17 | 9.20 |
| Cmax, ug/L | 11.18 | 11.09 | 0.81 |

Final (Study HLD200-104, 100 mg)

| | | | |
|---|---|---|---|
| AUC, ug · hr/L | 169.27 | 146.75 | 13.30 |
| Cmax, ug/L | 11.42 | 11.51 | 0.79 |

### Example 28. In Vitro-In Vivo Correlation (IVIVC) Analysis: External Validation

The in vitro dissolution data and the IVIVC model were employed to predict the in vivo PK profiles of the slow formulation from Studies HLD200-103 (slow100 formulation) and HLD200-104 (final formulation). The cumulative input prediction for 100 mg slow formulation is shown in Table 36. The cumulative input prediction for 100 mg final formulation is shown in Table 39.

TABLE 39
Cumulative Fraction Absorbed Predictions for
the Final Formulation (100 mg) Based on the
In Vitro Dissolution Data and the IVIVC Model:

| Time, | Fdiss | | Cumulative |
|---|---|---|---|
| hr | (Final) | Fabs | Input |
| 0.167 | 0.00 | 0.001729 | 172.8506 |
| 0.333 | 0.00 | 0.001729 | 172.8506148 |
| 0.5 | 0.00 | 0.001729 | 172.8506148 |
| 1 | 0.00 | 0.001729 | 344.6271 |
| 2 | 0.00 | 0.003446 | 344.6271 |
| 4 | 0.00 | 0.003446 | 344.6271 |
| 6 | 0.00 | 0.005153 | 515.3371 |
| 7 | 0.00 | 0.005153 | 515.3371 |
| 8 | 0.01 | 0.008536 | 853.5861 |
| 9 | 0.01 | 0.018435 | 1843.486 |
| 10 | 0.03 | 0.046026 | 4602.64 |
| 11 | 0.08 | 0.108783 | 10878.31 |
| 12 | 0.18 | 0.195394 | 19539.36 |
| 13 | 0.31 | 0.27182 | 27181.97 |
| 14 | 0.44 | 0.347935 | 34793.51 |
| 15 | 0.58 | 0.419076 | 41907.55 |
| 16 | 0.69 | 0.4661 | 46610.01 |
| 17 | 0.77 | 0.501501 | 50150.07 |
| 18 | 0.83 | 0.532305 | 53230.51 |
| 19 | 0.88 | 0.560699 | 56069.95 |
| 20 | 0.91 | 0.588414 | 58841.45 |
| 22 | 0.95 | 0.632184 | 63218.36 |
| 24 | 0.96 | 0.657075 | 65707.48 |
| 28 | 0.98 | 0.683841 | 68384.1 |
| 36 | 1.00 | 0.716348 | 71634.76 |

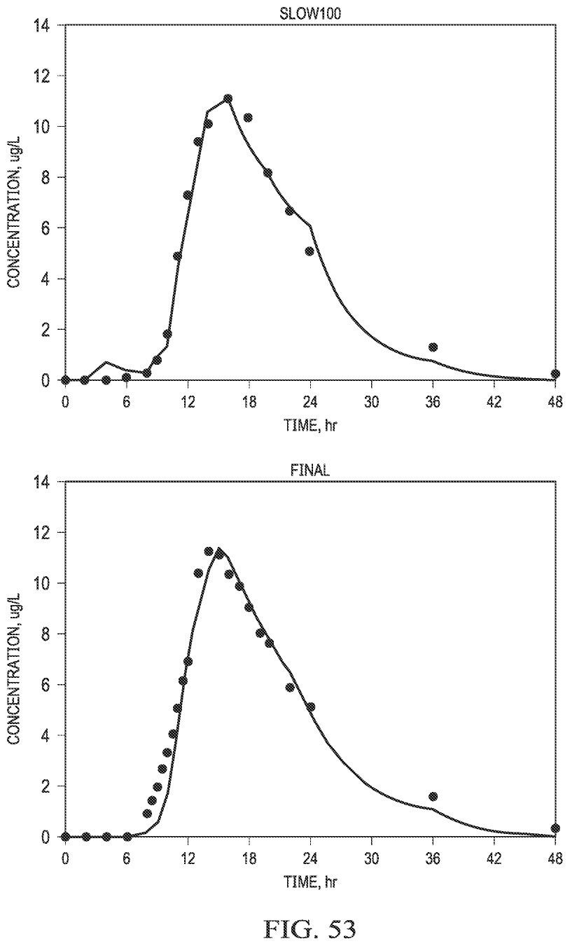

The in vivo predictions and the observed methylphenidate concentrations for the final formulation are shown in FIG. 53 and Table 37. The IVIVC model predictions are in close agreement with the observed concentrations. As shown in Table 38, the absolute prediction errors (APE) for the 100 mg slow100 formulation (Study HLD200-103) are 9.20% and 0.81% for AUC and Cmax, respectively. The APE for the 100 mg final formulation (Study HLD200-104) are 13.30% and 0.79% for AUC and Cmax, respectively.

In conclusion, Examples 23 to 28 describe the following:

The dissolution model yielded a good correlation to the fraction dissolved over time. The IVIVC model was developed using the fast and slow54 formulations from Study HLD200-101. The fraction dissolved in vitro was able to predict the fraction absorbed in vivo with a R² of 0.99. The f2 for these two formulations is 45.15%. The average IVIVC prediction errors of AUC and Cmax for final and slow54 formulations are 4.67% and 10.43%, close to the FDA suggested 10%. The prediction errors for each of the formulations are below within the allowed 15%. The average prediction error for both formulations is about 10% for AUC and close to 10% for Cmax.

An external validation was also conducted using the formulations tested in Study HLD200-103 and Study HLD200-104. The IVIVC model predictions are in close agreement with the observed concentrations. The absolute prediction errors (APE) for the 100 mg slow 100 formulation (Study HLD200-103) are 9.20% and 0.81% for AUC and Cmax, respectively. The APE for the 100 mg final formulation (Study HLD200-104) are 13.30% and 0.79% for AUC and Cmax, respectively. The AUC prediction for the final formulation is on the borderline, while the Cmax passes the 10% threshold. It is to be noted that in Study HLD200-104 subjects received a light breakfast which might contribute to slightly higher variability. The fed to fasted ratio of HLD200 in Study HLD200-104 was estimated to be 1.09 for the AUC and 1.004 for Cmax. The AUC for Study HLD200-104 when adjusted for the slight effect of breakfast is 155.29 ug·hr/L. Upon this adjustment, the APE for AUC of the final formulation (Study HLD200-104) becomes 5.5%. The slight effect of food (breakfast) might be a potential cause of the discrepancy. Overall a successful IVIVC model was developed and validated, as recommended by the FDA in its Guidance for Industry: Extended Release Oral Dosage Forms: Development, Evaluation, and Application of In Vitro/In Vivo Correlations.

### Example 29. Examples of Sustained Release Layers

Some examples of sustained release layers are shown in Table 40. Citric acid was added to a formula to keep the micro environment pH in the film low to inhibit the dissolution of HPMCAS-LF, which dissolves at ≥pH5.5 thus creating a lag at the beginning of the dissolution curve.

TABLE 40
Exemplary Sustained Release Lavers and core:

| | A | B | C | D | E | F |
|---|---|---|---|---|---|---|
| Component | (% w/w) | (% w/w) | (% w/w) | (% w/w) | (% w/w) | (% w/w) |
| Ethocel | 51.0 | 34.9 | 34.5 | 34.4 | 60.2 | 36.1 |
| API | | | | 47.2 | | |
| PEO | | | | | 36.1 | 60.2 |
| HPMC E5 P | 17.0 | 13.1 | | | | |
| HPMCAS-LF | | | 27.6 | 11.5 | | |
| Talc | 3.6 | 2.8 | 3.6 | 2.4 | | |
| Titanium Dioxide | 24.0 | 18.5 | 24.0 | | | |
| Citric acid | | | 6.9 | | | |
| Colloidal silicon | 0.4 | | | | | |
| dioxide | | | | | | |
| TEC | 4.0 | 26.2 | 3.4 | 4.6 | 3.6 | 3.6 |
| Totals* | 100.0 | 100.0 | 100.0 | 100.0 | 100.0 | 100.0 |

*Figures may not sum to 100, due to rounding

An exemplary core was synthesized as shown in Table 41. In this example, an osmotic agent is added to the core.

TABLE 41
Exemplary core:

| Component | Pellet Core (% w/w) |
|---|---|
| API | 20.0 |
| Avicell PH101 | 47.0 |
| Potassium chloride | 30.0 |
| Klucell EF | 3.0 |
| Totals | 100.0 |

A sustained release layer with the formula shown in the right hand column (F) of Table 40 was synthesized on an API containing bead. The formula was designated 2009-043-10A when the sustained release layer provided a 25% weight gain and 2009-043-10 when the sustained release layer pro-vided a 35% gain. Additional layers were synthesized as shown in columns A and B of Table 40. In column B, the formula of column A was modified such that the colloidal silicon dioxide was removed and the plasticizer was increased to 50% w/w of the polymer level. All of the other ratios as shown in column A were maintained.

The formula of column A was also modified to produce the sustained release layer of column C of Table 40. In this formula, the colloidal silicon dioxide was removed and citric acid was added. The ratio of Ethocel:HPMCAS was decreased from 75:25 to 56:44. This formula was expected to provide a lower pH in the microenvironment to increase the lag time. A sample layered to produce a 25% weight gain and another sample with a 45% weight gain were subjected to dissolution testing.

Another embodiment of a sustained release layer was produced in which the drug or API was included in the sustained release layer. This layer is described in column D of Table 40 In this formula, the ratio between Ethocel and HPMCAS was 75:25. A micronized drug was added to the formula as a suspension. A sample with a 25% weight gain was subjected to dissolution testing.

Core tablets as described in Table 41 were coated with a sustained release layer formulated as in column A of Table 40. This formulation exhibited an initial slow drug release (3% in the first 3 hours).

Another embodiment of a sustained release coating was designed with polyethyleneoxide (PEO) to ethyl cellulose ratio of 37.5:62.5. Talc was also added to one sample at 10% to improve the coating process. The presence of talc did not affect drug release. The release profiles for these formulations processed with a 25% weight gain and a 40% weight gain were also determined. The formulations exhibited a 1 hour lag and the drug was substantially completely released within 9 hours.

### Example 30. Example of a Sustained Release Coating

An example of a sustained release coating to be applied to the core pellet is prepared with the following components (see Table 42).

Core batch size—1100.0 g

Coating weight gain—30%

Solids—12.0%

TABLE 42

| | mg/1 g | solvent | | |
|---|---|---|---|---|
| Component | pellet | ratio | % w/w | g/batch |
| Ethocel S 10 | 14.75 | | 49.15 | 162.2 |
| Klucel EF | 3.69 | | 12.29 | 40.6 |
| Dibutyl Sebacate | 1.07 | | 3.56 | 11.7 |
| Mag Stearate 2257 | 10.50 | | 35.00 | 115.5 |
| | | | 100.00 | 330.0 |
| Ethanol | | 95 | | 2299.0 |
| DI Water | | 5 | | 121.0 |
| Theoretical amount of | 1430 | | | |
| coated pellets (g) | | | | |

### Example 31. Example of a pH Dependent Coating

An exemplary S100 pH dependent coating formulated for a 30% weight gain is formulated with the following components (see Table 43).

Coating weight gain—30%

Solids—10.0%

Batch size—715 g

Core pellet amount—550 g

TABLE 43

| | mg/1 g | solvent | | |
|---|---|---|---|---|
| Component | pellet | ratio | % w/w | g/batch |
| Eudragit S100 | 24.18 | | 80.6 | 133.0 |
| Imwitor 900K | 2.43 | | 8.1 | 13.4 |
| Dibutyl Sebacate | 2.43 | | 8.1 | 13.4 |
| Tween 80 | 0.96 | | 3.2 | 5.3 |
| Ethanol | | 94.4 | | 1402.5 |
| DI Water | | 5.6 | | 82.5 |
| | | | 100.0 | 1650.0 |

| | |
|---|---|
| Theoretical amount of | 880.0 |
| coated pellets (g) | |

### Example 32. Example of a pH Dependent Coating

An exemplary S100 pH dependent coating formulated for a 50% weight gain contains the following components (see Table 44).

Coating weight gain—50.0%

Solids—10.0%

Batch size—715 g

Core pellet amount—550 g

TABLE 44

| | mg/1 g | solvent | | |
|---|---|---|---|---|
| Component | pellet | ratio | % w/w | g/batch |
| Eudragit S100 | 40.30 | | 80.6 | 221.7 |
| Imwitor 900K | 4.05 | | 8.1 | 22.3 |
| Dibutyl Sebacate | 4.05 | | 8.1 | 22.3 |
| Tween 80 | 1.60 | | 3.2 | 8.8 |
| Ethanol | | 94.4 | | 2337.5 |
| DI Water | | 5.6 | | 137.5 |
| | | | 100.0 | 2750.0 |

| | |
|---|---|
| Theoretical amount of | 990.0 |
| coated pellets (g) | |

### Example 33. Example of a Sustained Release Coating

An example of a sustained release coating with an alternate ratio of water-soluble (Klucel) to water-insoluble polymer (Ethocel) is prepared with the following components to obtain a faster release profile (see Table 45).

Core batch size—1100.0 g

Coating weight gain—30%

Solids—12.0%

TABLE 45

| | mg/1 g | solvent | | |
|---|---|---|---|---|
| Component | pellet | ratio | % w/w | g/batch |
| Ethocel Std 10 | 12.48 | | 41.59 | 137.2 |
| Klucel EF | 3.12 | | 10.40 | 34.3 |
| Dibutyl Sebacate | 0.90 | | 3.01 | 9.9 |
| Mag Stearate 2257 | 13.50 | | 45.00 | 148.5 |
| | | | | 330.0 |
| Ethanol | | 95 | | 2299.0 |
| DI Water | | 5 | | 121.0 |
| | | | 100.00 | 2750.0 |

| | |
|---|---|
| Theoretical amount of | 1430 |
| coated pellets (g) | |

### Example 34. Example of a Sustained Release Coating

Another example of a sustained release coating according to the disclosure is prepared with the following components (see Table 46).

Core batch size—1100.0 g

Coating weight gain—30%

Solids—12.0%

TABLE 46

| | mg/1 g | solvent | | |
|---|---|---|---|---|
| Component | pellet | ratio | % w/w | g/batch |
| Ethocel Std 10 | 13.22 | | 44.08 | 145.5 |
| Klucel EF | 3.1 | | 11.02 | 36.4 |
| Dibutyl Sebacate | 1.47 | | 4.90 | 16.2 |
| Mag Stearate 2257 | 12.00 | | 40.00 | 132.0 |

Solvents

| | | | |
|---|---|---|---|
| Ethanol | 95 | | 2299.0 |
| DI Water | 5 | | 121.0 |
| | | 100.00 | 2750.0 |

| | |
|---|---|
| Theoretical amount of | 1430 |
| coated pellets (g) | |

### Example 35. Example of a Slow Sustained Release Coating

An example of a slow sustained release coating as described herein for use in Slow Release Formulation (1 and 2) 25% SR+20% or 30% pH Coating (see Table 47).

For Table 47, SR Coat Slow Release (1 and 2):

Coating Weight gain: 25.0

Solids %: 12.0

TABLE 47

| | mg/1 g | solvent | | |
|---|---|---|---|---|
| Component | pellet | ratio | % w/w | g/batch |
| Ethyl Cellulose, NF | 81.5 | | 8.15 | 123 |
| Hydroxypropyl | 20.4 | | 2.04 | 31 |
| Cellulose, NF | | | | |
| Dibutyl Sebacate, NF | 5.9 | | 0.59 | 9 |
| Magnesium | 58.0 | | 5.80 | 88 |
| Stearate, NF | | | | |
| Ethanol (denatured) | | 95 | | 1742 |
| DI Water | | 5 | | 92 |

| | |
|---|---|
| Theoretical amount of | 1250 |
| coated pellets (g) | |

### Example 36. Example of a Slow Enteric Coating

An example of slow enteric coatings as described herein for use in Slow Release Formulation (1 and 2) 25% SR+20% or 30% pH Coating (see Table 48 and Table 49).

For Table 48, S100 pH Dependent Coat Slow Release (1):

Coating Weight gain: 20%

Solids %: 10%

Batch size (g) 1500

Core pellet amount (g) 1250

TABLE 48

| | | solvent | | |
|---|---|---|---|---|
| Component | mg/g | ratio | % w/w | g/batch |
| Methacrylic Acid | 133.7 | | 13.37 | 202 |
| Copolymer Type-B | | | | |
| Mono-and Di-glycerides, NF | 13.4 | | 1.34 | 20 |
| Dibutyl Sebacate, NF | 13.4 | | 1.34 | 20 |
| Polysorbate 80, NF | 5.3 | | 0.53 | 8 |
| Ethanol (denatured) | | 94.4 | | 2138 |
| DI Water | | 5.6 | | 113 |
| Theoretical amount of | 1500 | | | |
| coated pellets (g) | | | | |

For Table 49, S100 pH Dependent Coat Slow Release (2):

Coating Weight gain: 30%

Solids %: 10%

Batch size (g) 1625

Core pellet amount (g) 1250

TABLE 49

| | | solvent | | |
|---|---|---|---|---|
| Component | mg/g | ration | % w/w | g/batch |
| Methacrylic Acid | 185.1 | | 18.51 | 302 |
| Copolymer Type-B | | | | |
| Mono-and Di- | 18.6 | | 1.86 | 30 |
| Clycerides, NF | | | | |
| Dibutyl Sebacate, NF | 18.6 | | 1.86 | 30 |
| Polysorbate 80, NF | 7.4 | | 0.74 | 12 |
| Ethanol (denatured) | | 94.4 | | 3206 |
| DI Water | | 5.6 | | 169 |

| | |
|---|---|
| Theoretical amount of | 1625 |
| coated pellets (g) | |

### Example 37. Example of a Medium Sustained Release Coating

An example of a medium sustained release coating as described herein for use in Medium Release Formulation (1 and 2) 20% SR+20% or 30% pH Coating (see Tables 50, 51 and 52).

For Table 50, SR Coat Medium Release (1 and 2):

Coating Weight gain: 20%

Solids %: 12%

TABLE 50

| | | solvent | | |
|---|---|---|---|---|
| Component | mg/g | ratio | % w/w | g/batch |
| Ethyl Cellulose, NF | 134.0 | | 13.40 | 98 |
| Hydroxypropyl | 33.5 | | 3.35 | 25 |
| Cellulose, NF | | | | |
| Dibutyl Sebacate, NF | 9.7 | | 0.97 | 7 |
| Magnesium Stearate, | 95.5 | | 9.55 | 70 |
| NF | | | | |
| Ethanol (denatured) | | 95 | | 1393 |
| DI Water | | 5 | | 73 |

| | |
|---|---|
| Amount of coated pellets (g) | 1200 |

For Table 51, S100 pH Dependent Coat Medium Release (1):

Coating Weight gain: 20.0

Solids %: 10.0

Batch size (g) 1440

Core pellet amount (g) 1000

TABLE 51

| | | solvent | | |
|---|---|---|---|---|
| Component | mg/g | ratio | % w/w | g/batch |
| Methacrylic Acid | 120.3 | | 12.03 | 193 |
| Copolymer Type-B | | | | |
| Mono-and Di- | 13.6 | | 1.36 | 19 |
| Glycerides, NF | | | | |
| Dibutyl Sebacate, NF | 13.6 | | 1.36 | 19 |
| Polysorbate 80, NF | 5.4 | | 0.54 | 8 |
| Ethanol (denatured) | | 94.4 | | 2077 |
| DI Water | | 5.6 | | 123 |

| | |
|---|---|
| Amount of coated pellets (g) | 1440 |

For Table 52, S100 pH Dependent Coat Medium Release (2):

Coating Weight gain: 30%

Solids %: 10%

Batch size (g) 1560

Core pellet amount (g) 1200

TABLE 52

| | | solvent | | |
|---|---|---|---|---|
| Component | mg/g | ratio | % w/w | g/batch |
| Methacrylic Acid | 185.1 | | 18.51 | 290 |
| Copolymer Type-B | | | | |
| Mono-and Di- | 18.6 | | 1.86 | 29 |
| Glycerides, NF | | | | |
| Dibutyl Sebacate, NF | 18.6 | | 1.86 | 29 |
| Polysorbate 80, NF | 7.3 | | 0.73 | 12 |
| Ethanol (denatured) | | 94.4 | | 3210 |
| DI Water | | 5.6 | | 190 |

| | |
|---|---|
| Amount of coated pellets (g) | 1560 |

### Example 38. Example of a Fast Release Coating

An example of fast release coatings for Fast Release Formulation 20% SR+20% pH Coating (see Tables 53 and 54).

For Table 53, SR Coat Fast Release:

Coating Weight gain: 20.0

Solids %: 12

TABLE 53

| | | solvent | | |
|---|---|---|---|---|
| Component | mg/g | ratio | % w/w | g/batch |
| Ethyl Cellulose, NF | 57.5 | | 5.75 | 83 |
| Hydroxypropyl Cellulose, NF | 14.4 | | 1.44 | 21 |
| Dibutyl Sebacate, NF | 4.2 | | 0.42 | 6 |
| Magnesium Stearate, NF | 6.22 | | 6.22 | 90 |
| Ethanol (denatured) | | 95 | | 1393 |
| DI Water | | 5 | | 73 |

| | |
|---|---|
| Amount of coated pellets (g) | 1200 |

For Table 54, S100 pH Dependent Coat Fast Release:

Coating Weight gain: 20%

Solids %: 10%

Batch size (g) 1440

Core pellet amount (g) 1200

TABLE 54

| | | solvent | | |
|---|---|---|---|---|
| Component | mg/g | ratio | % w/w | g/batch |
| Methacrylic Acid | 133.7 | | 13.37 | 193 |
| Copolymer Type-B | | | | |
| Mono-and Di- | 13.4 | | 1.34 | 19 |
| Glycerides, NF | | | | |
| Dibutyl Sebacate, NF | 13.4 | | 1.34 | 19 |
| Polysorbate 80, NF | 5.3 | | 0.53 | 8 |
| Ethanol (denatured) | | 94.4 | | 2077 |
| DI Water | | 5.6 | | 123 |

| | |
|---|---|
| Amount of coated pellets (g) | 1440 |

### Example 39. Example of a Methylphenidate Composition

This Example describes an exemplary composition of Methylphenidate, 54 mg Capsules (Slow Release Formulation, 25% SR Weight Gain+30% pH Dependent Weight Gain) (see Table 55).

TABLE 55

| | | Strength (label claim) | |
|---|---|---|---|
| | | 54 mg | |
| Component and Quality | | Quantity | |
| Standard (and Grade, if | | per unit | |
| applicable) | Function | (mg) | % |
| Methylphenidate Hydrochloride, | Active | 54.00 | 13.54 |
| USP, CII | Ingredient | | |
| Microcrystalline Cellulose, NF | Binder | 191.45 | 48.00 |
| (Avicel PH-101) | | | |
| Ethyl Cellulose, NF (Ethocel | Film Former | 30.16 | 7.56 |
| Standard 10 Premium) | | | |
| Hydroxypropyl Cellulose, NF | Film Former | 7.54 | 1.89 |
| (Klucel EF Pharm) | | | |
| Dibutyl Sebacate, NF | Film | 2.18 | 0.55 |
| | Plasticizer | | |
| Magnesium Stearate, NF | Hydrophobic | 21.48 | 5.39 |
| | Film | | |
| | Component | | |
| Methacrylic Acid Copolymer | Film Former | 74.19 | 18.60 |
| Type-B (Eudragit S 100) | | | |
| Mono-and Di-Glycerides, NF | Film | 7.46 | 1.87 |
| (Imwitor 900K) | Plasticizer | | |
| Dibutyl Sebacate, NF | Film | 7.46 | 1.87 |
| | Plasticizer | | |
| Polysorbate 80, NF | Solubilizer | 2.95 | 0.74 |

| | | |
|---|---|---|
| Total | 398.87 | 100.0 |

### Example 40. Example of a Methylphenidate Composition

This Example describes an exemplary composition of Methylphenidate, 54 mg Capsules (Slow Release Formulation, 20% SR Weight Gain+20% pH Dependent Weight Gain) (see Table 56).

TABLE 56

| | | Strength (label claim) | |
|---|---|---|---|
| | | 54 mg | |
| Component and Quality | | Quantity | |
| Standard (and Grade, if | | per unit | |
| applicable) | Function | (mg) | % |
| Methylphenidate Hydrochloride, | Active | 54.00 | 15.28 |
| USP, CII | Ingredient | | |
| Microcrystalline Cellulose, NF | Binder | 191.45 | 54.16 |
| (Avicel PH-101) | | | |
| Ethyl Cellulose, NF (Ethocel | Film | 20.42 | 5.78 |
| Standard 10 Premium) | Former | | |
| Hydroxypropyl Cellulose, NF | Film | 5.11 | 1.45 |
| (Klucel EF Pharm) | Former | | |
| Dibutyl Sebacate, NF | Film | 1.48 | 0.42 |
| | Plasticizer | | |
| Magnesium Stearate, NF | Hydrophobic | 22.09 | 6.25 |
| | Film | | |
| | Component | | |
| Methacrylic Acid Copolymer | File Former | 47.48 | 13.43 |
| Type-B (Eudragit S 100) | | | |
| Mono-and Di-Glycerides, NF | Film | 4.77 | 1.35 |
| (Imwitor 900K) | Plasticizer | | |
| Dibutyl Sebacate, NF | Film | 4.77 | 1.35 |
| | Plasticizer | | |
| Polysorbate 80, NF | Solubilizer | 1.89 | 0.53 |

| | | |
|---|---|---|
| Total | 353.46 | 100.0 |

### Example 41. Example of a Methylphenidate Composition

This Example describes an exemplary Composition of Methylphenidate, 54 mg Capsules (Slow Release Formulation, 20% SR Weight Gain+30% EC pH Dependent Weight Gain) (see Table 57).

TABLE 57

| | | Strength (label claim) | |
|---|---|---|---|
| | | 54 mg | |
| Component and Quality | | Quantity | |
| Standard (and Grade, if | | per unit | |
| applicable) | Function | (mg) | % |
| Methylphenidate Hydrochloride, | Active | 54.00 | 15.95 |
| USP, CII | ingredient | | |
| Microcrystalline Cellulose, NF | Binder | 162.00 | 47.84 |
| (Avicel PH-101) | | | |
| Ethyl Cellulose, NF (Ethocel | Film Former | 21.23 | 6.27 |
| Standard 10 Premium) | | | |
| Hydroxypropyl Cellulose, NF | Film Former | 5.31 | 1.57 |
| (Klucel EF Pharm) | | | |
| Dibutyl Sebacate, NF | Film | 7.84 | 2.32 |
| | Plasticizer | | |
| Magnesium Stearate, NF | Hydrophobic | 15.12 | 4.46 |
| | Film | | |
| | Component | | |
| Methacrylic Acid Copolymer | File Former | 62.68 | 18.51 |
| Type-B (Eudragit S 100) | | | |
| Mono-and Di-Glycerides, NF | Film | 6.30 | 1.86 |
| (Imwitor 900K) | Plasticizer | | |
| Polysorbate 80, NF | Solubilizer | 2.49 | 0.74 |
| Talc | Encapsulation | 1.68 | 0.50 |
| | Lubricant | | |

| | | |
|---|---|---|
| Total | 338.65 | 100.0 |

This Example also describes an exemplary composition of Methylphenidate, 54 mg Capsules (Fast Release Formulation, 20% SR Weight Gain+15% EC pH Dependent Weight Gain) (see Table 58).

TABLE 58

| | | Strength (label claim) | |
|---|---|---|---|
| | | 54 mg | |
| Component and Quality | | Quantity | |
| Standard (and Grade, if | | per Unit | |
| applicable) | Function | (mg) | % |
| Methylphenidate Hydrochloride, | Active | 54.00 | 18.03 |
| USP, CII | ingredient | | |
| Microcrystalline Cellulose, NF | Binder | 162.00 | 54.08 |
| (Avicel PH-101) | | | |
| Ethyl Cellulose, NF (Ethocel | Film Former | 21.23 | 7.09 |
| Standard 10 Premium) | | | |
| Hydroxypropyl Cellulose, NF | Film Former | 5.31 | 1.77 |
| (Klucel EF Pharm) | | | |
| Dibutyl Sebacate, NF | Film | 4.69 | 1.57 |
| | Plasticizer | | |
| Magnesium Stearate, NF | Hydrophobic | 15.12 | 5.05 |
| | Film | | |
| | Component | | |
| Methacrylic Acid Copolymer | File Former | 31.34 | 10.46 |
| Type-B (Eudragit S 100) | | | |
| Mono-and Di-Glycerides, NF | Film | 3.15 | 1.05 |
| (Imwitor 900K) | Plasticizer | | |
| Polysorbate 80, NF | Solubilizer | 1.24 | 0.41 |
| Talc | Encapsulation | 1.49 | 0.50 |
| | Lubricant | | |
| Total | | 299.57 | 100.0 |

### Example 42. Method of Processing Coated Methylphenidate Capsules

In an example of a manufacturing process, methylphenidate HCl and microcrystalline cellulose (Avicel PH-101) are blended in a Hobart Mixer. Purified water is added to the dry mixture and the wet granulation is extruded (MG-55 Multi granulator). The extrudate is then spheronized into pellets (Caleva Model #SPH250). The wet pellets are dried (Fluid Air Model #0050) and sieved (30 mesh<Acceptable<20 mesh).

The sustained release coating is added as follows: a dispersion of Ethyl Cellulose NF (Ethocel Standard 10 Premium), Klucel EF, Dibutyl Sebacate, NF, magnesium stearate, NF, ethanol and purified water, USP is prepared in an overhead stirrer. The dispersion is applied to the uncoated methylphenidate pellets in the fluid bed and the coated pellets are sieved as before. It is understood that in this context of this example, the term “dispersion” can refer to various two phase systems in which at least some solids are dispersed in a liquid phase. The term “dispersion”, as used herein, can thus include, but is in no way limited, either wholly or partly, the concepts of colloids, emulsions and/or suspensions.

The enteric coating is prepared as follows: a dispersion of Methacrylic Acid Copolymer Type B (Eudragit S100), Mono and di-glycerides, NF (Inwitor 900K), Dibutyl Sebacate, NF, Polysorbate 80, NF, ethanol and purified water, USP are mixed in an overhead stirrer to obtain a dispersion. The dispersion is applied to the sustained release coated methylphenidate pellets in the fluid bed. The enteric coated pellets are encapsulated to obtain the methylphenidate capsules.

### Example 43. IVIVC of Concerta® (ALZA Corporation)

FIG. 57 is a graph reporting exemplary data on the fraction of methylphenidate dissolved in vitro (FDISS) versus the fraction of methylphenidate absorbed in vivo (Fabs) from Concerta® (ALZA Corporation). From R. Gomeni, F. Bressolle, T. J. Spencer, S. V. Faraone. Meta-analytic approach to evaluate alternative models for characterizing the PK profiles of extended release formulations of MPH. ASCPT 2016 Annual Meeting, Mar. 8-12, 2016, Hilton Bayfront, San Diego, Calif., incorporated by reference herein.

### Example 44. Validation of Double Weibull IVIVC Model in Exemplary MPH Formulations

FIG. 58 is graphs reporting exemplary observed (dots) and predicted (line) mean methylphenidate concentrations after the administration of the indicated drugs. From R. Gomeni, F. Bressolle, T. J. Spencer, S. V. Faraone. Meta-analytic approach to evaluate alternative models for characterizing the PK profiles of extended release formulations of MPH. ASCPT 2016 Annual Meeting, Mar. 8-12, 2016, Hilton Bayfront, San Diego, Calif., incorporated by reference herein.

### Example 45. Determination of Exemplary Optimal MPH Release Profile for Products Releasing MPH Following a Double Weibull In Vivo Absorption, e.g. Concerta® (ALZA Corporation), Ritalin LA® (Novartis AG), Metadate CD® (UCB, Inc), and Quillivant XR® (NextWave Pharmaceuticals, Inc.)

Using the PK/PD model described herein, the methylphenidate absorption profile required to deliver a maximal clinical benefit over a 24 hour period was determined for products releasing MPH following a double Weibull in vivo absorption, e.g. Concerta® (ALZA Corporation), Ritalin LA® (Novartis AG), Metadate CD® (UCB, Inc), and Quillivant XR® (NextWave Pharmaceuticals, Inc.) (R. Gomeni, F. Bressolle, T. J. Spencer, S. V. Faraone. Meta-analytic approach to evaluate alternative models for characterizing the PK profiles of extended release formulations of MPH. ASCPT 2016 Annual Meeting, Mar. 8-12, 2016, Hilton Bayfront, San Diego, Calif., incorporated by reference herein).

Table 59 shows the values that were determined (indicated in the line labeled “initial”) for the products, e.g. Concerta® (ALZA Corporation), compared to the calculated “optimized” methylphenidate absorption profile.

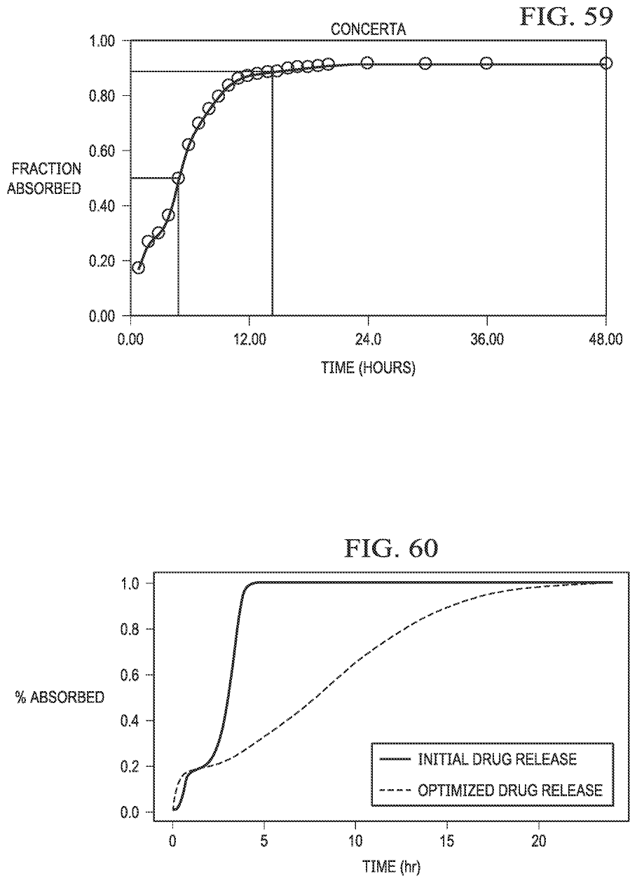

TABLE 59

| | td | ss | ss1 | td1 | ff |
|---|---|---|---|---|---|
| Initial | 0.76 | 3.18 | 6.33 | 3.40 | 0.19 |
| Optimized | 0.31 | 1.00 | 2.15 | 10.84 | 0.18 |

where td is the time to deliver 63.2% of the immediate release part and td1 is the time to release 63.2% of the controlled release part. The terms ss and ss1 refer to the sigmoidicity factors as described previously herein. Table 59 shows that optimal delivery has a much lower sigmoidicity factor (much shallower) release than what is observed for these drugs, and takes 10.8 hours after onset of delivery to reach 63.2% of the drug. FIG. 59 is a graph reporting exemplary data of the cumulative fraction absorbed for Concerta® (ALZA Corporation) and shows that 50% of the drug is absorbed in 4-6 hours and all the drug has been absorbed by approximately 10-12 hours (indicated by vertical and horizontal lines intersecting with the curve), which is much faster than optimal.

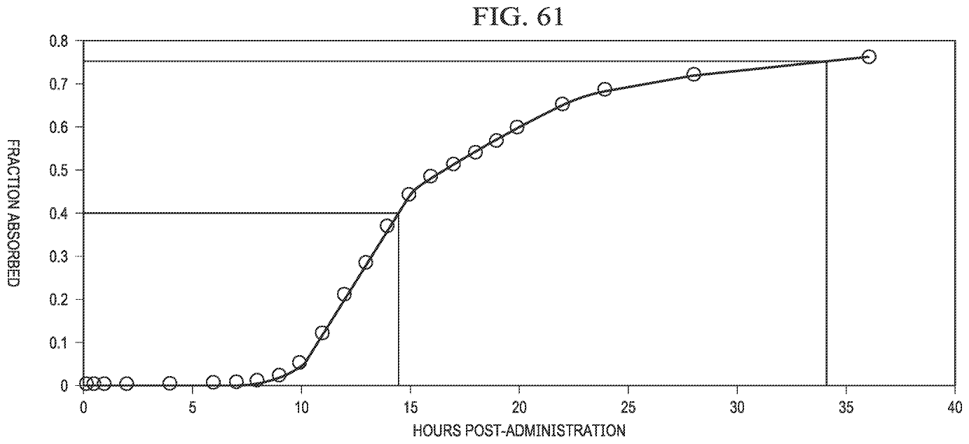

FIG. 60 is a graph reporting exemplary “initial” and “optimized” fractional absorption profiles as determined by R. Gomeni et al. (ASCPT 2016 Annual Meeting, Mar. 8-12, 2016, Hilton Bayfront, San Diego, Calif., incorporated by reference herein). FIG. 60 shows that optimized 50% release is achieved at approximately 7-8 hours after initiation of drug absorption with maximal cumulative absorption occurring at approximately 20 hours. A comparison of this profile to the fractional absorption profile of HLD200 (FIG. 61) indicates 50% absorption (correcting for 75-80% relative bioavailability) occurs at approximately 7 hours after onset of drug absorption for HLD200, and maximal cumulative absorption occurs at approximately 20-24 hours after onset of absorption.

Typical gastrointestinal transit times are: gastric emptying 90 minutes, small bowel transit time 4-6 hours, colon arrival time approximately 8 hours. Thus, products including Concerta® (ALZA Corporation), Ritalin LA® (Novartis AG), Metadate CD® (UCB, Inc), and Quillivant XR® (NextWave Pharmaceuticals, Inc.) appear to deliver most if not all of the product in the small bowel and they all exhibit the same mechanism of absorption, with an approximately linear IVIVC. In contrast, compositions of the present disclosure such as HLD200 deliver the product after approximately 8 hours and thus in the colon, and also exhibit a non-linear and time-variant IVIVC. Without being limited by theory, if absorption in the colon is associated with the non-linear IVIVC, and an optimal curve requires 50% of the absorption to occur after 10 hours, the compositions and methods of the present disclosure allow absorption in the colon, in contrast to the other products such as Concerta® (ALZA Corporation), Ritalin LA® (Novartis AG), Metadate CD® (UCB, Inc), and Quillivant XR® (NextWave Pharmaceuticals, Inc.). In particular, the delayed release formulation of the compositions described herein and methods of the present disclosure including administration during the previous evening allow delivery of the compositions described herein to the colon and absorption of methylphenidate to begin prior to waking in the morning, thereby providing an optimal methylphenidate release and absorption profile.

FIG. 64 is a graph reporting exemplary cumulative % colon arrival time for surrogate beads radio-labelled with not more than 1 MBq ¹¹¹Indium. The plots show results from two separate experiments, indicated as “F1” and “F2”. Subjects were administered with the radiolabeled beads and the arrival time in the colon was assessed using scintigraphy. FIG. 64 shows that all radio-labelled beads in the exemplary formulations are in the colon at 10 hours.

### Example 46. Mean Rate of Change of Methylphenidate Plasma Concentration Over Time

FIG. 62 is a graph reporting exemplary data on mean rate of change of methylphenidate plasma concentration over time (ng/mL/hour) for HLD20054 mg (study 200-101), HLD200100 mg (study 200-109) and CONCERTA® 54 mg.

FIG. 62 shows the rate of change of exemplary HLD200 formulations compared to CONCERTA®. In particular, comparison of HLD20054 mg with CONCERTA® 54 mg shows that HLD20054 mg has a maximum rate of change in increasing methylphenidate plasma concentration of about +1.0 ng/mL/hour and a maximum rate of change in decreasing methylphenidate plasma concentration of about −0.5 ng/mL/hour, whereas CONCERTA® 54 mg has a maximum rate of change in increasing methylphenidate plasma concentration of about +3.6 ng/mL/hour and a maximum rate of change in decreasing methylphenidate plasma concentration of about −1.0 ng/mL/hour.

FIG. 62 also shows HLD200100 mg has a maximum rate of change in increasing methylphenidate plasma concentration of about +2.5 ng/mL/hour and a maximum rate of change in decreasing methylphenidate plasma concentration of about −1.2 ng/mL/hour

Normalization of the CONCERTA® data to a dose of 100 mg CONCERTA® by multiplying by 100/54=1.85 would be expected to give a maximum rate of change in increasing methylphenidate plasma concentration of about +3.6×1.85=+6.7 ng/mL/hour and a maximum rate of change in decreasing methylphenidate plasma concentration of about −1.0×1.85=−1.85 ng/mL/hour for CONCERTA® 100 mg.

In addition, the graph of FIG. 62 shows the methylphenidate plasma concentration rate increase from the second release of CONCERTA® at 16 hrs, where in contrast HLD200 provides a smoother change in rate over the entire profile, having fewer peaks.

### Example 47. Comparative Data: IVIVC Model for Amphetamine Formulation

An exemplary delayed release, extended release formulation of amphetamine was prepared, referred to herein as HLD100-102, and having similar excipient coatings and bead physical characteristics as the medium methylphenidate formulation of HLD200. The exemplary amphetamine formulation has the same film coat components as the medium HLD200 methylphenidate formulation in the same relative ratios. The amount of coating applied (weight gain) is different, such that the amphetamine formulation has a weight gain of 25% for the ER film coat and 20% for the DR film coating. In contrast the exemplary methylphenidate HLD200 medium formulation has 20% ER weight gain and 30% DR weight gain.

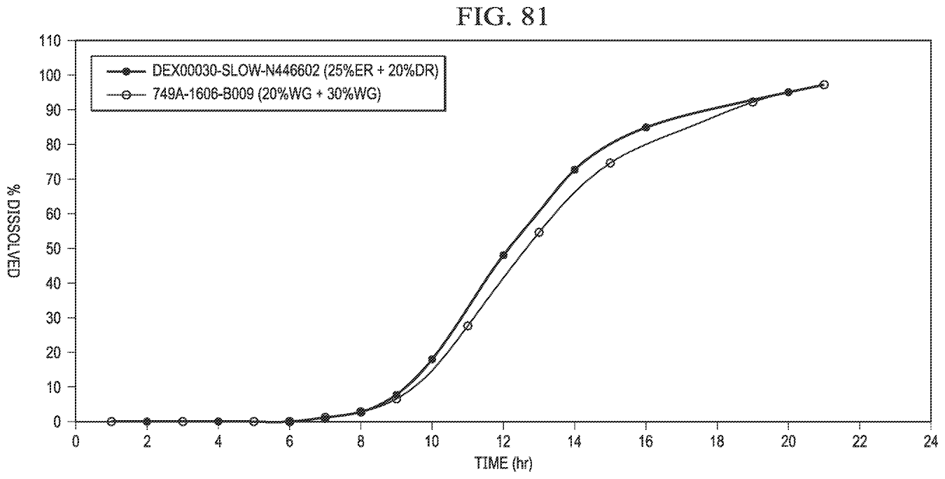

FIG. 81 is a graph reporting an exemplary in vitro dissolution plot of the HLD200 medium formulation together with an exemplary in vitro dissolution plot of the HLD100-102 amphetamine formulation. FIG. 81 shows that the in vitro dissolution profiles of the exemplary HLD100-102 amphetamine formulation and the HLD200 medium formulation are very similar.

The in vitro and in vivo release rates of the exemplary amphetamine formulation were obtained following a similar deconvolution-based IVIVC procedure as described above in Examples 23-28.

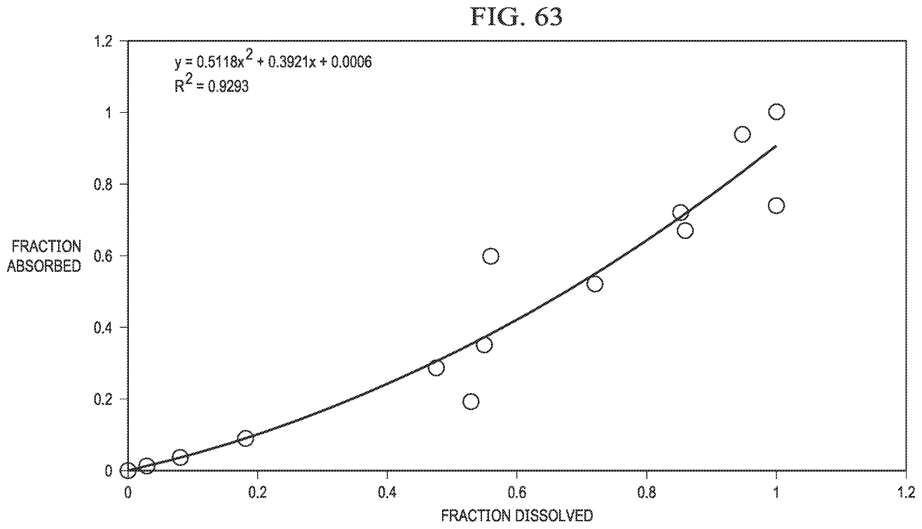

The in vitro and in vivo release rates of the amphetamine HLD100-102 formulation were correlated to derive an IVIVC model. FIG. 63 is a graph reporting exemplary fraction of amphetamine released In Vitro vs. In Vivo from the HLD100-102 formulation.

As suggested by FIG. 63, the IVIVC relationship for the amphetamine formulation is not linear. However, in contrast with the exemplary IVIVC for methylphenidate formulations described in Example 26, in which a 5th-degree polynomial function successfully described the relationship between the in vitro and in vivo release, for the exemplary amphetamine formulation a 2nd-degree polynomial function successfully described the relationship between the in vitro and in vivo release.

Accordingly, comparison of the IVIVC model that fits the exemplary amphetamine formulation with the IVIVC model that fits the exemplary methylphenidate formulation indicates that changing the active ingredient can change the mechanism of release and absorption, as the coatings and bead physical characteristics of the exemplary methylphenidate formulation and the exemplary amphetamine formulation are very similar.

Because the in vitro dissolution profiles of the exemplary amphetamine HLD100-102 formulation and the exemplary methylphenidate HLD200 medium formulation are very similar as shown in FIG. 81, the IVIVC models indicate that the in vivo absorption characteristics differ between the amphetamine and methylphenidate formulations.

### Example 48. Additional IVIVC Modeling of HLD200 Formulations

This Example describes additional IVIVC analysis for the fast, slow, and final formulations of HLD200.

The IVIVC was assessed using a convolution-based modeling approach

The following methods were used.

Convolution-based model. The HLD200 plasma concentration (Cp), resulting from an arbitrary dose, was be described by convolution as:

$${C}_{p} ( t ) = f ( t ) * I ( t )$$

$$f ( t ) = \frac{d r}{d t} ; \frac{d A}{d t} = F \cdot Dose \cdot \frac{dr}{dt} - {k}_{el} \cdot A ; {C}_{p} = \frac{A}{V}$$

where f(t) is the in-vivo input function, I(t) is the unitary impulse response (defined by the volume of distribution (V) and the first order elimination rate (kel) estimated using the IR formulation data), * is the convolution operator, r(t) is the time-varying fraction of the dose released defined by a Weibull model, A is the amount of drug, and F is the fraction of the dose absorbed.

IVIVC Modeling. The analysis presented in this Example is focused on the development of a Level A IVIVC by evaluating a point-to-point correlation between the fraction of drug absorbed in-vivo (rᵥᵢᵥₒ (t) and the fraction of drug dissolved (rᵥᵢₜᵣₒ r(t)). In the IVIVC assessment, the in-vitro dissolution and in-vivo input curves may be directly super-imposable or may be made to be super-imposable by using a “scaling factor”. A time scaling function was be included in the model to account for potential time differences in the in-vitro and the in-vivo processes (for example when the dissolution is faster than the in-vivo input rate). A general time scaling model was be applied in the assessment of the IVIVC:

rᵥᵢᵥₒ(t)=a₁+a₂·rᵥᵢₜᵣₒ(tt)

tt=b₁+b₂·t^{b}^{₃}

In case of absence of time scaling between rᵥᵢᵥₒ and rᵥᵢₜᵣₒ: a₁=0, a₂=1, b₁=0, b₂=1, and b₃=1. Otherwise, the time scaling can be defined by estimating the appropriate values of the parameters a1, a2, b1, b2, and b3. The Equation 3 includes a linear component (intercept of a₁ and slope of a₂), and a nonlinear component describing the time-shifting (b₁), time-scaling (b₂), and time-shaping factor (b3).

Model validation. The final step in the IVIVC analysis is to validate the model by providing quantified evidence of the predictive performance of the model. The model validation was accomplished using data from the formulations used to build the model (internal validation). The validation was implemented using the IVIVC model: the relevant exposure parameters (Cmax and AUCinf) were predicted using the model for each formulation and compared to the observed values. The prediction error (% PE) was calculated for each PK parameter using the equation:

$$\% PE = \frac{1}{n} {\sum }_{1}^{n} \frac{\vert Observed value - Predicted value \vert}{Observed value} \cdot 100$$

where n is the number of formulations. The criteria for assessing the level of predictability are for each PK parameter: average % PE≤10% with no individual values>15%. If criteria are not met, the evaluation of external predictability would be required.

Implementing IVIVC. The IVIVC analysis was conducted using a 6-step approach:

1. Fit the PK time-course of the IR formulation (Step 1);

2. Individually fit the mean in-vitro dissolution data of the slow, medium, and fast formulations using the release function defined by a Weibull model (Step 2);

3. Fit the in-vivo PK of the 3 formulations by fixing the disposition (V) and the elimination (kel) parameters estimated from the analysis of the IR formulation (Step 3);

4. Evaluate IVIVC by jointly applying the convolution model to the in-vivo data of the slow, medium, and fast formulations (Step 4) and by: (a) Fixing the in-vivo drug release parameters for each formulation to the values estimated in Step (2), (b) Estimating the time scaling factors common to all formulations, (c) Estimating the relative bioavailability of each formulation.

5. Evaluate the internal predictability by comparing the predicted (estimated in Step 3) Cmax, AUCinf with the observed values (Step 5).

6. Evaluate the external predictability of the final IVIVC model by predicting the in-vivo performance of a formulation not used in development of the IVIVC model.

The following data were used in the analysis.

Immediate release. The PK data for the Ritalin® IR formulation at the dose of 20 mg were utilized to derive the unit impulse function. These data were generated in the study no: HLD200-111: A Phase I, Single Center, Single-Dose, Open-Label, Randomized, Crossover, Comparative Bioavailability Study of HLD200, Methylphenidate HCl Delayed and Extended Release Capsules, to an Immediate Release Methylphenidate HCl Marketed Formulation in Healthy Adult Volunteers. In this study, a total of 12 subjects were randomly assigned to 2 treatment sequence cohorts (HLD200, 100 mg and Ritalin®, 20 mg) of 6 subjects each in crossover fashion.

In-vitro Dissolution. The lots of the formulations used in the dissolution testing were: 749A-1811-B003-FAST, 749A-1606-B008-MID, 749A-1747-B006-SLOW. The dissolution testing took place for 2 hours (T=0-2 hour) in 0.1N HCl, then in pH 6.0 phosphate buffer for 4 hours (T=2-6 hour), and finally in pH 7.2 phosphate buffer for the remaining time. Refer to Table 60, Table 61 and Table 62 for details of the dissolution method. Dissolution results for levels of drug release below 5% will be variable since the demonstrated range of the dissolution method is 5% to 130% drug release as determined during the dissolution method validation. Values below 5% would be below the limit of quantitation of the method which would lead to variability in the results at this low level.

TABLE 60
Dissolution Apparatus and Conditions:

| Apparatus | USP <711> Apparatus 1, Baskets (40-mesh) |
|---|---|
| Vessel Size/Type | 1000 mL, round bottom, clear glass |
| Rotation Speed | 75 rpm |
| Test Temperature | 37° C. ± 0.5° C. |
| Pull Volume | 5 mL |
| Replacement | No |
| Sinker | No |
| Cannula | Stainless Steel |
| Filter Type/Size | 10 μm, Polyethylene full flow filter |
| Volume Discard | None |
| Sampling Time | Stage 1: 2 hours |
| Points¹ | Stage 2: 4 and 6 hours |
| | Stage 3: 8, 10, 12, 14, 16, 18, 20, 22 and 24 |
| | hours |
| Prime | 60 seconds |
| Purge | 60 seconds |
| Repeat Prime/Purge | 2 times |

wherein ‘1’ indicates sampling up to 6 hours may be performed manually or with an autosampler. Samples after 6 hours were collected with an autosampler.

TABLE 61
HPLC Conditions for Dissolution:

| Column | Zorbax Eclipse ® |
|---|---|
| Dimension | 150 mm × 4.6 mm |
| | (i.d) |
| Packing | XDB-CN, 5 μm |
| Pump | Isocratic |
| Detection | UV @ 205 nm |
| Injection Volume | 5 μL |
| Flow Rate | 2.0 mL/min |
| Column Temperature | 30° C. |
| Autosampler | Ambient |
| Temperature | |
| Run Time | 7 minutes |

TABLE 62
Media, Mobile Phase and Diluent for Dissolution:

| | |
|---|---|
| Dissolution | Stage 1 (0-2 Hours) - 700 mL of 0.1N Hydrochloric Acid |
| Media | Stage 2 (2-6 Hours) - 700 mL of 0.1N Hydrochloric Acid |
| | and 200 mL of 0.2M Sodium Phosphate Tribasic Buffer, |
| | pH 6.0 ± 0.05 |
| | Stage 3 (6-24 hours) - 700 mL of 0.1N Hydrochloric Acid |
| | and 200 mL of 0.2M Sodium Phosphate Tribasic, and |
| | 10 mL of 2N Sodium Hydroxide, pH 7.2 ± 0.05 |
| Mobile | Sodium Octanesulfonate Buffer pH 3.0/Acetonitrile |
| Phase | (80:20 v/v) |
| Diluent | 0.1% Phosphoric Acid |

In-vivo PK. The PK data were generated in the HLD200-111 study. This was a Phase 1, Single-Center, Single-Dose, Open-Label, Randomized, Crossover, Comparative Bioavailability Study of Three Formulations of Methylphenidate Hydrochloride Delayed Release/Extended Release Capsules (HLD200) in Healthy Adults. A total of 18 subjects were randomly assigned to six treatment sequence cohorts of 3 subjects each in crossover fashion (Table 63). Different release rates of the active compound distinguished the three HLD200100 mg formulations, defined as: Treatment A (HLD200-F, fastest release profile), Treatment B (HLD200-M, the marketed Jornay PM® (Ironshore Pharmaceuticals & Development, Inc.) formulation and reference treatment), and Treatment C (HLD-200-S, slowest release profile). All subjects were dosed under fasted conditions.

TABLE 63
Study HLD200-111 - Crossover design:

| Treatment Sequence | Period 1 | Period 2 | Period 3 |
|---|---|---|---|
| 1 | A | B | C |
| 2 | A | C | B |
| 3 | B | A | C |
| 4 | B | C | A |
| 5 | C | A | B |
| 6 | C | B | A |

External Validation. The lot of the formulation used in the dissolution testing was: 749A-1510-B009. The dissolution testing was conducted according to the same methodology presented above.

The PK data of the study HLD200-109 at 100 mg in the fasted arm were used to assess the external validation. This study was a Phase I, Single Center, Clinical Trial Examining the Pharmacokinetic Effects of 100 mg of HLD200, Methylphenidate HCl Modified Release Capsules in Healthy Adult Volunteers in a Fasted, Fed and Sprinkled State under a Randomized Three-way Cross-over Design. A total of 18 subjects were randomized in equal proportions to one of the following six treatment sequences of a single 100 mg dose of HLD200 (Table 64), where Treatment A=fed, Treatment B=sprinkled (on applesauce), and Treatment C=fasted. The six subjects who withdrew or were withdrawn after administration of at least one dose of investigational product (IP) were replaced to achieve three evaluable subjects per treatment sequence, so a total of 24 subjects were enrolled in the study.

TABLE 64
Study HLD200-109 - Crossover design:

| Treatment | Treatment | Treatment | Treatment |
|---|---|---|---|
| Sequence | Period 1 | Period 2 | Period 3 |
| 1 | A | B | C |
| 2 | A | C | B |
| 3 | B | A | C |
| 4 | B | C | A |
| 5 | C | A | B |
| 6 | C | B | A |

Treatment A: fed (after a high-fat meal beginning 30 minutes and ending 5 minutes pre-dose)
Treatment B: sprinkled (contents of capsule on applesauce)
Treatment C: fasted (for a minimum of 8 hours)

The following results were observed.

Step 1—Fit the PK Time-Course of the IR Formulation.

The individual PK data for the IR formulation were utilized to characterize the disposition and elimination of MPH. The absorption, disposition and elimination of MPH following dosing of the IR formulation was best characterized by a one-compartment model with first order absorption with a lag time. The model parameters were: ka (first order absorption rate constant), kel (elimination rate constant), lag (lag time), V (volume of distribution, and a combination of additive and proportional residual error. The population PK analysis of the IR data was conducted in NONMEM using the ADVAN14 subroutine and the FOCE-I method. The estimated population parameter values are presented in Table 65. In this table SE represents the standard error and RSE represents the relative standard error of the estimated parameters.

TABLE 65
Estimated PK parameter values for the IR formulation:

| Fixed | Parameter | Value | SE | RSE |
|---|---|---|---|---|
| Effect | Ka(hr−1) | 4.28 | 0.0259 | 0.60% |
| | Kel(hr−1) | 0.256 | 0.0011 | 0.40% |
| | V(L) | 2.18 | 0.0357 | 1.60% |
| | Lag(hr) | 0.491 | 0.0015 | 0.30% |
| | prop-err | 0.128 | 0.0012 | 0.90% |
| | add-err | 0.0568 | 0.0004 | 0.70% |
| Random | Ka(hr−1) | 1.18 | 0.0645 | 5.50% |
| Effect | Kel(hr−1) | 0.0165 | 0.0024 | 14.70% |
| | V(L) | 0.165 | 0.108 | 65.50% |

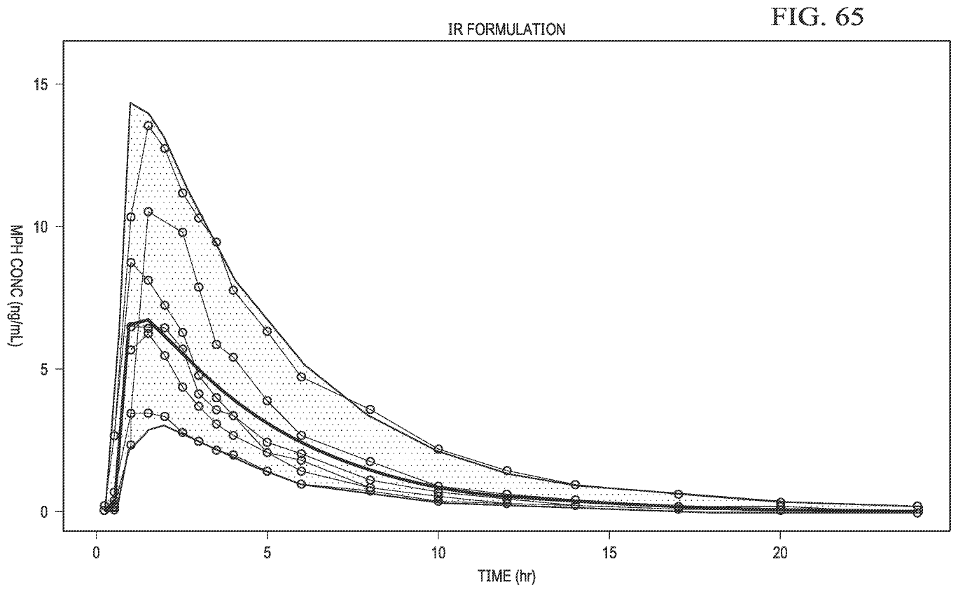

Visual predictive check method was utilized to evaluate the adequacy of model to describe the IR data. The basic premise is that a model and parameters derived from an observed data set should produce simulated data that are similar to the original observed data. Five hundred replicates of the original dataset were simulated, based on the final model, and the 90% prediction intervals were computed based on the simulated datasets. The observed drug concentration values versus time were plotted on the prediction interval to visually assess the concordance between the simulated and observed data. The visual predictive checks of the population PK modelling for the IR formulation is presented in FIG. 65.

The VPCs showed that the model developed to characterize the MPH plasma concentration time-course following an IR formulation of performed well: the distribution of the observed data was well predicted in term of typical profile (median curves identified by the solid heavy line) and inter-individual variability (90% prediction intervals identified by the shaded area), indicating that the population model properly described the observed data.

Step 2—Fit the Mean Dissolution Data.

The in-vitro dissolution data of each extended-release formulation was described by a double Weibull model:

$${r}_{v i t r o} ( t ) = Dose \cdot [ 1 - ( ff \cdot {e}^{- ( {( \frac{t i m e}{t d} )}^{SS} )} + ( 1 - ff ) \cdot {e}^{- ( {( \frac{t i m e}{t d 1} )}^{S S 1} )} ) ]$$

where ff is the fraction of the dose dissolved in the 1st process, td and td1 are the times to dissolve 63.2% of the dose in the 1st and in the 2nd process, ss and ss1 are the sigmoidicity factors for the 1st and the 2nd process.

The Weibull model (r(t)) was fitted to the mean in-vitro dissolution data of the Slow, Med, and Fast dissolution formulations. The following parameters were estimated for each formulation: ff (the fraction of the available dose released in the 1st process), td and td1 (the times to release 63.2% of the dose in the 1st and in the 2nd process), ss and ss1 (the sigmoidicity factors for the 1st and the 2nd process dissolution process). The analysis of the dissolution data was conducted in NONMEM using the FOCE-I method.

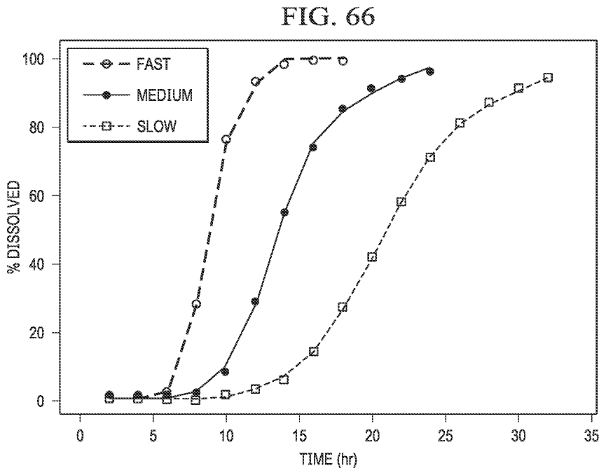

The mean dissolution data for each formulation with the model predicted curves are presented in FIG. 66. The estimated parameter values are presented in Table 66. In this table RSE represents the relative standard error of the estimated parameters.

TABLE 66
Estimated dissolution data parameters for the Slow, Med, and Fast
dissolution rate formulations (RSE = relative standard error):

| | Slow | | Med | | Fast | |
|---|---|---|---|---|---|---|
| Parameter | Value | RSE | Value | RSE | Value | RSE |
| td(hr) | 8.18 | <0.01% | 13.6 | <0.01% | 20.8 | <0.01% |
| ss(unit less) | 36.3 | 0.1920 | 6.71 | <0.01% | 5.91 | <0.01% |
| td1(hr) | 10.7 | <0.01% | 19.3 | <0.01% | 28.6 | <0.01% |
| ss1(unit less) | 5.37 | <0.01% | 4.28 | <0.01% | 5.11 | <0.01% |
| ff(%) | 0.518 | <0.01% | 0.668 | <0.01% | 0.677 | <0.01% |
| err0 | 0.67 | <0.01% | 1.12 | <0.01% | 0.442 | <0.01% |

Step 3—Fit the In-Vivo PK of the 3 Formulations Using a Convolution-Based Model by Fixing the Disposition and the Elimination Parameters Values Estimated from the IR Formulation.

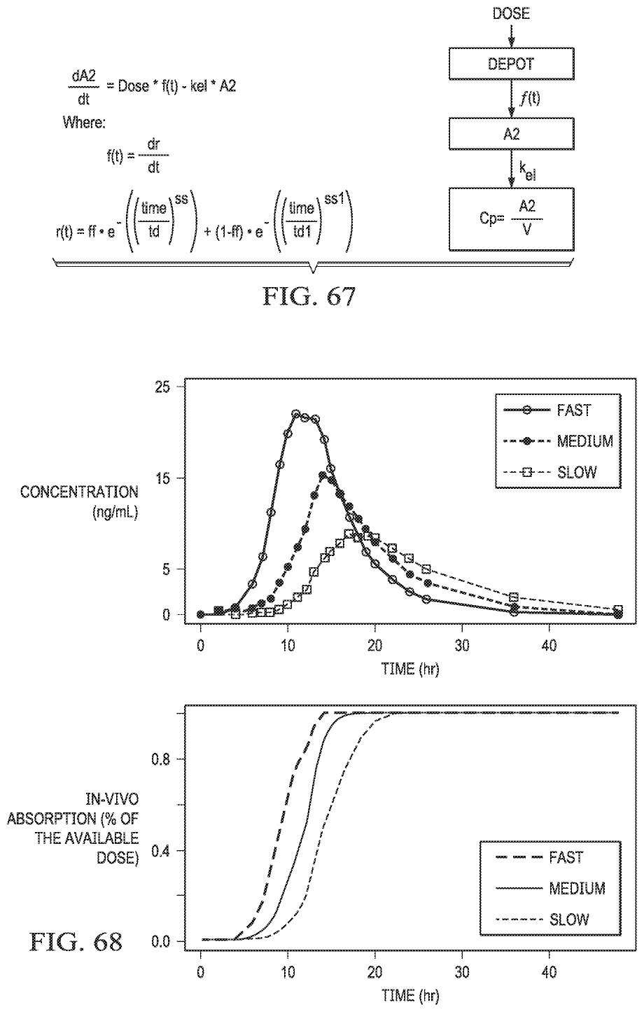

The objective of this analysis was to provide an estimate the in-vivo drug release rate together with an estimate the inter-individual variability of the elimination rate constant for the 3 formulations. At this purpose, the convolution model presented in FIG. 67 was used to jointly fit the data of the 3 formulations using a non-nonlinear mixed-effect approach. The in-vivo drug release was estimated fixing the mean values of the volume (V) and the mean value of the elimination rate (kel) to the values estimated in the analysis of the IR data.

The PK time course of in-vivo release of the different formulations was assumed to be characterized by two phases of drug release: a first phase concerning the initial release of a fraction of the dose, and a second phase to provide an extended release of the dose. The r(t) function for MPH was modelled using a double Weibull function. In this model, ff represents the fraction of the available dose released in the 1st process, td and td1 are the times to release 63.2% of the dose in the 1st and in the 2nd process, ss and ss1 are the sigmoidicity factors for the 1st and the 2nd process.

The estimated parameter values are presented in Table 67. In this table SE represents the standard error and RSE represents the relative standard error of the estimated parameters.

TABLE 67
Estimated in-vivo PK parameters for the Slow,
Med, and Fast dissolution rate formulations:

| Fixed | Parameter | Value | SE | RSE |
|---|---|---|---|---|
| Effect | Kel(hr−1) | 0.256* | | |
| | V(L) | 2.18* | | |
| | td(hr) | 12.200 | 1.470 | 12% |
| | ss(unit less) | 5.120 | 0.038 | 0.70% |
| | td1(hr) | 13.000 | 0.037 | 0.30% |
| | ss1(unit less) | 26.900 | 11.000 | 40.90% |
| | ff(%) | 0.855 | 0.016 | 1.90% |
| | add-err | 0.123 | 0.023 | 18.70% |
| | prop-err | 0.022 | 0.006 | 25.40% |
| Random | Kel(hr−1) | 0.438 | 0.209 | 47.70% |
| Effect | V(L) | 0.534 | 0.297 | 55.60% |
| | td(hr) | 0.0463 | 0.021 | 45.40% |

*fixed from the analysis of the IR data

The mean PK observations for each formulation with the model predicted curves (top panel) and the in-vivo release rate (bottom panel) are presented in FIG. 68.

Step 4—Evaluate the IVIVC Using the Convolution Modelling Approach.

The convolution model used for the assessment of the IVIVC relationship is presented in FIG. 67. The data of the 3 formulations were jointly fitted. The following parameters were fixed to the values estimated in the previous analyses: The mean kel, and V estimated from the analysis of the IR data (Step 1), the inter-individual variability of kel estimated for the analysis conducted in the Step 3, the dissolution data (ff, td, ss, td1, and ss1 for the 3 formulations) estimated in Step 2. The convolution model was implemented in NONMEM using the ADVAN13 subroutine and the FOCE-I method.

The data of the three formulations (Slow, Med, and Fast) were jointly fitted to estimate the following parameters: the time scaling parameters (a1, a2, b1, b2, and b3), the fraction of the dose administered available for the systemic circulation (F_Slow, F_Med, and F_Fast), and the additive residual error (err). The f(t) function was estimated by using the finite difference approach. The F_parameters represent a dose scaling value with respect to the reference IR formulation. The rational to use this parameter is to allow for any difference which might exist in the bioavailability between the reference formulation and the Slow, Med, and Fast release formulations.

The time-scaling parameters (a1, a2, b1, b2, and b3) were estimated as fixed effect parameters (without random effect) as these parameters were expected to take the same value for all formulations. No random effect was associated to any parameter of the convolution model.

The estimated parameter values are presented in Table 68. The mean observed and model predicted MPH in-vivo concentrations for the Slow, Med, and Fast release rate formulations are presented in FIG. 69.

TABLE 68
Estimated parameter values in the convolution analysis
for the IVIVC assessment. SE is the standard error and
RSE is the relative standard error of the parameters:

| Parameter | Value | FIX | SE | RSE |
|---|---|---|---|---|
| A1 | 0 | FIX | 0 | 0% |
| A2 | 1 | FIX | 0 | 0% |
| B1 | 7.540 | — | 0.019 | 0.30% |
| B2 | 0.069 | — | 0.004 | 5% |
| B3 | 5.520 | | 0.007 | 0.10% |
| F_Fast | 0.883 | — | 0.000 | 0% |
| F_Med | 0.478 | — | 0.014 | 2.80% |
| F_Slow | 0.238 | — | 0.004 | 1.70% |
| err | 1.290 | — | 0.005 | 0.40% |

The results of the analysis indicated that the relative bioavailability of the Slow, Med, and Fast release formulation with respect to the IR formulation where 0.88%, 0.48% and 0.24%, respectively.

An analysis was conducted to evaluate the correlation between the in-vitro release and the in-vivo release resulting from the IVIVC analysis with the inclusion of time scaling correction.

The regression analysis is presented in the FIG. 70 and the results of the regression analysis are presented in Table 69. The analysis indicated that a statistically significant correlation was found between the in-vitro dissolution and the in-vivo absorption data (p<0.001).

TABLE 69
Results of the regression analysis of the in-vivo versus in-vitro release:
Parameter Estimates

| | | Parameter | Standard | | | 95% Confidence | |
|---|---|---|---|---|---|---|---|
| Variable | DF | Estimate | Error | t Value | Pr > \|t\| | Limits | |
| Intercept | 1 | 0.01843 | 0.01728 | 1.07 | 0.2901 | −0.0161 | 0.05293 |
| Slope | 1 | 0.93561 | 0.02248 | 41.62 | <.0001 | 0.89073 | 0.9805 |

Step 5. Internal Validation.

a) Correlation Analysis Between Observed PK and Predicted PK from the IVIVC Analysis.

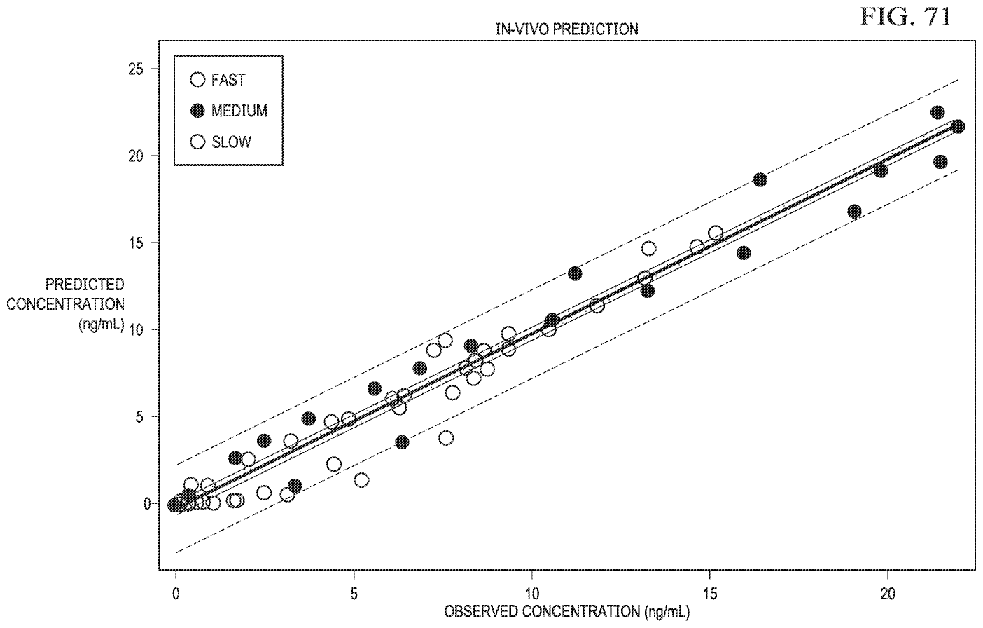

The in-vitro dissolution data and the convolution model were used to predict the in-vivo PK profiles of the three formulations. The convolution model predictions were in close agreement with the observed concentrations. FIG. 71 presents the plot of the predicted versus the observed concentrations with the regression line.

The results of the regression analysis are presented in Table 70. The analysis indicated that the intercept was not statistically different from zero and that the slope was not statistically different from one as the 95% confidence limits included 0 for the intercept and 1 for the slope.

TABLE 70
Results of the regression analysis of the predicted versus the observed concentrations:
Parameter Estimates

| | | Parameter | Standard | | | 95% Confidence | |
|---|---|---|---|---|---|---|---|
| Variable | DF | Estimate | Error | t Value | Pr > \|t\| | Limits | |
| Intercept | 1 | −0.3071 | 0.21724 | −1.41 | 0.1622 | −0.7408 | 0.12663 |
| Slope | 1 | 1.00394 | 0.0245 | 40.98 | <.0001 | 0.95503 | 1.05285 |

b) Internal Predictability Assessment.

The area under the plasma-concentration time curves between zero and the last measurable concentration (AUC) values were estimated using the log-linear trapezoidal rule for the observed and the model predicted data. The prediction errors (% PE) were estimated using the AUC and the Cmax values for observed and model predicted concentration. The estimated % PE values are presented in Table 71. The largest % PE (=11.05%) was estimated for the AUC of the Fast formulation. The average % PE across the three formulations were 6.0% and 4.31% for AUC and Cmax, respectively.

TABLE 71
Comparison of the Cmax and AUC values estimated on the observed
PK data with the values estimated on the convolution model predicted
PK data with the assessment of the prediction error (% PE):

| | Observed Values | | Predicted Values | | | |
|---|---|---|---|---|---|---|
| | Cmax | AUC | Cmax | AUC | Prediction Error | |
| Formulation | (ng/mL) | (ng*hr/mL) | (ng/mL) | (ng*hr/mL) | % PE_AUC | % PE_Cmax |
| Fast | 21.93 | 231.62 | 22.52 | 237.80 | 2.67 | 2.69 |
| Med | 15.14 | 177.29 | 15.55 | 169.73 | 4.26 | 2.72 |
| Slow | 8.71 | 151.27 | 9.36 | 167.99 | 11.05 | 7.51 |

| | | |
|---|---|---|
| Average | 6.00 | 4.31 |

Step 6—External Validation.

a) Refined IVIVC Model.

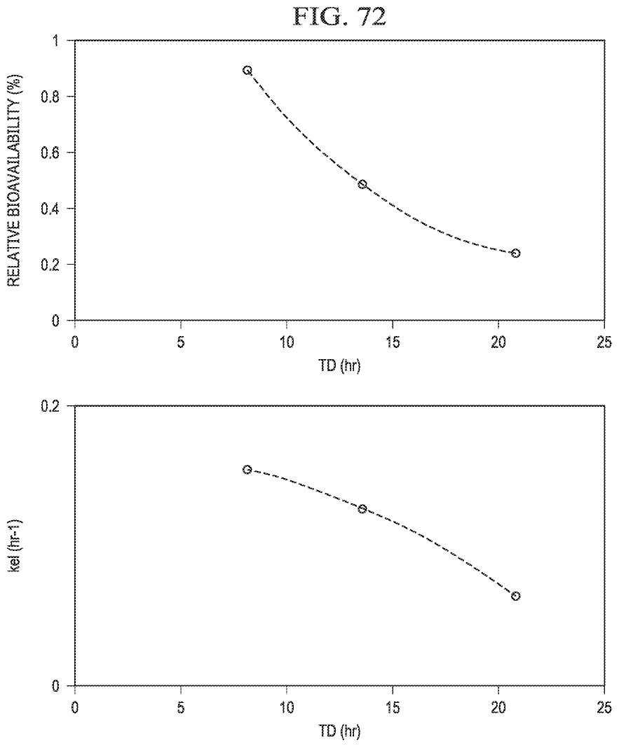

The previously defined IVIVC model, the disposition (V/F=2.11) and elimination (kel=0.256) parameter values were fixed to the values estimated in the Step 3. Furthermore, the inter-formulation variability for V/F was fixed to 0 and the inter-formulation variability for kel was fixed to 0.438 ‘Step 3). This model was appropriate for supporting internal validation. However, this model was not appropriate for predicting the in-vivo PK of a formulation different from the ones used in model development. This because no rule was established to estimate the in-vivo relative bioavailability (BI) and the kel value from the in-vitro dissolution data. To overcome this limitation, the initial IVIVC model was refined by including a sub-model describing the relationship between in-vitro properties, in-vivo relative bioavailability and in-vivo kel. As shown in FIG. 72, a polynomial relationship was empirically identified between the in-vitro estimated TD parameters (the time necessary for dissolving 63.2% of the dose in the 1st release process) and the in-vivo estimates of the relative bioavailability (BI) and Kel of each formulation (F_Slow, F_Med, and F_Fast). These models were suitable to estimate by interpolation the value of BI and Kel of a new formulation used in an external validation when the TD parameter of the novel formulation took values in the range of the TD values of the formulations used in the IVIVC model development: 818 to 20.8 (hr).

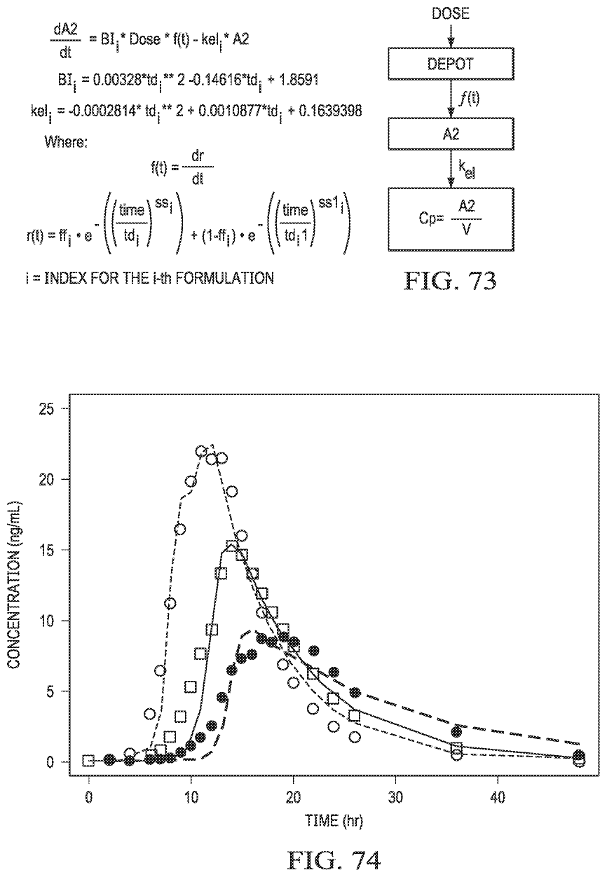

The refined convolution model including the dependency between the dissolution properties and the estimated in-vivo relative BI and kel is presented in FIG. 73.

The estimated parameter values are presented in Table 72. The mean observed and model predicted MPH in-vivo concentrations for the Slow, Med, and Fast release rate formulations are presented in FIG. 74.

TABLE 72
Estimated parameter values in the refined convolution analysis
for the IVIVC assessment. SE is the standard error and
RSE is the relative standard error of the parameters:

| Parameter | Value | FIX | SE | RSE |
|---|---|---|---|---|
| A1 | 0 | FIX | 0 | 0% |
| A2 | 1 | FIX | 0 | 0% |
| B1 | 7.550 | — | 0.058 | 0.80% |
| B2 | 0.060 | — | 0.029 | 48.10% |
| B3 | 5.580 | — | 0.177 | 3.20% |
| eff | 1.250 | — | 0.084 | 6.70% |

Model Validation of the Refined IVIVC Model.

Correlation Analysis Between Observed PK and Predicted PK from the IVIVC Analysis.

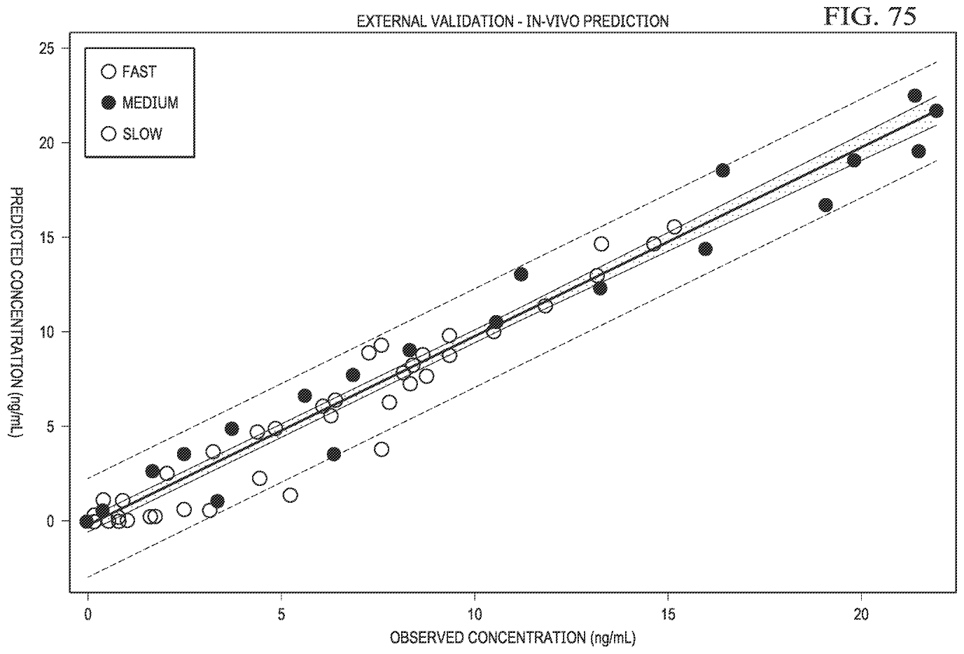

The in-vitro dissolution data and the convolution model were used to predict the in-vivo PK profiles of the three formulations. The convolution model predictions were in close agreement with the observed concentrations. FIG. 75 presents the plot of the predicted versus the observed concentrations with the regression line.

The results of the regression analysis are presented in Table 73. The analysis indicated that the intercept was not statistically different from zero and that the slope was not statistically different from one as the 95% confidence limits included 0 for the intercept and 1 for the slope.

TABLE 73
Results of the regression analysis of the predicted versus
the observed concentrations for the refined IVIVC model:
Parameter Estimates

| | | Parameter | Standard | | | 95% Confidence | |
|---|---|---|---|---|---|---|---|
| Variable | DF | Estimate | Error | t Value | Pr > \|t\| | Limits | |
| Intercept | 1 | −0.30133 | 0.21692 | −1.39 | 0.1695 | −0.7344 | 0.13176 |
| Slope | 1 | 1.00203 | 0.02446 | 40.96 | <.0001 | 0.95319 | 1.05087 |

Predictability Assessment

The area under the plasma-concentration time curves between zero and the last measurable concentration (AUC) values were estimated using the log-linear trapezoidal rule for the observed and the model predicted data. The prediction errors (% PE) were estimated using the AUC and the Cmax values for observed and model predicted concentration. The estimated % PE values are presented in Table 74. The largest % PE (=11.08%) was estimated for the AUC of the Slow formulation. The average % PE across the three formulations were 5.93% and 4.34% for AUC and Cmax, respectively.

TABLE 74
Comparison of the Cmax and AUC values estimated on the observed PK
data with the values estimated on the refined convolution model predicted
PK data with the assessment of the prediction error (% PE):

| | Observed Values | | Predicted Values | | | |
|---|---|---|---|---|---|---|
| | Cmax | AUC | Cmax | AUC | Prediction Error | |
| Formulation | (ng/mL) | (ng*hr/mL) | (ng/mL) | (ng*hr/mL) | % PE_AUC | % PE_Cmax |
| Fast | 21.93 | 231.62 | 22.49 | 237.14 | 2.3842 | 2.55416 |
| Med | 15.14 | 177.29 | 15.59 | 169.64 | 4.3141 | 3.00628 |
| Slow | 8.71 | 151.27 | 9.36 | 168.04 | 11.0857 | 7.45883 |

| | | |
|---|---|---|
| Average | 5.93 | 4.34 |

Dissolution Data

The in-vitro dissolution data formulation were described by the same Weibull model previously applied.

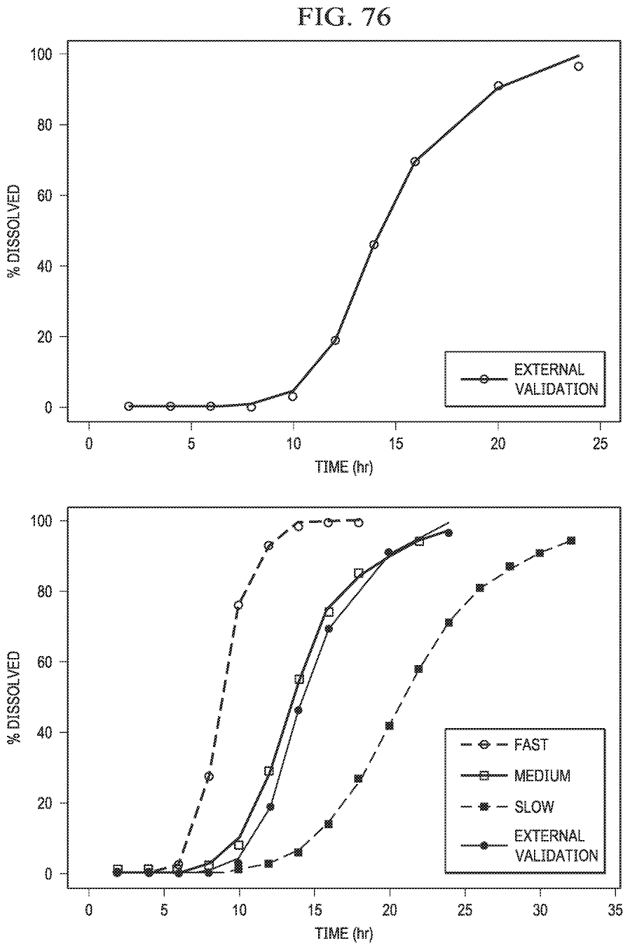

The mean dissolution data with the model predicted curve are presented in FIG. 76. The estimated parameter values are presented in Table 75. In this table RSE represents the relative standard error of the estimated parameters.

TABLE 75
Estimated dissolution data parameters for the formulation used
in the external validation (RSE = relative standard error):

External validation

| Parameter | Value | RSE |
|---|---|---|
| td(hr) | 13.7 | <0.01% |
| ss(unit less) | 8.84 | <0.01% |
| td1(hr) | 18.5 | <0.01% |
| ss1(unit less) | 5.53 | <0.01% |
| ff(%) | 0.54 | <0.01% |
| err0 | 1.19 | <0.01% |

In-Vivo PK Used in the External Validation

The PK data of the study HLD200-109 collected in 18 subjects at 100 mg in the fasted arm were used to assess the external validation.

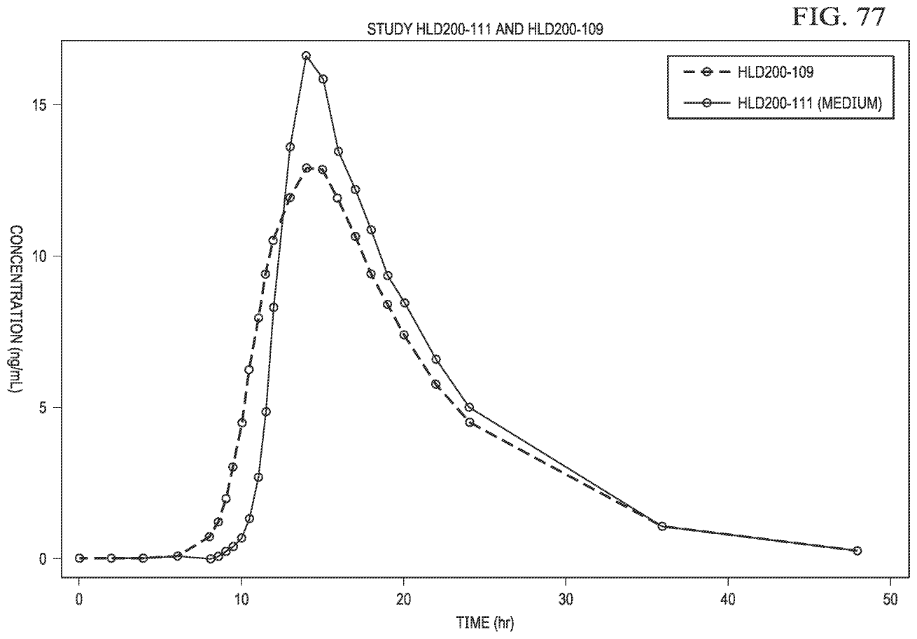

FIG. 77 shows the comparison of the mean concentrations time-course for the formulation with median dissolution rate in the study HLD200-111 and the PK concentrations time-course in the study HLD200-109 (Fasted arm).

While the dissolution data of the formulation with median dissolution rate in the study HLD200-111 and the dissolution data of the formulation used in the study HLD200-109 (Fasted arm) showed an almost superimposable profile (FIG. 76), the mean in-vivo Cmax in the HLD200-109 study was ˜30% lower than the value observed in the HLD200-111 study.

A population PK modelling approach was used to characterize the PK data using the same model described above: a one compartment model with a time varying absorption described by a double Weibull function and a first order elimination rate constant.

The estimated parameter values are presented in Table 76. In this table SE represents the standard error and RSE represents the relative standard error of the estimated parameters.

TABLE 76
Population PK parameters estimates for
the data of the study HLD200-109:

| Fixed | Parameter | Value | SE | RSE |
|---|---|---|---|---|
| Effect | Kel(hr−1) | 0.121 | 0.0103 | 8.50% |
| | V/F(L) | 4.89 | 0.0143 | 0.30% |
| | td(hr) | 13 | 0.0467 | 0.40% |
| | ss(unit less) | 7.74 | 0.014 | 0.20% |
| | td1(hr) | 10.5 | 0.0311 | 0.30% |
| | ss1(unit less) | 10.1 | 0.0511 | 0.50% |
| | ff(%) | 0.603 | 0.002 | 0.30% |
| | add-err | 0.145 | 0.0099 | 6.80% |
| | prop-err | 0.076 | 0.0062 | 8.10% |
| Random | Kel(hr−1) | 0.0657 | 0.0293 | 44.60% |
| Effect | V/F(L) | 0.2 | 0.0863 | 43.10% |
| | td(hr) | 0.0135 | 0.39% | 29% |
| | ss(unit less) | 0.0125 | 0.96% | 76.60% |
| | td1(hr) | 0.0243 | 0.62% | 25.70% |
| | ss1(unit less) | 0.27 | 36.10% | 133.70% |
| | ff(%) | 0.0586 | 0.40% | 6.80% |

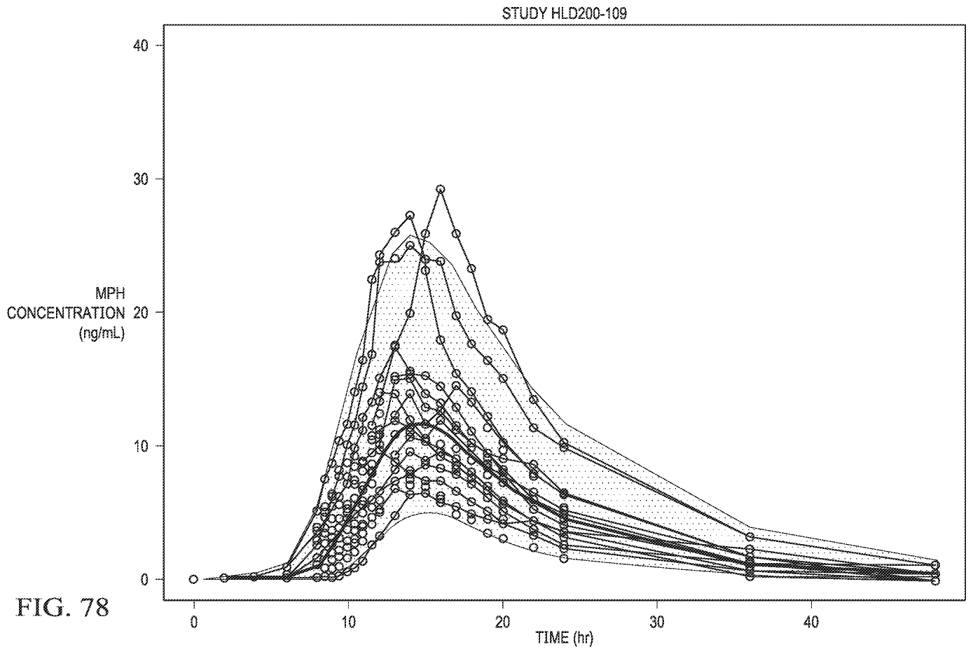

Five hundred replicates of the original dataset were simulated, based on the final model, and the 90% prediction intervals were computed based on the simulated datasets. The observed drug concentration values versus time were plotted on the prediction interval to visually assess the concordance between the simulated and observed data. The visual predictive checks of the population PK modelling is presented in FIG. 78.

The VPCs showed that the model developed to characterize the MPH plasma concentration time-course in the study HLD200-109 performed well: the distribution of the observed data was well predicted in term of typical profile (median curves identified by the solid thick line) and inter-individual variability (90% prediction intervals identified by the grey shaded area), indicating that the population model properly described the observed data.

External Predictability Assessment

The expected typical profile of the in-vivo PK in the study HLD200-109 was predicted using the refined IVIVC model (Table 76) jointly with the dissolution release parameters of the formulation used in the external validation (Table 75). In this model the in-vivo PK data were estimated by computing the model outcome when all the parameters values were fixed to the value shown in Table 77.

TABLE 77
Parameter values used in the assessment of the in-vivo
PK for the evaluation of the external predictability:

| | |
|---|---|
| TD | 13.7 |
| SS | 8.45 |
| TD1 | 18.5 |
| SS1 | 5.53 |
| FF | 0.54 |
| V/F | 2.18 |
| BI | 0.00328*TD**2 − 0.14616*TD + 1.8591 |
| KEL | −0.0002814*TD**2 + 0.0010877*TD + 0.1639398 |

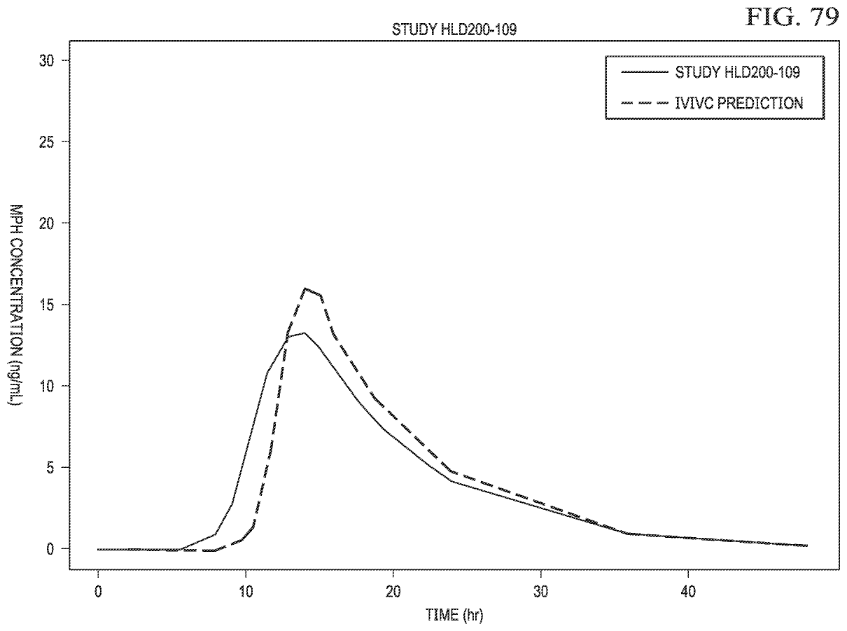

FIG. 79 shows the comparison of the typical PK time course of the study HLD200-109 (solid curve) with the convolution-based estimate of the expected PK profile (dashed curve).

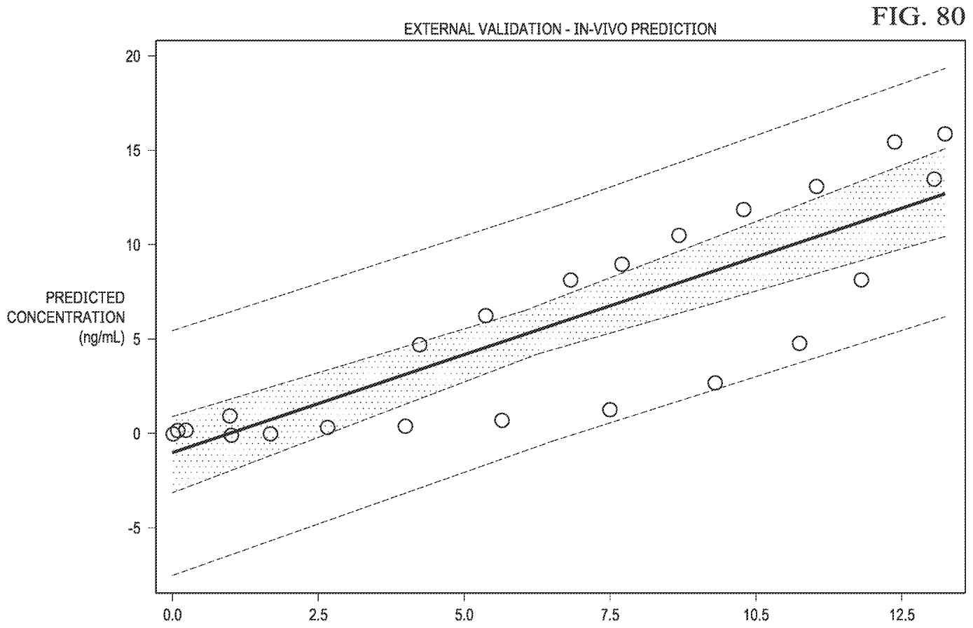

The in-vitro dissolution data and the convolution model were used to predict the in-vivo PK profiles of the formulation used in the external validation. FIG. 80 presents the plot of the predicted versus the observed concentrations with the regression line.

The results of the regression analysis are presented in Table 78. The analysis indicated that the intercept was not statistically different from zero and that the slope was not statistically different from one as the 95% confidence limits included 0 for the intercept and 1 for the slope.

TABLE 78
External validation: results of the regression analysis
of the predicted versus the observed concentrations:
Parameter Estimates

| | | Parameter | Standard | | | 95% Confidence | |
|---|---|---|---|---|---|---|---|
| Variable | DF | Estimate | Error | t Value | Pr > \|t\| | Limits | |
| Intercept | 1 | −1.03563 | 1.02561 | −1.01 | 0.3236 | −3.1626 | 1.09136 |
| Slope | 1 | 1.04295 | 0.13431 | 7.77 | <.0001 | 0.76441 | 1.3215 |

The external validation was assessed by comparing the area under the plasma-concentration time curves (AUC) and the Cmax values of the observed and the model predicted data.

The estimated % PE values are presented in Table 79.

TABLE 79
External validation: comparison of the Cmax and AUC values estimated and
model predicted with the assessment of the prediction error (% PE):

| | Observed Values | | Predicted Values | | | |
|---|---|---|---|---|---|---|
| | Cmax | AUC | Cmax | AUC | Prediction Error | |
| Formulation | (ng/mL | (ng*hr/mL) | (ng/mL | (ng*hr/mL) | % PE_AUC | % PE_Cmax |
| External | 12.946 | 169.287 | 15.992 | 168.338 | 0.56 | 23.53 |

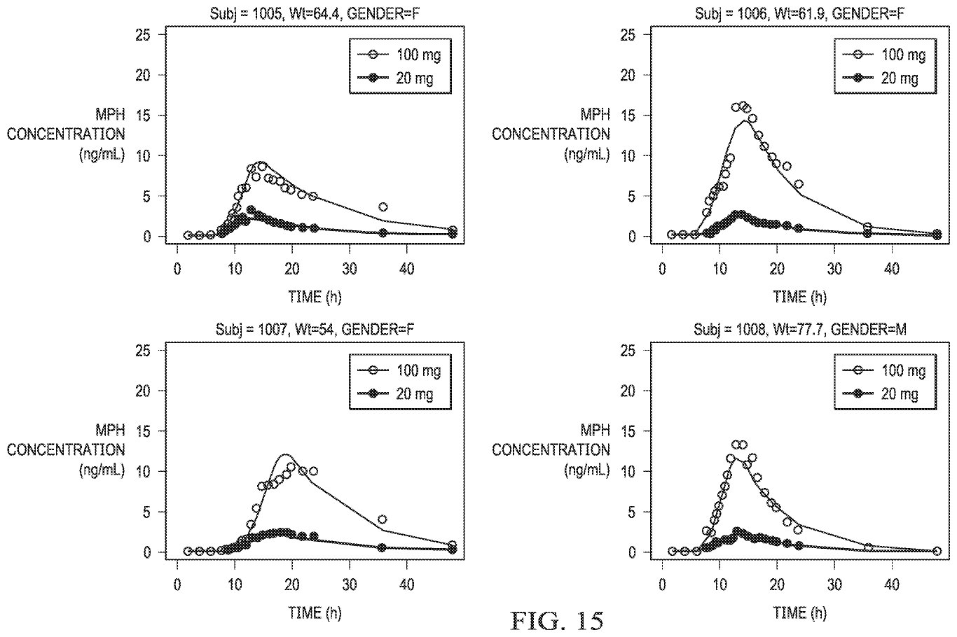

The predicted AUC value was characterized by a % PE<10% while the % PE for the Cmax was ˜24%. This value was consistent with comparison of the mean PK concentrations time-course for the formulation with median dissolution rate in the study HLD200-111 and the PK concentrations time-course in the study HLD200-109 (Fasted arm) as shown in FIG. 15. However, this value exceeded the maximum threshold of 10% established by the FDA to meet the criteria for external predictability.

In summary, the IVIVC analysis described in Example 48 was conducted using the convolution-based modelling approach.

The convolution-based method for assessing IVIVC has been shown to present a number of benefit with respect to the conventional assessment of IVIVC using convolution and deconvolution methodologies (Buchwald P (2003) Direct differential-equation-based in vitro-in vivo correlation (IVIVC) method. J Pharm Pharmacol 55:495-504; Gaynor C, Dunne A, Costello C, Davis J. A population approach to in vitro-in vivo correlation modelling for compounds with nonlinear kinetics. J Pharmacokinet Pharmacodyn. 2011 June; 38(3):317-32).

For example, using the convolution-based method, the overall IVIVC assessment can be implemented using a set of differential equations that are directly integrated without the need of applying convolution or deconvolution procedures.

In this framework, various functional dependencies (e.g., time scaling) can be easily introduced to describe or to connect dissolution and absorption profiles. This can provide improved performance and increased modeling flexibility as no specific software tools are required other than standard software for modeling (such as NONMEM).

Furthermore, the traditional methods based on the deconvolution and convolution approach require the assumption of linearity of the system being studied and are, therefore, unsuitable for use with compounds exhibiting nonlinear kinetics. At variance of this limitation, the convolution-based approach can easily accommodate of potential non-linearity in the pharmacokinetic processes by integrating the description of the potential non-linearity in the differential equation used to define the IVIVC model. (Gaynor et al, 2011).

The IVIVC model was developed using the in-vitro dissolution and the in-vivo PK of three extended-release formulations (Fast, Med, and Slow). The disposition of MPH was characterized using data of an IR formulation.

The in-vitro dissolution data were able to predict the in-vivo PK time course with high precision for each formulation evaluated.

The comparison of the refined IVIVC model predictions with the observed concentrations indicated the presence of strong correlation between the two measurements: the regression line was characterized by a zero intercept and unitary slope.

The average prediction errors of AUC and Cmax for the Slow, Med, and Fast release formulations were 5.93% and 4.34% for AUC and Cmax, respectively below the FDA suggested 10%. Furthermore, the prediction errors for each of the formulations were all below the allowed 15%. These results support the Level A IVIVC correlation.

The external predictability was evaluated using the refined IVIVC model to predict the in vivo performance of a formulation that was not used in developing the IVIVC model.

The predicted AUC value was characterized by a % PE<10% while the % PE for the Cmax was ˜24%. This value was consistent with comparison of the mean PK concentrations time-course for the formulation with median dissolution rate in the study HLD200-111 and the PK concentrations time-course in the study HLD200-109 (Fasted arm) as shown in FIG. 15. However, this value exceeded the maximum threshold of 10% established by the FDA to meet the criteria for external predictability.

Overall, a successful IVIVC model was developed and validated according to the internal predictability criteria, as recommended by the FDA in its Guidance to the Industry Extended Release Oral Dosage Forms: Development, Evaluation, and Application of In-Vitro/In-Vivo Correlations.

### Example 49. Polynomial Curve Fitting for Slow, Medium, and Fast Methylphenidate Formulations

The individual pharmacokinetic data for the immediate-release methylphenidate formulation were utilized to characterize the absorption, disposition and elimination of methylphenidate.

The Weibull model described above was fitted to the mean in vitro dissolution data of the Slow, Med, and Fast dissolution methylphenidate formulations.

The in vivo drug release rate was estimated together with an estimate of the inter-individual variability of the elimination rate constant for the three formulations. A convolution model was fit to the data of the three formulations using a nonlinear mixed-effect approach.

The in vivo drug release was estimated fixing the mean values of the volume (V) and the mean value of the elimination rate (kel) to the values estimated in the analysis of the immediate-release formulation data.

An analysis was conducted to evaluate the correlation between the in vitro release and the in vivo release resulting from the IVIVC analysis with the inclusion of time scaling correction, as described in Example 48.

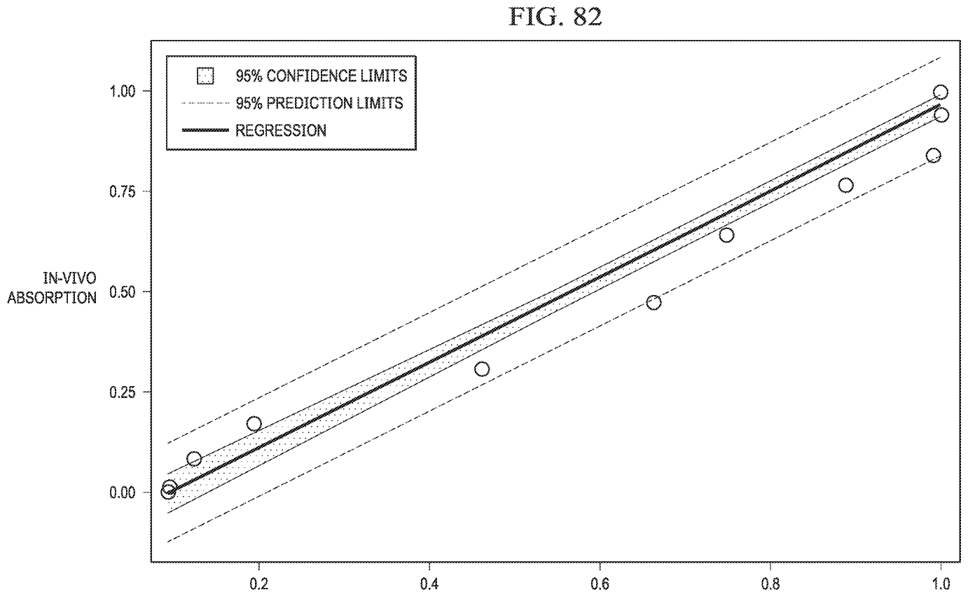

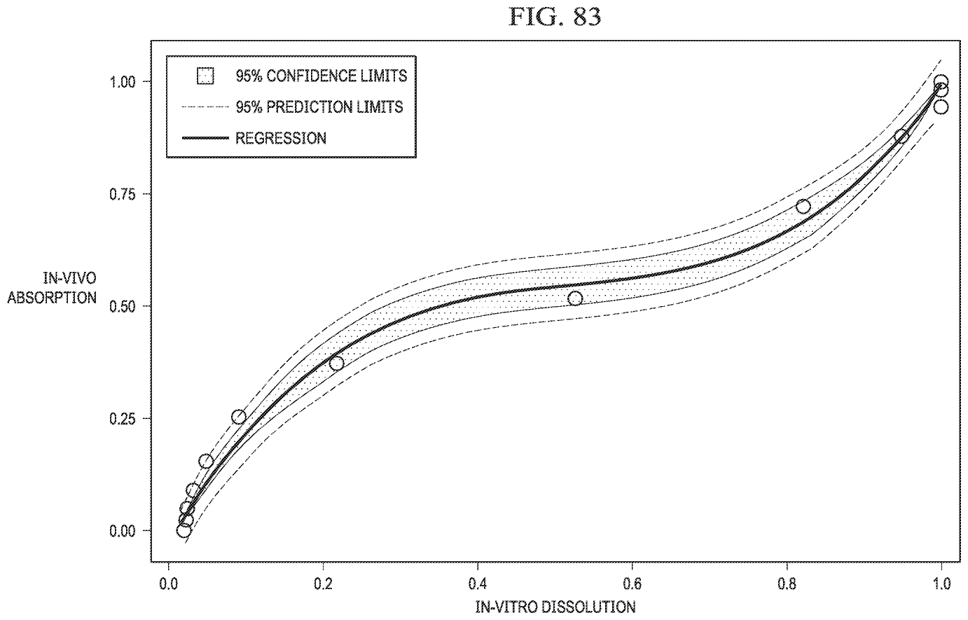

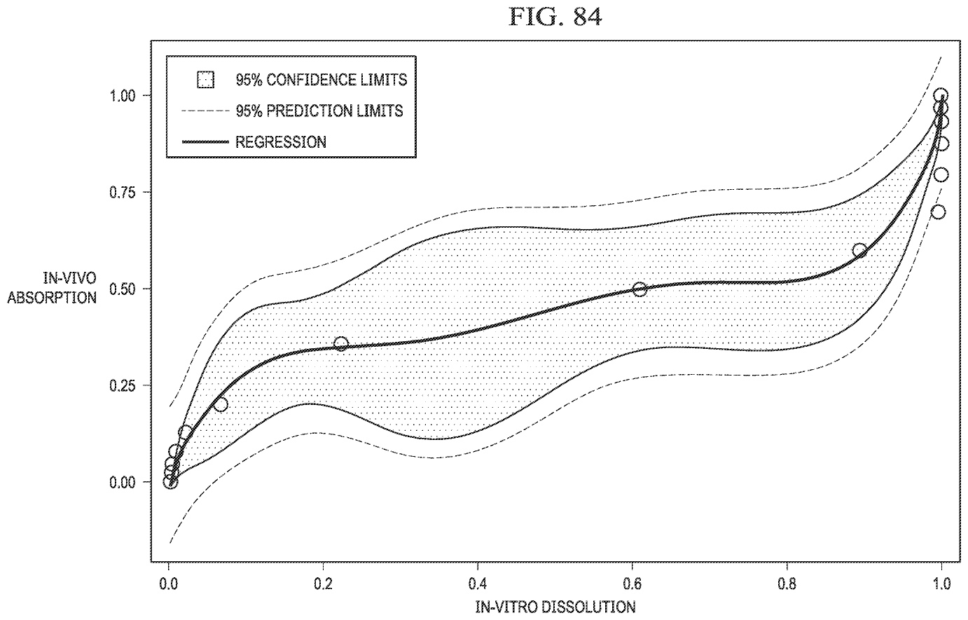

Graphs reporting the regression analysis for the fast, medium, and slow release MPH formulations are presented in FIG. 82, FIG. 83 and FIG. 84 and the results of the respective regression analysis are presented in Table 80, Table 81, and Table 82. The analysis indicated that a statistically significant correlation was found between the in-vitro dissolution and the in-vivo absorption data (p<0.001).

TABLE 80
Results of regression analysis of IVIVC for
the methylphenidate HLD200 fast formulation.
Number of observations read 23
Number of observations used 23
Analysis of Variance

| | | Sum of | Mean | | |
|---|---|---|---|---|---|
| Source | DF | Squares | Square | F value | Pr > F |
| Model | 1 | 3.42012 | 3.42012 | 1106.93 | <0.0001 |
| Error | 21 | 0.06488 | 0.00309 | | |
| Corrected Total | 22 | 3.48501 | | | |

| | | | |
|---|---|---|---|
| Root MSE | 0.05559 | R-Square | 0.9814 |
| Dependent Mean | 0.70672 | Adj. R-Square | 0.9805 |
| Coeff Var | 7.86524 | | |

Parameter Estimates

| | | Parameter | Std. | | | 95% confidence | |
|---|---|---|---|---|---|---|---|
| Variable | DF | Estimate | Error | t Value | Pr > \|t\| | limits | |
| Intercept | 1 | −0.10363 | 0.02697 | −3.84 | 0.0009 | −0.15972 | −0.04753 |
| x | 1 | 1.07412 | 0.03228 | 33.27 | <0.0001 | 1.00698 | 1.14126 |

Table 80 shows that the fast formulation shows a statistically significant correlation between in-vitro and in-vivo absorption (p<0.0001) using a linear model.

TABLE 81
Results of regression analysis of IVIVC for
the methylphenidate HLD200 medium formulation.
Number of observations read 23
Number of observations used 23
Analysis of Variance

| | | Sum of | Mean | | |
|---|---|---|---|---|---|
| Source | DF | Squares | Square | F value | Pr > F |
| Model | 3 | 4.13253 | 1.37751 | 1878.34 | <0.0001 |
| Error | 19 | 0.01393 | 0.00073336 | | |
| Corrected Total | 22 | 4.14646 | | | |

| | | | |
|---|---|---|---|
| Root MSE | 0.02708 | R-Square | 0.9966 |
| Dependent Mean | 0.60846 | Adj. R-Square | 0.9961 |
| Coeff Var | 4.45072 | | |

Parameter Estimates

| | | Parameter | Std. | | | 95% confidence | |
|---|---|---|---|---|---|---|---|
| Variable | DF | Estimate | Error | t Value | Pr > \|t\| | limits | |
| Intercept | 1 | −0.02099 | 0.01329 | −1.58 | 0.1308 | −0.04880 | 0.00683 |
| x | 1 | 2.88657 | 0.24359 | 11.85 | <0.0001 | 2.37673 | 3.39642 |
| x² | 1 | −5.15522 | 0.61929 | −8.32 | <0.0001 | −6.45140 | −3.85904 |
| x³ | 1 | 3.28417 | 0.39776 | 8.26 | <0.0001 | 2.45166 | 4.11669 |

Table 80 shows that the medium formulation shows a statistically significant correlation between in-vitro and in-vivo absorption (p<0.0001) using a 3rd degree polynomial model.

TABLE 82
Results of regression analysis of IVIVC for
the methylphenidate HLD200 slow formulation.
Number of observations read 22
Number of observations used 22
Analysis of Variance

| | | Sum of | Mean | | |
|---|---|---|---|---|---|
| Source | DF | Squares | Square | F value | Pr > F |
| Model | 5 | 3.67728 | 0.73546 | 124.90 | <0.0001 |
| Error | 16 | 0.09422 | 0.00589 | | |
| Corrected Total | 21 | 3.77150 | | | |

| | | | |
|---|---|---|---|
| Root MSE | 0.07674 | R-Square | 0.9750 |
| Dependent Mean | 0.50914 | Adj. R-Square | 0.9672 |
| Coeff Var | 15.07184 | | |

Parameter Estimates

| | | Parameter | Std. | | | 95% confidence | |
|---|---|---|---|---|---|---|---|
| Variable | DF | Estimate | Error | t Value | Pr > \|t\| | limits | |
| Intercept | 1 | 0.00756 | 0.03261 | 0.23 | 0.8197 | −0.06156 | 0.07668 |
| x | 1 | 4.59396 | 2.31465 | 1.98 | 0.0646 | −0.31288 | 9.50080 |
| x² | 1 | −24.23614 | 19.50712 | −1.24 | 0.2320 | −65.58938 | 17.11711 |
| x³ | 1 | 60.62207 | 52.96971 | 1.14 | 0.2693 | −51.66869 | 172.91283 |
| x⁴ | 1 | −67.92046 | 57.68659 | −1.18 | 0.2563 | −190.21057 | 54.36965 |
| x⁵ | 1 | 27.86252 | 21.99528 | 1.27 | 0.2234 | −18.76539 | 74.49043 |

Table 82 shows that the slow formulation shows a statistically significant correlation between in-vitro and in-vivo absorption (p<0.0001) using a 5th order polynomial model.

The methylphenidate HLD200 fast formulation has the same components and release mechanism as the methylphenidate HLD200 medium and the slow formulations, varying only in the % weight gain of the ER layer. However, the methylphenidate HLD200 fast formulation releases the methylphenidate earlier and thus more proximal in the upper GI tract, in comparison with the methylphenidate HLD200 medium and slow formulations that release the methylphenidate later and thus more distally, in the colon. Thus, the methylphenidate fast formulation shows IVIVC similar to Concerta® and other products. The methylphenidate HLD200 medium and slow formulations exhibit non-linear (higher order polynomial) IVIVC which can be attributed to the site of release and absorption. Furthermore, the further into the colon the release is initiated the farther the correlation deviates from linearity, with the methylphenidate HLD200 slow formulation showing an IVIVC fitting a higher order polynomial than the methylphenidate HLD200 medium formulation.

### Example 50: An In-Vitro/In-Vivo Correlation (IVIVC) Model for HLD200, an Evening-Dosed Delayed-Release and Extended-Release Methylphenidate

This Example includes information relevant to sigmoid Emax modeling which may be used as an alternative to Weibull function modeling, but results in the same clinical indications. Accordingly, the sigmoid Emax modeling discussed in this Example and the data presented regarding this modeling as well as any conclusions regarding that data may be combined with all other aspects of this disclosure, either in combination with or as an alternative to Weibull function modeling or information and conclusions resulting therefrom, unless clearly excluded or inoperative.

HLD200 is the first evening-dosed, delayed-release and extended-release formulation of methylphenidate (DR/ER-MPH) specifically designed to delay the initial release of MPH and provide an onset of clinically meaningful treatment effect upon awakening and lasting into the evening.

In some embodiments, the pharmacokinetic (PK) profile of DR/ER-MPH is characterized by an 8- to 10-hour delay in initial MPH release, followed by a period of extended, controlled release with absorption peaking ˜14 hours after dosing, and ≥50% of drug exposure occurring after peak plasma concentration is reached (Liu T, et al. J Child Adolesc Psychopharmacol. 2019; 29(3):181-191, incorporated by reference herein). In two pivotal phase 3 trials in children (6-12 years) with attention-deficit/hyperactivity disorder (ADHD), DR/ER-MPH demonstrated significant improvements versus placebo in ADHD symptom control and functional impairment during the early morning, throughout the day, and into the late afternoon/evening (Childress A C, et al. J Child Adolesc Psychopharmacol. 2019 Aug. 29. doi: 10.1089/cap.2019.0070. Epub ahead of print; and Pliszka S R, et al. J Child Adolesc Psychopharmacol. 2017; 27(6):474-482, both incorporated by reference herein). The US Food and Drug Administration (FDA) encourages the development of an in vitro/in vivo correlation (IVIVC), a prediction of the time course of in vivo plasma concentrations from an in vitro dissolution profile, for extended-release drug products (U.S. Department of Health and Human Services, Food and Drug Administration Center for Drug Evaluation and Research (CDER). Guidance for Industry. Extended Release Oral Dosage Forms: Development, Evaluation, and Application of In Vitro/In Vivo Correlations. September 1997, incorporated by reference herein). An IVIVC can be useful in establishing dissolution specifications and can permit some formulation and manufacturing changes without an in vivo bioequivalence study. Level A correlations, the most informative type of IVIVC model, describe the relationship between the entire in vitro dissolution time course and the time course of plasma drug concentration (a point-to-point relationship).

The purpose of this study was to establish and validate a Level A IVIVC for DR/ER-MPH.

The following methods were used in this Example.

Data Sources. In vitro MPH dissolution rates were measured in three formulations of DR/ER-MPH (Slow, Medium, and Fast) in a 3-stage environment (under conditions that mimic oral administration) using a United States Pharmacopeia (USP) apparatus type 1. In vivo plasma MPH concentrations were measured in a phase 1, single-center, single-dose, open-label, randomized, 3-way crossover, comparative bioavailability study of the three DR/ER-MPH formulations in 18 healthy adults (Ironshore Pharmaceuticals & Development, Inc. Data on File: HLD200-111, incorporated by reference herein). 100 mg of DR/ER-MPH was administered at 9:00 pm, approximately three hours after a standard low-fat meal. Blood samples were collected up to 48 hours post-dosing. A washout period of 96 hours took place between each dose. PK data from an immediate-release MPH (IR MPH) were used to characterize the disposition and elimination of MPH. Plasma levels were measured in a phase 1, single-center, single-dose, open-label, randomized, crossover study of 12 healthy adults, which has been described previously (Liu T, et al. J Child Adolesc Psychopharmacol. 2019; 29(3):181-191, incorporated by reference herein).

IVIVC Model Development.

A Level A IVIVC, a point-to-point correlation between the fraction of MPH dissolved in vitro [rᵥᵢₜᵣₒ(t)] (Eq. 9) and the fraction of MPH absorbed in vivo [rᵥᵢᵥₒ(t)] (Eq. 14), was developed and evaluated. The IVIVC model was developed and assessed by a convolution-based modeling approach (Gomeni R, et al. Pharmacometrics Syst Pharmacol. 2019; 8(2):97-106, incorporated by reference herein) using the following steps:

Step 1. Fit the PK time course of the IR MPH formulation: To characterize the absorption, disposition, and elimination of IR MPH, PK was best described by a one-compartment model with first-order absorption with a lag time. The model parameters were ka (first order absorption rate constant), kel (elimination rate constant), lag (lag time), and V (volume of distribution, and a combination of additive and proportional residual error).

Step 2. Individually fit the mean in vitro dissolution data: The in vitro dissolution data for the Slow, Medium, and Fast formulations of DR/ER-MPH were described by a sigmoid Emax model:

$${r}_{v i t r o} ( t ) = \frac{t i m {e}^{g a}}{{EC}^{ga} + tim {e}^{g a}} \qquad ( Eq . 9 )$$

where EC is the time to release 50% of the dose and ga is a parameter characterizing the shape of the absorption curve.

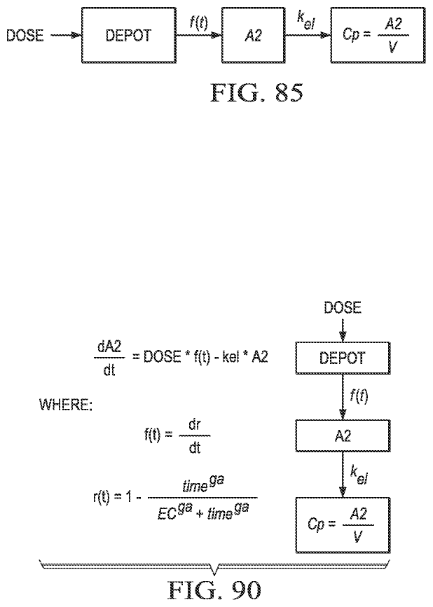

Step 3. Fit the in vivo PK data: To estimate the in vivo release rate and the inter-individual variability of the elimination rate constant, a convolution model was used to jointly fit the data of the three formulations using a nonlinear mixed-effect approach. In vivo PK data were fitted by fixing the mean values of the volume (V) and the mean value of the elimination rate (kel) to the values estimated in the analysis of the IR MPH data. The PK time course was assumed to be characterized by a sigmoid Emax model. DR/ER-MPH plasma concentration (Cp), resulting from an arbitrary dose, was described by convolution as (see FIG. 85):

$$\begin{matrix}\frac{d A 2}{d t} = B {I}_{i} * Dose * f ( t ) - {kel}_{i} * A 2 & ( Eq . 10 ) \\ B {I}_{i} = - 0.0019 * E {C}_{i} * * 2 + 0.0019 * E {C}_{i} + 1.1351 \\ where \text{:} & ( Eq . 11 ) \\ f ( t ) = \frac{d r}{d t} \\ and & ( Eq . 12 ) \\ r ( t ) = 1 - \frac{t i m {e}^{g {a}_{i}}}{E {C}_{i}^{{ga}_{i}} + t i m {e}^{g {a}_{i}}} & ( Eq . 13 )\end{matrix}$$

where BI is the in vivo relative bioavailability, i is the index for the i-th formulation, f(t) is the in vivo input function, V is the volume of distribution, kel is the first-order elimination rate estimated using the IR MPH data, * is the convolution operator, r(t) is the time-varying fraction of the dose released, A is the amount of drug, EC is the time to release 50% of the dose, and ga is a parameter characterizing the shape of the absorption curve.

FIG. 85 is an exemplary schematic depicting a refined IVIVC model including the dependency between dissolution properties and the Estimated in-vivo relative bioavailability.

Step 4. Evaluate the IVIVC using the convolution modeling approach by:

a. Fixing the in vivo drug release parameters for each DR/ER-MPH formulation to the values estimated in the previous analyses: (i) Mean kel and V, estimated from the analysis of the IR MPH data (Step 1); (ii) Estimated in vitro dissolution data (EC and ga) for the three formulations (Step 2); and (iii) Inter-individual variability of kel and V, estimated from the analysis of the in vivo PK data (Step 3).

b. Estimating the time-scaling-factors common to all formulations (fixed effects): (i) A time-scaling function was included in the model to account for potential time differences in the in vitro and the in vivo processes (eg, when the dissolution time is faster than the in vivo input rate (Gomeni R, et al. Pharmacometrics Syst Pharmacol. 2019; 8(2):97-106, incorporated by reference herein):

rᵥᵢᵥₒ(t)=a₁+a₂·rᵥᵢₜᵣₒ(tt) (Eq. 14)

tt=b₁+b₂·t^{b}^{₃} (Eq. 15)

The equations include a linear component (intercept of a₁ and slope of a2) and a nonlinear component describing the time-shifting (b₁), time-scaling (b₂), and time-shaping (b₃) factors. In case of absence of time scaling between rᵥᵢₜᵣₒ and rᵥᵢᵥₒ: a₁=0, a₂₌₁, b₁=0, b₂=1, and b₃=1; otherwise, time scaling was defined by estimating the appropriate values of the parameters.

c. Estimating the fraction of the dose administered available for the systemic circulation (Fslow, Fmed, and Ffast), and the additive residual error: The F parameters represent a dose scaling value with respect to the reference IR MPH formulation; this was done to allow for any difference which might exist in the bioavailability between the IR MPH and the DR/ER-MPH formulations.

Step 5. Internal validation: The in vitro dissolution data and the convolution model were used to predict the in vivo PK profiles of the three formulations. Peak plasma concentration (Cmax) and area under the plasma-concentration time curve between zero and infinity (AUC) were predicted using the model for each formulation and compared to the mean observed values. AUC values were estimated using the log-linear trapezoidal rule with extrapolation to infinity using the last measurable concentration and the elimination rate constant for the observed and model-predicted data. The prediction error (% PE) was calculated for each PK parameter using the following equation, where n is the number of formulations (Table 84):

$$\% PE = \frac{1}{n} {\sum }_{1}^{n} \frac{Observed value - Predicted value}{Observed value} \cdot 100 \qquad ( Eq . 16 )$$

The criteria for assessing the level of predictability for each PK parameter was an average % PE≤10% with no individual values≥15% (U.S. Department of Health and Human Services, Food and Drug Administration Center for Drug Evaluation and Research (CDER). Guidance for Industry. Extended Release Oral Dosage Forms: Development, Evaluation, and Application of In Vitro/In Vivo Correlations. September 1997, incorporated by reference herein).

Step 6. External validation: External validation of the IVIVC model was performed using PK data from a phase 1, single-center, open-label study of 18 healthy adult volunteers who received 100-mg DR/ER-MPH, which has been described previously (Liu T, et al. J Child Adolesc Psychopharmacol. 2019; 29(3):181-191, incorporated by reference herein). In vitro dissolution data of the formulation used in the study were described by the same sigmoid Emax model previously applied. The expected typical profile of the in vivo PK was predicted using the IVIVC jointly with the dissolution release parameters from the study Eq. 16 was used to calculate % PE from the observed and predicted Cmax and AUC values.

Software. All analyses were conducted in NONMEM® (Regents of the University of California Corporation, California) using the first-order conditional estimation with interaction (FOCE-I) method. The population PK analysis of the IR MPH data was conducted using the ADVAN14 subroutine. The convolution model was implemented using the ADVAN6 subroutine.

The following Results were obtained in this Example.

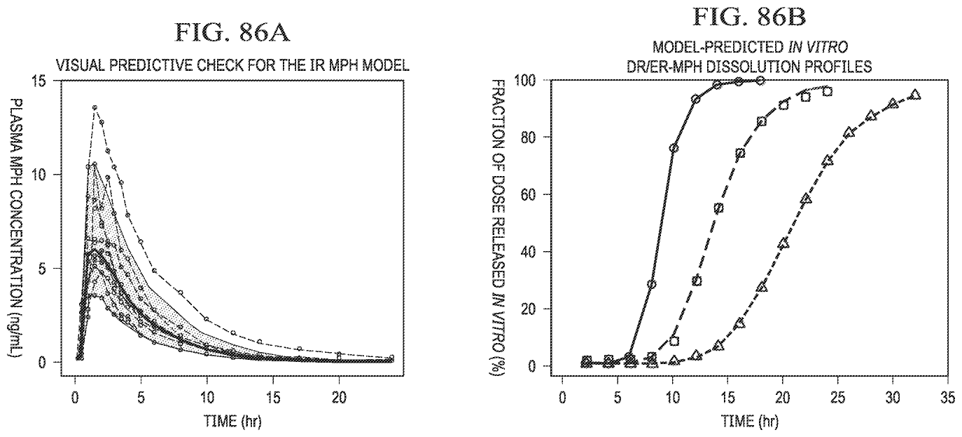

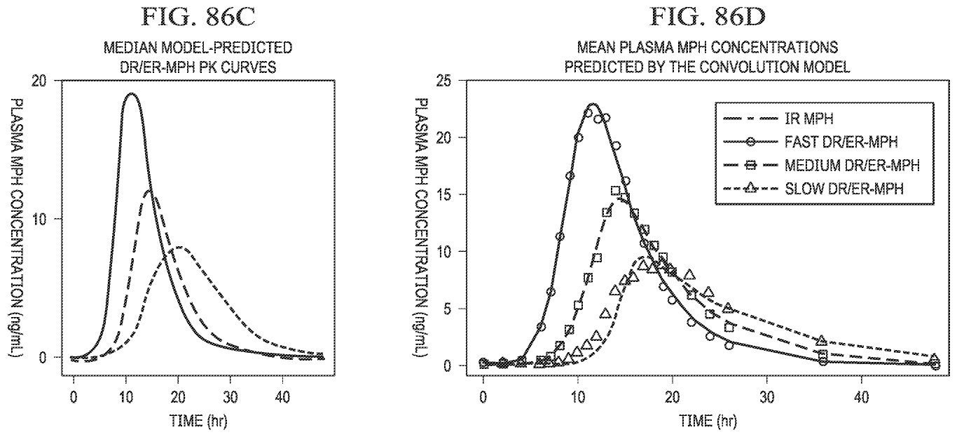

FIG. 86A is a graph reporting exemplary values for plasma MPH concentration versus time (hours) allowing visual predictive check for the immediate-release methylphenidate (IR MPH) model (step 1). Heavy solid line depicts median curve; shaded region depicts 90% prediction interval; circle dots depict individual participant data.

Step 1 provided values of MPH elimination (kₑᵢ, elimination rate) and disposition (V, volume of distribution).

FIG. 86B is a graph reporting exemplary values for fraction of dose released in vitro (%) versus time (hours) for model-predicted in vitro delayed-release and extended-release methylphenidate (DR/ER-MPH) Dissolution Profiles (step 2). Dots depict mean dissolution values; solid lines depict model-predicted profiles.

Step 2 provided values of in vitro dissolution, including EC (time to release 50% of the dose) and ga (parameter characterizing the shape of the curve).

FIG. 86C is a graph reporting exemplary values for Median Model-Predicted DR/ER-MPH PK Curves (step 3).

Step 3 provided values of interindividual variability of kₑᵢ and V.

FIG. 86D is a graph reporting exemplary values for Mean Plasma MPH Concentrations Predicted by the Convolution Model (step 4). Dots represent the mean observed PK concentrations and the solid lines represent the model-predicted PK profiles.

IVIVC Model Evaluation and Validation

In the IVIVC model, all three DR/ER-MPH formulations demonstrated a monophasic plasma-time concentration profile, with the Fast formulation exhibiting a shorter delay before initial MPH release, higher Cmax, faster time to peak absorption (Tmax), and higher relative bioavailability compared to the Medium formulation (FIG. 86D). The same trends were seen between the Medium and Slow formulations.

Relative bioavailability of the Fast, Medium, and Slow DR/ER-MPH formulations with respect to the IR MPH formulation was 100%, 80%, and 32%, respectively.

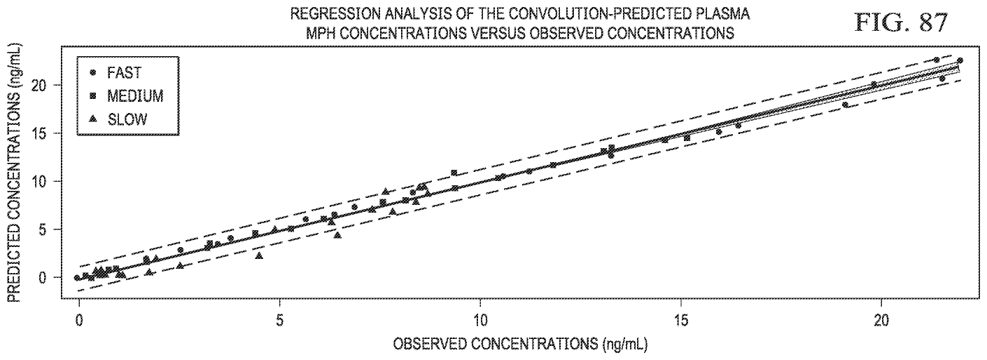

A regression analysis demonstrated a statistically significant correlation between the observed plasma MPH concentration versus convolution-predicted concentrations (predicted from in vitro dissolution data and the convolution model) with the inclusion of time scaling correction (P<0.001) (FIG. 87). In FIG. 87 Heavy solid line, regression analysis; dots, individual participant data; shaded region, 95% confidence interval; dashed lines, 95% prediction interval.

Parameter estimates associated with FIG. 87 were as shown in Table 83.

TABLE 83
Parameter Estimates

| | | Parameter | Standard | | | 95% Confidence | |
|---|---|---|---|---|---|---|---|
| Variable | DF | Estimate | Error | t Value | Pr > \|t\| | Limits | |
| Intercept | 1 | −0.07963 | 0.11146 | −0.71 | 0.4775 | −0.302 | 0.143 |
| Slope | 1 | 1.00081 | 0.01257 | 79.62 | <.0001 | 0.976 | 1.026 |

In Table 83, “DF” is degrees of freedom, “MPH” is methylphenidate, and “Pr” is probability.

The established IVIVC model was able to predict in vivo PK from in vitro dissolution data with high precision for each formulation evaluated; average % PEs for the Slow, Medium, and Fast formulations were 6.07% for Cmax and 1.64% for AUC (Table 84).

TABLE 84
Internal Validation: Prediction Error Assessments

| | Observed Values ^{a} | | Predicted Values | | | |
|---|---|---|---|---|---|---|
| | Cmax | AUC | Cmax | AUC | Prediction Error | |
| Formulation | (ng/mL) | (ng · hr/mL) | (ng/mL) | (ng · hr/mL) | % PE_Cmax | % PE_AUC |
| Fast | 25.23 | 231.00 | 22.54 | 232.98 | 10.65 | 0.86 |
| Medium | 15.64 | 177.00 | 14.48 | 180.97 | 7.42 | 2.24 |
| Slow | 9.54 | 151.00 | 9.53 | 153.73 | 0.15 | 1.81 |
| Average | | | | | 6.07 | 1.64 |

In Table 84, “a” indicates data source of observed values, LD200-111 Clinical Study Report (Ironshore Pharmaceuticals & Development, Inc. Data on File: HLD200-111), “AUC” refers to area under the plasma-concentration time curve between zero and infinity; “Cmax” refers to peak plasma concentration; “PE” refers to prediction error.

The average % PE for both AUC and Cmax was less than 10%, and the % PE for each formulation was less than 15% (see Table 84), meeting FDA criteria for a successful IVIVC (U.S. Department of Health and Human Services, Food and Drug Administration Center for Drug Evaluation and Research (CDER). Guidance for Industry. Extended Release Oral Dosage Forms: Development, Evaluation, and Application of In Vitro/In Vivo Correlations. September 1997, incorporated by reference herein).

Estimated % PEs for the external validation data were 0.79% for Cmax and 9.85% for AUC, which established the external predictability of the IVIVC ((U.S. Department of Health and Human Services, Food and Drug Administration Center for Drug Evaluation and Research (CDER). Guidance for Industry. Extended Release Oral Dosage Forms: Development, Evaluation, and Application of In Vitro/In Vivo Correlations. September 1997, incorporated by reference herein) (see Table 85).

TABLE 85
External Validation: Prediction Error Assessments:

| | Observed Values ^{a} | | Predicted Values | | | |
|---|---|---|---|---|---|---|
| | Cmax | AUC | Cmax | AUC | Prediction Error | |
| Formulation | (ng/mL) | (ng · hr/mL) | (ng/mL) | (ng · hr/mL) | % PE_Cmax | % PE_AUC |
| External | 14.17 | 183.00 | 14.06 | 164.98 | 0.79 | 9.85 |

In Table 85 “a” refers to Source of the observed values (Liu T, et al. J Child Adolesc Psychopharmacol. 2019; 29(3):181-191, incorporated by reference herein), “AUC” refers to area under the plasma-concentration time curve between zero and infinity; “Cmax” refers to peak plasma concentration; “PE” refers to prediction error.

In this Example, a convolution-based Level A IVIVC model for DR/ER-MPH was developed and validated. The in vitro dissolution data were able to predict the in vivo PK time course with a high degree of precision. Average prediction errors of AUC and Cₘₐₓ for the Slow, Medium, and Fast formulations of DR/ER-MPH were below 10% and below 15% for each formulation, meeting FDA criteria for a successful Level A IVIVC correlation. The IVIVC also met FDA criteria for external predictability.

### Example 51. Evaluation of the IVIVC Using the Convolution Modelling Approach

This Example further describes evaluation of the IVIVC using the convolution modelling approach (step 4 described in Example 50).

The convolution model used for the assessment of the IVIVC relationship is presented in FIG. 90. The convolution model presented in FIG. 90 was used to independently fit the data of the three formulations using a non-nonlinear mixed-effect approach. The in-vivo drug release was estimated fixing the mean values of the volume (V) and the mean value of the elimination rate (kel) to the values estimated in the analysis of the IR data. The PK time course of in-vivo release of the different formulations was assumed to be characterized by sigmoidal Emax model.

The mean data of the three formulations were jointly fitted. The following parameters were fixed to the values estimated in the previous analyses: The mean kel, and V (estimated from the analysis of the IR data in Step 1), and the variances (inter-individual variability) of kel and V (estimated in Step 3), the dissolution data (EC and GA for the 3 formulations) estimated in Step 2. The convolution model was implemented in NONMEM using the ADVAN6 subroutine and the FOCE-I method.

The mean data of the three formulations (Slow, Med, and Fast) were jointly fitted to estimate the following parameters: the time scaling parameters (a1, a2, b1, b2, and b3), the fraction of the dose administered available for the systemic circulation (F_Slow, F_Med, and F_Fast), and the additive residual error (err). The f(t) function was estimated by using the finite difference approach. The F_parameters represent a dose scaling value with respect to the reference IR formulation. The rational to use this parameter is to allow for any difference which might exist in the bioavailability between the reference formulation and the Slow, Med, and Fast release formulations.

The time-scaling parameters (a1, a2, b1, b2, and b3) were estimated as fixed effect parameters (without random effect) as these parameters were expected to take the same value for all formulations. No random effect was associated to any parameter of the convolution model.

The estimated parameter values are presented in Table 86.

TABLE 86
Estimated parameter values in the convolution analysis
for the IVIVC assessment. SE is the standard error and
RSE is the relative standard error of the parameters:

| | Parameter | Value | SE | RSE |
|---|---|---|---|---|
| Fixed | A1 | 0# | | |
| Effect | A2 | 1# | | |
| | B1 | 11.7 | 0.481 | 4.10% |
| | B2 | 0.252 | 0.0501 | 19.90% |
| | B3 | 4.8 | 0.108 | 2.30% |
| | F_CR1 | 1# | | |
| | F CR2 | 0.798 | 0.0212 | 2.70% |
| | F CR3 | 0.322 | 0.0136 | 4.20% |
| | Kel(hr−1) | 0.27* | | |
| | V(L) | 2.48* | | |
| | add-err | 0.777 | 0.238 | 30.60% |
| Random | Kel(hr−1) | 0.354** | | |
| Effect | V(L) | 0.334** | | |

*fixed from the analysis of the IR data
**fixed from the reassessment of the IIV
#estimated as fixed parameter

The mean observed and model predicted MPH in-vivo concentrations for the Slow, Med, and Fast release rate formulations are presented in FIG. 86D.

The results of the analysis indicated that the relative bioavailability of the Fast, Med, and Slow release formulation with respect to the IR formulation where 100%, 79.8% and 32.2%, respectively.

An analysis was conducted to evaluate the correlation between the in-vitro release and the in-vivo release resulting from the IVIVC analysis with the inclusion of time scaling correction.

The regression analysis is presented in FIG. 87 and the results of the regression analysis are presented in Table 83. The analysis indicated that a statistically significant correlation was found between the in-vitro dissolution and the in-vivo absorption data (p<0.001).

### Example 52. Comparison of Models Considered for Describing the In Vitro Dissolution Data of Each Formulation

Three different models have been considered for describing the in vitro dissolution data of each formulation. Table 87 shows the function equations associated with the three models.

TABLE 87

| Model | Function equation |
|---|---|
| Single Weibull function | ${r}_{vitro} ( t ) = [ 1 - {e}^{- ( {( \frac{time}{td} )}^{ss} )} ]$ |
| Double Weibull function | ${r}_{vitro} ( t ) = [ 1 - ( ff \cdot {e}^{- ( {( \frac{time}{td} )}^{ss} )} + ( 1 - ff ) \cdot {e}^{- ( {( \frac{time}{td 1} )}^{ss 1} )} ) ]$ |
| Sigmoid Emax function | ${r}_{vitro} ( t ) = \frac{{time}^{ga}}{{EC}^{ga} + {time}^{ga}}$ |

The comparison of alternative models was performed using the log-likelihood ratio test for nested models or the Akaike information criterion (AIC) for non-nested models. For nested models, an alternative model was considered as a significantly better descriptor of data when the reduction in the objective function value (OFV) associated with this model was ≥3.84, χ²<0.05 for 1 degree of freedom (df). For non-nested models, the model with the lower AIC value was considered as the preferred one. The AIC criterion was computed as: AIC=2LL+2*nₚ. Where nₚ is the total number of parameters in the model. Among two models, the most informative will be the one with the lowest AIC value.

The comparison of the performances of the single and double Weibull models has been done using the log-likelihood ratio test (Table 88) as the two models were nested models. The comparison of the performances of the double Weibull and the sigmoid Emax models was conducted using the AIC criterion as the two models were not nested models (Table 89).

TABLE 88
Comparison of the single and double Weibull model
performances using the log-likelihood ratio test:

| | Single Weibull | | | Double Weibull | | |
|---|---|---|---|---|---|---|
| | Fast | Med | Slow | Fast | Med | Slow |
| Objective function value | 24.547 | 43.975 | 46.27 | 1.784 | 14.778 | −10.117 |
| Number of parameters | 3 | 3 | 3 | 6 | 6 | 6 |
| Log-Likelihood test (p)* | | | | p < 0.0001 | p < 0.0001 | p < 0.0001 |
| Parameter Values | | | | | | |
| td(hr) | 9.51 | 15.2 | 23.2 | 8.18 | 13.6 | 20.8 |
| ss(unit less) | 6.52 | 4.49 | 4.46 | 36.3 | 6.71 | 5.91 |
| td1(hr) | | | | 10.7 | 19.3 | 28.6 |
| ss1(unit less) | | | | 5.37 | 4.28 | 5.11 |
| ff(%) | | | | 0.518 | 0.668 | 0.677 |
| err0 | 2.37 | 3.79 | 2.58 | 0.67 | 1.12 | 0.442 |

*Comparison of the single vs double Weibull function

TABLE 89
Comparison of the sigmoid Emax and the double Weibull
model performances using the AIC criterion:

| Sigmoid Emax | | | | Double Weibull | | | |
|---|---|---|---|---|---|---|---|
| | Fast | Med | Slow | | Fast | Med | Slow |
| Objective function | 1.324 | 19.746 | −23.456 | | 1.784 | 14.778 | −10.117 |
| Number of | 3 | 3 | 3 | | 6 | 6 | 6 |
| parameters | | | | | | | |
| AIC | 7.324 | 25.746 | −17.456 | | 13.784 | 26.778 | 1.883 |
| Parameter | | | | Parameter | | | |
| EC(hr) | 8.86 | 13.7 | 21 | td(hr) | 8.18 | 13.6 | 20.8 |
| ga(unit less) | 9.2 | 6.64 | 6.62 | ss(unit less) | 36.3 | 6.71 | 5.91 |
| err0 | 0.653 | 1.38 | 0.291 | td1(hr) | 10.7 | 19.3 | 28.6 |
| | | | | ss1(unit less) | 5.37 | 4.28 | 5.11 |
| | | | | ff(%) | 0.518 | 0.668 | 0.677 |
| | | | | err0 | 0.67 | 1.12 | 0.442 |

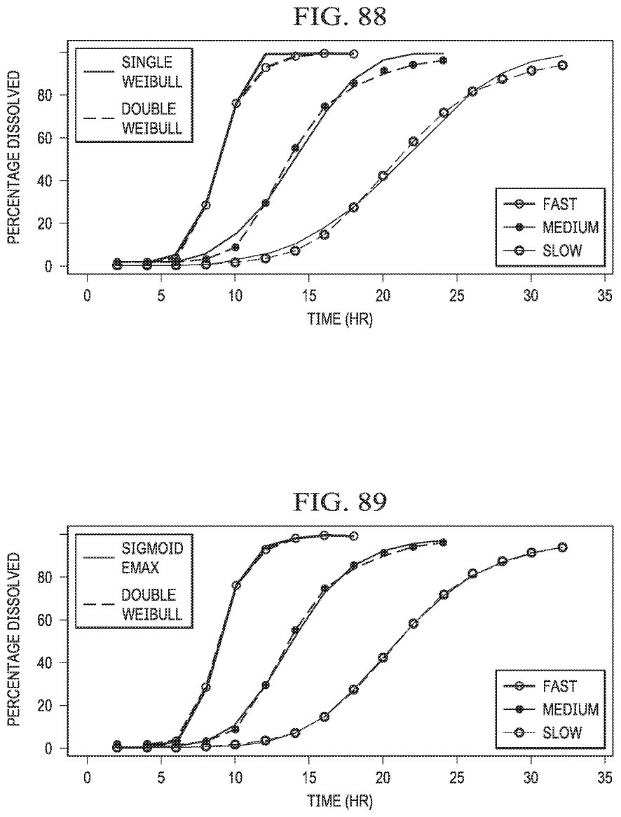

The comparison of the model performances indicated that the double Weibull model performed better than the single Weibull model but the sigmoid Emax model performed better than the double Weibull model. Therefore, the sigmoid Emax model was retained as the preferred model. FIG. 88 shows the observed and model predicted dissolution data resulting from the analysis with single and double Weibull model. FIG. 89 shows the observed and model predicted dissolution data resulting from the analysis with the sigmoid Emax model and the double Weibull model.

### Example 53. Use of Scaling Parameters as Sub-Models to Fit a Single Model to the Slow, Med, and Fast Release Formulations

This Example relates to the use of scaling parameters as sub-models to fit a single model to three formulations (the Slow, Med, and Fast release formulations) that exhibit different PK profiles, such as different bioavailability and/or site of absorption.

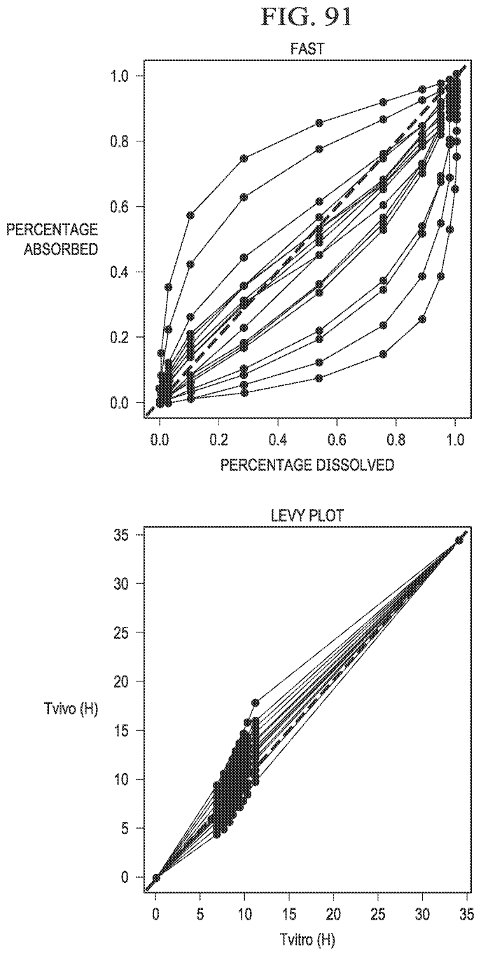

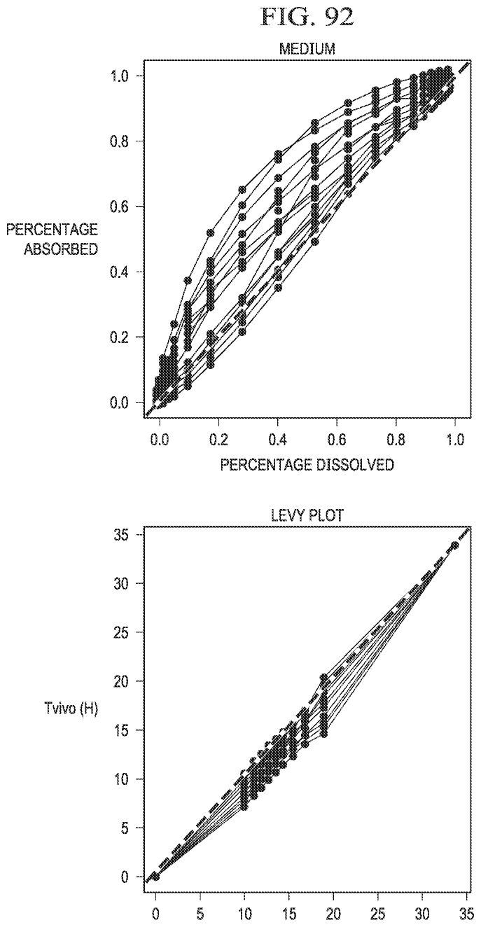

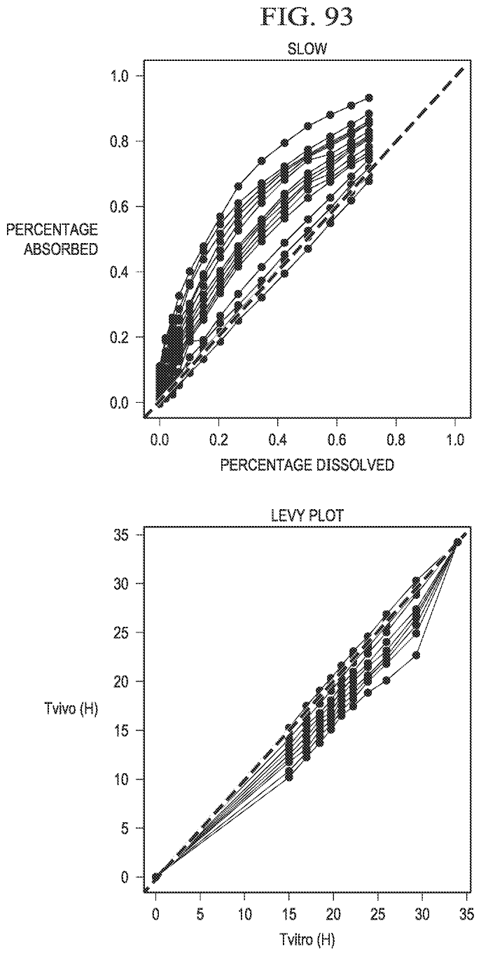

The plots on the fraction absorbed vs. fraction dissolved and Levy plots (Tvivo vs Tvitro)] for the Fast, Med, and Slow release formulations are presented in FIG. 91, FIG. 92, and FIG. 93, respectively.

For example, the Levy plots shown in FIG. 91, FIG. 92, and FIG. 93 provide further evidence of the non-linearity of colonic absorption. The Fast release formulation (which is not colonic absorbed) has an even distribution of points above and below the line of the Levy plot whereas for the Medium and Slow formulations which are more distally absorbed a majority of the points fall below the line.

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

What is claimed is:

1-18. (canceled)

19. A method of treating a condition in a subject having a disorder or condition responsive to the administration of methylphenidate, comprising:
orally administering to the subject an effective amount of a solid, oral pharmaceutical composition comprising:
methylphenidate or a pharmaceutical salt thereof,
wherein an in vivo absorption model of the solid, oral pharmaceutical composition has a function selected from the group consisting of:
(i) a single Weibull function:
$$r 1 ( t ) = {e}^{- ( {( \frac{time}{td} )}^{SS} )}$$
wherein td is a time necessary to absorb 63.2% of the methylphenidate or a pharmaceutical salt thereof released, and ss is a sigmoidicy factor;
(ii) a double Weibull function:
$$r 2 ( t ) = ff \cdot {e}^{- ( {( \frac{time}{td} )}^{SS} )} + ( 1 - ff ) \cdot {e}^{- ( {( \frac{time}{{td}_{1}} )}^{SS 2} )}$$
wherein ff is a fraction of a dose released in a 1st process, td is a time necessary to absorb 63.2% of the dose released in the 1st process, td1 is a time necessary to absorb 63.2% of a dose released in the 2nd process, ss is a sigmoidicy factor for the 1st process, and ss1 is a sigmoidicity factor for the 2nd process; and
(iii) a sigmoid eMax function:
$${r}_{v i t r o} ( t ) = \frac{t i m {e}^{g a}}{{EC}^{ga} + tim {e}^{g a}}$$
wherein EC is a time to release 50% of the methylphenidate or a pharmaceutical salt thereof, and ga is a parameter characterizing the shape of an absorption curve of the methylphenidate or a pharmaceutical salt thereof;
and
a correlation of a plurality of fractions of an in vitro dissolution of the solid, oral pharmaceutical composition with a same plurality of fractions of an in vivo absorption of the solid, oral pharmaceutical composition is non-linear;
thereby producing an improvement in a behavior or an ability related to the disorder or condition over a period of time,
wherein the administering reduces the variation in efficacy, or likelihood or severity of rebound, or both, of over the period of time.

20. The method of claim 19, wherein the period of time begins at 8:00 am, 9:00 am, 10:00 am, 11:00 am, 12:00 pm, 1:00 pm, 2:00 pm, 3:00 pm, 4:00 pm, 5:00 pm, 6:00 pm, or 7:00 pm.

21. The method of claim 19, wherein the period of time begins at 8, 9, 10, 11, 12, 13, 14, 15, or 16 hours after administration of the composition.

22. The method of claim 19, wherein the improvement is measured by a validated rating scale, score or combined score.

23. The method of claim 22, wherein the validated rating scale, score or combined score is a Swanson, Kotkin, Agler, M-Flynn and Pelham (SKAMP) score, or a SKAMP-CS combined score.

24. The method of claim 22, wherein the variation in efficacy is measured by a fluctuation index (FI):
$$FI = \frac{[ max ( C H P ) - min ( C H P ) ]}{average ( CHP )}$$
wherein CHP is a change from placebo of the SKAMP score during the period of time.

25. The method of claim 24, wherein the fluctuation index (FI) has an absolute value less than 1.0.

26. The method of claim 19, wherein the period of time ends at 9:00 am, 10:00 am, 11:00 am, 12:00 pm, 1:00 pm, 2:00 pm, 3:00 pm, 4:00 pm, 5:00 pm, 6:00 pm, 7:00 pm, or 8:00 pm.

27. The method of claim 19, wherein the period of time ends at 6, 7, 8, 9, 10, 11, 12, 13, 14, 15, or 16 hours after the start of the period of time.

28. The method of claim 19, wherein the period of time ends when a plasma concentration of methylphenidate in the subject is below 5 ng/mL.

29. The method of claim 19, wherein the period of time ends at 3, 4, 5, 6, 7, 8, 9, 10, 11, or 12 hours after a methylphenidate Tmax in the subject.

30. The method of claim 19, wherein the period of time ends when the subject falls asleep following Tmax.

31. The method of claim 23, wherein during the period of time, the value of the SKAMP scores do not change by more than, or than about, 6, 7, 8, 9, or 10.

32. The method of claim 19, wherein during the period of time, a rate of change of the methylphenidate plasma concentration over time is not greater than +2.5 ng·hr/mL following a dose of up to 100 mg methylphenidate.

33. The method of claim 19, wherein the period of time is between Tmax and 6 hours after Tmax, and the rate of change of the methylphenidate plasma concentration is not less than −1.2 ng·hr/mL following a dose of up to 100 mg methylphenidate.

34. The method of claim 19, wherein the period of time comprises a period wherein a methylphenidate plasma concentration is between Cmax and at least 40% Cmax and a rate of change of the methylphenidate plasma concentration is not greater than +1.5 ng·hr/mL and not less than −1.5 ng·hr/mL.

35. The method of claim 19, wherein the methylphenidate or pharmaceutical salt thereof is absorbed in the colon.

36. The method of claim 35, wherein at least 90% of the methylphenidate or pharmaceutical salt thereof is absorbed in the colon.

37. The method of claim 19, wherein the subject has attention deficit hyperactivity disorder (ADHD) or attention deficit hyperactivity disorder (ADD), and an autism spectrum disorder (ASD).

38. The method of claim 19, wherein the improvement is dose-dependent.

39. The method of claim 38, wherein the dose-dependent improvement comprises a dose-dependent increase in the period of time.

40. The method of claim 39, wherein the increase in the period of time comprises an increase in a period of time after Tmax.

41. The method of claim 19, wherein the solid, oral pharmaceutical composition is a multilayered solid, oral pharmaceutical composition comprising:
the methylphenidate or a pharmaceutical salt thereof;
a sustained release layer; and
a delayed release layer.

42. The method of claim 41, wherein the solid, oral pharmaceutical composition comprises:
a core comprising methylphenidate or a pharmaceutical salt thereof;
wherein
the core, the sustained release layer and the delayed release layer each have a surface; and
the sustained release layer and the delayed release layer enclose the core.

43. The method of claim 42, wherein the sustained release layer encloses the core and the delayed release layer encloses the sustained release layer.

44. The method of claim 42, wherein:
the sustained release layer and the delayed release layer incompletely encloses the core.

45. The method of claim 42, wherein:
the delayed release layer incompletely encloses the surface of the sustained release layer and/or the surface of the core.

46. The method of claim 44, wherein:
the sustained release layer and the delayed release layer enclose at least 90%, 91%, 92%, 93%, 94%, 95%, 96%, 97%, 98%, 99%, or 99.9% of the surface of the core.

47. The method of claim 45, wherein:
the delayed release layer encloses at least 90%, 91%, 92%, 93%, 94%, 95%, 96%, 97%, 98%, 99%, or 99.9% of the surface of the core and/or the surface of the sustained release layer.

48. The method of claim 19, wherein:
the multilayered solid, oral pharmaceutical composition comprises a multilayered core, wherein:
the multilayered core has a surface, and
the multilayered core comprises a first layer comprising the methylphenidate or a pharmaceutical salt thereof and a second layer comprising a swellable layer comprising a superdisintegrant or an osmotic agent.

49-54. (canceled)
